SEQUENCE LISTING (41 sequences)

SEQ ID NO: 1 - protein, 122 aa, Pseudomonas aeruginosa
MAWKGEVLANNEAGQVTSIIYNPGDVITIVAAGWASYGPTQKWGPQGDREHPDQGLICHDAFCGALVMKIGNSGTIPVNTGLFRWVAPNNVQGAITLIYNDVPGTYGNNSGSFSVNIGKDQS

SEQ ID NO: 2 - protein, 115 aa, Pseudomonas aeruginosa
MATQGVFTLPANTRFGVTAFANSSGTQTVNVLVNNETAATFSGQSTNNAVIGTQVLNSGSSGKVQVQVSVNGRPSDLVSAQVILTNELNFALVGSEDGTDNDYNDAVVVINWPLG

SEQ ID NO: 3 - protein, 4 aa, synthetic construct
  REGION 1..4: note = Linker variant 1
AGLA

SEQ ID NO: 4 - protein, 4 aa, synthetic construct
  REGION 1..4: note = Linker variant 2
FGLA

SEQ ID NO: 5 - protein, 4 aa, synthetic construct
  REGION 1..4: note = Linker variant 4
FGAK

SEQ ID NO: 6 - protein, 5 aa, synthetic construct
  REGION 1..5: note = Linker variant 4
LVLGA

SEQ ID NO: 7 - protein, 6 aa, synthetic construct
  REGION 1..6: note = Linker variant 5
LVLGGS

SEQ ID NO: 8 - protein, 6 aa, synthetic construct
  REGION 1..6: note = Linker variant 6
LLLGGS

SEQ ID NO: 9 - protein, 6 aa, synthetic construct
  REGION 1..6: note = Linker variant 7
VVLGGS

SEQ ID NO: 10 - protein, 4 aa, synthetic construct
  REGION 1..4: note = Linker variant 8
VLGS

SEQ ID NO: 11 - protein, 6 aa, synthetic construct
  REGION 1..6: note = Linker variant 9
LVFGGS

SEQ ID NO: 12 - protein, 6 aa, synthetic construct
  REGION 1..6: note = Linker variant 10
LVMTSG

SEQ ID NO: 13 - protein, 6 aa, synthetic construct
  REGION 1..6: note = Linker variant 11
RVRGHF

SEQ ID NO: 14 - protein, 4 aa, synthetic construct
  REGION 1..4: note = Linker variant 12
IAAG

SEQ ID NO: 15 - protein, 5 aa, synthetic construct
  REGION 1..5: note = Linker variant 13
LAFGA

SEQ ID NO: 16 - protein, 6 aa, synthetic construct
  REGION 1..6: note = Linker variant 14
IVFGGS

SEQ ID NO: 17 - protein, 6 aa, synthetic construct
  REGION 1..6: note = Linker variant 15
VVGGSG

SEQ ID NO: 18 - protein, 6 aa, synthetic construct
  REGION 1..6: note = Linker variant 16
VVAGGS

SEQ ID NO: 19 - protein, 4 aa, synthetic construct
  REGION 1..4: note = Linker variant 17
IFGA

SEQ ID NO: 20 - protein, 6 aa, synthetic construct
  REGION 1..6: note = Linker variant 18
ITYGAS

SEQ ID NO: 21 - protein, 6 aa, synthetic construct
  REGION 1..6: note = Linker variant 19
GSFGAR

SEQ ID NO: 22 - protein, 5 aa, synthetic construct
  REGION 1..5: note = Linker variant 20
LVFGA

SEQ ID NO: 23 - protein, 4 aa, synthetic construct
  REGION 1..4: note = Linker variant 21
IAKD

SEQ ID NO: 24 - protein, 4 aa, synthetic construct
  REGION 1..4: note = Linker variant 22
KGPA

SEQ ID NO: 25 - protein, 5 aa, synthetic construct
  REGION 1..5: note = Linker variant 23
PLGPR

SEQ ID NO: 26 - protein, 6 aa, synthetic construct
  REGION 1..6: note = Linker variant 24
AGPPGP

SEQ ID NO: 27 - protein, 9 aa, synthetic construct
  REGION 1..9: note = Linker variant 25
PRPPAPVFY

SEQ ID NO: 28 - protein, 7 aa, synthetic construct
  REGION 1..7: note = Linker variant 26
RRKKVYP

SEQ ID NO: 29 - protein, 5 aa, synthetic construct
  REGION 1..5: note = Linker variant 27
TPIQL

SEQ ID NO: 30 - protein, 4 aa, synthetic construct
  REGION 1..4: note = Linker variant 28
LPAL

SEQ ID NO: 31 - protein, 4 aa, synthetic construct
  REGION 1..4: note = Linker variant 29
LRIS

SEQ ID NO: 32 - protein, 4 aa, synthetic construct
  REGION 1..4: note = Linker variant 30
LKIS

SEQ ID NO: 33 - protein, 4 aa, synthetic construct
  REGION 1..4: note = Linker variant 31
LKLN

SEQ ID NO: 34 - protein, 4 aa, synthetic construct
  REGION 1..4: note = Linker variant 32
ARFT

SEQ ID NO: 35 - protein, 4 aa, synthetic construct
  REGION 1..4: note = Linker variant 33
LQLP

SEQ ID NO: 36 - protein, 4 aa, synthetic construct
  REGION 1..4: note = Linker variant 34
LGLP

SEQ ID NO: 37 - protein, 4 aa, synthetic construct
  REGION 1..4: note = Linker variant 35
LFGA

SEQ ID NO: 38 - protein, 4 aa, synthetic construct
  REGION 1..4: note = Linker variant 36
LYGA

SEQ ID NO: 39 - protein, 4 aa, synthetic construct
  REGION 1..4: note = Linker variant 37
IYGA

SEQ ID NO: 40 - protein, 18 aa, synthetic construct
LYIGLAGLATDVQTVAQR

SEQ ID NO: 41 - protein, 5 aa, synthetic construct
TVAGL

(19) United States
(12) Patent Application Publication
(10) Pub. No.: US 2025/0295790 A1
(43) Pub. Date: Sep. 25, 2025

(54) LECTIN-TARGETING CONJUGATES

(71) Applicant: Helmholtz-Zentrum für Infektionsforschung GmbH, Braunschweig (DE)

(72) Inventors: Alexander TITZ, Braunschweig (DE); Eva ZÁHORSKÁ, Braunschweig (DE); Joscha MEIERS, Braunschweig (DE); Lisa Marie DENIG, Braunschweig (DE); Stefanie WAGNER, Braunschweig (DE)

(21) Appl. No.: 18/717,387

(22) PCT Filed: Dec. 7, 2022
(86) PCT No.: PCT/EP2022/084864

§ 371 Date: Jun. 6, 2024

(30) Foreign Application Priority Data
Dec. 7, 2021 (EP) 21212989.4

(51) Int. Cl. A61K 47/54; A61K 47/65; A61P 31/04

(52) U.S. Cl. CPC A61K 47/549; A61K 47/65; A61P 31/04

(57) ABSTRACT

The present invention relates to a conjugate comprising a ligand specifically binding to a bacterial lectin, a linker comprising a peptide cleavable by a bacterial protease, and an anti-bacterial therapeutic agent or imaging agent. It further relates to the conjugate or the pharmaceutical composition or diagnostic composition for use in medicine.

### CROSS REFERENCE TO RELATED APPLICATIONS

This application is the US national phase under 35 U.S.C. § 371 of International Application No. PCT/EP2022/084864, filed Dec. 7, 2022, which claims the benefit of European Patent Application No. 21212989.4, filed Dec. 7, 2021, the contents of which are hereby incorporated in their entirety.

### SEQUENCE LISTING STATEMENT

A computer readable form of the Sequence Listing is filed with this application by electronic submission and is incorporated into this application by reference in its entirety. The Sequence Listing is contained in the file created on Jun. 6, 2024, having the file name “24-0761-WO-US.xml” and is 53,248 bytes in size.

### FIELD OF THE INVENTION

The present invention relates to a conjugate comprising a ligand specifically binding to a bacterial lectin, a linker comprising a peptide cleavable by a bacterial protease, and an anti-bacterial therapeutic agent or imaging agent. It further relates to the conjugate or the pharmaceutical composition or diagnostic composition for use in medicine.

### BACKGROUND OF THE INVENTION

Bacterial infections are still considered as a major health and economic problem due to the high morbidity and mortality rates, as well as the increased expenditure on patient management. Currently, the treatment of bacterial infections is facing a crisis since the current portfolio of antibiotics is impaired by the increasing numbers of multi-resistant pathogens and simultaneously limited efforts to discover new antibiotics (Balaban et al. 2019, Nat. Rev. Microbiol. 17, 441-448; Blaskovich et al. 2018, ACS Infect. Dis. 4, 868-870). According to a study conducted by the European Centre for Disease Prevention and Control (ECDC), it is estimated that about 33.000 people die each year in the European Union alone as a direct consequence of an infection due to bacteria resistant to antibiotics (Cassini et al. 2019, The Lancet, vol. 19, issue 1, 56-66).

The resistance occurs as a result of extensive misuse and overuse of antibiotics combined with factors such as agricultural use of antibiotics or easy travel routes that substantially contributed to the dissemination of antimicrobial resistance across the globe. In addition, antibiotic resistance has to be understood as an intrinsic part of bacterial evolution which may happen either via chromosomal mutation leading to viable mutants, or more commonly through an acquisition of resistance genes from other bacteria via horizontal gene transfer, by mobile plasmids, transposons or outer membrane vesicles (Dadgostar P. 2019, Infect Drug Resist., 12: 3903-3910; Klahn and Br6nstrup 2017, Nat. Prod. Rep, 34, 832).

The ongoing evolution results in bacteria which developed various protective mechanisms against the stagnating portfolio of antimicrobial therapeutics. These mechanisms are manifold and include, for example, biofilm formation, reduction of certain membrane proteins, changes in the composition of other membrane components, such as phospholipids or lipopolysaccharides (LPS) (Sommer et al. 2017, Nat Rev Microbiol. 15, 689-696).

Especially, gram-negative bacteria, such as Pseudomonas aeruginosa, Helicobacter pylori, Haemophilus influenzae, etc., are known for their intrinsic resistance to a wide range of antibiotics (Sommer et al. 2017, Nat Rev Microbiol. 15, 689-696; Ropponen et al. 2021, Advanced drug delivery reviews 172, 339-360). P. aeruginosa, for example, can form biofilms, that are described as complex hydrogels stabilized by extracellular polymeric substances like DNA, polysaccharides and a plethora of proteins (Fleming et al. 2010, Nat. Rev. Microbiol. 8, 623). These biofilms can lead to an additional barrier towards antibiotics (up to 1000-fold increase in resistance) and the host's immune system (Suci et al. 1994, Antimicrobial Agents and Chemotherapy, 38, 2125-2133).

To address the rising problem of antibiotic resistance, novel therapy modalities are under investigation, including the modification of existing drugs, design of pathoblockers, use of biological formats like antibodies or phages, or combination treatments. In particular, targeted drug-delivery systems represent promising approaches which are intended to facilitate the drug to reach its target site of action in appropriate quantity with specificity, thereby combining the interaction of two mechanisms: identifying and binding the target, and then providing the pharmacological response. Targeted-drug delivery allows the drugs to accumulate in the target organ or tissue selectively and quantitatively while preventing the drug from reaching nontarget organs and tissues. Thus, such systems provide potentially an efficacious and safe drug delivery (Yadav et al 2019, Basic Fundamentals of Drug Delivery, 269-305).

Currently, an antibody-antibiotic conjugate targeting S. aureus is under investigation. This conjugate combines a β-GlcNAc-WTA antibody, which binds specifically to bacterial β-GlcNAc residues of wall teichoic acid, with an ansamycin class antibiotic (rifampicin and dimethyl DNA31) by linkage via a MC-ValCit-PABQ linker (Mariathasan and Man-Wah Tan 2017, Trends in Molecular Medicine, Vol 23, no. 2).

However, it is well known that despite the best efforts to design linkers to be entirely stable in plasma, unanticipated chemical or enzymatic activity in vivo could lead to breakdown, chemical modification, or deconjugation of the drug conjugates (Lin and Tibbitts 2012, Pharmacetical Research 29, 2354-2366; Hamblett et al 2004, Clinical Cancer Research 10, 7063-7070). The release of the therapeutic cargo due to untimely degradation of the linker can lead to severe side effects, such as tendon ruptures, neuropathy or heart failures as for example in the case of fluoroquinolone antibiotics.

To address this problem, Meiers et al. 2020 developed lectin-targeted fluoroquinolone conjugates that are connected to lectin-probes via a non-cleavable linker. However, the antibiotic activity of the conjugated fluoroquinolone was strongly reduced compared to ciprofloxacin.

There is, thus, a need in the field for a system that provides safe delivery of a therapeutic to the site of infection and effective release of the therapeutic with its full antimicrobial efficacy.

Here, a lectin-targeted conjugate with a cleavable peptide-linker in a prodrug-like fashion has been created. The lectin-targeting ligand is specifically designed to bind to bacterial lectin and thus, to direct the conjugate to the site of infection. In addition, the lectin-targeting ligand is linked to a linker which is engineered to be cleaved by bacterial proteases, thereby releasing the therapeutic only in the presence of bacteria. The present invention provides conjugates that are less prone to hydrolysis then the conjugates of the prior art. These conjugates provide inter alia the following advantages: they have superior stability in a patient, they have improved targeting, they accumulate to higher concentrations at the site of disease, and they lead to a decrease in the systemic release of the drug attached to the targeting moiety.

### SUMMARY OF THE INVENTION

In a first aspect, the present invention relates to a conjugate having a structure according to formula (I)

R¹₍ₙ₎—Y—R² (I)

or a structure according to formula (II)

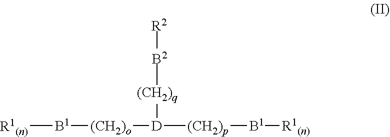

wherein
R¹ comprises one or more ligands specifically binding to a bacterial lectin;
Y is a linker comprising a peptide (Pep) cleavable by a bacterial protease;
R² is an anti-bacterial therapeutic agent in formula (I) or an anti-bacterial therapeutic agent or imaging agent in formula (II);
wherein n is between 1 to 10 and if n is 2 to 10 each R¹ can be the same or different
B¹ in each case is independently selected from a first bridging moiety;
B² is a second bridging moiety;
D is selected from the group consisting of amine, ammonium, phosphate, phosphine, phosphonate, tricarboxybenzoic acid, triaminomethyl benzene, citric acid, glycerol, trishydroxymethyl amine, lysine, cyclo oligolysine, 1,4,7,10-tetrazacyclododecan, 1,4,7-triazacyclononan, and 1,5,9-triazacyclododecan, and
o, p and q are independent of each other selected from 0 to 4.

In a second aspect, the present invention provides a pharmaceutical composition or diagnostic composition comprising the conjugate according to the first aspect, and optionally comprises one or more constituents selected from the group consisting of a pharmaceutically acceptable carrier, a diluent and an excipient.

In a third aspect, the present invention provides a conjugate according to the first aspect or a pharmaceutical composition according to the second aspect for use in medicine.

In a forth aspect, the present invention provides a conjugate according to the first aspect or a pharmaceutical or diagnostic composition according to the second aspect for use in treating or preventing or diagnosing a disease or infection associated with a bacterium of the phylum Firmicutes, preferably of the class of Bacilli or Clostridia; the phylum Actinobacteria, preferably of the order Corynebacteriales; or the phylum Proteobacteria, preferably of the class of Alphaproteobacteria, Betaproteobacteria or Gammaproteobacteria.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolbl, H, eds. (1995), Helvetica Chimica Acta, CH-4010 Basel, Switzerland).

Several documents (including all patents, patent applications, scientific publications, manufacturer's specifications, instructions etc.) are cited throughout the text of this specification. Nothing herein is to be construed as an admission that the invention is not entitled to antedate such disclosure by virtue of prior invention.

### Definitions

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise

The term “conjugate” may be used interchangeably and refer within the present invention to at least two compounds that have different functionalities and which are covalently linked with each other via a permanent or a labile linker. Typically, one of the two compounds is a small molecule drug or another therapeutic agent or an imaging agent that is covalently linked to the second substance which may be a natural or synthetic molecule which mediates specific binding to a bacterial lectin.

As such, the term “targeted drug delivery conjugate” refers to a preferred example of a conjugate of the present invention wherein the conjugate comprises a drug that is delivered to a subject and targeted to the area of infection, which results in an increased concentration of the drug in that particular region of the body when compared to other regions of the body of that subject.

The term “imaging conjugate” refers to a preferred example of a conjugate of the present invention comprising an imaging agent, such as a radioisotope or label.

The terms “ligand”, “targeting ligand” or “targeting moiety” can be used interchangeably and refer in the context of the specification to any molecule that provides an enhanced affinity for a selected target, e.g. a protein, a cell, cell type, tissue, organ, region of the body, or a compartment, e.g. a cellular, tissue or organ compartment. As used in the present specification, the term “ligand” refers to a molecule with an enhanced affinity for proteins or receptors, preferably glycoproteins or carbohydrates such as animal, plant or bacterial lectins. Preferably, the ligand is a saccharide, more preferably a mono-, di- or trisaccharide. Most preferably, the ligand is a monosaccharide selected from the group consisting of galactose, fucose, mannose, xylose or a derivative thereof.

The term “derivative” according to the present specification refers to a chemical substance derived from another substance either directly or by modification or partial substitution without significantly effecting the activity, e.g. the ability to bind to a bacterial lectin.

The term “bacterial lectin” refers in the context of the present invention to a bacterial protein that preferentially recognizes and binds to carbohydrate complexes protruding from glycolipids and glycoproteins. Apart from animals and plants, many viruses, fungi, protozoa can virtually all bacterial species and genera express lectins. Many gram-negative bacteria, e.g. Escherichia coli and Pseudomonas aeruginosa, and a few gram-positive ones, e.g. certain actinomyces, produce surface lectins that are occur commonly in the form of elongated, multisubunit protein appendages, also known as fimbriae (hairs) or pili (threads), which interact with glycoprotein and glycolipid receptors on host cells. The primary function of bacterial lectin is to facilitate the attachment or adherence of bacteria to host cells, a prerequisite for bacterial colonization and infection. Thus, bacterial lectins are often called adhesins and bind corresponding glycan receptors on the surface of the host cells via carbohydrate-recognition domains. Accordingly, the terms “lectin” and “adhesins” are used interchangeably throughout this specification.

Lectins are classified primarily into five specificity groups, according to the monosaccharide for which they exhibit the highest affinity: mannose, galactose/N-acetylgalactosamine, N-acetylglucosamine, fucose and N-acetylneuraminic acid. Their carbohydrate binding capacity is attributed to a typically globular domain termed the “carbohydrate recognition domain” (CRD), which is defined by a conserved group of residues that determine its conformation and function. The CRDs tend to be shallow indentations, grooves or pockets with lower affinities that are usually of the millimolar order located at the lectin surface. The lectins according to the present invention can be divided into the following four groups depending on the specific carbohydrates they recognize: i) mannose-specific lectins (type 1 fimbriae), expressed for example by most Enterobacteriae, such as Klebsiella pneumoniae; ii) sialic-acid-specific lectins, expressed for example by Escherichia coli or Helicobacter pylori; iii) Gal- and GalNAc-specific lectins, expressed for example by Pseudomonas aeruginosa or Myxobacteria; and iv) fucose-specific lectins, expressed for example by Vibrio cholerae or Pseudomonas aeruginosa.

The term “binding” as used in the context of the present invention means the formation of non-covalent bonds between two molecules.

The term “specific binding” as used in the context of the present invention means that a compound binds stronger to a target for which it is specific compared to the binding to another target. In the context of the present specification, “specifically binding” means that a lectin binder, preferably a saccharide binds to a carbohydrate recognition domain on a bacterial lectin. In the context of the present invention a ligand specifically binds to a lectin, if it exhibits a binding affinity (K_{D}) that is lower than 100 M, preferably lower than 50 M and more preferably lower than 10 M. The binding affinity can be measured by any art known method but is preferably measured by competitive binding assay based on fluorescence polarisation or surface plasmon resonance (SPR) or isothermal calorimetry (ITC) as described in the examples.

The term “linker” refers in the context of the present invention to a chemical moiety that is capable of covalently attaching or linking a compound, usually a drug, such as an antibiotic, to a lectin-binding molecule. The linkers used in the context of the present invention comprise a peptide that is cleavable by a bacterial protease. Thus, in a preferred embodiment of the invention, the linker comprises or consists of a stretch of amino acids that is recognized and cleaved by a protease released by a bacterium. Preferably, the core structure of a peptide linker comprises either a di-, tri or a tetra-peptide that is recognized and cleaved by proteases. Thus, when cleavage of the linker is induced, e.g. by the presence of a bacterial proteases due to infection, the therapeutic agent is released and takes effect.

The terms “peptide” as used in the context of the present invention refers to at least two amino acids linked by peptide bonds. Thus, the term “polypeptide” is also used to refer to amino acid chains with more than 50, more than 100 or more than 150 amino acids.

The term “bacterial protease” as used in the context of the present invention refers to a degradative enzyme of bacterial origin which hydrolyses the peptide bond present in a peptide. Proteases can be classified into groups based on their acidic or basic properties, presence of functional groups and the position of the peptide bond. They have a number of key roles in bacterial physiology and biochemistry, as well as in pathogenicity, and are also essential to the ability of many bacteria to infect the host and cause disease. For example, Pseudomonas aeruginosa produces and secretes a number of proteases such as elastase A (LasA), elastase B (LasB), protease IV, and alkaline protease, which are known to facilitate bacterial colonization and actively subverting immune responses.

The lectins and proteases can be produced by various bacterial phyla, such as Firmicutes, Actinobacteria or Proteobacteria. The term “phylum” refers to taxonomic ranking that comes third in the hierarchy of classification. Bacteria, including the archaea, are grouped into roughly 34 phyla. Organisms in a phylum share a set of characteristics that distinguishes them from organisms in another phylum.

The terms “therapeutic agent”, “drug” and “agent” are used interchangeably herein and refer in the context of the present invention to a compound that has a therapeutic effect, e.g. to any substance used in the diagnosis, treatment or prevention of a disease. Preferably, the therapeutic agent as it is referred to in the context of the present invention is an antibacterial agent, i.e. a molecule that selectively destroys bacteria by interfering with bacterial growth or survival. Typical examples of antibacterial agents include but are not limited to functional nucleic acids, e.g. antisense antimicrobial therapeutic agents, aptamers, or topoisomerase inhibitors; antimicrobial peptides; chitosan, e.g. chitosan derivatives (e.g. quaternized derivatives, sulfonated derivatives), chitosan nanoparticle complexes (e.g. chitosan-Ag complex, chitosan-ZnO complex), and antibiotics (Zhou et al. 2020). Preferably, the therapeutic agent in the context of the present invention is an antibiotic.

The term “amino acid” as used in the context of the present invention refers to one of the 20 naturally occurring amino acids or any non-natural analogues that may be present at a specific, defined position.

The terms “sequence identity” or “sequence homology” as referred to in the present specification are interchangeable and are used with regard to polypeptide and nucleotide sequence comparisons. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID NO, if not specifically indicated otherwise. For example, a polypeptide sequence consisting of 200 amino acids compared to a reference 300 amino acid long polypeptide sequence may exhibit a maximum percentage of sequence identity of 66.6% (200/300) while a sequence with a length of 150 amino acids may exhibit a maximum percentage of sequence identity of 50% (150/300). If 15 out of those 150 amino acids are different from the respective amino acids of the 300 amino acid long reference sequence, the level of sequence identity decreases to 45%. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/or http://www.ebi.ac.uk/Tools/clustalw2/index.html. The grade of sequence identity (sequence matching) may be calculated using e.g. BLAST, BLAT or BlastZ (or BlastX). BLAST protein searches are performed with the BLASTP program, score=50, word length=3. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl. 1: I54-I62) or Markov random fields. Structure based alignments for multiple protein sequences and/or structures using information from sequence database searches, available homologs with 3D structures and user-defined constraints may also be used (Pei J, Grishin NV: PROMALS: towards accurate multiple sequence alignments of distantly related proteins. Bioinformatics 2007, 23:802-808; 3DCoffee@igs: a web server for combining sequences and structures into a multiple sequence alignment. Poirot 0, Suhre K, Abergel C, O'Toole E, Notredame C. Nucleic Acids Res. 2004 Jul. 1; 32: W37-40.). When percentages of sequence identity are referred to in the present application, these percentages are calculated in relation to the full length of the longer sequence, if not specifically indicated otherwise.

The definitions presented below apply in an analogous manner to radicals having two bonds instead of only one bond to another moiety.

The term “carbocyclic group” as used in the context of the present invention refers to a saturated or unsaturated cyclic radical in which all of the ring members are carbon atoms. Carbocyclic groups are monocyclic, or are fused, spiro, or bridged ring systems with two, three or four cycles. Monocyclic carbocyclic groups contain 3 to 10 carbon atoms, preferably 4 to 7 atoms, and more preferably 5 to 6 carbon atoms in the ring. Preferred examples are C₃ to C₁₀ cycloalkyl, in particular cyclopentyl, cyclohexyl, and cycloheptyl, C₃ to C₁₀ cycloalkenyl, in particular cyclopentenyl, cyclohexenyl, and cycloheptenyl and phenyl. Bicyclic carbocyclic groups contain 8 to 12 carbon atoms, preferably 9 to 10 carbon atoms in the ring. Carbocyclic groups may be substituted or unsubstituted. The “phenylene group” is a preferred carboxylic group in the context of the present invention and refers to a di-substituted benzene ring. Examples of compounds with a phenylene group as a structural motif are ortho-, meta- and para-xylene, ortho-, meta- and para-phenylenediamine, ortho-, meta- and para-hydroxybenzoic acid as well as phthalic acid and phthalic anhydride. The “naphthalenediyl group” is a preferred carboxylic group in the context of the present invention and refers to a bivalent aromatic hydrocarbon comprising two fused benzene rings.

The term “heterocarbocyclic group” as used in the context of the present invention refers to a monovalent saturated or unsaturated hydrocarbon radical, wherein at least one of the carbon atoms is replaced by 1, 2, 3 or 4 (for the five-membered rings) or 1, 2, 3, 4, or 5 (for the six-membered ring) of the same or different heteroatoms, preferably selected from O, N and S. Heterocarbocyclic groups are monocyclic, or are fused, spiro, or bridged bicyclic ring systems. Monocyclic heterocarbocyclic groups contain 3 to 10 carbon atoms, preferably 4 to 7 carbon atoms, and more preferably 5 to 6 carbon atoms in the ring. Bicyclic heterocarbocyclic groups contain 8 to 12 carbon atoms, preferably 9 to 10 carbon atoms in the ring. Heterocarbocyclic groups may be substituted or unsubstituted. Suitable substituents include, but are not limited to, lower alkyl, hydroxyl, nitrile, halogen and amino. Substituents may also be themselves substituted. Examples of heterocarbocyclic groups include furanyl, thiophenyl, oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiphenyl, 2-benzothiphenyl, 1H-indazolyl, benzimidazolyl, benzoxazolyl, indoxazinyl, 2,1-benzoxazoyl, benzothiazolyl, 1,2-benziosthiazolyl, 2,1-benzisiothiazolyl, quinolinyl, isoquinolinyl, 2,3-benzodiazinyl, quinoxalinyl, quinazolinyl, quinolinyl, 1,2,3-benzotriazinyl, or 1,2,4-benzotriazinyl.

The term “alkyl” refers in the context of the present specification to a saturated straight or branched carbon chain radical. Preferably, the chain comprises from 1 to 10 carbon atoms, i.e. 1, 2, 3, 4, 5, 6, 7, 8, 9 or 10, e.g. methyl, ethyl propyl (n-propyl or iso-propyl), butyl (n-butyl, iso-butyl, secbutyl, tert-butyl), pentyl, hexyl, heptyl, octyl, nonyl, decyl. Alkyl groups are optionally substituted. Thus, the term “C₁-C₄ alkyl group” means a straight or branched alkyl group having 1 to 4 carbon atoms.

The term “alkoxy” according to the present specification refers to an alkyl radical that is singularly bonded to oxygen. The term “C₁-C₄ alkoxy group” as referred to according to the specification, means a straight or branched alkoxy radical having 1 to 4 carbon atoms.

The term “haloalkyl” refers in the context of the present invention to a saturated straight or branched carbon chain radical in which one or more hydrogen atoms are replaced by halogen atoms, e.g. by fluorine, chlorine, bromine or iodine. Preferably, the chain comprises from 1 to 10 carbon atoms, i.e. 1, 2, 3, 4, 5, 6, 7, 8, 9 or 10. In particular, “haloalkyl” refers to —CH₂F, —CHF₂, —CF₃, —C₂H₄F, —C₂H₃F₂, —C₂H₂F₃, —C₂HF₄, —C₂F₅, —C₃H₆F, —C₃H₅F₂, —C₃H₄F₃, —C₃H₃F₄, —C₃H₂F₅, —C₃HF₆, —C₃F,, —CH₂Cl, —CHCl2, —CCl₃, —C₂H₄Cl, —C₂H₃Cl₂, —C₂H₂Cl₃, —C₂HCl₄, —C₂Cl₅, —C₃H₆Cl, —C₃H₅Cl₂, —C₃H₄Cl₃, —C₃H₃Cl₄, —C₃H₂Cl₅, —C₃HCl₆, and —C₃Cl7. Haloalkyl groups are optionally substituted.

If two or more radicals can be selected independently from each other, then the term “independently” means that the radicals may be the same or may be different.

The term “pharmaceutical composition” as used herein refers to the combination of an active agent with a pharmaceutically acceptable carrier, inert or active, a diluent, and an excipient, making the composition suitable for therapeutic use. In addition, pharmaceutical compositions comprising the conjugate of the present invention can be formulated for oral, parenteral, topical, inhalative, rectal, sublingual, transdermal, subcutaneous or vaginal application routes according to their chemical and physical properties. Pharmaceutical compositions comprise solid, semisolid, liquid, or transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the conjugates of the present invention. Semisolid compositions that can be used in the context of the invention comprise emulsion, suspension, creams, lotions, gels, globules, buccal tablets and suppositories. The composition, if desired, can also contain minor amounts of wetting or emulsifying agents, or pH buffering agents.

These compositions can take the form of solutions, suspensions, emulsions, tablets, pills, capsules, powders, sustained-release formulations and the like. The composition can be formulated as a suppository, with traditional binders and carriers such as triglycerides.

The term “pharmaceutically” or “pharmaceutically acceptable” refers in the context of the present invention to molecular entities and compositions that do not lead to an adverse, allergic or other unwanted reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The term “carrier”, in the context of the present invention refers to a diluent, adjuvant, excipient, or vehicle with which the therapeutic agent is administered. Such pharmaceutical carriers can be liquid or solid. Liquid carriers include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

A “pharmaceutically acceptable carrier” in the context of the present invention may also be referred to as “pharmaceutically acceptable diluent” or “pharmaceutically acceptable vehicles” and may include solvents, bulking agents, stabilizing agents, dispersion media, coatings, antibacterial and antifungal agents, isotonic and absorption delaying agents, and the like which are physiologically compatible.

The term “excipient” in the context of the present invention refers to any substance other than the active substance, present in a medicinal product or used in the manufacture of the product. Excipients function as a carrier of the active substance and contribute to product attributes such as stability, biopharmaceutical profile, appearance and patient acceptability. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like.

As used herein, “teat”, “treating” or “treatment” of a disease or disorder means accomplishing one or more of the following: (a) reducing the severity of the disorder, (b) limiting or preventing development of symptoms characteristic of the disorder(s) being treated, (c) inhibiting worsening of symptoms characteristic of the disorder(s) being treated, (d) limiting or preventing recurrence of the disorder(s) in patients that have previously had the disorder(s); and (e) limiting or preventing recurrence of symptoms in patients that were previously symptomatic for the disorder(s).

As referred to in the present specification, the terms “prevent”, “preventing” or “prevention” of a disease or disorder mean preventing that a disorder occurs in a subject for a certain amount of time. For example, if a compound described herein is administered to a subject with the aim of preventing a disease or disorder, said disease or disorder is prevented from occurring at least on the day of administration and preferably also on one or more days (e.g. on 1 to 30 days; or on 2 to 28 days, or on 3 to 21 days; or on 4 to 14 days; or on 5 to 10 days) following the day of administration.

As referred to in the present invention, the terms “diagnose”, “diagnosing” or diagnosis” refers to the act of identifying a disease or illness by examining something or someone.

### Embodiments

In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In the work leading to the present invention, it was surprisingly found that conjugates comprising a ligand targeting bacterial lectin which is connected via a cleavable linker to a drug show a particularly good bioavailability and drug activity at the target site of bacterial infection compared to similar conjugates containing a non-cleavable linker (Meiers et al. 2020). In addition, the linker is designed to only be cleaved in the presence of a bacterial protease. The highly specific cleavage of the linker combined with an overall improved metabolic stability of the conjugates is highly beneficial since untimely or unspecific release of the drug cargo is prevented, thereby reducing off-target effects of the antibiotic.

The conjugate is further designed to specifically bind to bacterial lectin which assures the delivery of the conjugate to the site of infection, thereby also increasing the drug concentration at the site of infection.

Based on these results, the present invention provides in a first aspect a conjugate having a structure according to formula (I)

R¹₍ₙ₎—Y—R² (I)
or a structure according to formula (II)

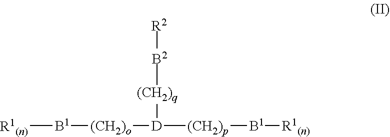

wherein
R¹ comprises one or more ligands specifically binding to a bacterial lectin;
Y is a linker comprising a peptide (Pep) cleavable by a bacterial protease;
R² is an anti-bacterial therapeutic agent in formula (I) or an anti-bacterial therapeutic agent or imaging agent in formula (II);
wherein n is between 1 to 10 and if n is 2 to 10 each R¹ can be the same or different
B¹ in each case is independently selected from a first bridging moiety;
B² is a second bridging moiety;
D is selected from the group consisting of amine, ammonium, phosphate, phosphine, phosphonate, tricarboxybenzoic acid, triaminomethyl benzene, citric acid, glycerol, trishydroxymethyl amine, lysine, cyclo oligolysine, 1,4,7,10-tetrazacyclododecan, 1,4,7-triazacyclononan, and 1,5,9-triazacyclododecan, and
o, p and q are independent of each other selected from 0 to 4.

In a preferred embodiment o and p are identical and selected from 0, 1, 2, 3, or 4, preferably o and p are 1.

In another preferred embodiment q is selected from 0, 1, 2, 3, or 4, preferably q is 1. In a more preferred embodiment, q is 2.

In yet another preferred embodiment, D is amine.

It is preferred that the conjugate of the present invention has a plasma half-life of 100 minutes or more, preferably 150 or more, or more preferably 200 minutes or more, if measured by incubating the conjugate with plasma and quantifying the remaining conjugate by LC-MS/MS quantification.

R¹ may comprise one ligand that specifically binds to a bacterial lectin or more. Preferably, one R¹ comprises two or three ligands that specifically bind to a bacterial lectin. Alternatively or additionally, the conjugate of the present invention can comprise between 1 to 10, preferably 2 to 10, i.e. 2, 3, 4, 5, 6, 7, 8, 9 or 10 R¹. Thus, the conjugates of the present invention preferably comprise between 1 to 30 ligand that specifically binds to a bacterial lectin. The ability of a given ligand to specifically bind to a bacterial lectin can be assessed as defined above or using the binding assays described in the appended examples. The binding affinity (K_{D}) of a typical ligand that specifically binds to a bacterial lectin is below 100 μM, more preferably below 50 μM and even more preferably below 10 μM. It will be appreciated by the person of skill in the art, that conjugates including two or more ligands that specifically binds to a bacterial lectin with these affinities will impart a lower K_{D} to the conjugate due to avidity effects. To measure such effects, it is no longer feasible to test the individual ligand that specifically binds to a bacterial lectin but to test the binding of the entire conjugate of the invention. It is preferred that the conjugate of the present invention by including two or more ligands that specifically binds to a bacterial lectin exhibits a binding affinity to lectin with a K_{D} value of 500 nM or less, preferably 400 nM or less, more preferably 300 nM or less, more preferably 200 nM or less, more preferably 100 nM or less, even more preferably 90 nM or less, even more preferably 80 nM or less, even more preferably 70 nM or less, even more preferably 60 nM or less, even more preferably 50 nM or less, even more preferably 40 nm or less, even more preferably 30 nM or less, even more preferably 20 nM or less, and most preferably 10 nM or less, if measured by competitive binding assay based on fluorescence polarisation or surface plasmon resonance (SPR) or isothermal titration calorimetry (ITC) as described in the examples.

The first and the second bridging moiety (B¹ and B², respectively) serve the purpose of providing sterical distance between the part of the conjugate binding to bacterial lectin, i.e. R¹ and the anti-bacterial therapeutic agent in formula (I) or the anti-bacterial therapeutic agent or imaging agent in formula (II) to avoid any interference with, e.g. the protease cleaving the Pep comprised in Y. Preferably, B² is hydrophilic.

The rational underlying the conjugates of the present invention, in particular according to Formula (I) is applicable to a wide range of bacteria since the majority of bacteria produce lectins and secrete proteases. According to one embodiment, the bacterial lectin and the bacterial protease is of a bacterium of the phylum: a) Firmicutes, b) Actinobacteria, or c) Proteobacteria. According to a preferred embodiment, the bacterium of the phylum Firmicutes is of the class of Bacilli or Clostridia. According to another preferred embodiment, the bacterium of the phylum Actinobacteria is of the order Corynebacteriales. According to a more preferred embodiment, the bacterium of the phylum Proteobacteria is of the class of Alphaproteobacteria, Betaproteobacteria or Gammaproteobacteria. According to a most preferred embodiment, the bacterium is of the class of Gammaproteobacteria.

It is understood by the skilled person that the ligand that specifically binds to a bacterial lectin and the Pep within Y are selected to match the respective bacterial species to be treated or diagnosed. In this case the ligand that specifically binds to a bacterial lectin will, e.g. bind to lectin A (lecA) and/or lectin B (lecB) of Pseudomonas aeruginosa and will also comprise a Pep cleaved by a protease released by Pseudomonas aeruginosa. Accordingly, depending on the bacterial infection to be treated or diagnosed the skilled person can readily select suitable ligands that specifically bind to a bacterial lectin produced by that bacterium as well as select a Pep that comprises an amino acid sequence cleaved by a protease released by that species. To be able to target multiple species within an order, preferably family and, thus the ligand that specifically binds to a bacterial lectin is selected to bind to lectins produced by two or more species within the order, preferably family and the Pep is selected to be cleavable by proteases secreted by two or more species within the order, preferably family.

According to one embodiment, the Bacilli are of the order Bacillales or Lactobacillales. According to a preferred embodiment, the Bacilli are of the family Streptococcaceae. According to another embodiment, the Clostridia are of the order Clostridiales. According to a preferred embodiment, the Clostridia are of the family Clostridiaceae. According to a further embodiment, the Corynebacteriales is of the family Mycobacteriaceae. According to a preferred embodiment, the Corynebacteriales are of the genus Mycobacterium. According to another embodiment, the Alphaproteobacteria are of the order Hyphomicrobiale. According to a preferred embodiment, the Alphaproteobacteria are of the family Phyllobacteriaceae or Brucellaceae. According to a further embodiment, the Betaproteobacteria is of the order Burkholderiales. In a preferred embodiment, the Betaproteobacteria are of the family Oxalobacteraceae, Burkholderiaceae, or Comamonadaceae. According to another embodiment, the Betaproteobacteria is of the order Neisseriales. According to a preferred embodiment, the Betaprotebacteria is of the family Neisseriaceae. According to one embodiment, the Gammaproteobacteria are of the order Pseudomonadales. According to a preferred embodiment, the Gammaproteobacteria are of the family Moraxellaceae. According to a further embodiment, the Gammaprotebacteria are of the order Enterobacterales. According to a preferred embodiment, the Gammaproteobacteria are of the family Enterobacteriaceae or Morganellaceae. According to yet another embodiment, the Gammaproteobacteria are of the order Oceanospirillales. According to a preferred embodiment, the Gammaproteobacteria are of the family Halomonadaceae. According to a further embodiment, the Gammaproteobacteria are of the order Pasteurellales. According to a preferred embodiment, the Gammaproteobacteria are of the family Pasteurellaceae. According to a further embodiment, the Gammaproteobacteria are of the order of Vibrionales. According to a preferred embodiment, the Gammaproteobacteria are of the family Vibrionaceae. According to yet another embodiment, the Gammaprotebacteria are of the order of Pseudomonadales. According to a more preferred embodiment, the Gammaproteobacteria are of the family Pseudomonadaceae.

According to an even more preferred embodiment, the bacterial lectin and the bacterial protease is of the bacterium Pseudomonas aeruginosa. According to an even more preferred embodiment, the protease is selected from the group consisting of LasA or LasB. According to the most preferred embodiment, the protease is LasB.

According to a further embodiment, the bacterial lectin bound by the ligand comprised in R¹ is:
(i) a galactose-binding lectin, or a homologue of LecA of Pseudomonas aeruginosa that has a sequence identity to the amino acid sequence according to SEQ ID NO: 1 of at least 60%, more preferably of at least 70%, more preferably of at least 80%, more preferably of at least 90% and even more preferably of at least 95%; and/or
(ii) a mannose- or fucose-binding lectin, or a homologue of LecB of Pseudomonas aeruginosa that has a sequence identity to the amino acid sequence according to SEQ ID NO: 2 of at least 60%, more preferably of at least 70%, more preferably of at least 80%, more preferably of at least 90% and even more preferably of at least 95%

According to a more preferred embodiment the galactose-binding lectin of a bacterium is selected from the genus Chromobacterium, Collimonas sp., Enterobacter sp., Klebsiella, Jejubacter, Mesorhizobium sp., Mycobacterium, Ochrobactrum sp., Photorhabdus sp., and Xenorhabdus sp. According to the most preferred embodiment, the galactose-binding lectin is of a bacterium of the Pseudomonas sp.

According to a more preferred embodiment, the mannose or fucose-binding lectin of a bacterium is selected from the genus Burkholderia sp., Klebsiella, Chromobacterium, Citrobacter, Diaphorobacter sp. Paraburkholderida, Ralstonia, Vibrio, Halomonas, and Listeria. In the most preferred embodiment, the mannose- or fucose-binding lectin is of a bacterium of the Pseudomonas sp.

In yet another embodiment, the ligand comprises or consists, preferably consists of one or more saccharide(s), wherein the —O-forming the glycosidic linkage between R¹ and Y in formula (I) or R¹ and B¹ in formula (II) may in each case may be replaced independently of each other by Z, wherein

Z is [saccharide]-S—, [saccharide]-CH₂—NH—SO₂—, [saccharide]-CH₂—NH—CO—, [saccharide]-NH—SO₂—, [saccharide]-NH—CO—, [saccharide]-NH— group, or [saccharide]-CH₂—. According to a preferred embodiment, Z is [saccharide]-S— or [saccharide]-NH—SO₂—. The replacement of the glycosidic bond with one of the groups indicated for Z leads to a bond that is less susceptible to hydrolysis. Preferably, all Z within a conjugate of the present invention are identical to impart similar stability to all bonds between R¹ and Y in formula (I) or R¹ and B¹ in formula (II).

In yet another more preferred embodiment, R¹ consists of a saccharide, wherein the —O-forming the glycosidic linkage between R¹ and Y in formula (I) or R¹ and B¹ in formula (II) may in each case may be replaced independently of each other by Z, wherein

Z is [saccharide]-S—, [saccharide]-CH₂—NH—SO₂—, [saccharide]-CH₂—NH—CO—, [saccharide]-NH—SO₂—, [saccharide]-NH—CO—, [saccharide]-NH— group, or [saccharide]-CH₂—. According to a preferred embodiment, Z is [saccharide]-S— or [saccharide]-NH—SO₂—.

According to an alternative embodiment, the saccharide is a di- or trisaccharide or a dendrimer. This is another possibility to allow the ligand specifically binding to the bacterial lectin to form multiple non-covalent interactions and thus to increase the binding affinity. In a further embodiment of the present invention the ligand specifically binding to the bacterial lectin has a dendrimeric structure, i.e. is a dendrimer comprising depending on whether the branch points are bidentate or tridentate 2″ or 3″ saccharides, wherein n is preferably between 2 to 5.

According to a preferred embodiment, the saccharide is monosaccharide. A particularly preferred monosaccharide is selected from the group consisting of galactose, fucose, mannose, xylose or a derivative thereof. In this context derivative refers to molecules which maintain the stereochemistry as well as the ring size of the galactose, fucose, mannose, xylose but in which the free hydroxygroups are either substituted with other groups or the hydrogen of the hydroxygroup is replaced by another group.

A preferred galactose derivative is selected from the group consisting of N-acetylgalactosamine (GalNAc), 2-deoxy-galactose, and epoxides of galactoheptose and esters, preferably acetates, wherein the —O— group forming the glycosidic linkage between R¹ and Y in formula (I) or R¹ and B¹ in formula (II) may be replaced by Z as defined above. According to a further preferred embodiment, the mannose derivative is selected from the group consisting of 6-deoxymannose, 6-deoxy-6-sulfonamido mannose, or N-acetylmannosamine (ManNAc), wherein the —O— group forming the glycosidic linkage between R¹ and Y in formula (I) or R¹ and B¹ in formula (11) may be replaced by Z as defined above. According to yet another preferred embodiment, the fucose derivative is selected from the group consisting of N-acetyl-L-fucosamine (FucNAc), 2-deoxy-L-fucose, and 6-hydroxy-L-fucose, wherein the —O— group forming the glycosidic linkage between R¹ and Y in formula (I) or R¹ and B¹ in formula (II) may be replaced by Z as defined above.

According to a more preferred embodiment, the monosaccharide is galactose, wherein the —O— group forming the glycosidic linkage between R¹ and Y in formula (I) is replaced by —S—. According to a further preferred embodiment, the monosaccharide is fucose, wherein the —O— group forming the glycosidic linkage between R¹ and Y in formula (I) is replaced by —NH—SO₂—.

According to another embodiment, R¹ comprises or consists, preferably consists of the formulae (III) to (XV): or each R¹ independent from each other comprises or consists, preferably consists of a monosaccharide selected from the group consisting of the formulae (III) to (XV):

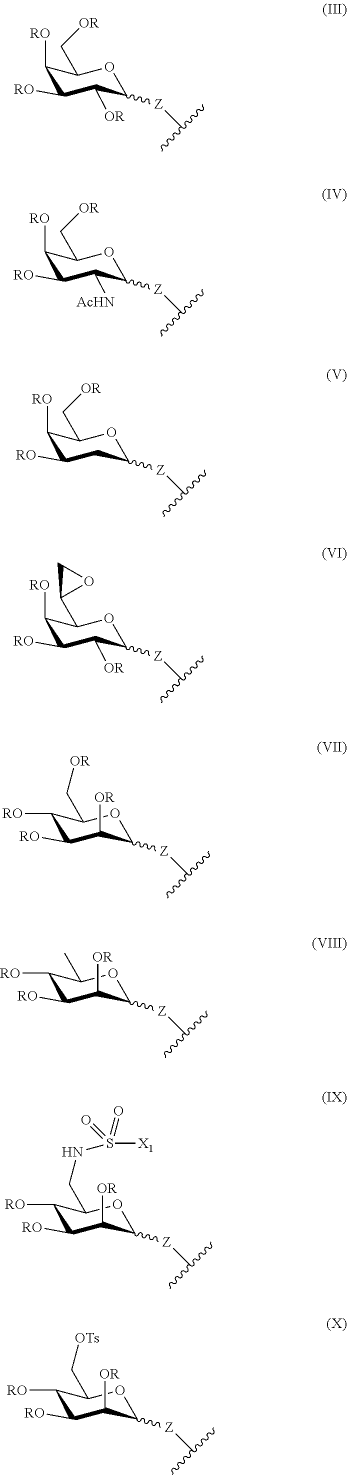

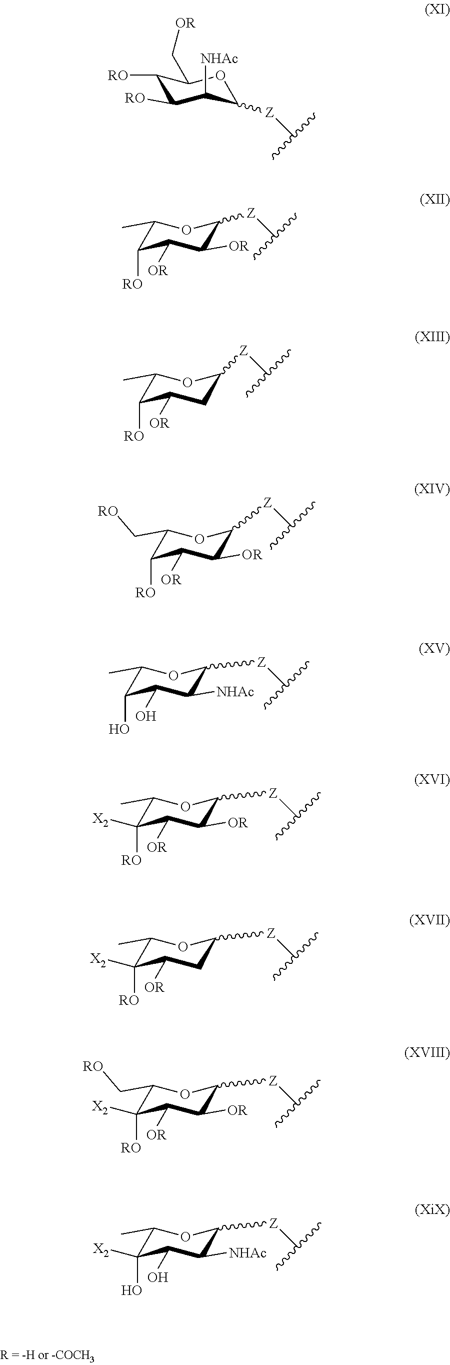

wherein;
Z is selected from the group consisting of —O—, —S—, [saccharide]-CH₂—NH—SO₂—, [saccharide]—CH₂—NH—CO—, [saccharide]-NH—SO₂—, [saccharide]-NH—CO—, [saccharide]-NH— group, or [saccharide]—CH₂—. According to a preferred embodiment, Z is —S—, in particular if the saccharide is galactose or a galactose derivate according to formulae (III) to (XI); or Z is [saccharide]—CH₂—NH—SO₂—, [saccharide]—CH₂—NH—CO— if the saccharide is fucose or a fucose derivative according to formulae (XII) to (XIX); and
X¹ and X² are independently selected from alkyl, aryl, alkylaryl, heteroaryl, or alkylheteroaryl; optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR⁴R⁵ wherein R⁴ and R⁵ is independent of each other a C₁-C₄ alkyl group, —NO₂, —CN, —COOH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H.

According to a preferred embodiment X¹ is aryl or heteroaryl, preferably phenyl.

According to another preferred embodiment, R¹ is a monosaccharide according to formulae (III), wherein Z is preferably S or O. According to yet another preferred embodiment, R¹ is a monosaccharide according to formulae (XII), wherein Z is preferably —CH₂—NH—SO₂—.

In yet another embodiment, Y comprises or consists, preferably consists of:
(i) -Pep-;
(ii) —B¹-Pep-;
(iii) —B¹-Pep-B²—
(iv) or comprises, one, two or three X, Pep and optionally at least one B¹ and/or at least one B², wherein X is directly or indirectly via B¹ bound to at least two R¹, and Pep is directly or indirectly via B² bound to R². B¹ and B² have the meaning defined above.

The most preferred arrangement is —B¹—Pep-.

Generally, it is preferred that the protease cleavable peptide is directly linked to the anti-bacterial therapeutic agent with a peptide bond. This provides the advantage that only very few moieties of the conjugate remain bound to the anti-bacterial therapeutic agent once released by cleavage of the peptide Pep. This avoids interference with antibiotic function. The Pep within Y may be in either orientation, i.e. the peptide bond may be between the N-terminal amine of the peptide and a carboxyl group of the anti-bacterial therapeutic agent or between the C-terminal carboxyl group of the peptide and an amine group of the antibacterial therapeutic agent. It is preferred that the C-terminal carboxyl group of the peptide forms a peptide bond with an amine group of the antibacterial therapeutic agent.

According to a preferred embodiment, the conjugate according to formula I has the following structure:

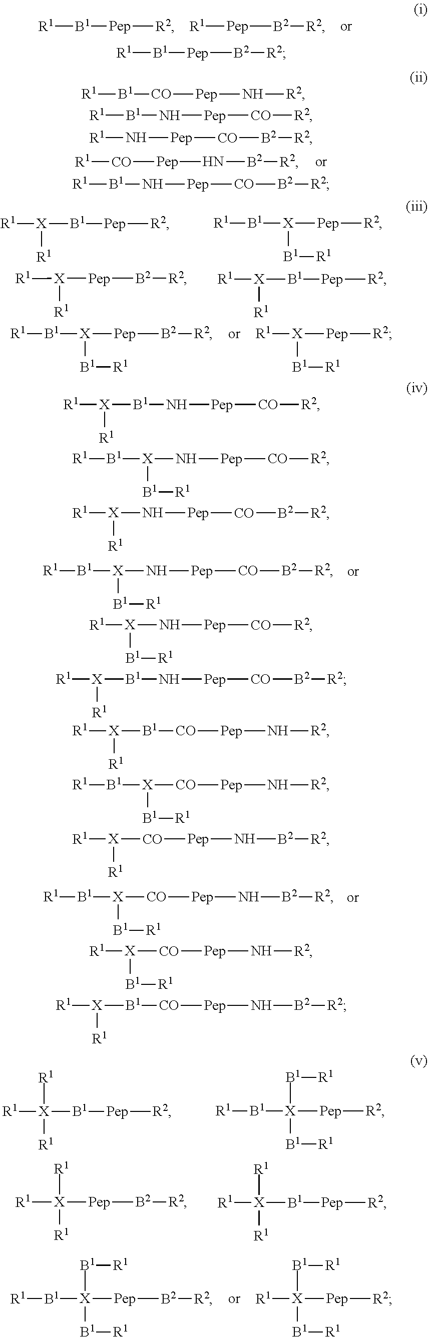

wherein
B¹ in each case is independently selected from a first bridging moiety;
B² in each case is independently selected from a second bridging moiety;
X is a branching moiety capable of forming at least three covalent bonds; and
NH is the amino group of the N-terminal amino acid of Pep and CO is the carboxy group of the C-terminal amino acid of Pep.

The branching moiety X is a radical that has at least three free valences and thus can from one bond directly or indirectly to the peptide and two or more bonds with two or more R¹.

According to a preferred embodiment, B¹ comprises or consists of A, R³, R³-A, A-R⁶, R³-A-R⁶, -A-R⁷-A-, R³-A-A-R⁶, -A-R⁷-A-R⁶, or R³-A-R⁷-A-R⁶, preferably -A-R⁷-A, wherein each A may be the same or different; wherein preferably R³, if present, is bound to R¹ and wherein preferably R⁶, if present, is bound to X or Pep;
B² comprises or consists of A, R¹, R-A, A-R⁹, or R¹-A-R⁹, -A-A-, -A-A-R⁹, R¹-A-A-, -A-A-R⁹, R¹-A-A-R⁹ or R¹-A-R⁷-A-R⁹, wherein each A may be the same or different, preferably is -A-A-R⁹;
wherein
A is selected from the group consisting of:
   (a) a carbocyclic group, preferably a C₅-Cᵣ-cycloalkyl, a phenylene group or a naphthalenediyl, most preferably a phenylene group, which is optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR⁴R⁵ wherein R⁴ and R⁵ is independent of each other a C₁-C₄ alkyl group, —NO₂, —CN, —COGH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H, and
   (b) a heterocarbocyclic group, preferably that is optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR⁴R⁵ wherein R⁴ and R⁵ is independent of each other a C₁-C₄ alkyl group, —NO₂, —CN, —COOH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H; or
   (c) —[O—CH₂—CH₂]m-, wherein m is between 1 to 10;
   R³ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—; —CH═CH—NH—CO—; —NH—CO—CH═CH—; and —CH₂—CH₂—NH—CO—, wherein R³, if present is bound to R¹;
   R⁶ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—; —CH═CH—NH—CO—; —NH—CO—CH═CH—; and —CH₂—CH₂—NH—CO—, preferably —CO— and —NH—, wherein R⁶, if present is bound to X or Pep;
   R⁷ is selected from the group consisting of —CO—, NH—, —NH—CO—, —CO—NH—, —CO—NH—(C₁-C₄ alkyl), —NH—CO—(C₁-C₄ alkyl), —(C₁-C₄ alkyl)-CO—NH—; or —CO—NH—(C₁-C₄ alkyl);
   R⁸ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—; —CH═CH—NH—CO—; —NH—CO—CH═CH—; and —CH₂—CH₂—NH—CO—, wherein R⁸, if present is bound to Pep or (CH₂)q;
   R⁹ is selected from the group consisting of —CO—; —NH—, NH—S—NH, preferably —CO— and —NH—CO—NH—(C₁-C₄ alkyl), —NH—CO—(C₁-C₄ alkyl), —(C₁-C₄ alkyl)-CO—NH—; or —CO—NH—(C₁-C₄ alkyl), wherein R⁹, if present is bound to R²;
   and/or
   X is selected from the group amine, ammonium, phosphate, phosphine, phosphonate, tertiary or quarternary carbon, tricarboxybenzoic acid, triaminomethyl benzene, citric acid, glycerol, trishydroxymethyl amine, lysine, cyclo oligolysine, 1,4,7,10-tetrazacyclododecan, 1,4,7-triazacyclononan, 1,5,9-triazacyclododecan.

In the conjugate according to formula (I) it is preferred that B¹ comprises or consists, preferably consists of -A-R¹, wherein R⁶ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—; —CH═CH—NH—CO—; —NH—CO—CH═CH—; and —CH₂—CH₂—NH—CO—, preferably —CO— and —NH—, wherein R⁶, if present is bound to X or Pep.

In the conjugate according to formula (I) it is preferred that B² is not present, if Y does not comprise a branching moiety. If Y comprises a branching moiety, it is preferred that B² is present. In this case B² preferably comprises -A-R⁶.

In the conjugate according to formula (II) it is preferred that B¹ comprises or consists, preferably consists of -A-R⁷-A-, wherein preferably A in each case is identical even more preferably is a phenylene group.

In the conjugate according to formula (II) it is preferred that B² is A-A or A-R⁹, wherein it is preferred that one A is triazolyl and the other is —[O—CH₂—CH₂]m-, wherein m is between 1 to 10. In another preferred embodiment, it is preferred that B²is A-A or A-R⁹, wherein it is preferred that one A is triazolyl and the other is an alkyl chain with one amide bond.

In yet another embodiment, A is selected from the group consisting of
(i) —[O—CH₂—CH₂]m-, wherein m is between 1 to 10;
(ii) a naphthalenediyl group;
(iii) a five-membered aromatic or nonaromatic monocyclic ring, wherein 1, 2, 3, or 4 of the ring atoms are the same or different heteroatoms, said heteroatoms being selected from O, N, or S, preferably triazolyl,
(iv) a six-membered aromatic or nonaromatic monocyclic ring, wherein 1, 2, 3, 4, or 5 of the ring atoms are the same or different heteroatoms, said heteroatoms being selected from O, N, or S, preferably a phenylene group; or
(v) an aromatic or nonaromatic bicyclic ring system with 8 to 12 members, wherein 1, 2, 3, 4, 5, or 6 of the ring atoms are the same or different heteroatoms, said heteroatoms being selected from O, N, or S;
   wherein each one of the above mentioned groups (i) to (v) is optionally substituted by one, two or three substituents that are independently from each other selected from the group consisting of a C₁-C₄ alkyl group, halogen, a C₁-C₄ haloalkyl group, —OH, a C₁-C₄ alkoxy group, —NH₂, —NHR¹³ with R¹³ being a C₁-C₄ alkyl group, —NR¹³R¹⁴ with R¹³ and R¹⁴ each being independently from each other a C₁-C₄ alkyl group, —NO₂, —CN, —COOH, —COOR¹³ with R¹³ being a C₁-C₄ alkyl group, —CONH₂, —CONHR¹³ with R¹³ being a C₁-C₄ alkyl group, —CONR¹³R¹⁴ with R¹³ and R¹⁴ each being independently from each other a C₁-C₄ alkyl group, and —SO₃H.

In the conjugate according to formula (II) it is preferred that B¹ is A-R⁷-A. According to a further preferred embodiment A is in each case a phenylene group.

In the conjugate according to formula (I) it is preferred that B is selected from the group consisting of:

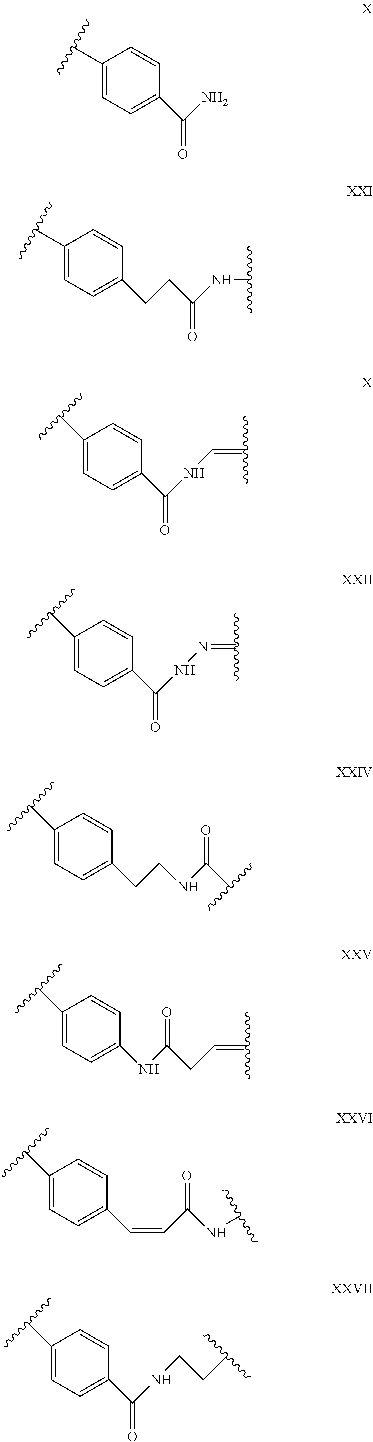

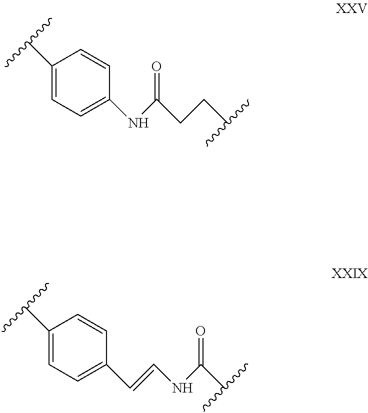 [SMILES: *CCC(=O)Nc1ccc(*)cc1 ; *C(=O)N/C=C/c1ccc(*)cc1]

According to a more preferred embodiment, in the conjugate according to formula (I), B¹ is a molecule according to formula (XX) or formula (XXI).

In the conjugate according to formula (II), in some embodiments B¹ is selected from the group consisting of

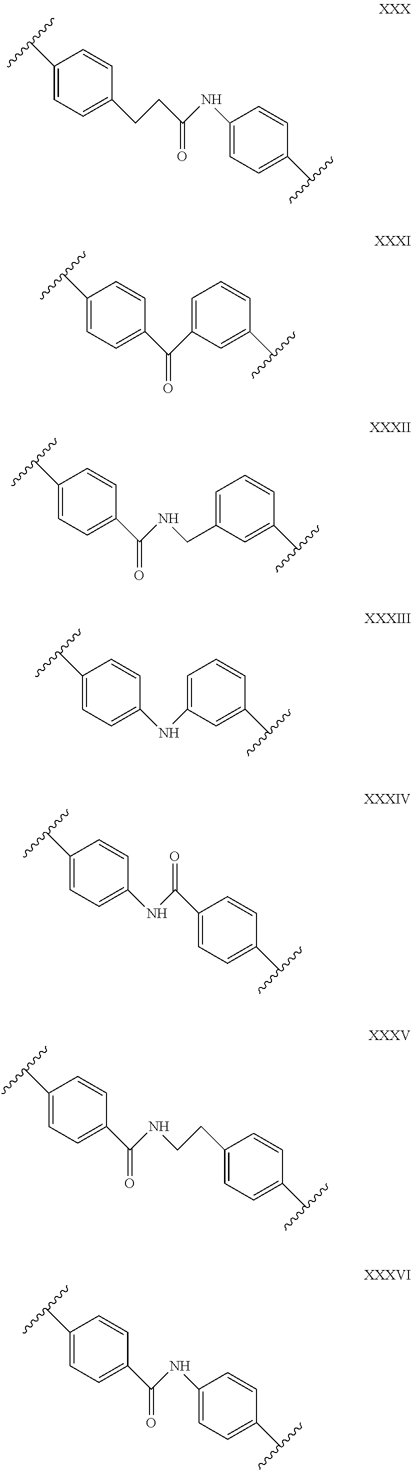

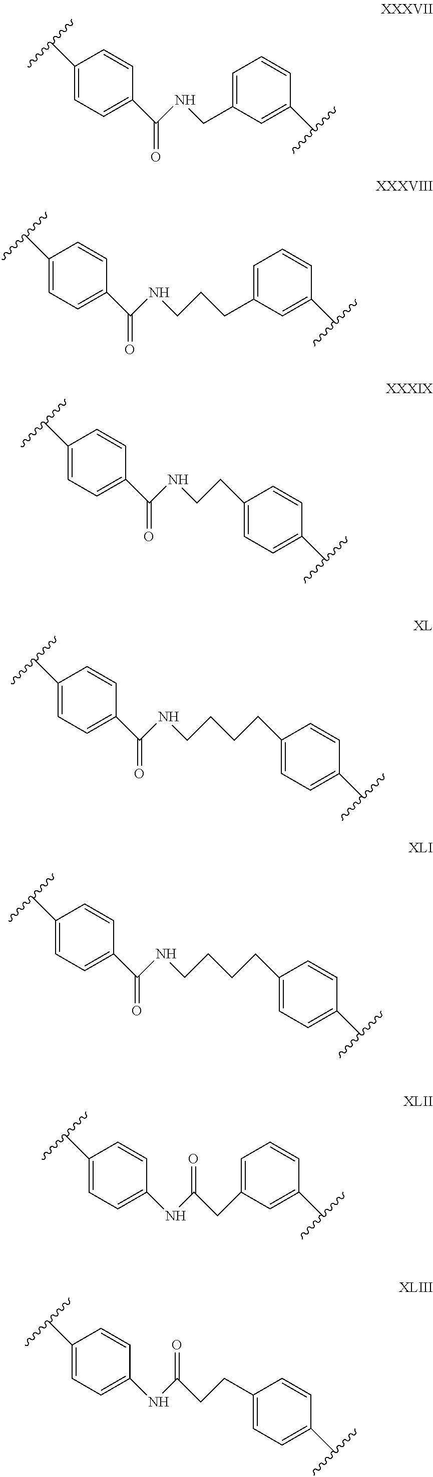

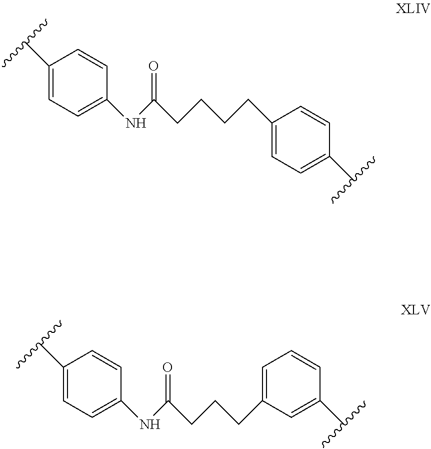 [SMILES: *c1ccc(CCCCC(=O)Nc2ccc(*)cc2)cc1 ; *c1ccc(NC(=O)CCCc2cccc(*)c2)cc1]

In preferred embodiments, in the conjugate according to formula (II), B¹ is -A-R⁷-A, wherein R⁷ is selected from the group consisting of —CO—, NH—, —NH—CO—, —CO—NH—, —CO—NH—(C₁-C₄ alkyl), —NH—CO—(C₁-C₄ alkyl), —(C₁-C₄ alkyl)-CO—NH—; or —CO—NH—(C₁-C₄ alkyl), and A is a 4- to 8-membered, in particular 5- or 6-membered carbocyclic group or heterocarbocyclic group, preferably a 4- to 8-membered, more preferably 5- or 6-membered carbocyclic group or heterocarbocyclic group.

It is preferred that, in the conjugate according to formula (II), B¹ is -A-R⁷-A, wherein R⁷ is selected from the group consisting of —NH—CO—, —CO—NH—, —CO—NH—(C₁-C₄ alkyl), —NH—CO—(C₁-C₄ alkyl), —(C₁-C₄ alkyl)-CO—NH—; or —CO—NH—(C₁-C₄ alkyl), and A is a phenylene group, which is optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR 4R⁵ wherein R⁴ and R⁵ is independent of each other a C₁-C₄alkyl group, —NO₂, —CN, —COOH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H, preferably —SO₃H.

It is further preferred that in the conjugate according to formula (II), B¹ is -A-R⁷-A, wherein R⁷ is selected from the group consisting of —NH—CO—, —CO—NH—, —CO—NH—(C₁-C₄ alkyl), —NH—CO—(C₁-C₄ alkyl), —(C₁-C₄ alkyl)-CO—NH—; or —CO—NH—(C₁-C₄ alkyl), and A is a substituted or unsubstituted, preferably unsubstituted heterocarbocyclic group, preferably A is a pyridine.

Even more preferably, in the conjugate according to formula (II), B¹ is -A-R⁷-A, wherein R⁷ is selected from —CO—NH—(C₁-C₄ alkyl), —NH—CO—(C₁-C₄ alkyl), —(C₁-C₄ alkyl)-CO—NH—; or —CO—NH—(C₁-C₄ alkyl) and A is selected from a pyridine, benzenesulfonate or benzene.

In most preferred embodiments, in the conjugate according to formula (II), B¹ is -A-R⁷-A, wherein R⁷ is selected from —CO—NH—(C₁-C₄ alkyl), —NH—CO—(C₁-C₄ alkyl), —(C₁-C₄ alkyl)-CO—NH—; or —CO—NH—(C₁-C₄ alkyl) and each A is benzene.

Most preferably, in the conjugate according to formula (II), B¹ is characterized by formula (XXX) or (XLIII), most preferably formula (XLIII). Most preferably, in the conjugate according to formula (II), B¹ is characterized by formula (XLIII).

In a preferred embodiment, B² in formula (II) is A-R⁹, wherein A is triazolyl optionally substituted by C₁-C₄ alkyl, preferably C₂ alkyl.

In a preferred embodiment, B² in formula (II) is A-A-R⁹, wherein the first A is triazolyl optionally substituted by C₁-C₄ alkyl, preferably C₂ alkyl, and the second A is-[O—CH₂—CH₂]m, wherein m is between 1 to 10. Preferably, m is between 2 to 8, more preferably between 3 to 5, and most preferably m is 2 or 3.

In some embodiments, B² in formula (II) is

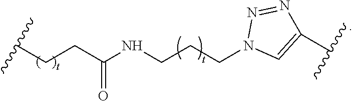

wherein each t is independently selected from 0, 1 or 2, preferably each t is 1.

In yet another preferred embodiment, B² is

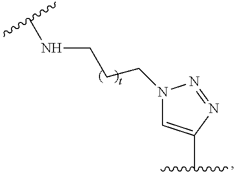

wherein t is selected from 0, 1 or 2, preferably t is 1.

In yet another preferred embodiment, B² is

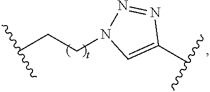

wherein t is selected from 0, 1 or 2, 3, 4 or 5, preferably t is 5.

In the conjugate according to formula (II), it is most preferred that B² is selected from the group consisting of:

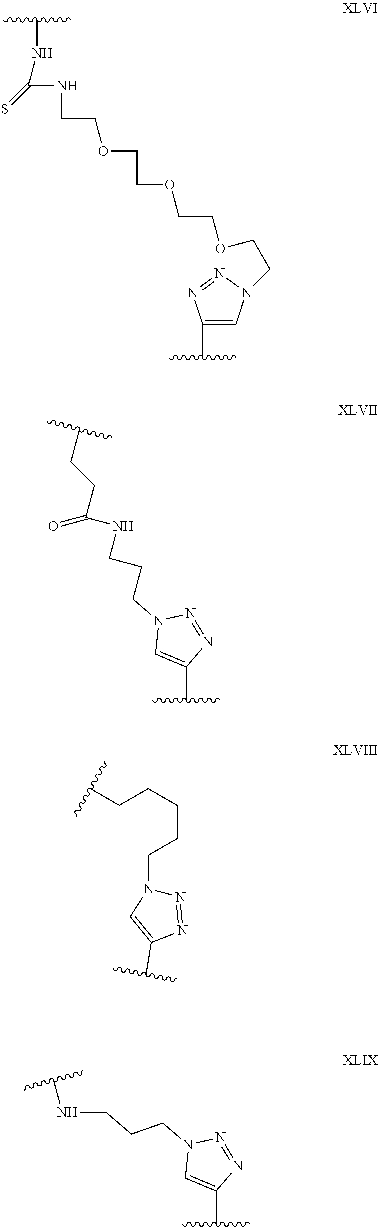 [SMILES: *NC(=S)NCCOCCOCCOCCn1cc(*)nn1 ; *CCC(=O)NCCCn1cc(*)nn1 ; *CCCCCn1cc(*)nn1 ; *NCCCn1cc(*)nn1]

In a further embodiment, Pep comprised in the linker:
(i) has a length between 2 to 20 amino acids; and/or
(ii) has a direct covalent bond to the anti-bacterial therapeutic agent; and/or
(iii) comprises or consists of an amino acid sequence selected from the group consisting of

(SEQ ID NO: 3)
FFA, ALA, GGG, GGA, GGF, AGLA,
(SEQ ID NO: 4)
FGLA,
(SEQ ID NO: 5)
FGAK,
(SEQ ID NO: 6)
LVLGA,
(SEQ ID NO: 7)
LVLGGS,
(SEQ ID NO: 8)
LLLGGS,
(SEQ ID NO: 9)
VVLGGS,
(SEQ ID NO: 10)
VLGS,
(SEQ ID NO: 11)
LVFGGS,
(SEQ ID NO: 12)
LVMTSG,
(SEQ ID NO: 13)
RVRGHF,
(SEQ ID NO: 14)
IAAG,
(SEQ ID NO: 15)
LAFGA,
(SEQ ID NO: 16)
IVFGGS,
(SEQ ID NO: 17)
VVGGSG,
(SEQ ID NO: 18)
VVAGGS,
(SEQ ID NO: 19)
IFGA,
(SEQ ID NO: 20)
ITYGAS,
(SEQ ID NO: 21)
GSFGAR,
(SEQ ID NO: 22)
LVFGA,
(SEQ ID NO: 23)
IAKD,
(SEQ ID NO: 24)
KGPA,
(SEQ ID NO: 25)
PLGPDR,
(SEQ ID NO: 26)
AGPPGP,
(SEQ ID NO: 27)
PRPPAPVFY,
(SEQ ID NO: 28)
RRKKVYP,
(SEQ ID NO: 29)
TPIQL,
(SEQ ID NO: 30)
LPAL,
(SEQ ID NO: 31)
LRIS,
(SEQ ID NO: 32)
LKIS,
(SEQ ID NO: 33)
LKLN,
(SEQ ID NO: 34)
ARFT,
(SEQ ID NO: 35)
LQLP,
(SEQ ID NO: 36)
LGLP,
(SEQ ID NO: 37)
LFGA,
(SEQ ID NO: 38)
LYGA
and
(SEQ ID NO: 37)
IYGA;
and/or

(iv) wherein one or more of the peptide bonds between the amino acids of Pep that are not cleaved by the bacterial protease are bioisosters of the peptide bonds, preferably N-alkyl/cycloalykl peptides, retropeptides, 5-membered heteroaromatic rings, preferably like triazoles/oxadiazoles/thiazoles/, ureas, thioureas, beta-peptides, sulfonamides, thioamides, carbamates.

Preferably, the Pep comprised the linker has a length between 2 to 20 amino acids, between 3 to 18 amino acids, between 4 to 16 amino acids, between 5 to 14 amino acids, between 6 to 12 amino acids, or between 7 to 10 amino acids. According to a more preferred embodiment, the linker has a length between 3 to 6 amino acids.

According to more preferred embodiment, the Pep comprised in the linker comprises or consists of the amino acid sequence selected from AGLA according to SEQ ID NO: 3, FFA (linker variant 38, table 1), ALA (linker variant 39, table 1) or FGLA according to SEQ ID NO: 4 (table 1). According to the most preferred embodiment, the Pep comprised in the linker comprises or consists of the amino acid sequence AGLA according to SEQ ID NO: 3 (table 1).

TABLE 1
Sequence listing

| Name | SEQ ID NO | Amino acid sequence |
|---|---|---|
| LecA | 1 | MAWKGEVLAN NEAGQVTSII YNPGDVITIV |
| | | AAGWASYGPT QKWGPQGDRE HPDQGLICHD |
| | | AFCGALVMKI GNSGTIPVNT GLFRWVAPNN |
| | | VQGAITLIYN DVPGTYGNNS GSFSVNIGKD QS |
| LecB | 2 | MATQGVFTLP ANTRFGVTAF ANSSGTQTVN |
| | | VLVNNETAAT FSGQSTNNAV IGTQVLNSGS |
| | | SGKVQVQVSV NGRPSDLVSA QVILTNELNF AL- |
| | | VGSEDGTD NDYNDAVVVI NWPLG |
| Linker variant 1 | 3 | AGLA |
| Linker variant 2 | 4 | FGLA |
| Linker variant 3 | 5 | FGAK |
| Linker variant 4 | 6 | LVLGA |
| Linker variant 5 | 7 | LVLGGS |
| Linker variant 6 | 8 | LLLGGS |
| Linker variant 7 | 9 | VVLGGS |
| Linker variant 8 | 10 | VLGS |
| Linker variant 9 | 11 | LVFGGS |
| Linker variant 10 | 12 | LVMTSG |
| Linker variant 11 | 13 | RVRGHF |
| Linker variant 12 | 14 | IAAG |
| Linker variant 13 | 15 | LAFGA |
| Linker variant 14 | 16 | IVFGGS |
| Linker variant 15 | 17 | VVGGSG |
| Linker variant 16 | 18 | VVAGGS |
| Linker variant 17 | 19 | IFGA |
| Linker variant 18 | 20 | ITYGAS |
| Linker variant 19 | 21 | GSFGAR |
| Linker variant 20 | 22 | LVFGA |
| Linker variant 21 | 23 | IAKD |
| Linker variant 22 | 24 | KGPA |
| Linker variant 23 | 25 | PLGPR |
| Linker variant 24 | 26 | AGPPGP |
| Linker variant 25 | 27 | PRPPAPVFY |
| Linker variant 26 | 28 | RRKKVYP |
| Linker variant 27 | 29 | TPIQL |
| Linker variant 28 | 30 | LPAL |
| Linker variant 29 | 31 | LRIS |
| Linker variant 30 | 32 | LKIS |
| Linker variant 31 | 33 | LKLN |
| Linker variant 32 | 34 | ARFT |
| Linker variant 33 | 35 | LQLP |
| Linker variant 34 | 36 | LGLP |
| Linker variant 35 | 37 | LFGA |
| Linker variant 36 | 38 | LYGA |
| Linker variant 37 | 39 | IYGA |
| Linker variant 38 | | FFA |
| Linker variant 39 | | ALA |
| Linker variant 40 | | GGG |
| Linker variant 41 | | GGA |
| Linker variant 42 | | GGF |

According to a further embodiment, the anti-bacterial therapeutic agent is an antibiotic. Preferably, the antibiotic can be selected from the group comprising β3-lactam antibiotics, e.g. penicillins comprising benzylpenicillin, phenoxymethylpenicillin, piperacillin, mezlocillin, ampicillin, amoxicillin, flucloxacillin, methicillin, oxacillin; β3-lactamase inhibitors e.g. clavulanic acid, sulbactam, tazobactam, sultamicillin; monobactams e.g. aztreonam; cephalosporins comprising cefazolin, cefalexin, loracarbef, cefuroxime, cefotiam, cefaclor, cefotaxime, ceftriaxone, cefepime, ceftazidime, cefixime, cefpodoxime, ceftibuten; carbapenems comprising imipenem, meropenem, ertapenem; fluoroquinolones, e.g. ciprofloxacin, levofloxacin, gatifloxacin, moxifloxacin, ofloxacin, norfloxacin; lipopeptides e.g. daptomycin, glycopeptides e.g. bleomycin, vancomycin, teicoplanin, aminoglycosides e.g. gentamicin, dibekacin, sisomicin, tobramycin, amikacin, kanamycin, neomycin, streptomycin, netilmicin, apramycin, paromomycin, spectinomycin, geneticin; oxazolidinediones e.g. linezolid; glycylcyclines e.g. tigecycline; polypeptides e.g. polymyxin, polyketides, e.g. tetracyclines comprising tetracycline, oxytetracycline, minocycline, doxycycline, chlortetracycline, rolitetracycline or macrolides comprising erythromycin, azithromycin, clarithromycin, roxythromycin; ketolides e.g. telithromycin; quinolones e.g. ciprofloxacin norfloxacin, ofloxacin; moxifloxacin, enoxacin, gatifloxacin, sparfloxacin, pefloxacin, fleroxacin, levofloxacin, trovafloxacin; sulfonamides e.g. sulfamethoxazole, sulfacarbamide, sulfacetamide, sulfamethylthiazole, sulfadiazine, sulfamethoxozole, sulfasalazine, argyrins, and cystobactamids. Also comprised are organic or anorganic salts of above listed molecules as well as prodrugs thereof. The antibacterial therapeutic agent comprised in the conjugates of the present invention is covalently coupled to further moieties, e.g. B² or Y, and, thus, it comprises this additional bond that is not comprised in the active molecules mentioned above. Preferably, upon cleavage of the peptide linker the antibiotic is selected from the group consisting of fluoroquinolones. Thus, it is preferred that antibacterial activity of the respective antibiotics is not negatively affected by the remainder of the conjugate. Is it nevertheless preferred that the antibiotic is released in an active form upon cleavage of the peptide (Pep) cleavable by a bacterial protease. Alternatively, it is preferred that only a part of the remainder of the conjugate remains coupled to the antibiotic after protease cleavage that may be subsequently cleaved by chemical processes as, e.g. hydrolysis or exopeptidases present at the site of cleavage. Particularly preferred antibiotics are selected from the group consisting of aminopyrrolidine fluoroquinolone (compound 2 of FIG. 4) or aminomethylpyrrolidine fluoroquinolone (compound 3 of FIG. 5), ciprofloxacin, levofloxacin, moxifloxacin, ofloxacin, gemifloxacin or delafloxacin. According to a most preferred embodiment, the antibiotic is selected from ciprofloxacine, aminopyrrolidine fluoroquinolone (compound 2 of FIG. 4) or aminomethylpyrrolidine fluoroquinolone (compound 3 of FIG. 5).

In yet another embodiment, the imaging agent is a chemical group detectable by fluorescence spectroscopy, by positron-emission tomography (PET), or by magnetic resonance imaging (MRI). According to a preferred embodiment, the imaging agent is fluorescein, sulfoCy7, TAMRA, BODIPY, ¹⁸F or Gd(III)-DOTA. In a more preferred embodiment, the imaging agent is selected from fluorescein, sulfoCy7 and BODIPY.

In one embodiment, the conjugate according to the present invention has a structure selected from the following group:

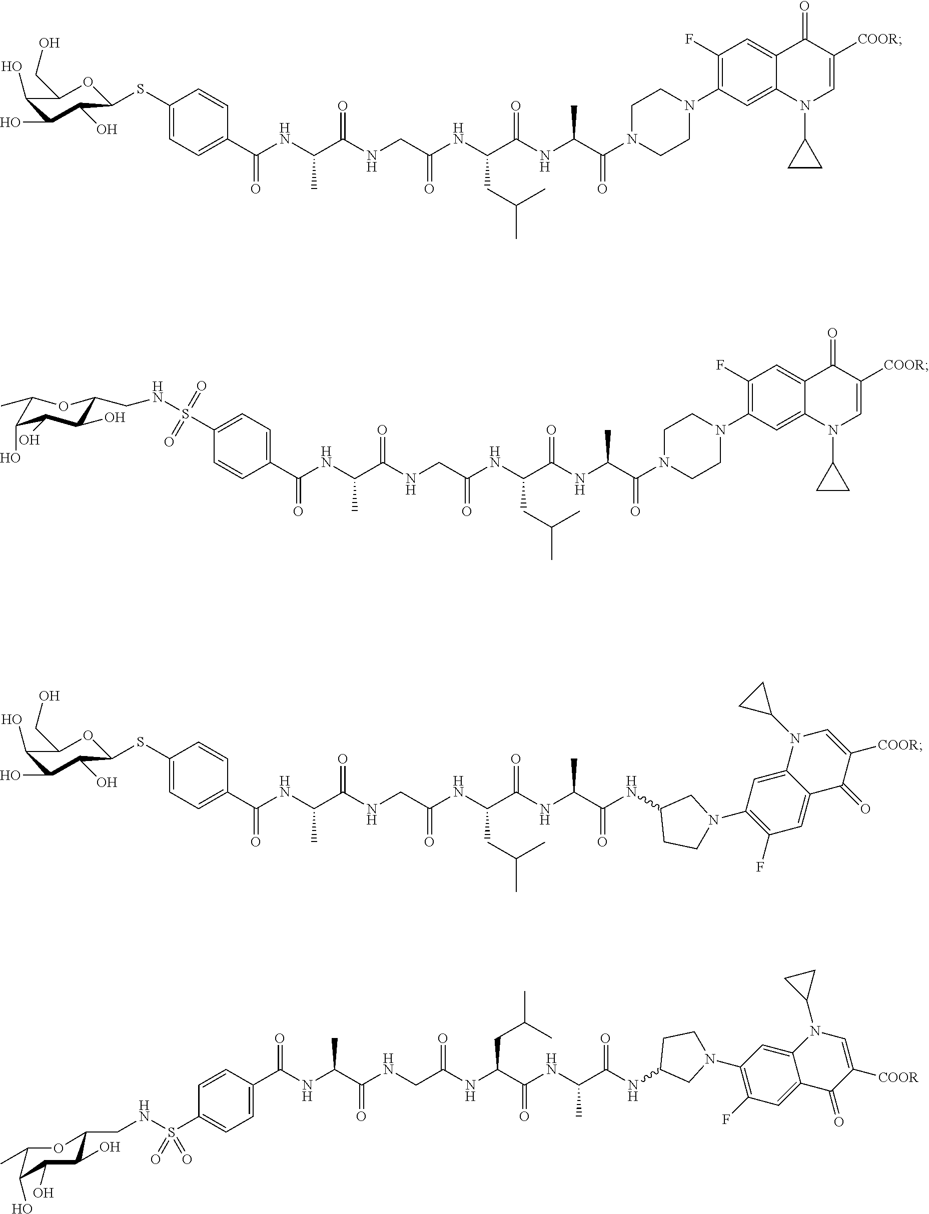

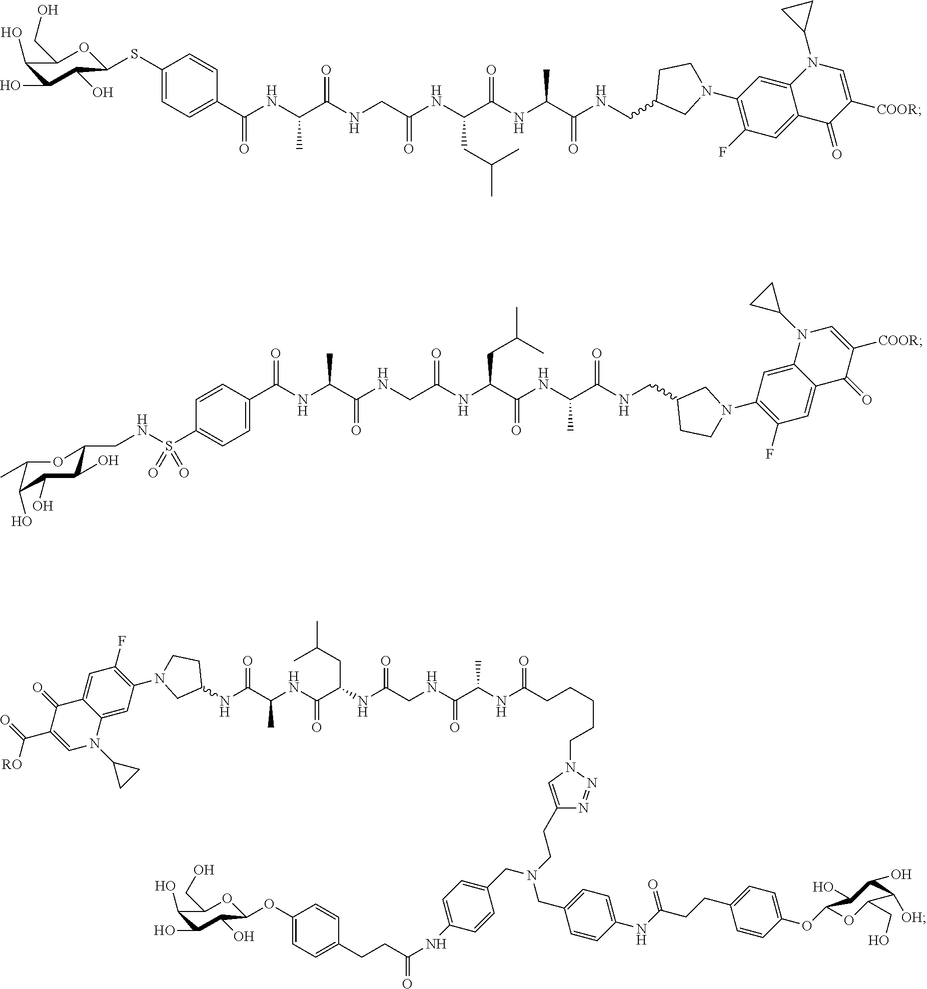

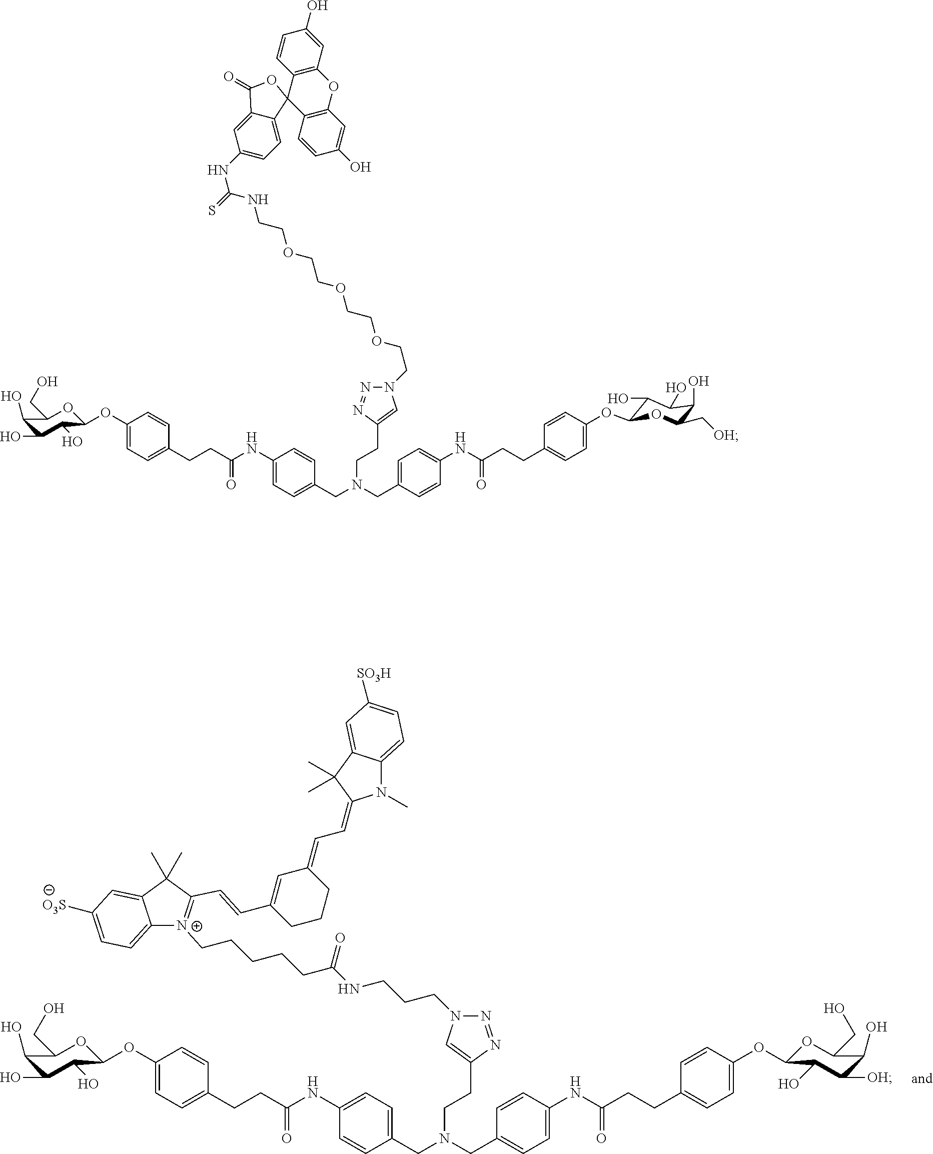

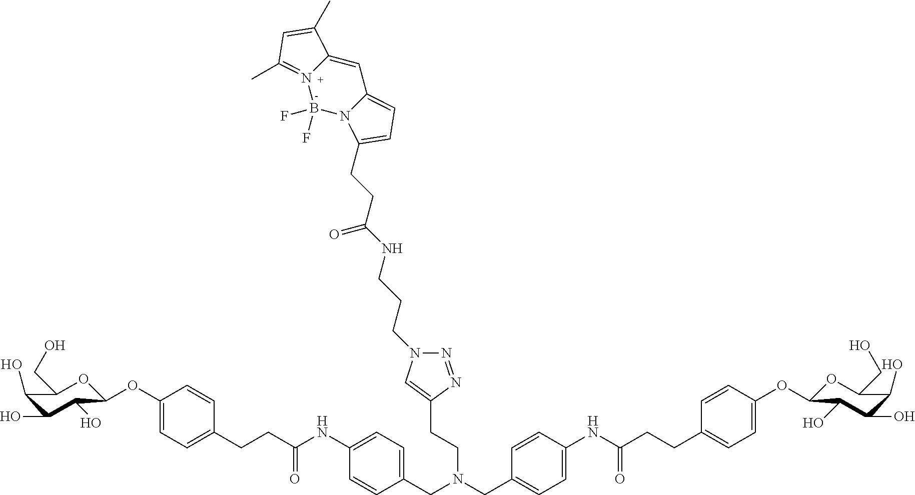

wherein R is H or an alkyl group, preferably C₄ to C₁-alkyl.

In a second aspect, the invention provides a pharmaceutical or diagnostic composition comprising the conjugate according to the first aspect, and optionally comprising one or more constituents selected from the group consisting of a pharmaceutically acceptable carrier, a diluent and an excipient.

In a third aspect, the invention provides a conjugate according to the first aspect or a pharmaceutical or diagnostic composition according the second aspect for use in medicine.

In a forth aspect, the invention provides a conjugate according to the first aspect or a pharmaceutical or diagnostic composition according to the second aspect for use in treating, preventing or diagnosing a disease or infection associated with a bacterium of the phylum Firmicutes, preferably of the class of Bacilli or Clostridia; the phylum Actinobacteria, preferably of the order Corynebacteriales; or the phylum Proteobacteria, preferably of the class of Alphaproteobacteria, Betaproteobacteria or Gammaproteobacteria. According to a preferred embodiment, the Clostridia are of the family Clostridiaceae. According to a further embodiment, the Corynebacteriales is of the family Mycobacteriaceae. According to a preferred embodiment, the Corynebacteriales are of the genus Mycobacterium. According to another embodiment, the Alphaproteobacteria are of the order Hyphomicrobiale. According to a preferred embodiment, the Alphaproteobacteria are of the family Phyllobacteriaceae or Brucellaceae. According to a further embodiment, the Betaproteobacteria is of the order Burkholderiales. In a preferred embodiment, the Betaproteobacteria are of the family Oxalobacteraceae, Burkholderiaceae, or Comamonadaceae. According to another embodiment, the Betaproteobacteria is of the order Neisseriales. According to a preferred embodiment, the Betaprotebacteria is of the family Neisseriaceae. According to one embodiment, the Gammaproteobacteria are of the order Pseudomonadales. According to a preferred embodiment, the Gammaproteobacteria are of the family Moraxellaceae. According to a further embodiment, the Gammaprotebacteria are of the order Enterobacterales. According to a preferred embodiment, the Gammaproteobacteria are of the family Enterobacteriaceae or Morganellaceae. According to yet another embodiment, the Gammaproteobacteria are of the order Oceanospirillales. According to a preferred embodiment, the Gammaproteobacteria are of the family Halomonadaceae. According to a further embodiment, the Gammaproteobacteria are of the order Pasteurellales. According to a preferred embodiment, the Gammaproteobacteria are of the family Pasteurellaceae. According to a further embodiment, the Gammaproteobacteria are of the order of Vibrionales. According to a preferred embodiment, the Gammaproteobacteria are of the family Vibrionaceae. According to yet another embodiment, the Gammaprotebacteria are of the order of Pseudomonadales. According to a more preferred embodiment, the Gammaproteobacteria are of the family Pseudomonadaceae.

In a particularly preferred embodiment, the conjugate according to the first aspect or a pharmaceutical or diagnostic composition according to the second aspect is for use in treating, preventing or diagnosing a disease or infection associated with a bacterium selected from the group consisting of Pseudomonas sp., Chromobacterium, Collimonas sp., Enterobacter sp., Inquilinus limosus, Klebsiella pneumoniae, Jejubacter calystegiae, Mesorhizobium sp., Mycobacterium vulneris, Ochrobactrum sp., Photorhabdus sp., Xenorhabdus sp., Burkholderia sp., Citrobacter freundii, Diaphorobacter sp., Paraburkholderida, Ralstonia, Vibrio parahaemolyticus, Halomonas anticariensis, Staphylococcus aureus, Clostridium histolyticum, Neisseria gonorrhoeae, Neisseria meningitis, Haemophilus influenza, Streptococcus sanguinis, Streptococcus pneumoniae, Bacillus anthracis, Acinetobacter baumannii, and Listeria monocytogenes.

In a preferred embodiment, the conjugate, the pharmaceutical or the diagnostic composition is for use in treating preventing or diagnosing a disease or infection associated with Pseudomonas aeruginosa. Preferably, the disease or infection associated with Pseudomonas aeruginosa is selected from the group consisting of dermatitis, bacteraemia, sepsis, cystic fibrosis, acute bacterial endocarditis, folliculitis; skin infection and subcutaneous tissue infection, e.g. folliculitis, acute external otitis, ecthyma gangrenosum; bone infection, ear infection, eye infection, urinary tract infection, respiratory tract infection, e.g. ventilator-associated pneumonia, bronchitis; and gastrointestinal infection.

The invention is described by way of the following examples which are to be construed as merely illustrative and not limitative of the scope of the invention.

### FIGURES

FIG. 1. Anticipated antibiotic cargo for the lectin-targeted conjugate. (1) Ciprofloxacin is a very potent approved drug. (2) Aminopyrrolidine and (3) aminomethylpyrrolidine also show high antibiotic activity and carry a primary amine which serves as a handle for conjugation to the peptide linker.

FIG. 2. Scheme for the chemical synthesis of the lectin-targeted carbohydrate-peptide conjugates 10 (Lec-A-targeted) and 16 (LecB-targeted)^{a}, a Reagents and conditions: (a) BF₃·Et₂O, 0° C. to room temperature, 16 h, 58%; (b) first NaOMe, MeOH, room temperature, 30 min, then LiOH, MeOH/H₂O (20:3), 1 h, quant.; (c) TBTU, DIPEA, DMF, room temperature, 1 h, 58%; (d) LiOH, DMF/H₂O, 50° C., 49%; (e) Et₃N, DMF, 0° C. to room temperature, 16 h, 67%; (f) LiOH, THF/MeOH/H₂O (3/1/1), room temperature, 95%; (g) TBTU, DIPEA, DMF, room temperature, 1 h 80%; (h) H₂, cat. Pd/C, MeOH, room temperature, 16 h, 95%.

FIG. 3. Scheme for the chemical synthesis of the ciprofloxacin-based lectin-targeted drug delivery conjugates 24 (LecA-targeted) and 25 (LecB-targeted)^{a} and control compound 19, a Reagents and conditions: (a) first 17, Ibef, NMM, THF, −15° C., then 1, THF, room temperature, 3 h, 25%; (b) HCL, dioxane, room temperature, 1 h, 54%; (c) first Boc₂O, KHCO₃, DMF, 40° C., 90 min, then BnBr, 115° C., 86% over 2 chemical steps; (d) HCl, dioxane, room temperature, 1 h, quant.; (e) TBTU, DIPEA, room temperature, 1 h, 84% for 23; (f) H₂, cat. Pd MeOH, room temperature, 6d 22% over two chemical steps; (g) H₂, cat. Pd, MeOH, room temperature, 24 h, 74%.

FIG. 4. Scheme for the chemical synthesis of the aminopyrrolidine-based lectin-targeted drug delivery conjugates 36 (LecA-targeted) and 37 (LecB-targeted) and control compounds 2 and 35^{a}, ^{a} Reagents and conditions: (a) pyridine, reflux, 16 h, 32-50%; (b) BnBr, K₂CO₃, DMF, 110° C., 60 min, 86%; (c) HCl, dioxane, room temperature, 1 h, 97% for 2, quant, for 30; (d) TBTU, DIPEA, room temperature, 1 h, 74% for 31, 49% for 35; (e) H₂, cat. Pd, MeOH, room temperature, 16 h; (f) HCl, dioxane, room temperature, 1 h, 61% over two chemical steps; (g) H₂, cat. Pd, MeOH, room temperature, 24 h, 48% over two chemical steps; (h) H₂, cat. Pd/C, MeOH, room temperature, 41%.

FIG. 5. Scheme for the chemical synthesis of the aminomethylpyrrolidine-based lectin-targeted drug delivery conjugates 48 (LecA-targeted) and 49 (LecB-targeted) and control compounds 3 and 47^{a}, ^{a} Reagents and conditions: (a) pyridine, reflux, 16 h, 62%; (b) (R S)-CSA, MeOH, reflux, 72 h, 96%; (c) DIAD, P(Ph)₃, DPPA, THF, 1 h, 65%; (d) H₂, cat. Pd/C, MeOH, 16 h, then HCl, dioxane/Et₂O, 0° C. 78%; (e) LiOH, THF/MeOH/H₂O (3:1:1), 2d, 43%; (f) TBTU, DIPEA, DMF, room temperature, 1 h, 74% for 47, 72% for 45; (g) LiOH, THF/H₂O/MeOH (5:5:1), room temperature, 3 h, 86%; (h) HCl, dioxane, room temperature, 1 h, 54%; LiOH, H₂O/THF (5:1), room temperature, 3 h, 70% over two chemical steps; j) LiOH, THF/H₂O/MeOH (3:1:1), room temperature, 12 h, 96%.

FIG. 6. Scheme for the chemical synthesis of tetrapeptide linker 8 and dipeptide building block 17^{a}. ^{a} Reagents and conditions: (a) EDC-HCl, HOBt, DIPEA, CH₂Cl₂, room temperature, 6-24 h, 88% for S3, 98% for S7; (b) Pd/C, H₂, THF, room temperature, 16 h, quant.; (c) HCl, dioxane, room temperature, 2 h, quant.; (d) EDC-HCl, HOBt, DIPEA, CH₂Cl₂, room temperature, 2 h, 51%; (e) HCl, dioxane, room temperature, 4 h, quant.; (f) HCl, HOBt, DIPEA, CH₂Cl₂, room temperature, 3 h, 72%; (g) LiOH, THF/MeOH/H₂O (3:1:1), room temperature, 1.5 h, 95%.

FIG. 7. Competitive binding assay of the lectin-targeted drug delivery conjugates and reference carbohydrates with (A) LecA, (B) LecB_{PA14} and (C) LecB_{PAO1}. One representative titration of triplicates on plate is shown for each compound. The corresponding IC₅₀-values were determined from at least three independent experiments and are given as a mean standard deviation (K₁ in table 2).

FIG. 8. Activation of the lectin-targeted conjugates in 50% human blood plasma spiked with 10% P. aeruginosa culture supernatant (CS): The ciprofloxacin-based prodrugs 24 and 25 do not release ciprofloxacin whilst the primary amide-based prodrugs 36, 37, 48 and 49 release their antibiotic cargo within the same time frame. PP=plasma proteins.

FIG. 9. Stability of the lectin-targeted conjugates in 50% human blood plasma spiked with 10% LB: All conjugates (24, 25, 36, 37, 48 and 49) show no significant release of their antibiotic cargo within the observed time frame.

FIG. 10. Stability of the dipeptide-conjugates 19 and 35 in PA14-filtrate without the addition of human blood plasma. The release of the free corresponding drugs by proteolysis was very slow compared to the presence of PA14-filtrate and human blood plasma. The experiment was performed in technical triplicates. The results are given as mean and standard deviation.

FIG. 11. Intracellular drug accumulation assay of the lectin-targeted conjugates 36/47 and their antibiotic cargo 2 on A549-cells at 10 μg/ml concentration. Data is shown as mean and standard deviation from two biological replicates with each two technical replicates. Statistical analysis calculated with two-way ANOVA and Tukey post-hoc test (p >0.05, ns; p<0.05, *).

FIG. 12. Exemplary H¹ and ¹³C-NMR spectrum of synthesized lectin-targeted conjugate 23.

FIG. 13. HPLC-UV analysis of purity of key compounds.

FIG. 14. Diagram of divalent fluorescent LecA ligands based on compound 50.

FIG. 15. Scheme for the chemical synthesis of divalent fluorescent LecA ligand. Reagents and conditions: (i) 4-nitrobenzyl bromide, K₂CO₃, room temperature, DMF, overnight; (ii) Fe, CaCl₂, EtOH/H₂O, 40° C., room temperature, 9d; (iii) 54, HBTU, DIPEA, DMF, room temperature, 2d; (iv) 56, CuSO₄, sodium ascorbate, DMF/H₂O, room temperature, 35° C., 6d.

FIG. 16. Evaluation of the divalent ligands binding with a central nitrogen atom to LecA in fluorescence polarization-based assays and in SPR. Averages and standard deviation from at least two independent experiments. One representative experiment is shown for each.

FIG. 17. Evaluation of the divalent fluorescent ligands 57 binding to LecA in ITC.

FIG. 18. HPLC-MS chromatogram of divalent fluorescent ligand 57 (m/z 754.672+) in TBS/Ca²⁺ buffer after 24 h.

FIG. 19. P. aeruginosa PAO1 staining with LecA targeting. P. aeruginosa PAO1 wildtype labelled with the red fluorescent protein mCherry (pMP7605) was grown in a flow system. Bacterial cells were diluted from an exponential phase culture to an optical density (OD600 nm) of 0.1 and injected into a six-channel μ-slide (ibidi, Germany). After 30 min of bacterial settling (no flow), diluted LB medium (1:5) was constantly flowing with a rate of 3 mL/h at 30° C. in a heat system (ibidi, Germany) to grow biofilms. After 48 h, 500 nM of compound were supplemented to the medium and constantly added to the biofilm: A) divalent fluorescent LecA ligand (57) and B) fluorescein disodium salt (58) as a control. Fluorescence microscopy was performed for the analysis. Representative snapshots are shown after 4 h of staining under a constant flow. mCherry signals were detected by an excitation wavelength of 540 to 580 nm and an emission wavelength of 592 to 668 nm. The fluorescein signal was detected by an excitation wavelength of 460 to 500 nm and an emission wavelength of 512 to 542 inn. Snapshots were normalized using the Fiji software based on intrinsic fluorescent intensities of 57 and 58. Scale bars=50 μm.

FIG. 20. Structure of Fluorescein disodium salt (58) that was used as a control in biofilm staining experiments.

FIG. 21. Scheme for chemical synthesis of divalent fluorescent LecA ligands. Reagents and conditions: (i) BDP-FL-azide/sulfoCy7-azide, CuSO₄, sodium ascorbate, DIPEA, DMF/H₂O, room temperature, 3 h, overnight.

FIG. 22. Exemplary H¹ and ¹³C-NMR spectrum of synthesized lectin-targeted conjugate 52.

FIG. 23. Exemplary H¹ and ¹³C-NMR spectrum of synthesized lectin-targeted conjugate 53.

FIG. 24. Exemplary H¹ and ¹³C-NMR spectrum of synthesized lectin-targeted conjugate 53.

FIG. 25. Exemplary H¹ and ¹³C-NMR spectrum of synthesized lectin-targeted conjugate 57.

FIG. 26. Exemplary H¹ and ¹³C-NMR spectrum of synthesized lectin-targeted conjugate 59.

FIG. 27. Exemplary H¹ and ¹³C-NMR spectrum of synthesized lectin-targeted conjugate 60.

FIG. 28. Scheme for chemical synthesis of the divalent LecA-targeted fluoroquinolone prodrug. Reagents and conditions: (a) HCl, dioxane, room temperature, 1 h; (b) TBTU, DIPEA, CH₂Cl₂, room temperature, 16 h; (c) LiOH, THF/MeOH/H₂O (3/2/2), room temperature, 30 min; (d) TBTU, DIPEA, DMF, room temperature, 4 h; (e) LiOH, HF/MeOH/H₂O (3/1/1), room temperature, 24 h; (f) cat. CuSO₄, cat. Sodium ascorbate, DMF/H₂O, room temperature, 2 h.

FIG. 29. Exemplary H¹ and ¹³C-NMR spectrum of synthesized lectin-targeted conjugate 61.

FIG. 30. Exemplary H¹ and ¹³C-NMR spectrum of synthesized lectin-targeted conjugate 62.

FIG. 31. Exemplary H¹ and ¹³C-NMR spectrum of synthesized lectin-targeted conjugate 64.

FIG. 32. Exemplary H¹ and ¹³C-NMR spectrum of synthesized lectin-targeted conjugate 65.

FIG. 33. Exemplary H¹ and ¹³C-NMR spectrum of synthesized lectin-targeted conjugate 66.

FIG. 34. Exemplary H¹ and ¹³C-NMR spectrum of synthesized lectin-targeted conjugate 67.

FIG. 35. Exemplary H¹ and ¹³C-NMR spectrum of synthesized lectin-targeted conjugate 68.

FIG. 36. Affinity measurement of bivalent precursor 55 (FIG. 36, part 1) and corresponding prodrug 68 (FIG. 36, part 2) by ITC.

FIG. 37. Result list from MEROPS database search for xxx-xxx-Ala-Gly-Leu-Ala-xxx-xxx (P3-P2-P1-/-P1′-P2′-P3′) (SEQ ID NO: 3) as substrate. Downward arrows indicate the cleavage site. Many of the listed hits are bacterial metalloproteases, e.g. thermolysin, bacillolysin, pseudolysin, griselysin and stearolysin. MMP-2, MMP-3, Cathepsin B and Granzyme B are mammalian proteases.

FIG. 38. UV-chromatograms of LecA-targeted prodrug 36 incubated in absence (top) or presence (middle) of activated MMP-2 in PBS. The prodrug showed high stability under both conditions. FRET-based cleavage assay with SensoLyte 520 generic MMP substrate showed successful activation of the protease (bottom).

FIG. 39. FRET-based cleavage assay of LasB substrate (A) or generic MMP substrate (B) in presence of a serial dilution of PA14-filtrate or MMP-2. While the LasB-substrate is rather stable against MMP-2, PA14 filtrate rapidly cleaves both substrates. Final concentrations are shown.

FIG. 40: Staining of P. aeruginosa PAO1 biofilms in the flow system by compound supplementation to the medium. Biofilm staining with (A) BODIPY divalent imaging probe 60 and (B) BDP FL azide control was observed after 4 h of constant flow (3 mL/h) of LB media containing 500 nM of respective compound (accumulation period). Retention of imaging compounds is shown after 30 min wash period with medium (3 mL/h, no compound). Images were recorded using fluorescence microscope. Green signal (the two photographs to the left in diagram A) corresponds to fluorescently labelled divalent LecA imaging probe (60) and the respective control. Signals were detected at an excitation wavelength of 460 to 500 nm and an emission wavelength of 512 to 542 nm. P. aeruginosa expressing mCherry shown in red (the two photographs to the right in diagram A and the two photographs to the right in diagram B) was detected at an excitation wavelength of 540 to 580 nm and an emission wavelength of 592 to 668 n. Scale bars=50 μm.

The invention is described by way of the following examples which are to be construed as merely illustrative and not limitative of the scope of the invention.

### EXAMPLES

### Example 1—Chemical synthesis

### 1. Synthesis of Lectin-Targeted Carbohydrate Peptide Conjugates

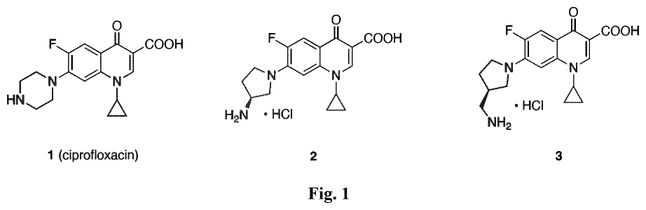

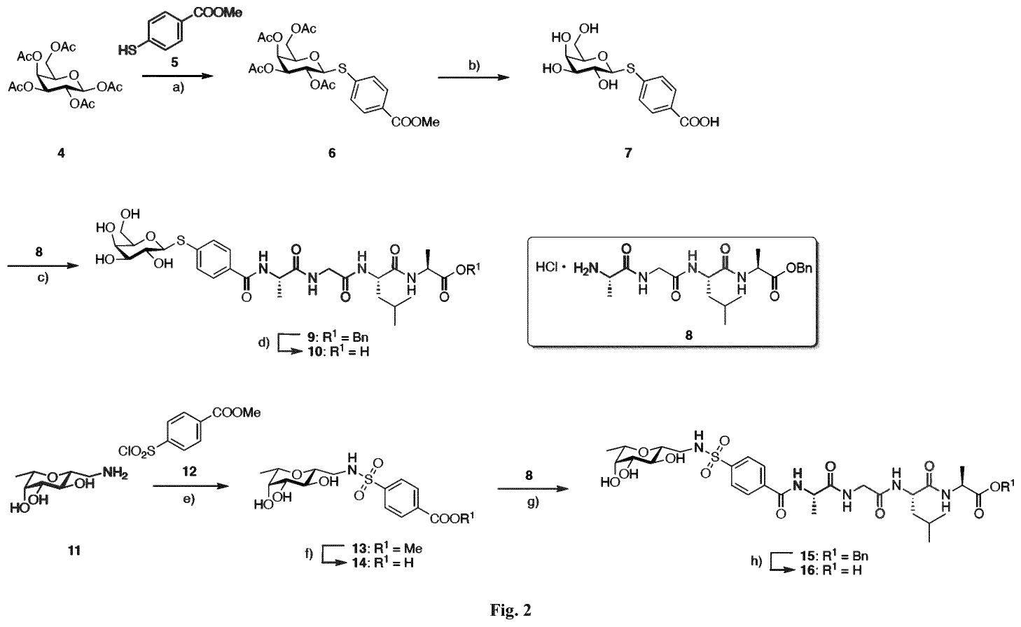

The LecA-targeted galactoside precursor 7 was synthesised in analogy to Novoa et al. (FIG. 2). In brief, Lewis acid-mediated glycosylation of methyl-4-mercaptobenzoate 5 with galactose pentaacetate 4 gave galactoside 6 in good yield. After subsequent global deprotection in two steps, LecA probe 7 was obtained quantitatively. Tetrapeptide 8 was synthesised in 5 chemical steps by solution-phase peptide synthesis. Peptide coupling of linker 8 to LecA-probe 7 was performed with TBTU yielding the protected conjugate 9. The following debenzylation proved to be problematic: Only small amounts of product 10 could be isolated by hydrogenolysis, even under elevated H₂-pressure (3.5 bar) and catalysis with Pd black. Eventually, saponification with LiOH gave the desired compound 10 in 49% yield (FIG. 2). LecB-targeted β-C-glycoside 11 was synthesised as reported in Sommer et al. 1995. Reaction with sulfonylchloride 12 resulted in sulfonamide 13, which was subsequently saponified with LiOH to yield the corresponding carboxylic acid 14. Conjugation to tetrapeptide 8 was again performed with TBTU to give benzyl-protected intermediate 15. In contrast to the troublesome deprotection of benzylester 9, debenzylation towards compounds 16 was achieved by hydrogenolysis with catalytic amounts of palladium on charcoal in excellent yields (FIG. 2). These lectin-targeted molecules were now available for the conjugation to the antibiotic cargo compounds 1-3 (FIG. 1).

### 2. Synthesis of Ciprofloxacin Series

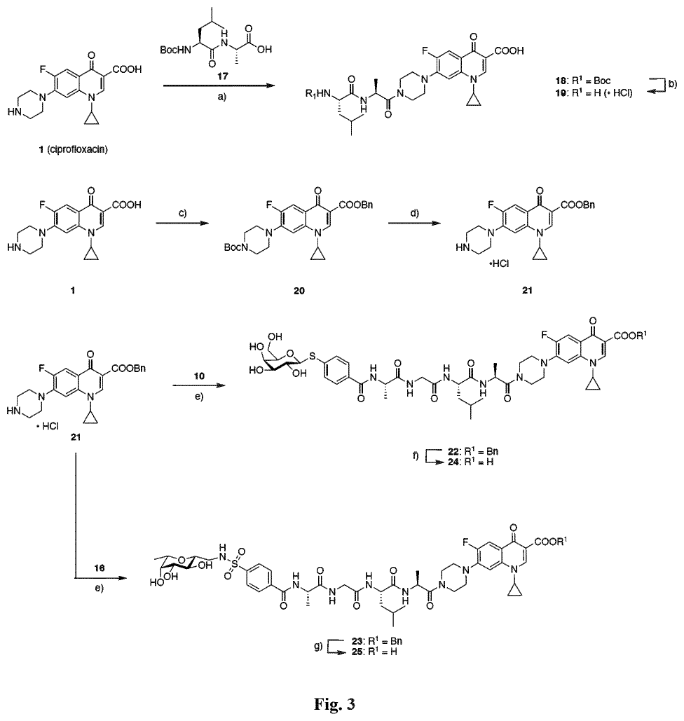

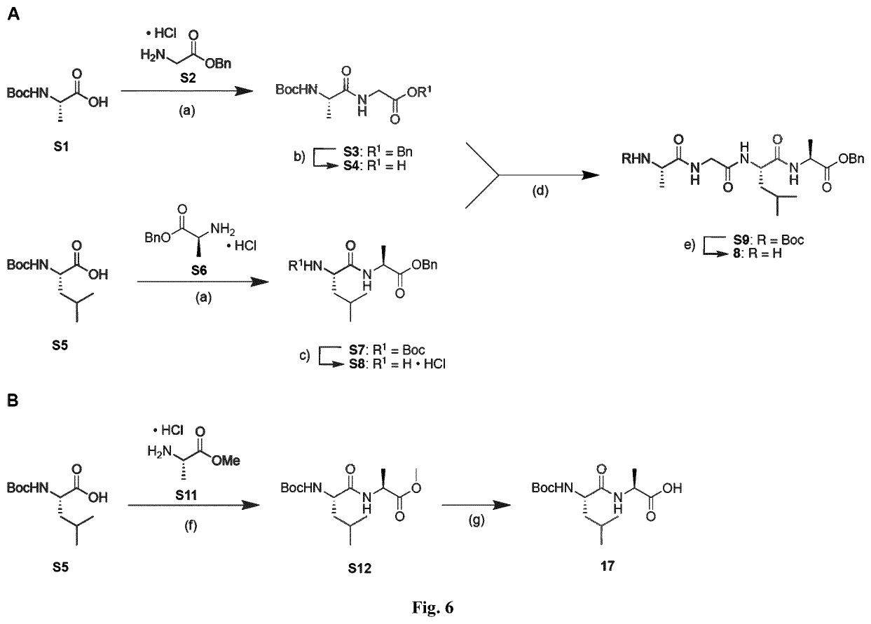

Boc-Leu-Ala 17 (FIG. 6) was coupled to ciprofloxacin 1 after activation with isobutyl chloroformate to give Boc-protected intermediate 18 which subsequently was deprotected by HCl to give dipeptidyl-ciprofloxacin as reference compound 19. The conjugation of the lectin-targeted peptide precursors 10 (LecA-targeted) and 16 (LecB-targeted) with benzyl-protected ciprofloxacin 21 towards the protected prodrugs 22 and 23 was performed by activation with TBTU. Hydrogenolytic deprotection resulted in the ciprofloxacin-based lectin-targeted prodrugs 24 and 25 (FIG. 3).

### 3. Synthesis of Aminopyrrolidine Series

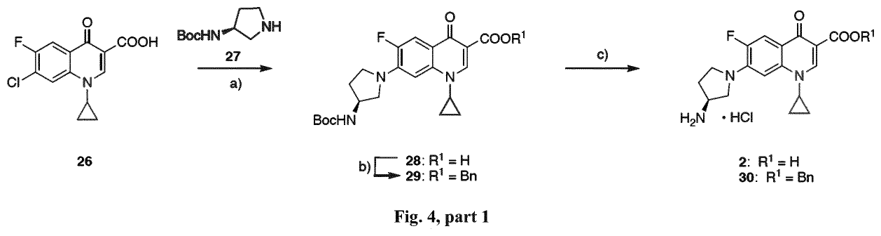

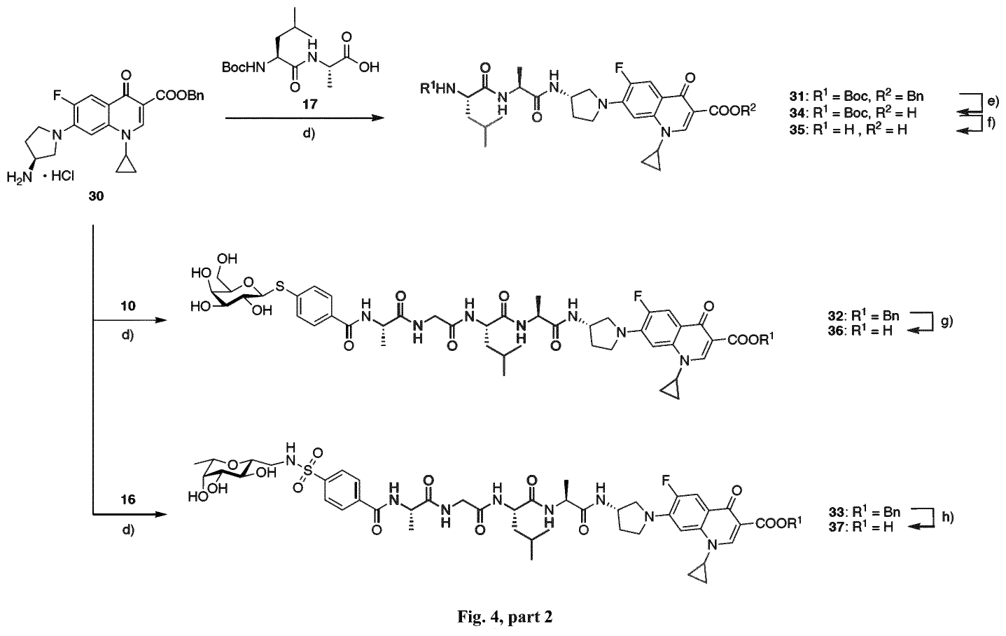

For the aminopyrrolidine series (FIG. 4), the fluoroquinolone core structure 26 was refluxed with aminopyrrolidin 27 in dry pyridine, according to Sanchez et al. After chromatographic separation of the regioisomers, Boc-protected fluoroquinolone 28 was reacted with benzylbromide to give fully protected aminomethylpyrrolidine-FQ 29 in good yield. On the other hand, a small amount of 28 was directly boc-deprotected under acidic conditions to obtain aminopyrrolidine-FQ 2 in excellent yield. In parallel, 29 was deprotected towards the free amine 30 and subsequently conjugated to Boc-Leu-Ala 17, LecA-targeted tetrapeptide precursor 10 and LecB-targeted tetrapeptide precursor 16 by activation with TBTU to result in the protected conjugates 31, 32 and 33, respectively. Dipeptidyl-FQ 31 was first deprotected by hydrogenolysis towards intermediate compound 34, which was then Boc-deprotected under acidic conditions to yield the aminopyrrolidine-FQ 35. The protected lectin-targeted conjugates 32 and 33 were hydrogenolytically deprotected to yield the two aminopyrrolidine-based prodrugs 36 (LecA-targeted) and 37 (LecB-targeted).

### 4. Synthesis of Aminomethylpyrrolidine Series

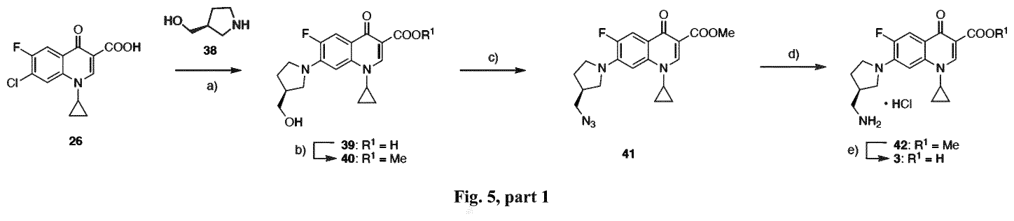

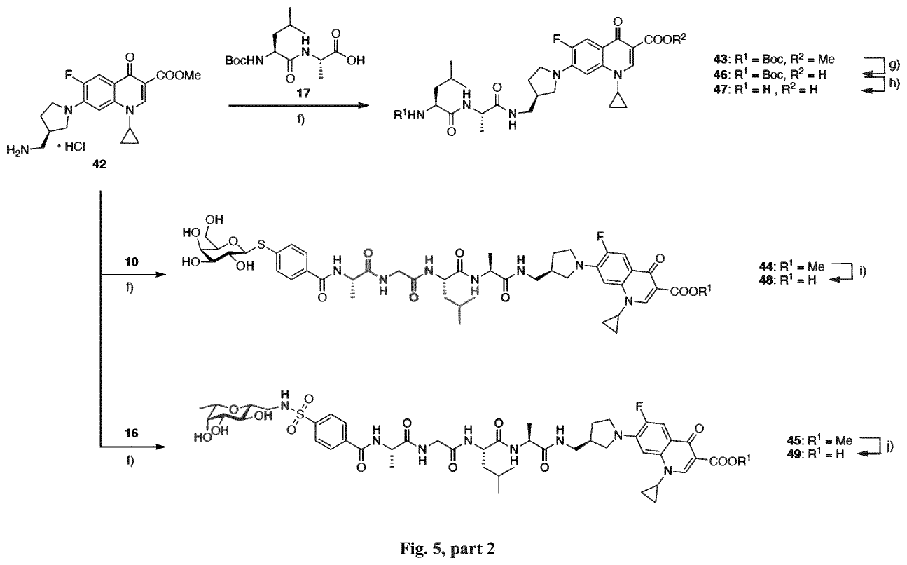

For the aminomethylpyrrolidine series (FIG. 5), the fluoroquinolone core structure 26 was refluxed with (S)-beta-prolinol 38 in dry pyridine. The desired regioisomer 39 precipitated from the reaction at room temperature. After CSA-catalysed esterification, methylester 40 was obtained in excellent yield. The following step towards the corresponding azide 41 contained several pitfalls: Transformation of the primary alcohol to a leaving group, e.g. with PBr₃ or TsCl led to decomposition of the starting material. Eventually, Bose-Mitsunobu conditions (diphenylphosphoryl azide, DPPA; diisopropyl azodicarboxylate, DIAD; PPh₃) gave azide 41 in one step. After hydrogenation, amine 42 was trapped as its HCl salt to prevent side-reaction with the methylester during workup. Saponification with LiOH yielded the corresponding reference antibiotic 3 in 43% yield. As in the aminopyrrolidine series, methyl-protected fluoroquinolone 42 was coupled to the peptides Boc-Leu-Ala 17, LecA-targeted tetrapeptide precursor 10 and LecB-targeted tetrapeptide precursor 16 after activation with TBTU to yield the protected intermediates 43, 44 and 45, respectively. Dipeptide 43 was then saponified with LiOH towards carboxylic acid 46. After deprotection under acidic conditions, reference aminomethylpyrrolidine-FQ 47 was obtained. The methyl-protected lectin-targeted conjugates 44 and 45 were deprotected by saponification to yield the two aminomethylpyrrolidine-based prodrugs 48 (LecA-targeted) and 49 (LecB-targeted) in good yields.

### 5. Synthesis of Intermediate Compounds of Lectin-Targeted Cleavable Fluoroquinolone Conjugates

### 5.1 Methyl 4-mercaptobenzoate (5)

5 was synthesised according to the protocol from Novoa et al. 2014: 4-mercapto benzoic acid (5.2 g, 34 mmol, 1 eq.) was dissolved in 50 mL dry MeOH (purged with Ar) and treated to 6 drops conc. H₂SO₄. The mixture was refluxed for three days. After cooling to room temperature, the pH was adjusted to 5 using NaOMe (1 M in MeOH) and the solution was loaded on silica in vacuo. The product was eluted with CH₂Cl₂. After evaporation of the solvent, the product (4.8 g, 80%, 93% purity determined by ¹H-NMR) was used without further purification. ¹H NMR (500 MHz, CHCl3-d) δ 7.89 (d, J=8.4 Hz, 2H, Ar—H), 7.29 (d, J=8.4 Hz, 2H, Ar—H), 3.90 (s, 3H, COOMe), 3.62 (s, 1H, SH).

### 5.2 p-Methylbenzoyl 2,3,4,6-Tetra-O-acetyl-β-d-thiogalactopyranoside (6)

6 was synthesised according to a previously reported protocol from Novoa et al. 2014: β-d-Galactose pentaacetate (3 g, 7.7 mmol, 1 eq.) was dissolved in 20 mL dry CH₂Cl₂ in a heat-dried flask under a N₂ atmosphere. The solution was cooled (0° C.) and BF₃·Et₂O (3.8 mL, 30.7 mmol, 4 eq.) was added dropwise under vigorous stirring. A solution of 5 (3 eq., 0.4 M) was added dropwise to the reaction. The reaction was allowed to warm to room temperature and stirred overnight. After the reaction was quenched with ice water, the organic phase was subsequently washed with satd. aqueous NaHCO₃ (2×), water (2×) and satd. brine (2×). The combined organic layers were dried over Na₂SO₄ and the solvent was evaporated in vacuo. After purification by MPLC (SiO₂, EtOAc in toluene, 5-30%), the title compound was crystallised from a mixture of EtOAc in hexane (1:1) and obtained as white crystals (2.2 g, 58%). ¹H NMR (500 MHz, MeOH-d4) δ 7.96 (d, J=8.5 Hz, 2H, Ar—H), 7.58 (d, J=8.5 Hz, 2H, Ar—H), 5.46 (dd, J=2.9, 1.1 Hz, 1H, glyco-H-4), 5.26-5.17 (m, 2H, glyco-H₂, glyco-H-3), 5.15 (dd, J=8.7, 1.4 Hz, 1H, glyco-H-1), 4.25 (td, J=6.4, 1.2 Hz, 1H, glyco-H-5), 4.16 (d, J=6.0 Hz, 2H, glyco-H₆), 3.90 (s, 3H, COOMe), 2.14 (s, 3H, Ac—CH₃), 2.05 (s, 3H, Ac—CH₃), 2.03 (s, 3H, Ac—CH₃), 1.94 (s, 3H, Ac—CH₃). ¹³C NMR (126 MHz, MeOH-d₄) δ 172.03 (Ac—C═O), 171.88 (Ac—C═O), 171.36 (Ac—C═O), 171.18 (Ac—C═O), 168.01(C═OOMe), 141.30 (Ar—C), 131.07 (Ar—C), 130.85 (Ar—C), 129.99 (Ar—C), 85.58 (glyco-C-1), 75.60 (glyco-C-5), 73.25 (glyco-C-2), 69.02 (glyco-C-2), 68.41 (glyco-C-4), 63.01 (glyco-C-3), 52.71 (COOCH₃), 20.63 (Ac—CH₃), 20.60 (Ac—CH₃), 20.48 (2× Ac—CH₃). LR-MS: m/z=521.1 [M+Na]⁺.

### 5.3 p-Methylbenzoyl-Q-d-thiogalactopyranoside (7)

6 (1 g, 2.0 mmol, 1 eq.) was dispersed in 20 mL dry MeOH. A solution of NaOMe (300 μL, 1 M, 1.5 eq.) in MeOH was added dropwise while cooling on ice. The reaction was allowed to warm to room temperature and stirred for 30 min. A solution of LiOH (50 mg, 2 mmol, 1 eq.) in 3 mL water was added to the reaction. After 1 h, the pH was adjusted to 4 with Amberlite IR-120 H⁺ exchange resin while cooling on ice. The resin was removed by filtration and the solvent was evaporated in vacuo. The title compound was obtained as a white amorphous solid (630 mg, quant.) containing approximately 17% NaOAc as an impurity. ¹H NMR (500 MHz, MeOH-d4) δ 7.88 (d, J=8.5 Hz, 2H, Ar—H), 7.51 (d, J=8.4 Hz, 2H, Ar—H), 4.70 (d, J=9.8 Hz, 1H, glyco-H-1), 3.94 (dd, J=3.3, 1.0 Hz, 1H, glyco-H-4), 3.77 (dd, J=11.5, 6.8 Hz, 1H, glyco-H-6), 3.72 (dd, J=11.5, 5.3 Hz, 1H, glyco-H-6), 3.68-3.58 (m, 2H, glyco-H-2, glyco-H-5), 3.54 (dd, J=9.2, 3.4 Hz, 1H, glyco-H₃). ¹³C NMR (126 MHz, MeOH)-d4 δ 174.26 (COOH), 139.62 (Ar—C), 136.33 (Ar—C), 130.78 (Ar—CH), 130.10 (Ar—CH), 89.51 (glyco-C-1), 80.55 (glyco-C-5), 76.27 (glyco-C-3), 70.95 (glyco-C-2), 70.42 (glyco-C-4), 62.56 (glyco-C-6). LR-MS: m/z=315.1 [M−H]⁻.

### 5.4 Bn-protected. LecA-targeted peptide linker (9)

7 (316 mg, 1 mmol, 1 eq.), 8 (590 mg, 1.3 mmol, 1.3 eq.) and TBTU (414 mg, 1.3 mmol, 1.3 eq.) were dissolved in 10 mL dry DMF. DIPEA (360 μL, 2 mmol, 2 eq.) was added dropwise and the reaction was stirred for 1 h. The solvent was evaporated in vacuo and the reaction was purified by MPLC (MeCN in EtOH/H₂O (1:1) 5-15%). The title compound was obtained as a white amorphous solid (420 mg, 58%), approximately 15% contaminated with coupling reagents. ¹H NMR (500 MHz, MeOH-d4) δ 8.33 (d, J=7.1 Hz, 1H, NH), 7.98 (d, J=8.1 Hz, 1H, NH), 7.88-7.81 (m, 2H, glyco-Ar—H), 7.65-7.55 (m, 2H, glyco-Ar—H), 7.39-7.25 (m, 5H, Bn), 5.16 (d, J=12.3 Hz, 2H, Bn), 5.12 (d, J=12.3 Hz, 1H, Bn), 4.72 (d, J=9.7 Hz, 1H, glyco-H-1), 4.49-4.37 (m, 2H, Ala-Cα-H, Ala′-Cα-H, Leu-Cα-H), 3.95 (d, J=16.9 Hz, 1H, gly-Cα-H), 3.92 (dd, J=3.4, 0.9 Hz, 1H, glyco-H-4), 3.79 (d, J=17.0 Hz, 1H, gly-Cα-H), 3.79-3.68 (m, 2H, glyco-H-6), 3.66 (t, J=9.4 Hz, 1H, glyco-H-2), 3.62 (ddd, J=6.7, 5.1, 1.1 Hz, 1H, glyco-H-5), 3.52 (dd, J=9.1, 3.3 Hz, 1H, glyco-H-3), 1.74-1.50 (m, 3H, Leu-CH, Leu-CH₂), 1.48 (d, J=7.2 Hz, 3H, Ala-CH₃), 1.41 (d, J=7.3 Hz, 3H, Ala′—CH₃), 0.84 (d, J=6.0 Hz, 3H, Leu-CH₃), 0.80 (d, J=5.9 Hz, 3H, Leu-CH₃). ¹³C NMR (126 MHz, MeOH-d4) δ 176.17 (CONH), 174.63 (CONH), 173.76 (CONH), 171.59 (CONH), 169.71 (COOBn), 142.01 (Ar—C), 137.26 (Ar—C), 132.53 (Ar—C), 130.03 (Ar—C), 129.59 (Ar—C), 129.30 (Ar—C), 129.24 (Ar—C), 129.15 (Ar—C), 89.04 (glyco-C-1), 80.76 (glyco-C-5), 76.31 (glyco-C-3), 70.90 (glyco-C-2), 70.41 (glyco-C-4), 67.93, 62.65 (glyco-C-6), 52.93 (Leu-Ca), 52.00 (Ala-Cα), 49.70 (Leu-Ca), 43.75 (Gly-Ca), 41.73 (Leu-CH), 25.64 (Leu-CH₂), 23.44 (Leu-CH₃), 21.77 (Leu-CH₃), 17.27 (Ala′—CH₃), 17.22 (Ala-CH₃). LR-MS: m/z=719.3 [M+H]⁺.

### 5.5 LecA-targeted peptide linker (10)

9 (116 mg, 0.16 mmol, 1 eq.) was dissolved in 2 mL DMF at 50° C. LiOH (35 mg, 9 eq.) was dissolved in 1 mL H₂O and added stepwise over three days until full turnover was observed. The reaction was neutralised with Amberlite IR-120 H⁺ exchange resin. After filtration, the solvent was removed via lyophilisation. The product was purified by preparative HPLC (MeCN:H₂O, 5-30%, 0.1% formic acid) and obtained as a white amorphous solid (49 mg, 49%). ¹H NMR (500 MHz, DMSO-d6) δ 12.44 (br s, 1H, COOH), 8.60 (d, J=6.6 Hz, 1H, NH), 8.25 (t, J=5.8 Hz, 1H, NH), 8.16 (d, J=7.1 Hz, 1H, NH), 7.84 (d, J=8.5 Hz, 2H, Ar—H), 7.76 (d, J=8.5 Hz, 1H, NH), 7.50 (d, J=8.5 Hz, 2H, Ar—H), 5.21 (d, J=6.1 Hz, 1H, OH), 4.91 (br s, 1H, OH), 4.71 (d, J=9.6 Hz, 1H, glyco-H-1), 4.66 (br s, 1H, OH), 4.52 (d, J=4.4 Hz, 1H, OH), 4.45-4.31 (m, 2H, Ala-Cα-H), 4.16 (dq, J=7.3, 7.3 Hz, 1H, Ala-Cα-H), 3.76-3.60 (m, 3H, Gly-CH₂, glyco-H-4), 3.57-3.42 (m, 4H, glyco-H-6, glyco-H-2, glyco-H-5), 3.38 (dd, J=9.5, 3.1 Hz, 1H, glyco-H-3), 1.56 (dh, J=13.3, 6.5 Hz, 1H, Leu-CH), 1.51-1.41 (m, 2H, Leu-CH₂), 1.34 (d, J=7.2 Hz, 3H, Ala-CH₃), 1.27 (d, J=7.3 Hz, 3H, Ala-CH₃), 0.82 (d, J=2.5 Hz, 3H, Leu-CH₃), 0.80 (d, J=2.6 Hz, 3H, Leu-CH₃). ¹³C NMR (126 MHz, DMSO-d6) δ 174.04 (C═O), 173.03 (C═O), 171.73 (C═O), 168.51 (C═O), 165.95 (COOH), 140.30 (Ar—C), 130.82 (Ar—C), 128.06 (Ar—C), 127.53 (Ar—C), 86.63 (glyco-C-1), 79.32 (glyco-C-5), 74.68 (glyco-C-3), 69.16 (glyco-C-2), 68.41 (glyco-C-4), 60.62 (glyco-C-6), 50.43 (Leu-Ca), 49.55 (Ala-Cα), 47.50 (Ala-Cα), 42.17 (Gly-Ca), 40.91 (Leu-CH₂), 23.94 (Leu-CH), 23.14 (Leu-CH₃), 21.62 (Leu-CH₃), 17.52 (Ala-CH₃), 17.00 (Ala-CH₃). LR-MS: m/z=629.2 [M+H]⁺.

### 5.6 N-9-1-Fucopyranosylmethyl-2-(p-carboxvbenzyl-methyl)-sulfonamide (13)

11 (400 mg, 2.26 mmol, 1 eq.) was dissolved in dry DMF (15 mL) and K₂CO₃ (625 mg, 4.52 mmol, 2 eq.) was added while cooling on ice. Sulfonylchloride 12 (970 mg, 4.13 mmol, 1.8 eq.) was dissolved in dry DMF (5 mL) and added dropwise to the starting material. The ice batch was removed and the reaction was stirred at room temperature overnight. The reaction was quenched with MeOH (1 mL) and neutralised to pH 7 with HCl (1 M) while cooling on ice. The solvent was removed in vacuo and the reaction was purified by MPLC (SiO₂, MeOH in CH₂Cl₂,1-10%). The title compound was obtained as a white amorphous solid (228 mg, 27%). ¹H NMR (500 MHz, DMSO-d6) δ 8.13 (d, J=8.4 Hz, 2H, Ar—H), 7.94 (d, J=8.5 Hz, 2H, Ar—H), 7.81 (br s, 1H, NHSO2), 4.78 (d, J=5.3 Hz, 1H, OH), 4.59 (d, J=5.7 Hz, 1H, OH), 4.26 (d, J=5.4 Hz, 1H, OH), 3.89 (s, 3H, COOMe), 3.39-3.35 (m, 1H, glyco-H-4), 3.29 (q, J=6.2 Hz, 1H, glyco-H-5), 3.24 (d, J=13.4 Hz, 1H, linker-CH₂), 3.18 (ddd, J=9.0, 5.7, 3.2 Hz, 1H, glyco-H-3), 3.11 (td, J=9.2, 4.7 Hz, 1H, glyco-H-1), 2.96 (td, J=8.8, 2.2 Hz, 1H, glyco-H-2), 2.78-2.68 (m, 1H, linker-CH₂), 1.01 (d, J=6.4 Hz, 6H, glyco-H-6). ¹³C NMR (126 MHz, DMSO-d6) δ 165.28 (COOMe), 145.10 (Ar—C), 132.63 (Ar—C), 129.88 (Ar—C), 126.89 (Ar—C), 78.33 (glyco-C-2), 74.58 (glyco-C-3), 73.57 (glyco-C-5), 71.51 (glyco-C-4), 68.22 (glyco-C-1), 52.61 (COOCH₃), 44.56 (linker-CH₂), 16.86 (glyco-C-6). LR-MS: m/z=376.1 [M+H]⁺. 5.7 N-9-1-Fucopyranosylmethyl-2-(p-carboxvbenzyl)-sulfonamide (14) 13 (224 mg, 0.60 mmol, 1 eq.) was dissolved in a mixture of THF, MeOH and H₂O (3:1:1, 7 mL) and LiOH (72 mg, 3 mmol, 5 eq.) was added. The reaction was stirred overnight at room temperature until disappearance of the starting material. After neutralisation with Amberlite IR-120 H⁺ to pH 7, the solvents were removed in vacuo. The title compound was obtained after lyophilisation as white powder (206 mg, 95%). The compound was also poorly soluble in MeOH and water. 1H NMR (500 MHz, D20) δ 8.19 (d, J=8.6 Hz, 1H, Ar—H), 7.99 (d, J=8.5 Hz, 1H, Ar—H), 3.69 (d, J=3.3 Hz, 1H, glyco-H-2), 3.49 (dd, J=9.6, 3.4 Hz, 1H, glyco-H-1), 3.44 (q, J=6.5 Hz, 1H, glyco-H-5), 3.42-3.37 (m, 2H, glyco-linker-CH₂, glyco-H-4), 3.20-3.05 (m, 2H, glyco-linker-CH₂, glyco-H-3), 1.10 (d, J=6.5 Hz, 3H, glyco-H-6). ¹³C NMR (126 MHz, D20) δ 169.46 (COOH), 142.73 (Ar—C), 134.86 (s, Ar—C), 130.49 (Ar—C), 126.84 (Ar—C), 77.43 (glyco-C-3), 74.11 (glyco-C-1), 73.92 (glyco-C-5), 71.66 (glyco-C-2), 68.03 (glyco-C-4), 43.92 (linker-CH₂), 15.65 (glyco-C-6).

¹H NMR (500 MHz, DMSO-d4) δ 8.15-8.05 (m, 2H, Ar—H), 7.96-7.86 (m, 2H, Ar—H), 7.77 (t, J=6.0 Hz, 1H, NHSO₂), 4.79 (br s, 1H, OH), 4.60 (br s, 1H, OH), 4.26 (br s, 1H, OH), 3.24 (ddd, J=13.3, 6.2, 2.2 Hz, 1H, linker-CH₂), 3.18 (dd, J=9.1, 3.4 Hz, 1H, glyco-H-3), 3.11 (t, J=9.1 Hz, 1H, glyco-H-1), 2.97 (td, J=8.8, 2.4 Hz, 1H, glyco-H-2), 2.73 (ddd, J=13.7, 8.5, 5.6 Hz, 1H, linker-CH₂), 1.02 (d, J=6.4 Hz, 3H, glyco-C-6). ¹³C NMR (126 MHz, DMSO-d6) δ 166.34 (COOH), 144.68 (Ar—C), 134.00 (Ar—C), 129.95 (Ar—C), 126.74 (Ar—C), 78.34 (glyco-C-2), 74.60 (glyco-C-3), 73.59 (glyco-C-5), 71.53 (glyco-C-4), 68.24 (glyco-C-1), 44.57 (linker-CH₂), 16.86 (glyco-C-6).

### 5.8 Bn-protected LecB-targeted peptide linker (15)

14 (200 mg, 0.55 mmol, 1 eq.), 8 (302 mg, 0.66 mg, 1.2 eq.) and TBTU (267 mg, 0.83 mmol, 1.5 eq.) were dissolved in dry DMF (10 mL). DIPEA (288 μL, 1.65 mmol, 3 eq.) was added dropwise and the reaction was stirred for 1 h. The solvent was evaporated in vacuo and the reaction was purified by RP-MPLC (C₁₈-phase, MeCN in Water, 10-35%, 0.1% formic acid). After lyophilisation, the title compound was isolated as a white powder (336 mg, 80%). ¹H NMR (500 MHz, MeOH-d4) δ 8.06 (d, J=8.5 Hz, 2H, ArH), 7.95 (d, J=8.4 Hz, 2H, ArH), 7.41-7.27 (m, 5H, Bn), 5.17 (d, J=12.3 Hz, 1H, Bn), 5.12 (d, J=12.3 Hz, 1H, Bn), 4.49-4.40 (m, 3H, Ala-Cα-H, Ala′-Ca—H, Leu-Cα-H), 3.97 (d, J=16.8 Hz, 1H, Gly-Cα-H), 3.79 (d, J=16.9 Hz, 1H, Gly-Cα-H), 3.58 (dd, J=2.9, 0.7 Hz, 1H, glyco-H-4), 3.44 (qd, J=6.4, 1.1 Hz, 1H, glyco-H-5), 3.41-3.36 (m, 2H, glyco-H-3, glyco-H-1), 3.34 (dd, J=8.3, 2.9 Hz, 1H, linker-CH₂), 3.11 (ddd, J=9.0, 7.1, 2.4 Hz, 1H, glyco-H-2), 3.04 (dd, J=12.9, 7.1 Hz, 1H, linker-CH₂), 1.68-1.52 (m, 3H, Leu-CH, Leu-CH₂), 1.50 (d, J=7.2 Hz, 3H, Ala′—CH₃), 1.41 (d, J=7.4 Hz, 3H, Ala′—CH₃), 1.17 (d, J=6.4 Hz, 3H, glyco-C₆—H), 0.84 (d, J=5.9 Hz, 3H, Leu-CH₃), 0.78 (d, J=6.0 Hz, 3H, Leu-CH₃). ¹³C NMR (126 MHz, MeOH-d4) δ 175.85 (CONH), 174.62 (COOBn), 173.76 (CONH), 171.53 (CONH), 168.83 (CONH), 145.04 (Ar—C), 138.56 (Ar—C), 137.25 (Bn-C), 129.59 (Bn-C), 129.51 (Ar—C), 129.32 (Bn-C), 129.25 (Bn-C), 128.10 (Ar—C), 79.61 (glyco-C-2), 76.33 (glyco-C-3), 75.52 (glyco-C-5), 73.56 (glyco-C-4), 69.62 (glyco-C-1), 67.93 (Bn-CH₂), 52.88 (Ala-Cα), 52.04 (Ala′-Ca), 49.67 (Leu-Ca), 45.55 (linker-CH₂), 43.72 (Gly-Ca), 41.75 (Leu-CH₂), 25.63 (Leu-CH), 23.44 (Leu-CH₃), 21.78 (Leu-CH₃), 17.24 (Ala-CH₃), 17.21 (Ala-CH₃), 17.10 (glyco-C₆—H). LR-MS: m/z=764.2 [M+H]⁺.

### 5.9 LecB-targeted peptide linker (16)

15 (300 mg, 0.39 mmol, 1 eq.) was dissolved in MeOH (4 mL). Pd/C (10% m/m, 41 mg, 10 mol %) was added and the atmosphere was changed to H₂ (1 atm.). The reaction was stirred at room temperature for 16 h until full transformation of the starting material. Pd/C was removed by centrifugation (17600 ref, 10 min) and the solvent was removed in vacuo. The title compound was obtained as a white amorphous solid (250 mg, 95%). H NMR (500 MHz, MeOH-d4) δ 8.87 (d, J=5.5 Hz, 1H, NH), 8.56 (t, J=5.9 Hz, 1H, NH), 8.22 (d, J=7.2 Hz, 1H, NH), 8.07 (d, J=8.5 Hz, 2H, Ar—H), 7.98 (s, 1H, NH), 7.96 (d, J=8.6 Hz, 2H, Ar—H), 4.56-4.43 (m, 2H, Leu-Cα-H, Ala-Cα-H), 4.37 (qd, J=7.3, 2.3 Hz, 1H, Ala-Cα-H), 3.99 (d, J=17.0 Hz, 1H, Gly-Cα-H), 3.81 (d, J=16.8 Hz, 1H, Gly-Cα-H), 3.59 (d, J=2.9 Hz, 1H, glyco-H-4), 3.44 (q, J=6.6 Hz, 1H, glyco-H-5), 3.41-3.32 (m, 3H, glyco-H-3, glyco-H-1, linker-CH₂), 3.11 (ddd, J=9.0, 7.2, 2.4 Hz, 1H, glyco-H-2), 3.03 (dd, J=12.9, 7.2 Hz, 1H, linker-CH₂), 1.74-1.58 (m, 3H, Leu-CH, Leu-CH₂), 1.51 (d, J=7.2 Hz, 3H, Ala-CH₃), 1.41 (d, J=7.3 Hz, 3H, Ala′—CH₃), 1.17 (d, J=6.6 Hz, 3H, glyco-H-6), 0.88 (d, J=6.3 Hz, 3H, Leu-CH₃), 0.82 (d, J=6.3 Hz, 3H, Leu-CH₃). ¹³C NMR (126 MHz, MeOH-d4) δ 175.85 (CONH), 175.80 (COOH), 174.44 (CONH), 171.55 (CONH), 168.85 (CONH), 145.04 (Ar—C), 138.56 (Ar—C), 129.51 (Ar—C), 128.11 (Ar—C), 79.62 (glyco-C-2), 76.32 (glyco-C-3), 75.52 (glyco-C-5), 73.56 (glyco-C-4), 69.63 (glyco-C-1), 52.96 (Ala-Cα), 52.03 (Ala′-Ca), 49.85 (Leu-Ca), 45.55 (linker-CH₂), 43.72 (Gly-Ca), 41.73 (Leu-CH₂), 25.65 (Leu-CH), 23.46 (Leu-CH₃), 21.81 (Leu-CH₃), 17.61 (Ala-CH₃), 17.23 (Ala′—CH₃), 17.09 (glyco-C₆—H). LR-MS: m/z=674.2 [M+H]⁺.

### 5.10 Boc-protected Leu-Ala-ciprofloxacin-conjugate (18)

Dipeptide 17 (100 mg, 0.33 mmol, 1 eq.) and NMM (36 μL, 0.33 mmol, 1 eq.) were dissolved in 3 mL. The solution was cooled to −20° C. with a cooling bath (ice, sodium chloride) and Ibef (43 μL, 0.33 mmol, 1 eq.) was added dropwise under vigorous stirring. The reaction was stirred for 20 min. This solution was then added dropwise to a dispersion of Ciprofloxacin (119 mg, 0.36 mmol, 1.1 eq.) and NMM (51 μL, 0.46 mmol, 1.4 eq.) in dry THF (4 mL) via a transfer channel. The reaction was allowed to warm to room temperature and stirred for 2.5 h. The reaction was poured on ice-water (20 mL) and acidified with aqueous HCl (1 M) to pH=4. The aqueous phase was extracted with CH₂Cl₂(3×20 mL). The combined organic layers were washed with with satd. aqueous NH₄Cl and dried over Na₂SO₄. The solvent was removed in vacuo and the product was purified by MPLC (CHCl₃/PE (9: 1): MeOH, 1-10%), yielding the product as a beige amorphous solid (50 mg, 25%). ¹H NMR (500 MHz, CHCl3-d) δ 8.78 (s, 1H, cipro-C2-H), 8.05 (d, J=12.6 Hz, 1H, cipro-C5-H), 7.45 (d, J=5.3 Hz, 1H, cipro-C8-H), 7.01 (d, J=7.0 Hz, 1H, Ala-NH), 5.03-4.89 (m, 1H, Ala-Cα-H), 4.88-4.83 (m, 1H, Leu-NH), 4.14 (s, 1H, Leu-Cα-H), 4.09-3.98 (m, 1H, piperazine-C—H), 3.95-3.80 (m, 1H, piperazine-CH), 3.74-3.67 (m, 2H, piperazine-C—H), 3.56 (s, 1H, cPr-H), 3.39 (s, 3H, piperazine-C—H), 3.30-3.15 (m, 1H, piperazine-C—H), 1.75-1.59 (m, 3H, Leu-CH₂+Leu-CH—CH₃CH₃), 1.44 (s, 12H, cPr-CH₂+Boc-CH₃+Leu-CH₂′), 1.37 (d, J=6.6 Hz, 3H, Ala-CH₃), 1.21 (s, 2H, cPr-CH₂), 1.00-0.81 (m, 6H, Leu-CH³). ¹³C NMR (126 MHz, CDCl3-d) δ 177.21 (cipro-C4=0), 172.07 (C═O), 170.77 (C═O), 166.99 (cipro-COOH), 155.69 (carbamate-C═O), 153.77 (d, J=251.8 Hz, cipro-C-6), 147.85 (cipro-C-2), 145.13 (cipro-C-7), 139.09 (cipro-C-8a), 120.85 (d, J=7.8 Hz, cipro-C-4a), 112.92 (d, J=23.2 Hz, cipro-C-5), 108.45 (cipro-C-3), 105.71 (cipro-C-8), 80.34 (Boc), 53.40 (Leu-Ca), 50.16 (piperazine-C), 49.84 (piperazine-C), 45.27 (piperazine-C), 45.07 (Leu-Ca), 41.91 (piperazine-C), 41.57 (Leu-CH₂), 35.54 (cPr-CH), 28.42 (Boc-CH₃), 24.92 (Leu-CH—CH₃CH₃), 23.20 (Leu-CH₃), 21.89 (Leu-CH₃), 18.96 (Ala-CH³), 8.46 (cPr-CH₂).

### 5.11 Leu-Ala-ciprofloxacin-conjugate (19)

Boc-Protected conjugate 18 (43 mg, 0.07 mmol, 1 eq.) was dissolved in 3 mL HCl in dioxane (4 N) while cooling on ice. The ice bath was removed and the reaction was stirred at room temperature for 4 h. The solvent was evaporated in vacuo and the residue was dissolved in 1 mL MeOH. The product was precipitated with ice-cold Et₂O (20 mL) and the resulting precipitate was washed three times with ice-cold Et₂O. The precipitate was dried in vacuo and obtained as a yellow solid (30 mg, 78%). ¹H NMR (500 MHz, DMSO-d6) δ 15.17 (s, 1H, cipro-COOH), 8.85 (d, J=7.5 Hz, 1H, Ala-NH), 8.67 (s, 1H, cipro-C2-H), 8.27 (s, 1H, Leu-NH³⁺), 7.93 (d, J=13.1 Hz, 1H, cipro-C5-H), 7.58 (d, J=7.2 Hz, 1H, cipro-C8-H), 4.92-4.82 (m, 1H, Ala-Cα-H), 3.82 (s, 2H, Leu-Cα-H⁺ cPr-CH), 3.78-3.73 (m, 2H, piperazine-C—H), 3.73-3.69 (m, 2H, piperazine-C—H), 1.68 (dp, J=13.2, 6.6 Hz, 1H, Leu-CH—CH₃CH₃), 1.59-1.49 (m, 2H, Leu-CH₂), 1.32 (d, J=6.3 Hz, 2H, cPr-CH₂), 1.28 (d, J=6.9 Hz, 2H, Ala-CH₃), 1.19 (s, 2H, cPr-CH₂), 0.91 (d, J=6.5 Hz, 3H, Ala-CH₃), 0.89 (d, J=6.5 Hz, 3H, Ala-CH₃). ¹³C NMR (126 MHz, DMSO-d6) δ 176.39 (cipro-C4), 169.95 (C═O), 168.29 (C═O), 165.92 (cipro-COOH), 152.96 (d, J=249.3 Hz, cipro-C6), 148.15 (cipro-C2), 144.80 (d, J=10.1 Hz, cipro-C7), 139.15 (cipro-C8a), 118.92 (d, J=7.6 Hz, cipro-C4a), 111.08 (d, J=23.0 Hz, cipro-C5), 106.79 (cipro-C3), 106.71 (d, J=2.7 Hz, cipro-C8), 50.62 (Leu-Ca), 49.70 (piperazine-C), 49.19 (piperazine-C), 44.65 (piperazine-C), 44.57 (Ala-Cα), 41.21 (piperazine-C), 40.23 (Leu-CH₂), 35.94 cPr-CH, 23.53 (Leu-CH—CH₃CH₃), 22.68 (Leu-CH₃), 22.04 (Leu-CH₃), 17.75 (Ala-CH₃), 7.64 (cPr-CH₂). HR-MS calculated [C₂₆H₃₅FN₅O₅]⁺: 516.2617, found 516.2610.

### 5.12 (N-boc)-ciprofloxacin-benzylester (20)

Ciprofloxacin (1) (1000 mg, 3.02 mmol, 1 eq.) and KHCO₃ (1511 mg, 15.1 mmol, 5 eq.) and Boc₂0 (775 μL, 3.62 mmol, 1.2 eq.) were dispersed in 12 mL dry DMF. The reaction was heated to 40° C. and stirred for 2 h. Then, BnBr (430 μL, 3.62 mmol, 1.2 eq.) was added and the reaction was heated to 120° C. and stirred for 90 min. The reaction was allowed to cool to room temperature and poured on 100 mL ice cold water. The precipitate was filtered off and dried in vacuo. The product was obtained as a beige amorphous solid (1.36 g, 86%). No NMR measured due to solubility issues: the sample degraded in CDCl₃ and was not soluble in other common solvents. LR-MS: m/z=522.3 [M+H]⁺.

### 5.13 Ciprofloxacin-benzylester·HCl (21)

20 (500 mg, 0.96 mmol, 1 eq.) was partially dissolved in 2 mL CH₂Cl₂ and cooled with an ice-bath. 10 mL HCl in dioxane (4 N) was added slowly under vigorous stirring and the reaction was allowed to warm to room temperature and stirred for 1 h. The solvent was evaporated in vacuo and the product was obtained as a yellow solid (448 mg, quant.). ¹H NMR (500 MHz, DMSO-d6) δ 9.54 (s, 2H, piperazine-NH₂+), 8.48 (s, 1H, cipro-C2-H), 7.81 (d, J=13.2 Hz, 1H, cipro-C5-H), 7.51-7.46 (m, 3H, cipro-C8-H⁺ Bn-phenyl), 7.42-7.37 (m, 2H, Bn-phenyl), 7.35-7.30 (m, 1H, Bn-phenyl), 5.27 (s, 2H, Bn-CH₂), 3.73-3.64 (m, 1H, cPr-CH), 3.52-3.45 (m, 4H, piperazine-C—H), 3.30 (s, 4H, piperazine-C—H), 1.25 (dd, J=6.9 Hz, 2H, cPr-CH₂), 1.15-1.04 (m, 2H, cPr-CH₂). ¹³C NMR (126 MHz, DMSO-d6) δ 172.05 (C═O), 164.96 (COOBn), 152.94 (d, J=246.5 Hz, cipro-C6), 149.06 (cipro-C2), 143.28 (d, J=10.9 Hz, cipro-C7), 138.50 (cipro-C8a), 137.10 (Bn), 128.86 (Bn), 128.25 (Bn), 128.09 (Bn), 123.03 (d, J=6.3 Hz, cipro-C4a), 112.30 (d, J=22.7 Hz, cipro-C5), 109.42 (cipro-C3), 107.19 (cipro-C8), 65.73 (piperazine-C), 46.93 (piperazine-C), 46.90 (piperazine-C), 43.01 (piperazine-C), 35.39 (cPr-CH), 8.05 (cPr-CH₂). LR-MS: m/z=422.1 [M+H]⁺.

### 5.14 LecA-targeted ciprofloxacin-prodrug 22

The title compound was synthesised in two chemical steps: First, 10 (31 mg, 0.049 mmol, 1 eq.), 21 (34 mg, 0.074 mmol, 1.5 eq.) and TBTU (24 mg, 0.074 mmol, 1.5 eq.) were dissolved in 1 mL dry DMF. DIPEA (17 μL, 0.098 mmol, 2 eq.) was added dropwise and the reaction was stirred for 1 h. After evaporation of the solvent, the residue was taken up in 1.5 mL MeOH/DMF (2:1). Pd black (10 mg, 0.05 mmol, 1 eq.) was added and the reaction was stirred under H₂ atmosphere for 6 d. Afterwards, the reaction was filtered over celite and further purified by preparative HPLC (MeCN:H₂O, 20-33%, 0.1% formic acid). The title compound was obtained as a beige amorphous solid (10 mg, 22% over 2 chemical steps). ¹H NMR (500 MHz, MeOH-d4) δ 8.79 (s, 1H, FQ-H-2), 7.91 (d, J=13.0 Hz, 1H, FQ-H-5), 7.81 (d, J=8.1 Hz, 2H, Ar—H), 7.61 (d, J=6.8 Hz, 1H, FQ-C₈—H), 7.54 (d, J=8.0 Hz, 2H, Ar—H), 4.92-4.87 (m, 1H, Ala-Cα-H), 4.72 (d, J=9.7 Hz, 1H, glyco-H-1), 4.48 (dd, J=10.7, 3.8 Hz, 1H, Leu-Cα-H), 4.40 (q, J=6.0 Hz, 1H, Ala-Cα-H), 4.06-3.88 (m, 3H, pip-CH, pip-CH, Gly-CH, glyco-H-4), 3.83-3.59 (m, 8H, pip-CH, pip-CH, Gly-CH, cPr-CH, glyco-H₆, glyco-H-2, glyco-H-5), 3.53 (dd, J=9.2, 3.4 Hz, 1H, glyco-H-3), 3.43 (s, 3H, pip-CH₂, pip-CH), 1.77-1.66 (m, 1H, Leu-CH₂), 1.67-1.57 (m, 2H, Leu-CH₂, Leu-CH), 1.48 (d, J=7.1 Hz, 3H, Ala-CH₃), 1.42 (d, J=6.3 Hz, 2H, cPr-CH₂), 1.36 (d, J=6.8 Hz, 3H, Ala-CH₃), 1.27-1.18 (m, 2H, cPr-CH₂), 0.87 (d, J=5.7 Hz, 3H, Leu-CH₃), 0.82 (d, J=5.8 Hz, 3H, Leu-CH₃). ¹³C NMR (126 MHz, MeOH-d4) δ 177.03 (C═O), 174.85 (C═O), 172.80 (C═O), 171.14 (C═O), 170.36 (C═O), 168.31 (C═O), 168.26 (C═O), 153.70 (d, J=250.0 Hz, FQ-C-6), 148.03 (FQ-C), 145.50 (d, J=10.1 Hz, FQ-C-7), 140.79 (FQ-C), 139.38 (Ar—C), 130.87 (Ar—C), 128.35 (Ar—C), 127.77 (Ar—C), 119.59 (d, J=8.1 Hz, FQ-C-4a), 111.15 (d, J=23.5 Hz, FQC-5), 106.80 (FQ-C), 106.31 (d, J=2.7 Hz, FQ-C-8), 87.53 (glyco-C-1), 79.38 (glyco-C-5), 74.86 (glyco-C-3), 69.47 (glyco-C-2), 68.99 (glyco-C-4), 61.26 (glyco-C-6), 51.70 (Leu-Ca), 50.77 (Ala-Cα), 49.66 (d, J=3.7 Hz, pip-C), 49.28 (d, J=2.6 Hz, pip-C), 45.25 (Ala-Cα), 45.08 (Gly-Ca), 42.49 (pip-C), 41.80 (pip-C), 40.17 (Leu-CH₂), 35.67 (cPr-CH), 24.31 (Leu-CH), 22.16 (Leu-CH₃), 20.18 (Leu-CH₃), 16.40 (Ala-CH₃), 15.87 (Leu-CH₃), 7.24 (cPr-CH₂), 7.17 (cPr-CH₂). HR-MS calculated [C₄₄H₅₇FN₇O₁₃S]⁺: 942.3714, found 942.3689.

### 5.15 LecB-targeted ciprofloxacin-prodrug-benzylester (23)

16 (70 mg, 0.10 mmol, 1 eq.), 21 (55 mg, 0.12 mg, 1.2 eq.) and TBTU (48 mg, 0.15 mmol, 1.5 eq.) were dissolved in dry DMF (2 mL). DIPEA (52 μL, 0.3 mmol, 3 eq.) was added dropwise and the reaction was stirred for 1 h. The solvent was evaporated in vacuo and the reaction was purified by pHPLC (MeCN in Water, 25-40%, 0.1% formic acid). After lyophilisation, the title compound was isolated as an off-white powder (90 mg, 84%). ¹H NMR (500 MHz, MeOHd4) δ 8.65 (s, 1H, FQ-H-2), 8.04 (d, J=8.5 Hz, 2H, glyco-Ar—H), 7.93 (d, J=8.5 Hz, 2H, glyco-Ar—H), 7.88 (d, J=13.3 Hz, 1H, FQ-H-5), 7.52 (d, J=7.2 Hz, 1H, FQ-H-8), 7.48 (d, J=7.4 Hz, 2H, Bn), 7.42-7.35 (m, 2H, Bn), 7.34-7.29 (m, 1H, Bn), 5.33 (s, 2H, Bn-CH₂), 4.92-4.88 (m, 1H, Ala-Cα-H), 4.49-4.42 (m, 2H, Leu-Cα-H, Ala′-Cα-H), 4.00 (d, J=16.8 Hz, 1H, gly-Cα-H), 3.94-3.84 (m, 2H, pip-H, pip-H), 3.78 (d, J=16.8 Hz, 1H, glycin-Cα-H), 3.77-3.70 (m, 1H, pip-H), 3.70-3.61 (m, 2H, cPr-CH, pip-H), 3.58 (d, J=2.9 Hz, 1H, glyco-H-4), 3.43 (q, J=6.2 Hz, 1H, glyco-H-5), 3.40-3.34 (m, 4H, pip-H, pip-H, pip-H, glyco-H-3, glyco-H-1), 3.34-3.27 (m, 1H, glyco-linker-CH₂), 3.28-3.22 (m, 1H, pip-H), 3.10 (ddd, J=9.0, 7.1, 2.4 Hz, 1H, glyco-H-2), 3.02 (dd, J=12.9, 7.1 Hz, 1H, glyco-linker-CH₂), 1.76-1.56 (m, 3H, Leu-CH, Leu-CH₂), 1.50 (d, J=7.2 Hz, 3H, Ala-CH₃), 1.35 (d, J=6.9 Hz, 3H, Ala-CH₃), 1.34-1.25 (m, 2H, cPr-CH₂), 1.16 (d, J=6.5 Hz, 3H, glyco-H-6), 1.14-1.06 (m, 2H, cPr-CH₂), 0.87 (d, J=5.8 Hz, 3H, Leu-CH₃), 0.82 (d, J=5.7 Hz, 3H, Leu-CH₃). ¹³C NMR (126 MHz, MeOH-d4) δ 175.83 (CONH), 175.43 (FQ-C═O), 174.11 (CONH), 172.55 (CONH), 171.68 (CONH), 165.93 (COOBn), 154.88 (d, J =248.4 Hz, FQC-6), 150.13 (FQ-Ar—C), 145.93 (d, J=10.6 Hz, FQ-C-7), 145.06 (Ar—C), 139.87 (Ar—C), 138.52 (Ar—C), 137.93 (Ar—C), 129.56 (glyco-Ar—C), 129.53 (Bn), 129.24 (Bn), 129.15 (Bn), 128.08 (glyco-Ar—C), 123.70 (d, J=6.5 Hz, FQ-C-4a), 113.18 (d, J=24.3 Hz, FQ-C-5), 110.28 (Ar—C), 107.58 (d, J=2.9 Hz, FQ-C-8), 79.62 (glyco-C-2), 76.33 (glyco-C-3), 75.51 (glyco-C-5), 73.55 (glyco-C-4), 69.62 (glyco-C-1), 67.16 (Bn), 53.11 (Leu-Ca), 52.13 (Ala-Cα), 51.25 (pip-C), 50.78 (pip-C), 46.58 (pip-C), 45.55 (glyco-linker-CH₂), 43.89 (gly-Ca), 43.22 (pip-C), 41.60 (Leu-CH₂), 36.38 (cPr-CH), 25.74 (Leu-CH), 23.53 (Leu-CH₃), 21.63 (Leu-CH₃), 17.87 (Ala-CH₃), 17.23 (Ala-CH₃), 17.11 (glyco-C-6), 8.60 (cPr-CH₂), 8.54 (cPr-CH₂). LR-MS: m/z=539.2 [M+2H]².

### 5.16 LecB-targeted ciprofloxacin-prodrug (25)

23 (57 mg, 0.052 mmol, 1 eq.) was dissolved in MeOH (1 mL). Pd/C (10% m/m, 5 mg, 10 mol %) was added and the atmosphere was changed to H₂ (1 atm.). The reaction was stirred at room temperature for 24 h until full consumption of the starting material. Pd/C was removed by centrifugation (17600 ref, 10 min) and the solvent was removed in vacuo. After purification by pHPLC (MeCN in Water, 22-35%, 0.1% formic acid), the title compound was obtained as an off-white amorphous solid (38 mg, 74%). ¹H NMR (500 MHz, MeOH-d4) δ 8.60 (s, 1H, FQH-2), 8.05 (d, J=8.0 Hz, 2H, glyco-Ar—H), 7.94 (d, J=8.0 Hz, 2H, glyco-Ar—H), 7.72 (d, J=14.1 Hz, 1H, FQ-H-5), 7.05 (s, 1H, FQ-H-8), 4.47 (s, 1H, pyrr-H), 4.41 (q, J=7.3 Hz, 1H, Ala-Cα-H), 4.36-4.23 (m, 2H, Ala′-Cα-H), 3.97-3.77 (m, 3H, pyrr-H, pyrr-H, gly-Cα-H), 3.74-3.61 (m, 4H, pyrr-H, pyrr-H, gly-Cα-H, cPr-CH), 3.58 (d, J=1.9 Hz, 1H, glyco-H-4), 3.44 (q, J=6.4 Hz, 1H, glyco-H-5), 3.41-3.27 (m, 4H, glyco-H-3, glyco-H-1, pyrr-H, glyco-linker-CH₂), 3.10 (ddd, J=8.9, 7.5, 2.4 Hz, 1H, glyco-H-2), 3.02 (dd, J=12.9, 7.1 Hz, 1H, glyco-linker-CH₂), 2.34-2.22 (m, 1H, pyrr-H), 2.21-2.10 (m, 1H, pyrr-H), 1.78-1.69 (m, 1H, Leu-CH), 1.69-1.56 (m, 2H, Leu-CH₂), 1.51 (d, J=7.1 Hz, 3H, Ala-CH₃), 1.44-1.35 (m, 5H, Ala′-CH₃, cPr-CH₂), 1.18 (s, 2H, cPr-CH₂), 1.16 (d, J=6.5 Hz, 3H, glyco-C-6), 0.86 (d, J=4.6 Hz, 3H, Leu-CH₃), 0.85 (d, J=4.6 Hz, 3H, Leu-CH₃). ¹³C NMR (126 MHz, MeOH-d4) δ 177.54 (d, J=2.3 Hz, FQ-C═O), 176.27 (CONH), 175.08 (CONH), 174.75 (CONH), 172.35 (CONH), 170.05 (CONH), 169.06 (COOH), 152.04 (d, J=247.5 Hz, FQ-C-6), 148.59 (FQ-C-2), 145.13 (Ar—C), 143.54 (d, J=12.1 Hz, FQ-C-7), 141.38 (Ar—C), 138.36 (Ar—C), 129.54 (glyco-Ar—C), 128.11 (glyco-Ar—C), 111.96 (d, J=23.9 Hz, FQC-5), 101.66 (FQ-C-8), 79.62 (glyco-C-2), 76.33 (glyco-C-3), 75.52 (glyco-C-5), 73.55 (glyco-C-4), 69.61 (glyco-C-1), 56.00 (d, J=6.4 Hz, pyrr-C), 53.88 (Leu-Ca), 52.48 (Ala-Cα), 50.93 (pyrr-H), 50.84 (Ala′—CH₃), 49.24 (extracted from HSQC, pyrr-C) 45.55 (glycolinker-CH₂), 44.20 (gly-Ca), 41.19 (Leu-CH₂), 36.80 (cPr-CH), 31.80 (pyrr-C), 25.81 (Leu-CH), 23.53 (Leu-CH₃), 21.56 (Leu-CH₃), 17.85 (Ala-CH₃), 17.27 (Ala′—CH₃), 17.11 (glyco-C-6), 8.49 (cPr-CH₂), 8.46 (cPr-CH₂). HR-MS calculated [C₄₅H₆oFN₈O1₄S]⁺: 987.3928, found 987.3908.

### 5.17 (S)-7-(3-Tertbutoxycarbonylamino-1-pyrrolidinyl)-1-cyclopropyl-6-fluoro-1,4-dihydro-4-oxo-1,8-naphthyridine-3-carboxylic acid (28)

7-chloro-1-cyclopropyl-6-fluoro-4-oxo-1,4-dihydroquinoline-3-carboxylic acid (26, 500 mg, 1.78 mmol, 1 eq.) and (S)-3-(Boc-amino)-pyrrolidine (995 mg, 5.34 mmol, 3 eq.) were dispersed in 10 mL dry pyridine. The mixture was heated with an oil bath to 160° C. and refluxed overnight. After cooling to room temperature, the solvent was evaporated in vacuo and purified by NP-MPLC (CH₂C₂: n-Hex (70:28): MeOH, 2-5%). The product was obtained as a beige amorphous solid (383 mg, 50%). ¹H NMR (500 MHz, DMSO-d6) δ 15.54 (s, 1H, COOH), 8.56 (s, 1H, ArH-2), 7.78 (d, J=14.1 Hz, 1H, ArH-5), 7.30 (d, J=6.7 Hz, 1H, BocNH), 7.03 (d, J=7.6 Hz, 1H. ArH-8), 4.17 (q, J=6.3 Hz, 1H), 3.86-3.77 (m, 1H), 3.77-3.67 (m, 1H), 3.64-3.56 (m, OH), 3.44 (dt, J=10.7, 3.9 Hz, 1H), 2.15 (dq, J=13.3, 7.1 Hz, 1H), 1.93 (dq, J=12.6, 6.0 Hz, 1H), 1.39 (s, 9H), 1.34-1.25 (m, 1H), 1.18-1.10 (m, 1H). ¹³C NMR (126 MHz, DMSO-d6) δ 175.87 (d, J=3.3 Hz, C-4), 166.32 (COOH), 155.29 ((Boc-C═O), 149.96 (d, J=246.3 Hz, C-6), 147.44 (C), 141.65 (d, J=11.6 Hz, C-7), 139.83 (C), 114.42 (d, J=7.0 Hz, C-4a), 110.71 (d, J=22.6 Hz, C-5), 106.17 (C), 100.43 (d, J=5.7 Hz, C-8), 78.04 (Boc-C), 55.18 (d, J=6.8 Hz, aminopyrrolidine-C), 49.84 (aminopyrrolidine-C), 48.15 (d, J=3.8 Hz, aminopyrrolidine-C), 35.74 (cPr-C), 30.43 (aminopyrrolidine-C), 28.28 (Boc-CH₃), 7.59 (cPr-CH₂), 7.53 (cPr-CH₂). LR-MS: m/z=432.2 [M+H]⁺.

### 5.18 (S)-7-(3-Amino-1-pyrrolidinyl)-1-cyclopropyl-6-fluoro-1,4-dihydro-4-oxo-1,8-naphthyridine-3-carboxylic acid (2)

28 (59 mg, 0.14 mmol, 1 eq.) was dissolved in 3 mL HCl in dioxane (4 N) while cooling on ice. The reaction was allowed to warm to room temperature and stirred for 90 min until full consumption of the starting material. After the solvent was evaporated in vacuo, the remaining solid was taken up in 2 mL MeOH and the product was precipitated with Et₂O. The precipitate was first washed three times with Et₂O, then dried in vacuo and the product was obtained as a yellow amorphous solid (m=45 mg, 86%). 1H NMR (500 MHz, DMSO-d6) δ 15.46 (s, 1H, COOH), 8.60 (s, 1H, H-2), 8.33 (s, 3H, NH₃+), 7.86 (d, J=14.2 Hz, 1H, H-5), 7.10 (d, J=7.6 Hz, 1H, H-8), 4.10-3.88 (m, 2H, aminopyrrolidine-CH +cPr-CH), 3.88-3.69 (m, 3H, aminopyrrolidine-CH₂, aminopyrrolidine-CH), 3.69-3.61 (m, 1H, aminopyrrolidine-CH), 2.34 (ddt, J=14.0, 8.0, 7.5 Hz, 1H, aminopyrrolidine-CH), 2.15 (ddt, J=12.2, 7.8, 4.5 Hz, 1H, aminopyrrolidine-CH), 1.34-1.27 (m, 2H, cPr-CH₂), 1.23-1.11 (m, 2H, cPr-CH₂). ¹³C NMR (126 MHz, D20) δ 173.95 (d, C-4), 169.31 (C), 150.05 (d, J=249.4 Hz, C-6), 147.02 (C), 141.31 (d, J=11.0 Hz, C-7), 139.14 (C), 113.20 (d, J=7.1 Hz, C-4a), 109.51 (d, J=23.2 Hz, C-5), 104.62 (C), 100.57 (C-8), 52.62 (d, J=7.8 Hz, aminopyrrolidine-C), 50.11 (aminopyrrolidine-C), 47.27 (d, J=3.2 Hz, aminopyrrolidine-C), 35.79 (cPr-CH), 28.36 (aminopyrrolidine-C), 7.18 (2× cPr-CH₂). HR-MS calculated [Cl7H19FN303]⁺: 332.1405, found 332.1397.

### 5.19 (S)-7-(3-Tertbutoxycarbonylamino-1-pyrrolidinyl)-1-cyclopropyl-6-fluoro-1,4-dihydro-4-oxo-1,8-naphthyridine-3-carboxylic acid benzyl ester (29)

28 (364 mg, 0.84 mmol, 1 eq.) and freshly grinded KHCO₃ were dried on high vacuum for 15 min. After dispersion in 10 mL dry DMF, BnBr (150 μL, 1.26 mmol, 1.5 eq.) was added and the reaction was heated to 110° C. Full conversion was achieved after 60 min and the reaction was allowed to cool to room temperature. The solvent was reduced in vacuo and diluted with CH₂C₂. The organic phase was washed with water, KHSO₄ (1 M) and satd. brine. The combined organic layers were dried over Na₂SO₄ and the solvent was evaporated in vacuo, giving the title compound as a white amorphous solid (217 mg, 50%). ¹H NMR (500 MHz, DMSO-d6) δ 8.40 (s, 1H, H-2), 7.68 (d, J=14.5 Hz, 1H, H-5), 7.51-7.46 (m, 2H, OBn), 7.42-7.36 (m, 2H, OBn), 7.36-7.29 (m, 1H, OBn), 7.28 (d, J=6.7 Hz, 1H, NH), 6.94 (d, J=7.6 Hz, 1H, H-8), 5.25 (s, 2H, OBn-CH₂), 4.23-4.08 (m, 1H, aminopyrrolidine-CH), 3.78-3.71 (m, 1H, aminopyrrolidine-CH), 3.69-3.62 (m, 1H, aminopyrrolidine-CH), 3.59 (tt, J=7.2, 4.0 Hz, 1H, cPr-CH), 3.56-3.49 (m, 1H, aminopyrrolidine-CH), 3.43-3.36 (m, 1H, aminopyrrolidine-CH), 2.13 (dddd, J=13.5, 6.9, 6.9, 6.9 Hz, 1H, aminopyrrolidine-CH), 1.91 (dddd, J=12.5, 6.2, 6.2, 6.2 Hz, 1H, aminopyrrolidine-CH), 1.39 (s, 9H, Boc-CH₃), 1.25-1.20 (m, 2H, cPr-CH₂), 1.09-1.03 (m, 2H, cPr-CH₂). ¹³C NMR (126 MHz, DMSOd6) δ 171.51 (d, J=2.0 Hz, C-4), 164.77 (COOBn), 155.29 (Boc-C═O), 149.47 (d, J=242.8 Hz, C-6), 148.15 (C), 140.37 (d, J=11.8 Hz, C-7), 138.64 (C), 136.79 (Bn-C), 128.42 (Bn-C), 127.77 (Bn-C), 127.62 (Bn-C), 117.82 (d, J=5.9 Hz, C-4a), 111.54 (d, J=22.8 Hz, C-5), 108.53 (C), 100.53 (d, J=5.3 Hz, C-8), 77.98 (Boc-C), 65.14 (Bn-CH₂), 55.09 (d, J=5.0 Hz, aminopyrrolidine-C), 49.85 (aminopyrrolidine-C), 47.98 (d, J=4.7 Hz, aminopyrrolidine-C), 34.73 (cPr-CH), 30.47 (aminopyrrolidine-C), 28.28 (Boc-CH₃), 7.57 (cPr-CH), 7.52 (cPr-CH). LR-MS: m/z=522.2 [M+H]⁺.

### 5.20 (S)-7-(3-Amino-1-pyrrolidinyl)-1-cyclopropyl-6-fluoro-1,4-dihydro-4-oxo-1,8-naphthyridine-3-carboxylic acid benzyl ester (30)

29 (187 mg, 0.36 mmol, 1 eq.) was dissolved in 4 mL HCl in dioxane (4 N) while cooling on ice. The reaction was allowed to warm to room temperature and stirred for 1 h until full consumption of the starting material. After the solvent was evaporated in vacuo, the remaining solid was taken up in 2 mL MeOH and the product was precipitated with Et₂O. The precipitate was first washed three times with Et₂O, then dried in vacuo and the product was obtained as a yellow amorphous solid (160 mg, 97%). ¹H NMR (500 MHz, MeOH-d4) δ 8.85 (s, 1H, H-2), 7.81 (d, J=14.1 Hz, 1H, H-5), 7.53 (d, J=7.0 Hz, 2H, Bn-H), 7.47-7.32 (m, 3H, Bn-H), 6.96 (d, J=7.3 Hz, 1H, H-8), 5.49 (d, J=8.4 Hz, 1H, Bn-CH₂), 5.39 (d, J=12.1 Hz, 1H, Bn-CH₂), 4.18-4.07 (m, 2H, aminopyrrolidine-CH+aminopyrrolidine-CH), 4.05-3.98 (m, 1H, aminopyrrolidine-CH), 3.81-3.67 (m, 3H, aminopyrrolidine-CH₂+cPr-CH), 2.50 (dddd, 1H, aminopyrrolidine-CH), 2.27 (dddd, J=16.0, 5.8, 3.0 Hz, 1H, aminopyrrolidfine-CH), 1.49-1.30 (m, 2H, cPr-CH₂), 1.23-1.07 (m, 2H, cPr-CH₂). ¹³C NMR (126 MHz, MeOH-d4) δ 171.63 (d, J=3.6 Hz, C-4), 167.13 (COOBn), 152.61 (d, J=250.1 Hz, C-6), 149.77 (C), 144.39 (d, J=12.0 Hz, C-7), 141.62 (C), 136.83 (Bn-C), 129.92 (Bn-C), 129.84 (Bn-C), 129.79 (Bn-C), 115.09 (d, J=8.2 Hz, C-4a), 112.27 (d, J=24.7 Hz, C-5), 106.16 (C), 101.91 (d, J=5.9 Hz, C-8), 68.76 (Bn-CH₂), 54.92 (d, J=9.2 Hz, aminopyrrolidine-C), 51.82 (d, J=3.0 Hz, aminopyrrolidine-C), 38.04 (cPr-CH), 29.90 (aminopyrrolidine-C), 8.69 (2× cPr-CH₂). LRMS: m/z=422.2 [M+H]⁺.

### 5.21 7-((S)-3-((S)-2-((S)-2-((tert-butoxycarbonyl)amino)-4-methylpentanamido) propanamido)pyrrolidin-1-yl)-1-cyclopropyl-6-fluoro-4-oxo-1,4-dihydroquinoline-3-carboxylate (31)

30 (55 mg, 0.12 mg, 1 eq.), dipeptide 17 (54 mg, 0.18 mmol, 1.5 eq.) and DIPEA (100 μL, 0.6 mmol, 5 eq.) were dissolved in 900 μL dry DMF. TBTU (77 mg, 0.24 mmol, 2 eq.) was added and the reaction was heated to 40° C. Reaction progress was monitored by TLC (CH₂Cl₂:MeOH, 95:5) and full turnover was achieved after 1 h. The reaction concentrated in vacuo and diluted with CH₂Cl₂. The organic phase was washed with KHSO₄ (1 M), aq. satd. NaHCO₃ and satd. brine. The combined organic layers were dried over Na₂SO₄ and the solvent was evaporated in vacuo. After purification via NP-MPLC (CH₂Cl₂:MeOH, 1-5%), the title compound was obtained as a beige amorphous solid (63 mg, 74%). ¹H NMR (500 MHz, Acetone-d6) δ 8.47 (s, 1H, FQ-H-2), 8.21 (d, J=7.0 Hz, 1H, NH), 7.62-7.51 (m, 3H, Bn-H, FQ-H-5), 7.47-7.38 (m, 2H, Bn-H), 7.37-7.31 (m, 1H, Bn-H), 6.73 (d, J=7.5 Hz, 1H, FQ-H-8), 6.25 (d, J=8.1 Hz, 1H, NH), 5.35 (d, J=12.6 Hz, 1H, Bn-CH₂), 5.26 (d, J=12.6 Hz, 1H, Bn-CH₂), 4.57-4.52 (m, 1H, aminopyrrolidine-H), 4.48 (dq, J=7.2, 7.1 Hz, 1H, Ala-Cα-H), 4.09-4.00 (m, 1H, Leu-Cα-H), 3.92 (ddd, J=10.3, 6.0, 3.8 Hz, 1H, aminopyrrolidine-H), 3.62 (dt, J=9.7, 3.1 Hz, 1H, aminopyrrolidine-H), 3.56-3.46 (m, 1H, aminopyrrolidine-H), 3.45-3.37 (m, 2H, aminopyrrolidine-H, cPr-CH), 2.24-2.11 (m, 1H, aminopyrrolidine-H), 2.03-1.96 (m, 1H, aminopyrrolidine-H), 1.65 (ddd, J=13.1, 13.1, 6.6 Hz, 1H, Leu-CH₂), 1.57-1.49 (m, 2H, Leu-CH₂, Leu-CH), 1.40 (s, 9H, Boc-CH₃), 1.33 (d, J=7.1 Hz, 3H, Ala-CH₃), 1.26-1.20 (m, 1H, cPr-CH₂), 1.20-1.09 (m, 2H, cPr-CH₂, cPr-CH₂), 1.00-0.93 (m, 1H, cPr-CH₂), 0.85 (d, J=6.6 Hz, 3H, Leu-CH₃), 0.80 (d, J=6.5 Hz, 3H, Leu-CH₃). ¹³C NMR (126 MHz, Acetone-d6) δ 172.11 (d, J=4.6 Hz, FQC═O), 171.85 (C═O), 171.78 (C═O) 164.70 (COOBn), 155.69 (Boc-C═O), 149.71 (d, J=240.1 Hz, FQ-C-6), 147.77 (FQ-C), 140.87 (d, J=11.6 Hz, (FQ-C-7), 138.53 (FQ-C), 137.13 (Bn-C), 128.42 (Bn-C), 128.18 (Bn-C), 127.87 (Bn-C), 118.04 (d, J=9.4 Hz, FQC-4a), 111.94 (d, J=22.3 Hz, FQ-C-5), 109.01 (FQ-C), 100.34 (FQ-C-8), 78.46 (Boc-C), 65.43 (Bn-CH₂), 55.12 (aminopyrrolidine-C), 53.35 (Leu-Ca), 49.65 (aminopyrrolidine-C), 48.77 (Ala-Cα), 47.44 (Leu-Ca), 40.69 (Leu-CH₂), 34.43 (cPr-CH), 31.18 (aminopyrrolidine-C), 27.68 (Boc-CH₃), 24.50 (Leu-CH), 22.58 (Leu-CH₃), 20.91 (Leu-CH₃), 18.24 (Ala-CH₃), 7.51 (cPr-CH₂), 7.43 (cPr-CH₂). LR-MS: m/z=706.4 [M+H]⁺.

### 5.22 7-((S)-3-((S)-2-((S)-2-amino-4-methylpentanamido)propanamido)pyrrolidin-1-vl)-1-cyclopropyl-6-fluoro-4-oxo-1,4-dihydroquinoline-3-carboxylic acid (35)

The title compound was synthesized from 31 over two chemical steps. 31 (60 mg, 0.09 mmol, 1 eq.) was dissolved in 1 mL MeOH and Pd (5 mg, 0.05 mmol, 0.5 eq.) was added. The reaction was stirred under H₂ atmosphere (1 atm) over night at room temperature. The Palladium was removed via centrifugation (17,600 ref, 5 min) and the solvent was evaporated in vacuo. Residual solid was dissolved in HCl in dioxane (4 N) while cooling on ice. The reaction was allowed to warm to room temperature. After disappearance of the starting material (1 h), the solvent was evaporated in vacuo. The remaining solid was taken up in 2 mL MeOH and the product was precipitated with Et₂O. The precipitate was isolated by decantation and further purified by preparative HPLC (H₂O: MeCN, 15-30%, 0.1% formic acid). After lyophilisation, the product was obtained as an off-white solid (28 mg, 61%). ¹H NMR (500 MHz, MeOH-d4) δ 8.53 (s, 0.5 H, HCOOH), 8.49 (s, 1H, FQ-H-2), 7.64 (d, J=14.1 Hz, 1H, FQ-H-5), 6.99 (d, J=7.3 Hz, 1H, FQ-H-8), 4.52 (s, 1H, Leu-Cα-H), 4.40 (q, J=6.2 Hz, 1H, Ala-Cα-H), 4.00-3.84 (m, 1H, aminopyrrolidine-H), 3.84-3.74 (m, 1H, aminopyrrolidine-H), 3.74-3.55 (m, 4H, cPr-CH, aminopyrrolidine-H, aminopyrrolidine-H, aminopyrrolidine-H), 2.38-2.25 (m, 1H, aminopyrrolidine-H), 2.20-2.07 (m, 1H, aminopyrrolidine-H), 1.82-1.71 (m, 1H, Leu-CH), 1.68 (ddd, J=13.9, 8.1, 5.8 Hz, 1H, Leu-CH₂), 1.53 (ddd, J=14.0, 8.3, 6.1 Hz, 1H, Leu-CH₂), 1.40 (d, J=7.0 Hz, 3H, Ala-CH₃), 1.37 (s, 2H, cPr-CH₂), 1.20 (s, 2H, cPr-CH₂), 1.00 (d, J=6.5 Hz, 3H, Leu-CH₃), 0.98 (d, J=6.5 Hz, 3H, Leu-CH₃). ¹³C NMR (126 MHz, MeOH-d4) δ 177.28 (d, J=2.8 Hz, FQ-C-4), 174.73 (COOH), 173.91 (HCOOH), 170.09 (C═O), 169.91 (C═O), 151.97 (d, J=247.4 Hz, FQ-C-6), 148.41 (C), 143.39 (d, J=11.8 Hz, FQ-C-7), 141.24 (C), 116.00 (d, J=7.3 Hz, FQ-C-4a), 111.77 (d, J=23.5 Hz, FQ-C-5), 107.33 (C), 101.55 (d, J=5.9 Hz, FQ-C-8), 55.98 (d, J=7.1 Hz, aminopyrrolidine-C), 53.56 (aminopyrrolidine-C), 50.93 (d, J=1.8 Hz, aminopyrrolidine-C), 50.49 (cPr-CH), 43.46 (Leu-CH₂), 36.72 (cPr-CH), 31.88 (aminopyrrolidine-C), 25.56 (Leu-CH), 23.32 (Leu-CH₃), 22.27 (Leu-CH₃), 18.27 (Ala-CH₃), 8.39 (2× cPr-CH₂). HR-MS calculated [C₂₆H₃₅FN₅051: 516.2617, found 516.2610.

### 5.23 LecA-targeted aminopyrrolidine-RQ-prodrug (36)

The title compound was synthesised in two chemical steps: First, 10 (31 mg, 0.049 mmol, 1 eq.), 30 (34 mg, 0.074 mmol, 1.5 eq.) and TBTU (24 mg, 0.074 mmol, 1.5 eq.) were dissolved in 1 mL dry DMF. DIPEA (27 μL, 0.16 mmol, 3.2 eq.) was added dropwise and the reaction was stirred for 1 h. After evaporation of the solvent, the residue was taken up in 2 mL MeOH. Pd black (10 mg, 0.05 mmol, 1 eq.) was added and the reaction was stirred under H₂ atmosphere for 2 d. Afterwards, the reaction was filtered over celite and further purified by preparative HPLC (MeCN:H₂O, 20-33%, 0.1% formic acid). The title compound was obtained as a beige amorphous solid (22 mg, 48% over 2 chemical steps). 1H NMR (500 MHz, MeOH-d4) δ 8.60 (s, 1H, FQ-H-2), 7.82 (d, J=8.2 Hz, 2H, Ar—H), 7.71 (d, J=14.2 Hz, 1H, FQ-C5-H), 7.56 (d, J=8.3 Hz, 2H, Ar—H), 7.04 (d, J=7.4 Hz, 1H, FQ-H-8), 4.71 (d, J=9.8 Hz, 1H, glyco-H-1), 4.54-4.44 (m, 1H, aminopyrrolidine-CH), 4.36 (q, J=7.2 Hz, 1H, Ala-Cα-H), 4.33-4.25 (m, 2H, Ala-Cα-H, Leu-Cα-H), 3.91 (d, J=3.5 Hz, 1H, glyco-H-4), 3.90-3.59 (m, 11H, cPr-CH, 2× aminopyrrolidine-CH₂, Gly-CH₂, glyco-H-2, glyco-H-5, glyco-H-6), 3.52 (dd, J=9.2, 3.3 Hz, 1H, glyco-H-3), 2.37-2.20 (m, 1H, aminopyrrolidine-H), 2.20-2.10 (m, 1H, aminopyrrolidine-H), 1.75 (ddd, J=14.6, 11.2, 3.6 Hz, 1H, Leu-CH₂), 1.70-1.56 (m, 2H, Leu-CH₂+Leu-CH), 1.49 (d, J=7.2 Hz, 3H, Ala-CH₃), 1.41 (d, J=7.2 Hz, 3H, Ala-CH₃), 1.38 (d, J=7.1 Hz, 2H, cPr-CH₂), 1.30-1.14 (m, 2H, cPr-CH₂), 0.87 (d, J=6.2 Hz, 3H, Leu-CH₃), 0.85 (d, J=6.2 Hz, 3H, Leu-CH₃). ¹³C NMR (126 MHz, MeOHd4) δ 177.52 (d, J=2.8 Hz, FQ-C═O), 176.71 (C═O), 175.15 (C═O), 174.86 (C═O), 172.46 (C═O), 170.07 (C═O), 169.97 (C═O), 152.04 (d, J=247.4 Hz, FQ-C-6), 148.62 (FQ-C), 143.56 (d, J=11.9 Hz, FQ-C-7), 142.33 (FQ-C), 141.38 (Ar—C), 132.15 (Ar—C), 129.81 (Ar—C), 129.18 (Ar—C), 116.06 (d, J=6.5 Hz, FQ-C-4a), 111.92 (d, J=23.6 Hz, FQ-C-5), 107.34 (FQ-C), 101.64 (d, J=5.9 Hz, FQ-C-8), 88.88 (glyco-C-1), 80.79 (glyco-C-5), 76.26 (glyco-C-3), 70.85 (glyco-C-2), 70.39 (glyco-C-4), 62.67 (glyco-C-6), 55.95 (d, J=5.3 Hz, aminopyrrolidine-C), 53.91 (Leu-Ca), 52.60 (Ala-Cα), 50.94 (aminopyrrolidine-C, Ala-Cα), 44.21 (Gly-Ca), 41.12 (Leu-CH₂), 36.82 (cPr-CH), 31.81 (aminopyrrolidine-C), 25.79 (Leu-CH), 23.55 (Leu-CH₃), 21.50 (Leu-CH₃), 17.78 (Ala-CH₃), 17.25 (Ala-CH₃), 8.49 (cPr-CH₂), 8.47 (cPr-CH₂). HR-MS calcd [C₄₄H₅₇FN₇O₁₃S]⁺: 942.3714, found 942.3694.

### 5.24 LecB-targeted aminopyrrolidine-FQ-prodrug benzyl ester (33)

16 (70 mg, 0.10 mmol, 1 eq.), 30 (55 mg, 0.12 mg, 1.2 eq.) and TBTU (48 mg, 0.15 mmol, 1.5 eq.) were dissolved in dry DMF (2 mL). DIPEA (52 μL, 0.3 mmol, 3 eq.) was added dropwise and the reaction was stirred for 1 h. The solvent was evaporated in vacuo and the reaction was purified by pHPLC (MeCN in Water, 25-40%, 0.1% formic acid). After lyophilisation, the title compound was isolated as an off-white powder (53 mg, 49%). ¹H NMR (500 MHz, MeOHd4) δ 8.56 (s, 1H, FQ-H-2), 8.04 (d, J=8.2 Hz, 2H, glyco-Ar—H), 7.93 (d, J=8.3 Hz, 2H, glyco-Ar—H), 7.75 (d, J=14.5 Hz, 1H, FQ-H-5), 7.47 (d, J=7.2 Hz, 2H, Bn), 7.39-7.34 (m, 2H, Bn), 7.34-7.29 (m, 1H, Bn), 6.98 (d, J=7.5 Hz, 1H, FQ-H-8), 5.31 (s, 2H, Bn), 4.47-4.38 (m, 2H, Ala-Cα-H, pyrr-H), 4.36-4.26 (m, 2H, Ala-Cα-H, Leu-Cα-H), 3.91-3.82 (m, 1H, pyrr-H), 3.79 (d, J=16.7 Hz, 1H, gly-Cα-H), 3.80-3.74 (m, 1H, pyrr-H), 3.66 (d, J=16.7 Hz, 1H, gly-Cα-H), 3.64-3.59 (m, 1H, pyrr-H), 3.58 (d, J=2.8 Hz, 1H, glyco-H-4), 3.58-3.51 (m, 2H, cPr-CH, pyrr-H), 3.43 (dq, J=6.5, 0.5 Hz, 1H, glyco-H-5), 3.40-3.35 (m, 2H, glyco-H-3, glyco-H-1), 3.34-3.27 (m, 1H, linker-CH₂), 3.10 (ddd, J=9.0, 7.1, 2.4 Hz, 1H, glyco-H-2), 3.02 (dd, J=12.9, 7.1 Hz, 1H, linker-CH₂), 2.35-2.20 (m, 1H, pyrr-H), 2.15-2.07 (m, 1H, pyrr-H), 1.81-1.69 (m, 1H, Leu-CH), 1.69-1.55 (m, 2H, Leu-CH), 1.50 (d, J=7.2 Hz, 3H, Leu-CH₃), 1.39 (d, J=7.2 Hz, 3H, Leu-CH₃), 1.33-1.28 (m, 1H, cPr-CH₂), 1.16 (d, J=6.4 Hz, 3H, glyco-H-6), 1.12-1.05 (m, 2H, cPr-CH₂), 0.85 (d, J=6.4 Hz, 3H, Leu-CH₃), 0.84 (d, J=6.3 Hz, 3H, Leu-CH₃). ¹³C NMR (126 MHz, MeOH-d4) δ 176.30 (CONH), 175.32 (d, J=2.7 Hz, FQ-C═O), 175.05 (CONH), 174.75 (CONH), 172.38 (CONH), 169.09 (CONH), 166.10 (COOBn), 151.73 (d, J=244.9 Hz, FQ-C-6), 149.61 (FQ-Ar—C), 145.13 (Ar—C), 142.67 (d, J=12.0 Hz, FQ-C-7), 140.44 (Ar—C), 138.34 (Ar—C), 138.00 (Ar—C), 129.56 (glyco-Ar—C), 129.53 (Bn), 129.25 (Bn), 129.13 (Bn), 128.10 (glyco-Ar—C), 119.19 (d, J=6.7 Hz, FQ-C-4a), 112.91 (d, J=23.1 Hz, FQ-C-5), 109.81 (Ar—C), 101.62 (d, J=5.1 Hz, FQ-C-8), 79.62 (glyco-C-2), 76.32 (glyco-C-3), 75.51 (glyco-C-5), 73.54 (glyco-C-4), 69.60 (glyco-C-1), 67.07 (Bn), 55.94 (d, J=6.7 Hz, pyrr-C), 53.94 (Leu-Ca), 52.54 (Ala-Cα), 50.86 (pyrr-C), 50.76 (Ala-Cα), 48.84 (pyrr-C) 45.55 (pyrr-C), 44.19 (gly-Ca), 41.16 (Leu-CH₂), 36.18 (cPr-CH), 31.88 (pyrr-C), 25.80 (Leu-CH), 23.52 (Leu-CH₃), 21.53 (Leu-CH₃), 17.88 (Ala-CH₃), 17.26 (Ala-CH₃), 17.11 (glyco-C-6), 8.51 (cPr-CH₂), 8.49 (cPr-CH₂). LR-MS: m/z=539.2 [M+2H]z.

### 5.24 LecB-targeted aminopyrrolidine-FQ-prodrug (37)

33 (40 mg, 0.037 mmol, 1 eq.) was dissolved in MeOH (1 mL). Pd/C (10% m/m, 4 mg, 10 mol %) was added and the atmosphere was changed to H₂ (1 atm.). The reaction was stirred at room temperature for 24 h until full consumption of the starting material. Pd/C was removed by centrifugation (17600 ref, 10 min) and the solvent was removed in vacuo. After purification by pHPLC (MeCN in Water, 22-35%, 0.1% formic acid), the title compound was obtained as an off-white amorphous solid (15 mg, 41%). ¹H NMR (500 MHz, MeOH-d4) δ 8.78 (s, 1H, FQH-2), 8.05 (d, J=8.1 Hz, 2H, glyco-Ar—H), 7.93 (d, J=8.3 Hz, 2H, glyco-Ar—H), 7.90 (d, J=13.1 Hz, 1H, FQ-H-5), 7.61 (d, J=7.2 Hz, 1H, FQ-C-8), 4.90 (q, J=6.9 Hz, 1H, Ala-Cα-H), 4.54-4.37 (m, 2H, Ala′-Cα-H, Leu-Cα-H), 4.00 (d, J=16.9 Hz, 1H, gly-Cα-H), 3.96-3.84 (m, 2H, pyrr-H), 3.83-3.72 (m, 2H, pyrr-H, gly-Cα-H), 3.72-3.63 (m, 1H, pyrr-H), 3.58 (d, J=2.9 Hz, 1H, glyco-H-4), 3.50-3.27 (m, 7H, pyrr-H, pyrr-H, pyrr-H, glyco-H-5, glyco-H-1, glyco-H-3, glyco-linker-CH₂), 3.10 (ddd, J=9.0, 7.1, 2.4 Hz, 1H, glyco-H-2), 3.02 (dd, J=12.9, 7.2 Hz, 1H, glyco-linker-CH₂), 1.78-1.57 (m, 3H, Leu-CH, Leu-CH₂), 1.50 (d, J=7.1 Hz, 3H, Ala-CH₃), 1.42 (d, J=6.9 Hz, 2H, cPr-CH₂), 1.36 (d, J=6.8 Hz, 3H, Ala′—CH₃), 1.26-1.20 (m, 2H, cPr-CH₂), 1.16 (d, J=6.4 Hz, 3H, glyco-H-6), 0.87 (d, J=5.6 Hz, 3H, Leu-CH₃), 0.83 (d, J=5.6 Hz, 3H, Leu-CH₃). ¹³C NMR (126 MHz, MeOH-d4) δ 178.37 (d, J=2.0 Hz, FQ-C═O), 175.82 (CONH), 174.14 (CONH), 172.57 (CONH), 171.68 (CONH), 169.67 (CONH), 168.86 (COOH), 155.08 (d, J=250.0 Hz, FQ-C-6), 149.36 (FQ-H-2), 146.84 (d, J=9.9 Hz, FQ-C-7), 145.06 (Ar—C), 140.78 (Ar—C), 138.51 (Ar—C), 129.53 (glyco-Ar—C), 128.08 (glyco-Ar—C), 121.07 (d, J=7.0 Hz, FQ-C-4a), 112.56 (d, J=23.4 Hz, FQ-C-5), 107.68 (Ar—C), 79.62 (glyco-C-2), 76.33 (glyco-C-3), 75.52 (glyco-C-5), 73.55 (glyco-C-4), 69.61 (glyco-C-1), 53.12 (Leu-Ca), 52.12 (Ala-Cα), 51.06 (pyrr-C), 50.67 (d, J=3.8 Hz, pyrr-C), 46.60 (Ala′-Ca), 46.46 (pyrr-H), 45.55 (glyco-linker-CH₂), 43.91 (gly-Ca), 43.17 (pyrr-C), 41.61 (Leu-CH₂), 37.02 (cPr-CH), 25.74 (Leu-CH), 23.54 (Leu-CH₃), 21.63 (Leu-CH₃), 17.85 (Ala-CH₃), 17.26 (Ala′—CH₃), 17.10 (glyco-C-6), 8.61 (cPr-CH₂), 8.56 (cPr-CH₂). HR-MS calculated [C₄₅H₆₀FN₈O₁₄S]⁺: 987.3928, found 987.3903.

5.25 (S)-7-(3-(hydroxymethyl)-1-pyrrolidinyl)-1-cyclopropyl-6-fluoro-1,4-dihydro-4-oxo-1,8-naphthyridine-3-carboxylic acid (39)

7-chloro-1-cyclopropyl-6-fluoro-4-oxo-1,4-dihydroquinoline-3-carboxylic acid (26, 1000 mg, 3.55 mmol, 1 eq.) was dispersed in dry pyridine (10 mL) and heated to 80° C. to fully dissolve. 1-β-prolinol (38, 732 μL, 7.1 mmol, 2 eq.) was added and the temperature was increased to 140° C. The reaction was refluxed overnight. After cooling to room temperature, the product precipitated from the reaction and was isolated by filtration. The precipitate was washed with ice-cold MeOH and obtained as a yellow solid (750 mg, 62%). ¹H NMR (500 MHz, MeOH-d+CHCl3-d, 1:1) δ 8.78 (s, 1H, FQ-H-2), 7.87 (d, J=13.9 Hz, 1H, FQ-H-5), 7.03 (d, J=7.3 Hz, 1H, FQ-H-8), 3.88-3.76 (m, 2H, 2× pyrrolidinyl-methanol-H), 3.76-3.67 (m, 3H, 2× pyrrolidinylmethanol-H, cPr-CH), 3.60 (dd, J=10.8, 7.2 Hz, 1H, pyrrolidinyl-methanol-H), 3.51 (ddd, J=10.1, 7.4, 2.1 Hz, 2H, pyrrolidinyl-methanol-H), 2.59 (hept, J=7.2 Hz, 1H, pyrrolidinylmethanol-H), 2.18 (dtd, J=11.9, 7.1, 4.5 Hz, 1H, pyrrolidinyl-methanol-H), 1.88 (dq, J=12.6, 8.2 Hz, 1H, pyrrolidinyl-methanol-H), 1.45 (q, J=6.8 Hz, 2H, cPr-CH₂), 1.21 (q, J=6.6 Hz, 2H, cPr-CH₂). ¹³C NMR (126 MHz, MeOH-d+CHCl3-d, 1:1) δ 173.16 (FQ-C═O), 169.65 (COOH), 152.06 (d, J=252.5 Hz, FQ-C-6), 148.01 (FQ-C-2), 144.44 (d, J=15.8 Hz, FQ-C-7), 141.51 (FQ-C), 128.36 (FQ-C), 111.48 (d, J=24.1 Hz, FQ-C-5), 105.36 (d, J=2.6 Hz, FQ-C-8), 100.04 (d, J=6.3 Hz, FQ-C-4a), 63.69 (pyrrolidinyl-methanol-C), 53.78 (d, J=5.7 Hz, pyrrolidinyl-methanol-C), 50.71 (d, J=6.6 Hz, pyrrolidinyl-methanol-C), 41.38 (d, J=1.9 Hz, pyrrolidinyl-methanol-C), 37.18 (s, cPr-CH), 28.25 (pyrrolidinylmethanol-C), 8.47 (cPr-CH₂). LR-MS: m/z=347.2 [M+H]⁺.

### 5.26 (S)-7-(3-(hydroxymethyl)-1-pyrrolidinyl)-1-cyclopropyl-6-fluoro-1,4-dihydro-4-oxo-1,8-naphthyridine-3-carboxylic acid methyl ester (40)

39 (500 mg, 1.44 mmol, 1 eq.) and (R/S)-camphor-10-sulfonic acid (668 mg, 2.88 mmol, 2 eq.) were dried on high vacuum and subsequently dissolved in dry MeOH (15 mL). The reaction was refluxed until a clear solution was obtained (72 h). After cooling to room temperature, the solvent was evaporated in vacuo. The residual solid was dissolved in CH₂Cl₂(10 mL) and the organic phase was washed with satd. aqueous NaHCO₃ (3×) and satd. brine (2×) and dried over Na₂SO₄. After evaporation of the solvent, the product was obtained as a yellow solid (510 mg 96%), containing 4% of the starting material as an impurity. ¹H NMR (500 MHz, DMSO-d6) δ 8.35 (s, 1H, FQ-C-2), 7.64 (d, J=14.6 Hz, 1H, FQ-C-5), 6.94 (d, J=7.6 Hz, 1H, FQ-C-8), 4.80 (t, J=5.2 Hz, 1H, OH), 3.72 (s, 3H, COOMe), 3.66-3.52 (m, 4H, 3× pyrrolidinyl-methanol-H, cPr-CH), 3.49 (td, J=10.8, 5.0 Hz, 1H, pyrrolidinyl-methanol-H), 3.46-3.39 (m, 1H, pyrrolidinyl-methanol-H), 3.35-3.30 (m, 1H, pyrrolidinyl-methanol-H), 2.44 (p, J=7.0 Hz, 1H, pyrrolidinyl-methanol-H), 2.04 (dtd, J=11.8, 7.1, 4.8 Hz, 1H, pyrrolidinyl-methanol-H), 1.76 (dq, J=11.8, 7.7 Hz, 1H, pyrrolidinyl-methanol-H), 1.27-1.20 (m, 1H, cPr-CH₂), 1.11-1.02 (m, 1H, cPr-CH₂). ¹³C NMR (126 MHz, DMSO-d6) δ 171.46 (FQ-C═O), 165.20 (COOMe), 149.43 (d, J=242.8 Hz, FQ-C-6), 147.93 (FQ-C-2), 140.48 (d, J=11.3 Hz, FQ-C-7), 138.63 (FQ-C), 117.62 (d, J=5.5 Hz, FQ-C-4a), 111.44 (d, J=22.5 Hz, FQ-C-5), 108.57 (FQ-C), 100.47 (d, J=5.3 Hz, FQ-C-8), 62.55 (pyrrolidinyl-methanol-C), 52.50 (d, J=5.4 Hz, pyrrolidinyl-methanol-C), 51.24 (COOMe), 49.23 (d, J=5.3 Hz, pyrrolidinyl-methanol-C), 40.68 (d, J=1.7 Hz, pyrrolidinyl-methanol-C), 34.63 (cPr-CH), 27.46 (pyrrolidinyl-methanol-C), 7.53 (cPr-CH₂). LR-MS: m/z=361.2 [M+H]⁺.

### 5.27 (S)-7-(3-(azidomethyl)-1-pyrrolidinyl)-1-cyclopropyl-6-fluoro-1,4-dihydro-4-oxo-1,8-naphthyridine-3-carboxylic acid methyl ester (41)

40 (400 mg, 1.1 mmol, 1 eq.) and PPh₃ (577 mg, 2.2 mmol, 2 eq.) were dispersed in dry THF (10 mL) at room temperature. DIAD (473 μL, 2.2 mmol, 2 eq.) was added dropwise under vigorous stirring, resulting in a clear solution. Afterwards, DPPA (432 μL, 2.2 mmol, 2 eq.) was added dropwise, which resulted in a precipitation after 10 min. The reaction was stirred for 1 h and subsequently quenched with MeOH. After evaporation of the solvent in vacuo, the product was purified via NP-MPLC (CH₂Cl2/PE (9/5): EtOH, 1-5%), yielding the title compound as an off-white solid (277 mg, 65%). ¹H NMR (500 MHz, MeOH-d) δ 8.46 (s, 1H, FQ-H-2), 7.64 (d, J=14.7 Hz, 1H, FQ-H-5), 6.89 (d, J=7.6 Hz, 1H, FQ-H-8), 3.82 (s, 3H, COOMe) 3.71 (ddd, J=10.3, 7.3, 3.1 Hz, 1H, azidomethylpyrrolidine-H), 3.65 (ddq, J=10.8, 7.6, 3.5 Hz, 1H, azidomethylpyrrolidine-H), 3.62-3.55 (m, 1H, azidomethylpyrrolidine-H), 3.55-3.50 (m, 2H, azidomethylpyrrolidine-H, cPr-CH), 3.47 (dd, J=12.3, 7.3 Hz, 1H, azidomethylpyrrolidine-H), 3.35 (ddd, J=10.2, 7.4, 2.7 Hz, 1H, azidomethylpyrrolidine-H), 2.61 (hept, J=7.2 Hz, 1H, azidomethylpyrrolidine-H), 2.20 (dtd, J=11.6, 7.0, 4.1 Hz, 1H, azidomethylpyrrolidine-H), 1.84 (dq, J=12.4, 8.2 Hz, 1H, azidomethylpyrrolidine-H), 1.40-1.24 (m, 2H, cPr-CH₂), 1.20-1.06 (m, 2H, cPr-CH₂). ¹³C NMR (126 MHz, MeOH-d4) δ 175.19 (FQ-C═O), 166.90 (COOMe), 151.68 (d, J=244.4 Hz, FQ-C-6), 149.64 (FQ-C-2), 142.60 (d, J=11.8 Hz, FQ-C-7), 140.45 (FQ-C), 119.15 (d, J=6.4 Hz, FQ-C-4a), 112.85 (d, J=23.1 Hz, FQ-C-5), 109.77 (FQ-C), 101.46 (d, J=5.4 Hz, FQ-C-8), 54.73 (azidomethylpyrrolidine-C), 54.24 (d, J=6.2 Hz, azidomethylpyrrolidine-C), 52.01 (COOMe), 50.46 (d, J=5.6 Hz, azidomethylpyrrolidine-C), 39.86 (azidomethylpyrrolidine-C), 36.11 (cPr-CH), 29.77 (azidomethylpyrrolidine-C), 8.44 (cPr-CH₂). LR-MS: m/z=385.2 [M+H]⁺.

### 5.28 (S)-7-(3-(azidomethyl)-1-pyrrolidinyl)-1-cyclopropyl-6-fluoro-1,4-dihydro-4-oxo-1,8-naphthyridine-3-carboxylic acid·HCl (42)

41 (150 mg, 0.39 mmol, 1 eq.) and Pd/C (10% m/m, 42 mg, 10 mol %) were stirred in MeOH (10 mL) under H₂-atmosphere (1 atm) overnight. The reaction was filtered over celite and concentrated in vacuo. HCl in dioxane (4 N, 100 μL) was mixed with 40 mL Et₂O and carefully added to the solution of product in MeOH while stirring on ice, yielding the title compound as a yellow solid (120 mg, 78%). ¹H NMR (500 MHz, H2O-d2) δ 8.34 (s, 1H, FQ-H-2), 7.27 (d, J=14.6 Hz, 1H, FQ-H-5), 6.54 (d, J=7.4 Hz, 1H, FQ-H-8), 3.82 (s, 3H, COOMe), 3.69-3.60 (m, 1H, aminomethylpyrrolidine-H), 3.55-3.40 (m, 1H, aminomethylpyrrolidine-H), 3.30 (s, 1H), 3.25-3.08 (m, 1H), 2.72-2.58 (m, OH), 2.35-2.20 (m, OH), 1.94-1.64 (m, 1H), 1.25 (d, J=5.6 Hz, 1H), 0.98 (s, OH). ¹³C NMR (126 MHz, H₂O-d2) δ 173.63 (FQ-C═O), 166.68 (COOMe), 149.62 (d, J=245.1 Hz, FQ-C-6), 148.66 (FQ-C-2), 140.59 (d, J=11.7 Hz, FQC-7), 138.43 (FQ-C), 116.50 (d, J=6.5 Hz, FQ-C-4a), 110.83 (d, J=23.6 Hz, FQ-C-5), 107.09 (FQ-C), 99.97 (d, J=5.4 Hz, FQ-C-8), 52.65 (d, J=5.8 Hz, aminomethylpyrrolidine-C), 51.90 (COOMe), 48.98 (d, J=6.0 Hz, aminomethylpyrrolidine-C), 41.69 (aminomethylpyrrolidine-C), 36.30 (cPr-CH), 35.01 (aminomethylpyrrolidine-C), 28.55 (aminomethylpyrrolidine-C), 7.13 (cPr-CH₂). LR-MS: m/z=360.2 [M+H]⁺.

### 5.29 (S)-7-(3-(aminomethyl)-1-pyrrolidinyl)-1-cyclopropyl-6-fluoro-1,4-dihydro-4-oxo-1,8-naphthyridine-3-carboxylic acid (3)

Methylester 42 (39 mg, 0.1 mmol, 1 eq.) was dissolved in a mixture of THF, MeOH and H₂O (3/1/1) at room temperature. LiOH (13 mg, 0.5 mmol, 5 eq.) was added and the reaction was stirred overnight until full transformation. After evaporation of the solvent in vacuo, the product was purified by pHPLC (MeCN in Water, 10-25%, 0.1% formic acid), yielding the title compound as an off-white amorphous solid (15 mg, 43%). H NMR (500 MHz, H2O-d2) δ 8.31 (s, 1H, FQ-H-2), 6.93 (d, J=13.8 Hz, 1H, FQ-H-5), 6.56 (d, J=7.4 Hz, 1H, FQ-H-8), 3.75-3.67 (m, 1, aminomethylpyrrolidine-H), 3.63-3.51 (m, 1H, aminomethylpyrrolidine-H), 3.54-3.48 (m, 1H, aminomethylpyrrolidine-H), 3.42 (s, 1H, aminomethylpyrrolidine-H), 3.29-3.12 (m, 3H, cPr —CH, aminomethylpyrrolidine-CH₂), 2.69 (dt, J=14.2, 5.8 Hz, 1H, aminomethylpyrrolidine-H), 2.35-2.23 (m, 1H, aminomethylpyrrolidine-H), 1.89-1.75 (m, 1H, aminomethylpyrrolidine-H), 1.35 (d, J=6.2 Hz, 2H, cPr-CH₂), 1.08 (s, 2H, cPr-CH₂). ¹³C NMR (126 MHz, H2O-d2) δ 173.86 (d, J=3.5 Hz, FQ-C═O), 169.38 (COOH), 149.71 (d, J =249.2 Hz, FQ-C-6), 146.83 (FQ-C), 141.48 (d, J=11.1 Hz, FQ-C-7), 139.22 (FQC), 112.55 (d, J=7.3 Hz, FQ-C-4a), 109.34 (d, J=23.5 Hz, FQ-C-5), 104.99 (d, J=3.5 Hz, FQ-C-8a), 99.64 (d, J=5.6 Hz, FQ-C-8), 52.70 (d, J=6.8 Hz, aminomethylpyrrolidine-C), 49.19 (d, J=4.9 Hz, aminomethylpyrrolidine-C), 41.51 (aminomethylpyrrolidine-C), 36.32 (aminomethylpyrrolidine-C), 35.72 (cPr-CH), 28.39 (aminomethylpyrrolidine-C), 7.22 (cPr-CH₂). HR-MS calcd [C₁₈H₂₁FN₃O₃]⁺: 346.1561, found 346.1555.

### 5.30 1-cyclopropyl-6-fluoro-7-((R)-3-((4S,7S)-7-isobutyl-4,11,11-trimethyl-3,6,9-trioxo-10-oxa-2,5,8-triazadodecyl)pyrrolidin-1-yl)-4-oxo-1,4-dihydroquinoline-3-carboxylic acid methyl ester (43)

42 (50 mg, 0.13 mmol, 1 eq.), 17 (48 mg, 0.16 mmol, 1.2 eq.) and TBTU (51 mg, 0.16 mmol, 1.2 eq.) were dissolved in dry DMF (1.5 mL) and cooled on ice. DIPEA (45 μL, 0.26 mmol, 2 eq.) was added dropwise and the reaction was allowed to warm to room temperature. After 16 h, the solvent was concentrated in vacuo and diluted with CH₂Cl₂ (20 mL). The organic phase was washed with aq. KHSO₄ (1 M, 2×), neutralised with aq. satd. NaHCO₃ (1×), washed with satd. brine (2×) and dried over Na₂SO₄. After purification via NP-MPLC (CH₂Cl₂: MeOH, 1-10%), the title compound was obtained as a white solid (42 mg, 50%). The compound was directly used for global deprotection without NMR-spectroscopic characterisation. LR-MS: m/z=630.4 [M+H]⁺.

### 5.31 1-cyclopropyl-6-fluoro-7-((R)-3-((4S.7S)-7-isobutyl-4,11,11-trimethyl-3,6,9-trioxo-10-oxa-2,5,8-triazadodecyl)pyrrolidin-1-yl)-4-oxo-1,4-dihydroquinoline-3-carboxylic acid (47)

43 (42 mg, 0.065 mmol, 1 eq.) and LiOH (7.8 mg, 0.325 mmol, 5 eq.) were dissolved in a mixture of THF/H₂O/MeOH (5/5/1, 1.5 mL) and stirred at room temperature until full consumption of the starting material (3 h). The reaction was diluted with MeOH (5 mL) and neutralised with Amberlite IR-120 H⁺ exchange resin. After evaporation of the solvent, the residuum (35 mg) was dissolved in a mixture of dioxane/MeOH (8/2, 1 mL). While cooling on ice, HCl in dioxane (4 N, 2 mL) was added dropwise. The reaction was allowed to warm to room temperature and stirred until full consumption of the starting material (1 h). After removal of the solvent in vacuo, the product was purified via preparative HPLC (H₂O: MeCN, 18-30%, 0.1% formic acid). The title compound was obtained as a white solid (16 mg, 46% over two steps). ¹H NMR (500 MHz, MeOH-d4) δ 9.01 (s, 1H, FQ-H-2), 7.96 (d, J=13.9 Hz, 1H, FQ-H-5), 7.23 (d, J=7.4 Hz, 1H, FQ-H-8), 4.40 (q, J=7.1 Hz, 1H, Ala-Cα-H), 4.04-3.83 (m, 4H, cPr-CH, Ala-Cα-H, Leu-Cα-H, aminomethylpyrrolidine-H), 3.81-3.70 (m, 1H, aminomethylpyrrolidine-H), 3.61-3.53 (m, 1H, aminomethylpyrrolidine-H), 3.36 (d, J=6.9 Hz, 2H, aminomethylpyrrolidine-CH₂), 2.63 (tt, J=7.1, 7.1 Hz, 1H, aminomethylpyrrolidine-H), 2.38-2.09 (m, 1H, aminomethylpyrrolidine-H), 1.97-1.84 (m, 1H, aminomethylpyrrolidine-H), 1.83-1.72 (m, 2H, Leu-CH₂, Leu-CH), 1.72-1.63 (m, 1H, Leu-CH₂), 1.53-1.48 (m, 2H, cPr-CH₂), 1.42 (d, J=7.1 Hz, 3H, Ala-CH₃), 1.34-1.24 (m, 2H, cPr-CH₂), 1.02 (d, J=6.2 Hz, 3H, Leu-CH₃), 1.00 (d, J=6.1 Hz, 3H, Leu-CH₃). ¹³C NMR (126 MHz, MeOH-d4) δ 174.81 (C═O), 171.62 (d, J=4.2 Hz, FQ-C═O), 170.69 (C═O), 170.36 (COOH), 153.15 (d, J=252.6 Hz, FQ-C-6), 149.44 (FQ-C-2), 145.64 (d, J=12.1 Hz, FQ-C-7), 142.75 (FQ-C), 112.62 (d, J=8.4 Hz, FQ-C-4a), 111.28 (d, J=24.9 Hz, FQ-C-5), 104.76 (FQ-C), 101.14 (d, J=6.3 Hz, FQ-C-8), 55.03 (d, J=6.5 Hz, aminomethylpyrrolidine-C), 52.84 (Ala-Cα), 51.17 (d, J=5.9 Hz, aminomethylpyrrolidine-C), 50.74 (Ala-Cα), 42.15 (aminomethylpyrrolidine-C), 41.66 (Leu-CH₂), 40.25 (aminomethylpyrrolidine-C), 38.49 (cPr-CH), 29.67 (aminomethylpyrrolidine-C), 25.35 (Leu-CH), 23.18 (Leu-CH₃), 22.07 (Leu-CH₃), 18.30 (Ala-CH₃), 8.64 (cPr-CH₂). HR-MS calculated [C₂₇H37FN₅O5s]: 530.2773, found 530.2766.LecA

### 5.32 LecA-targeted aminomethylpyrrolidine-FQ-Prodrug (48)

The title compound was synthesised in two chemical steps: First, 10 (27 mg, 0.049 mmol, 1 eq.), 42 (30 mg, 0.076 mmol, 1.8 eq.) and TBTU (21 mg, 0.065 mmol, 1.8 eq.) were dissolved in 2 mL dry DMF. DIPEA (15 μL, 0.086 mmol, 2 eq.) was added dropwise and the reaction was stirred for 1 h. After evaporation of the solvent, the residue was taken up in 1 mL H₂O/THF (1:1). LiOH (10 mg, 0.4 mmol, 10 eq.) was dissolved in 1 mL water and added stepwise to the reaction until a full transformation was observed (3 h). Afterwards, the reaction was neutralised with Amberlite IR-120 H⁺ exchange resin and further purified by preparative HPLC (MeCN:H₂O, 20-33%, 0.1% formic acid). The title compound was obtained as a beige amorphous solid (33 mg, 70% over 2 chemical steps). 1H NMR (500 MHz, MeOH-d4) δ 8.65 (s, 1H, FQH-2), 7.79 (d, J=8.0 Hz, 2H, Ar—H), 7.72 (d, J=14.2 Hz, 1H, FQ-H-5), 7.52 (d, J=8.0 Hz, 2H, Ar—H), 7.02 (d, J=7.2 Hz, 1H), 4.71 (d, J=9.8 Hz, 1H, glyco-H-1), 4.43-4.32 (m, 2H, Ala-Cα-H, Leu-Cα-H), 4.29 (q, J=7.3 Hz, 1H, Ala-Cα-H), 3.97-3.89 (m, 2H, glyco-H-4, Gly-CH), 3.82-3.58 (m, 9H, Gly-CH, 3× aminomethylpyrrolidine-H, cPr-CH, glyco-H-6, glyco-H-2, glyco-H-5), 3.52 (dd, J=9.2, 3.3 Hz, 1H, glyco-H-3), 3.47-3.33 (m, 2H, 2× aminomethylpyrrolidine-H), 3.29-3.22 (m, 1H, aminomethylpyrrolidine-H), 2.60 (tt, J=13.4, 6.6 Hz, 1H, aminomethylpyrrolidine-H), 2.18 (td, J=12.0, 6.2 Hz, 1H, aminomethylpyrrolidine-H), 1.86-1.71 (m, 1H, aminomethylpyrrolidine-H, Leu-CH₂), 1.71-1.58 (m, 2H, Leu-CH₂, Leu-CH), 1.49 (d, J=7.3 Hz, 3H, Ala-CH₃), 1.42 (d, J=7.2 Hz, 3H, Ala-CH₃), 1.39-1.36 (m, 2H, cPr-CH₂), 1.18 (s, 2H, cPr-CH₂), 0.88 (d, J=5.5 Hz, 6H, 2× Leu-CH₃). ¹³C NMR (126 MHz, MeOH-d4) δ 177.62 (C═O), 176.64 (C═O), 175.27 (C═O), 174.88 (C═O), 172.68 (C═O), 170.26 (C═O), 169.79 (C═O), 152.02 (d, J=248.5 Hz, FQ-C-6), 148.66 (FQ-C), 142.31 (FQ-C), 141.49 (Ar—C), 132.09 (Ar—C), 129.69 (Ar—C), 129.13 (Ar—C), 111.98 (d, J=23.3 Hz, FQ-C-5), 101.44 (d, J=6.1 Hz, FQ-C-8), 88.86 (glyco-C-1), 80.79 (glyco-C-5), 76.26 (glyco-C-3), 70.85 (glyco-C-2), 70.40 (glyco-C-4), 62.68 (glyco-C-6), 54.49 (d, J=5.9 Hz, aminomethylpyrrolidine-C), 53.94 (Leu-Ca), 52.42 (Ala-Cα), 51.07 (Ala-Cα), 50.67 (d, J=6.2 Hz, aminomethylpyrrolidine-C), 44.22 (Gly-Ca), 42.42 (aminomethylpyrrolidine-C), 41.16 (Leu-CH₂), 40.00 (aminomethylpyrrolidine-C), 36.85 (cPr-CH), 29.90 (aminomethylpyrrolidine-C), 25.80 (Leu-CH), 23.57 (Leu-CH₃), 21.49 (Leu-CH₃), 17.66 (Ala-CH₃), 17.31 (Ala-CH₃), 8.53 (cPr-CH₂), 8.49 (cPr-CH₂). HRMS calcd [C₄₅H₅₉FN70₁₃S]⁺: 956.3870, found 956.3852.

### 5.33 LecB-targeted aminomethylpyrrolidine-FQ-Prodrug methyl ester (45)

16 (70 mg, 0.10 mmol, 1 eq.), 42 (48 mg, 0.12 mg, 1.2 eq.) and TBTU (48 mg, 0.15 mmol, 1.5 eq.) were dissolved in dry DMF (2 mL). DIPEA (64 μL, 0.36 mmol, 3.6 eq.) was added dropwise and the reaction was stirred for 1 h. The solvent was evaporated in vacuo and the reaction was purified by pHPLC (MeCN in Water, 25-40%, 0.1% formic acid). After lyophilisation, the title compound was isolated as an off-white powder (73 mg, 72%). 1H NMR (500 MHz, MeOHd4) δ 8.55 (s, 1H, FQ-H-2), 8.02 (d, J=8.5 Hz, 2H, glyco-Ar—H), 7.91 (d, J=8.5 Hz, 2H, glyco-Ar—H), 7.75 (d, J=14.6 Hz, 1H, FQ-H-5), 6.98 (d, J=7.5 Hz, 1H, FQ-H-8), 4.43 (q, J=7.1 Hz, 1H, Ala-Cα-H), 4.36-4.26 (m, 2H, Ala′-Cα-H, Leu-Ca), 3.95 (d, J=16.6 Hz, 1H, gly-Cα-H), 3.83 (s, 3H, COOCH3), 3.79 (d, J=16.6 Hz, 1H, gly-Cα-H), 3.74-3.65 (m, 2H, pyrr-H, pyrr-H), 3.64-3.54 (m, 3H, cPr-CH, glyco-H-4, pyrr-H), 3.44 (q, J=6.5 Hz, 1H, glyco-H-5), 3.41-3.35 (m, 4H, glyco-H-1, glyco-H-3, pyrr-H, pyrr-H), 3.35-3.28 (m, 1H, glyco-linker-CH₂), 3.25 (dd, J=13.6, 7.5 Hz, 1H, pyrr-H), 3.10 (ddd, J=9.0, 7.1, 2.5 Hz, 1H, glyco-H-2), 3.02 (dd, J=12.9, 7.1 Hz, 1H, glyco-linker-CH₂), 2.68-2.52 (m, 1H, pyrr-H), 2.32-2.10 (m, 1H, pyrr-H), 1.85-1.76 (m, 1H, pyrr-H), 1.76-1.58 (m, 3H, Leu-CH, Leu-CH₂), 1.50 (d, J=7.2 Hz, 3H, Ala-CH₃), 1.41 (d, J=7.2 Hz, 3H, Ala′—CH₃), 1.37-1.29 (m, 2H, cPr-CH₂), 1.16 (d, J=6.4 Hz, 3H, glyco-H-6), 1.14-1.04 (m, 2H, cPr-CH₂), 0.88 (d, J=4.0 Hz, 3H, Leu-CH₃), 0.87 (d, J=4.1 Hz, 3H, Leu-CH₃). ¹³C NMR (126 MHz, MeOH-d4) δ 176.22 (CONH), 175.30 (d, J=2.7 Hz, FQ-C═O), 175.17 (CONH), 174.76 (CONH), 172.58 (CONH), 168.94 (CONH), 166.97 (COOMe), 151.72 (d, J=244.6 Hz, FQC-6), 149.61 (Ar—C), 145.07 (Ar—C), 142.80 (d, J=12.0 Hz, FQ-C-7), 140.51 (Ar—C), 138.35 (Ar—C), 129.50 (glyco-Ar—C), 128.07 (glyco-Ar—C), 118.97 (d, J=6.4 Hz, FQ-C-4a), 112.82 (d, J=23.3 Hz, FQ-C-5), 109.75 (Ar—C), 101.40 (d, J=5.4 Hz, FQ-C-8), 79.63 (glyco-C-2), 76.33 (glyco-C-3), 75.51 (glyco-C-5), 73.54 (glyco-C-4), 69.61 (glyco-C-1), 54.47 (d, J=5.9 Hz, pyrr-C), 53.93 (Leu-Ca), 52.35 (Ala-Cα), 52.04 (pyrr-C), 50.96 (Ala′-Ca), 50.53 (d, J=6.2 Hz, pyrr-C), 45.55 (glyco-linker-CH₂), 44.23 (gly-Ca), 42.61 (pyrr-C), 41.20 (Leu-CH₂), 40.01 (RHNCH₂—CHpyrr), 36.17 (cPr-CH), 29.93 (RHNCH₂—CHpyrr), 25.81 (Leu-CH), 23.56 (Leu-CH₃), 21.54 (Leu-CH₃), 17.77 (Ala-CH₃), 17.31 (Ala-CH₃), 17.11 (glyco-C-6), 8.53 (cPr-CH₂), 8.50 (cPr-CH₂). LR-MS: m/z=508.2 [M+2H]²+

### 5.34 LecB-targeted aminomethylpyrrolidine-FQ-Prodrug methyl ester (49)

45 (50 mg, 0.049 mmol, 1 eq.) was dissolved in a mixture of THF, MeOH and H₂O (3:1:1, 1 mL) and LiOH (9 mg, 0.368 mmol, 7.5 eq.) was added. The reaction was stirred over night at room temperature until disappearance of the starting material. After neutralisation with Amberlite IR-120 H⁺ to pH 7, the solvents were removed in vacuo. The title compound was obtained after lyophilisation as an off-white powder (47 mg, 96%). ¹H NMR (500 MHz, MeOH-d4) δ 8.64 (s, 1H, FQ-H-2), 8.03 (d, J=8.1 Hz, 2H, glyco-Ar—H), 7.91 (d, J=8.1 Hz, 2H, glyco-Ar—H), 7.69 (d, J=14.2 Hz, 1H, FQ-H-5), 7.03 (d, J=7.4 Hz, 1H, FQ-H-8), 4.43 (q, J=7.1 Hz, 1H, Ala-Cα-H), 4.34 (dd, J=10.6, 4.2 Hz, 1H, Leu-Cα-H),4.30 (q, J=7.2 Hz, 1H, Ala-Cα-H), 3.95 (d, J=16.6 Hz, 1H, gly-Cα-H), 3.79 (d, J=16.6 Hz, 1H, gly-Cα-H), 3.77-3.60 (m, 4H, pyrr-H, pyrr-H, pyrr-H, cPr-CH), 3.58 (d, J=1.9 Hz, 1H, glyco-H-4), 3.49-3.22 (m, 7H, pyrr-H, pyrr-H, pyrr-H, glyco-H-3, glyco-H-5, glyco-H-1, glyco-linker-CH₂), 3.10 (ddd, J=9.0, 7.0, 2.6 Hz, 1H, glyco-H-2), 3.02 (dd, J=12.9, 7.1 Hz, 1H, glyco-linker-CH₂), 2.67-2.55 (m, 1H, RHNCH₂—CHpyrr), 2.23-2.13 (m, 1H, pyrr-H), 1.82 (ddd, J=12.2, 7.8 Hz, 1H, pyrr-H), 1.77-1.58 (m, 3H, Leu-CH, Leu-CH₂), 1.50 (d, J=7.1 Hz, 3H, Ala-CH₃), 1.41 (d, J=7.2 Hz, 3H, Ala′—CH₃), 1.39-1.37 (m, 2H, cPr-CH₂), 1.20-1.18 (m, 2H, cPr-CH₂), 1.16 (d, J=6.4 Hz, 3H, glyco-H-6), 0.88 (d, J=5.0 Hz, 3H, Leu-CH₃), 0.87 (d, J=5.2 Hz, 3H, Leu-CH₃). ¹³C NMR (126 MHz, MeOH-d4) δ 177.58 (d, J=2.8 Hz, FQ-C═O), 176.19 (CONH), 175.21 (CONH), 174.78 (CONH), 172.56 (CONH), 170.13 (CONH), 168.92 (COOH), 151.98 (d, J=247.3 Hz, FQ-C-6), 148.61 (FQ-C-2), 145.07 (Ar—C), 143.64 (d, J=11.2 Hz FQ-C-7), 141.45 (Ar—C), 138.34 (Ar—C), 129.50 (glyco-Ar—C), 128.08 (glyco-Ar—C), 111.95 (d, J=23.5 Hz, FQ-C-5), 101.44 (d, J=6.0 Hz, FQ-C-8), 79.63 (glyco-C-2), 76.33 (glyco-C-3), 75.52 (glyco-C-5), 73.55 (glyco-C-4), 69.61 (glyco-C-1), 54.51 (d, J=5.6 Hz, pyrr-C), 53.92 (Leu-Ca), 52.33 (Ala-Cα), 50.98 (Ala-Cα), 50.64 (d, J=6.3 Hz, pyrr-C), 45.55 (glyco-linker-CH₂), 44.22 (gly-Ca), 42.46 (pyrr-H), 41.22 (Leu-CH₂), 40.00 (RHNCH₂—CHpyrr), 36.80 (cPr-CH), 29.89 (pyrr-C), 25.81 (Leu-CH), 23.56 (Leu-CH₃), 21.56 (Leu-CH₃), 17.76 (Ala-CH₃), 17.31 (Ala′—CH₃), 17.11 (glyco-C-6), 8.51 (cPr-CH₂), 8.48 (cPr-CH₂). HR-MS calculated [C₄₆H₆₂FNsO1₄S]⁺: 1001.4085, found 1001.4063.

### 5.35 Tetrapeptide linker (8)

8 was synthesised by conventional solution phase peptide synthesis (FIG. 6, A). Boc-protected alanine (Si) and benzyl-protected glycine (S2) were coupled with EDC/HOBt to obtain dipeptide S3 in high yields. S3 was then benzyl-deprotected by hydrogenolysis towards S4. Boc-protected leucine (S5) was coupled to benzyl-protected alanine (S6) as described above. The resulting dipeptide S7 was then boc-deprotected under acidic conditions to obtain compound S8 in excellent yields. The building blocks S4 and S8 were again coupled under activation with EDC/HOBt towards the bis-protected tetrapeptide S9. After acidic deprotection, the title compound 8 was obtained in quantitative yields. The dipeptide linker 17 was synthesised by conventional solution phase peptide synthesis (FIG. 6, B). Boc-protected alanine (S1) and benzyl-protected glycine (S2) were coupled with EDC/HOBt to obtain dipeptide S3 in high yields. S3 was then benzyl-deprotected by hydrogenolysis towards S4.

Compound S9: ¹H NMR (500 MHz, CDCl3) δ 7.40-7.28 (m, 5H, Bn), 7.11-7.01 (m, 1H, NH), 6.99 (d, J=6.6 Hz, 1H, NH), 6.89 (d, J=7.6 Hz, 1H, NH), 5.19 (d, J=12.3 Hz, 1H, Bn-CH₂), 5.13 (d, J=12.3 Hz, 1H, Bn-CH₂), 4.60 (dq, J=7.3, 7.3 Hz, 1H, Ala-Cα-H), 4.52 (td, J=8.9, 5.4 Hz, 1H, Leu-Cα-H), 4.14 (dq, J=6.9, 6.9 Hz, 1H, Ala-Cα-H), 4.02 (dd, J=16.7, 5.9 Hz, 1H, Gly-CH₂), 3.89 (dd, J=16.6, 5.1 Hz, 1H, Gly-CH₂), 1.74-1.58 (m, 2H, Leu-CH+Leu-CH₂), 1.57-1.49 (m, 1H, Leu-CH₂), 1.43 (s, Boc-CH₃), 1.40 (d, J=7.3 Hz, 3H, Ala-CH₃), 1.35 (d, J=7.1 Hz, 3H, Ala-CH₃), 0.90 (d, J=6.5 Hz, 3H, Leu-CH₃), 0.88 (d, J=6.4 Hz, 3H, Leu-CH₃). ¹³C NMR (126 MHz, CDCl3) δ 173.72 (C═O), 172.66 (C═O), 171.82 (C═O), 169.11 (C═O), 155.84 (Boc-C═O), 135.36 (Bn-C), 128.63 (Bn-C), 128.43 (Bn-C), 128.15 (Bn-C), 80.53 (Boc-C), 67.17 (Bn-CH₂), 52.00 (Ala-Cα), 50.63 (Leu-Ca), 48.18 (Ala-Cα), 43.25 (Gly-CH₂), 40.94 (Leu-CH₂), 28.35 (Boc-CH₃), 24.69 (Leu-CH), 22.84 (Leu-CH₃), 21.86 (Leu-CH₃), 18.17 (Ala-CH₃) 17.94 (Ala-CH₃). LR-MS: m/z=521.32117

Compound 17: ¹H NMR (500 MHz, MeOH-d4) δ 7.48-7.11 (m, 5H, Bn-H), 5.17 (d, J=12.3 Hz, 1H, Bn-CH₂), 5.12 (d, J=12.3 Hz, 1H, Bn-CH₂), 4.43 (dq, J=11.3, 3.1 Hz, 2H, 2× Ala-Cα-H), 4.07-3.85 (m, 3H, Leu-Cα-H⁺ Gly-CH₂), 1.72-1.62 (m, 1H, Leu-CH), 1.59-1.45 (m, 1H, Leu-CH₂), 1.52 (d, J=7.1 Hz, 3H, Ala-CH₃), 1.41 (d, J=7.3 Hz, 3H, Ala-CH₃), 0.91 (d, J=6.6 Hz, 3H, Leu-CH₃), 0.89 (d, J=6.5 Hz, 3H, Leu-CH₃). ¹³C NMR (126 MHz, MeOH-d4) δ 174.55 (C═O), 173.78 (C═O), 171.49 (C═O), 170.84 (C═O), 137.21 (Bn-C), 129.59 (Bn-C), 129.36 (Bn-C), 129.31 (Bn-C), 67.98 (Bn-CH₂), 52.87 (Ala-Cα), 50.26 (Leu-Ca), 49.63 (Ala-Cα) 43.11 (Gly-Ca), 42.21 (Leu-CH₂), 25.75 (Leu-CH), 23.43 (Leu-CH₃), 21.97 (Leu-CH₃), 17.39 (Ala-CH₃), 17.14 (Ala-CH₃). LR-MS: m/z=421.26 Compound S12: Boc-L-Leu·H₂O (1.82 g, 7.88 mmol, 1.1 eq.), EDC·HCl (2.06 g, 10.74 mmol, 1.5 eq.) and HOBt·H₂O (1.65 g, 10.74 mmol, 1.5 eq.) were dissolved in 60 mL dry DMF and stirred for 30 min at 4° C. L-Ala-COOMe·HCl (1 g, 7.16 mmol, 1 eq.) and DIPEA (2.49 mL, 14.32 mmol, 2 eq.) were dispersed in 5 mL dry DMF and added to the the activated acid. The ice-bath was removed and the reaction was allowed to warm to room temperature. Reaction progress was monitored by TLC (CH₂C₁₂: MeOH, 95: 5) and full turnover was achieved after 3 h. The reaction was concentrated in vacuo and diluted with 100 mL H2O. The aqueous phase was extracted with CH2Cl2 (3×100 mL) and the combined org. phases were washed with 100 mL aq. HCl (0.2 M), half satd. brine (100 mL) and brine (100 mL). The organic phase was dried over Na2SO4 and solvent was evaporated in vacuo. The product was purified by MPLC (CH₂Cl₂:MeOH, 1-10%) and obtained as a white amorphous solid (1.64 g, 72

Compound 17: (acc. to Jiang et al. JACS 2003, 7, 1877-1887) xy (640 mg, 2.02 mmol, 1 eq.) was dissolved in 20 mL solvent mixture (THF/MeOH/H₂O, 3: 1: 1). LiOH (145.14 mg, 6.06 mmol, 3 eq.) was added and the reaction was stirred at room temperature for 1.5 h. The reaction was cooled to 4° C. with an ice bath and quenched with 1 M aq. HCl to pH=4. The reaction was diluted with EtOAc until the phases separated and the organic phase was collected. The organic phase was washed with satd. brine (pH=4) and dried over Na₂SO₄. After evaporation of the solvent, the product was obtained as a white amorphous solid (578 mg, 95%).

### Example 2—Biophysical and Microbiological Evaluation of Drug Conjugates

I. Lectin-binding affinity of lectin-targeted conjugates for their respective lectins

Lectin-binding was confirmed by a fluorescence polarization based competitive binding assay.

1. Competitive binding assays

LecA (according to Joachim et al.): A serial dilution of the test compounds was prepared in TBS/Ca (8.0 g/L NaCl, 2.4 g/L Tris, 0.19 g/L KCl, 0.15 g/L CaCl₂·2 H₂O), with 30% DMSO as co-solvent. A concentrated solution of LecA was diluted in TBS/Ca together with the fluorescent reporter ligand (N-(fluorescein-5-yl)-N′-(3-d-(m-aminophenyl)-galactopyranosyl)-thiocarbamide) to yield concentrations of 40 μM and 20 nM, respectively. 10 μL of this mix was added to 10 μL serial dilutions of the test compounds in a black 384-well microtiter plates (Greiner Bio-One, Germany, cat. no. 781900) in triplicates. After centrifugation (2680 ref, 1 min, room temperature), the reactions were incubated for 30-60 min at room temperature in a humidity chamber. Fluorescence (excitation 485 nm, emission 535 nm) was measured in parallel and perpendicular to the excitation plane on a PheraStar FS plate reader (BMG Labtech GmbH, Germany). The measured intensities were reduced by the values of only LecA in TBS/Ca and fluorescence polarization was calculated. The data was analysed with the MARS Data Analysis Software (BMG Labtech GmbH, Germany) and fitted according to the four-parameter variable slope model. Bottom and top plateaus were fixed according to the control compounds in each assay ((p-nitrophenyl)-p-D-galactoside) and the data was re-analysed with these values fixed. A minimum of three independent measurements on three plates was performed for each inhibitor.

LecB (LecB PAO1 according to Hauck et al. and LecB PA14 according to Sommer et al.): A serial dilution of the test compounds was prepared in TBS/Ca, with 20% DMSO as cosolvent. A concentrated solution of LecB PAO1 or PA14 was diluted in TBS/Ca together with the fluorescent reporter ligand (N-(fluorescein-5-yl)-N′-(α-l-fucopyranosyl ethylen)-thiocarbamide) to yield concentrations of 300 nM and 20 nM, respectively. 10 μL of this mix was added to 10 μL serial dilutions of the test compounds in a black 384-well microtiter plates (Greiner Bio-One, Germany, cat. no. 781900) in triplicates. After centrifugation (2680 ref, 1 min, room temperature), the reactions were incubated for 4-8 h at room temperature in a humidity chamber. Fluorescence was measured and analysed as for LecA. Bottom and top plateaus were fixed according to the control compound in each assay (1-fucose) and the data was re-analysed with these values fixed. A minimum of three independent measurements on three plates was performed for each inhibitor.

### 2. Results

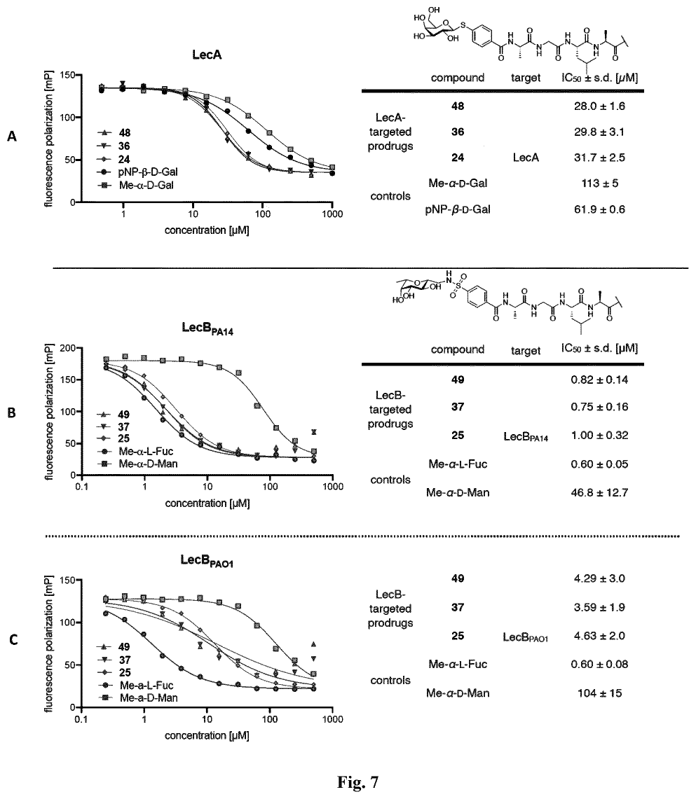

In case of LecA (FIG. 7A), the lectin-targeted prodrugs 24 (ciprofloxacin-based), 36 (amino-pyrrolidine-based) and 48 (aminomethylpyrrolidine-based) showed very similar binding affinities around 30 μM. Methyl-α-D-galactoside (Me-α-D-Gal, IC₅₀=113±5 μM) and p-nitrophenyl-β-thio-galactoside (pNP-β-D-Gal, IC₅₀=61.9±0.6 μM) were used as positive controls. The two P. aeruginosa strains PAO1 and PA14 and their respective lectin homologues represent a broad range of clinical isolates. LecB PAO1 bound the LecB-targeted prodrugs 25 (ciprofloxacin-based), 37 (aminopyrrolidine-based) and 49 (aminomethylpyrrolidine-based) with high affinity in the one-digit micromolar range (FIG. 7B), comparable to L-fucose (IC₅₀=2.63±1.7 M). As observed for LecA, the different prodrugs possessed comparable affinity independent of their cargo. Terminal mannosides and fucosides are the natural ligands of LecB. Thus, methyl-α-D-mannoside (Me-α-D-Man, IC₅₀=104±15 μM) and methyl α-L-fucoside (Me-α-L-Fuc, IC₅₀=0.60±0.08 μM) were used as control compounds.

We further tested the LecB-homologue from P. aeruginosa PA14. As observed before 36 for mannose- and fucose-based carbohydrates, LecB PA14 bound all conjugates and the control compounds with higher affinity compared to LecB PAO1, reaching IC₅₀ values in the low micromolar to high nanomolar range (e.g. 3.59±1.92 μM vs 0.75±0.16 μM for 37) (FIG. 7C, table 2).

TABLE 2
Lectin inhibition, calculated as Kᵢ from IC₅₀ according to Huant et al. [1] N.a. = not applicable,
if IC₅₀ ≤ K_{d} of reporter ligand.

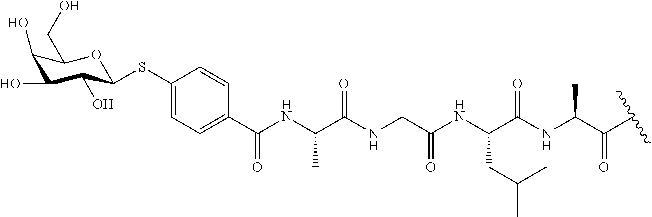

| | compound | target | IC₅₀ ± s.d. [μM] | K [symbol] ± s.d. |
|---|---|---|---|---|
| LecA- | 48 | LecA | 28.0 ± 1.61 | 11.2 ± 0.73 |
| targeted | 38 | | 29.8 ± 3.08 | 12.0 ± 1.41 |
| prodrugs | 24 | | 31.7 ± 2.46 | 12.9 ± 1.12 |
| controls | Me-α- [symbol] -GaI | | 113 ± 5 | 50.0 ± 2.13 |
| | pNP-β- [symbol] -GaI | | 61.9 ± 0.6 | 26.9 ± 0.25 |

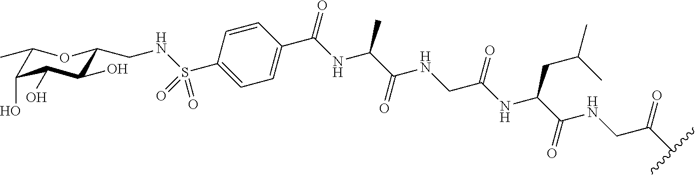

| | | | | |
|---|---|---|---|---|
| LecB-targeted | 49 | LecB [symbol] | 4.29 ± 2.99 | 1.75 ± 1.40 |
| prodnigs | 37 | | 3.59 ± 1.92 | 1.43 ± 0.90 |
| | 25 | | 4.63 ± 1.99 | 1.92 ± 0.93 |
| controls | Me-α-L-Fuc | | 0.60 ± 0.08 | na |
| | Me- [symbol] -Man | | 104 ± 15 | 48.6 ± 6.9 |
| LecB-targeted | 40 | LecB [symbol] | 0.82 ± 0.14 | 0.29 ± 0.05 |
| prodrugs | 37 | | 0.75 ± 0.16 | 0.27 ± 0.06 |
| | 25 | | 1.00 ± 0.32 | 0.36 ± 0.12 |
| controls | Me-α- [symbol] -Fuc | | 0.60 ± 0.05 | 0.21 ± 0.02 |
| | Me- [symbol] -Man | | 46.8 ± 12.7 | 17.6 ± 4.8 |

[symbol]

indicates data missing or illegible when filed

In conclusion, the lectin-targeted prodrugs have the potential to target a broad field of P. aeruginosa strains.

II. Release of antibiotic cargo
1. Prodrug activation assay

In the prodrug activation assay, the bacterial strain Pseudomonas aeruginosa PA14 wt (DSM 19882) was used. The strain was obtained from the German Collection of Microorganisms and Cell Cultures (DSMZ).

P. aeruginosa PA14 was streaked on LB-agar plates (1% agar) from glycerol stocks and incubated at 37° C. overnight. 2-5 colonies were picked and dispersed in 10 mL LB (10 g/L trypton, 10 g/L NaCl, 5 g/L yeast extracts). This dispersion was incubated overnight at 37° C., 180 rpm under high humidity. The culture was centrifuged (4000 ref, room temperature, 10 min) and the supernatant was filtered (0.22 m pore size). 1 mL filtrate was mixed with 9 mL human plasma (BioJVT—West Sussex, United Kingdom, LiHep-treated, pooled, mixed gender) to result in the matrix for this experiment.

A 1 mM solution of the studied compound was prepared in PBS (150 mM NaCl, 2.6 mM KCl, 1.4 mM KH₂PO₄, 10 mM Na₂HPO₄, pH 7.4) with 20% DMSO. 50 μL of this solution was diluted in 950 μL matrix or human plasma (spiked with 10% LB) on ice. After brief vortex, 100 μL were immediately treated with 100 μL ice-cold MeCN (spiked with 1.5 M diphenhydramine as internal standard). The rest of the solution was incubated at 37° C. and 500 rpm in an eppendorf thermomixer. At various time points (30, 60, 120, 180 min), 100 L sample were taken and treated as described above. After extensive vortexing, the samples were centrifuged (17600 ref, 10 min, 10° C.) and the supernatant was analysed by HPLC-MS. The AUC of the parent drug and its cleavage products and the internal standard was quantified using Compass QuantAnalysis quantification software. The relative AUC was calculated by AUC (compound)/AUC(ISTD). Procaine was used as a positive control as it readily degrades in human plasma.

### 2. Results

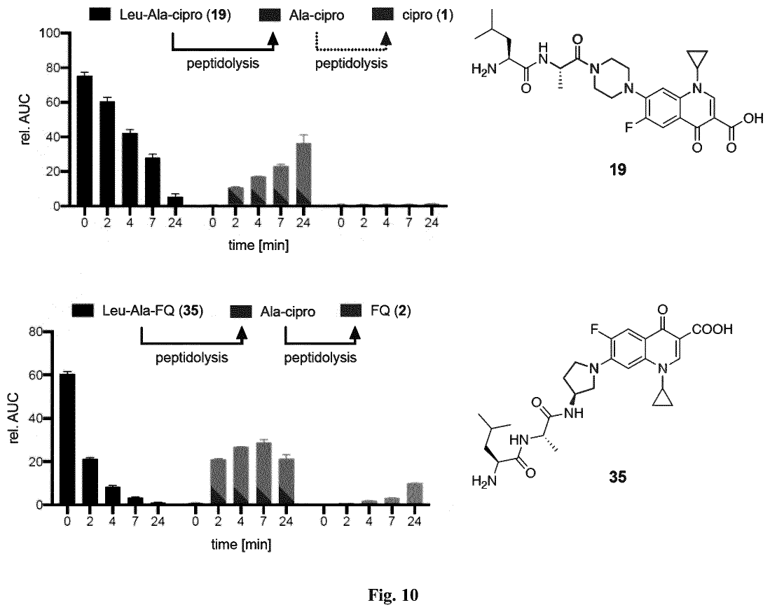

The peptide linker of the prodrugs was designed to be cleaved in presence of LasB, an endopeptidase expressed by P. aeruginosa. In order to resemble the complex variety of proteases, primary and secondary metabolites during an infection, a sterile filtrate of an overnight culture from P. aeruginosa PA14 was used instead of purified LasB. This matrix contains a plethora of enzymes, some of them being able to process the intermediate dipeptides from the first LasB-mediated cleavage and finally release the naked antibiotic cargo. LasB-mediated cleavage of the tetrapeptide was generally very fast and therefore not a rate limiting step. In contrast, preliminary scouting experiments showed that dipeptide 19 and 35 only slowly released their antibiotic cargo in PA14-filtrate (no full release after 24 h, FIG. 10).

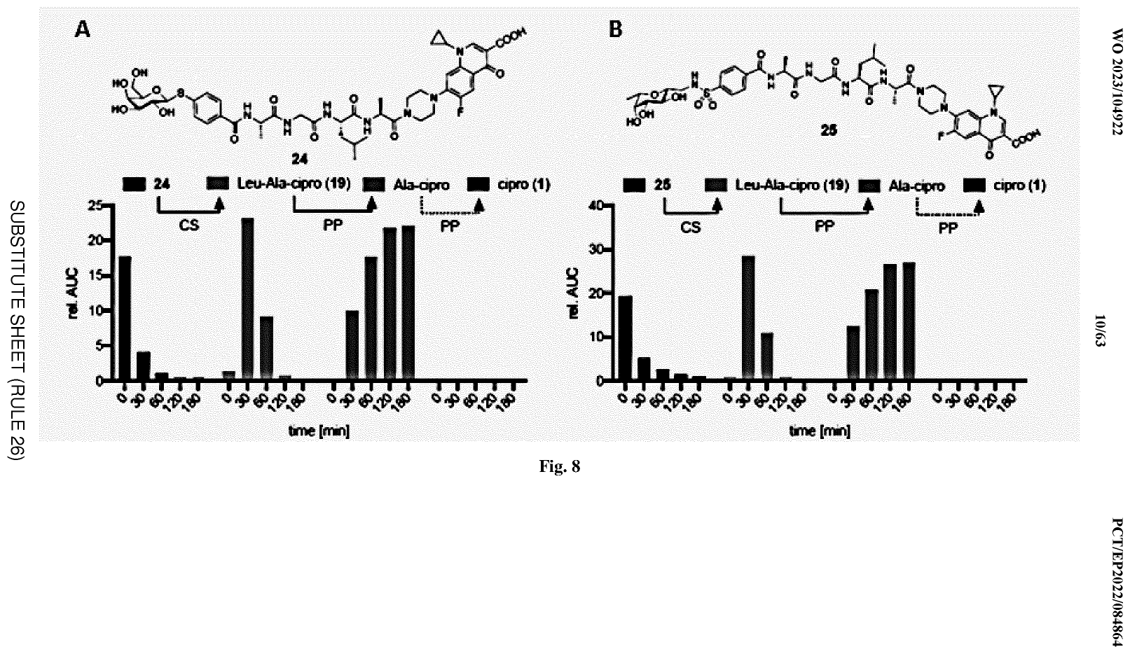

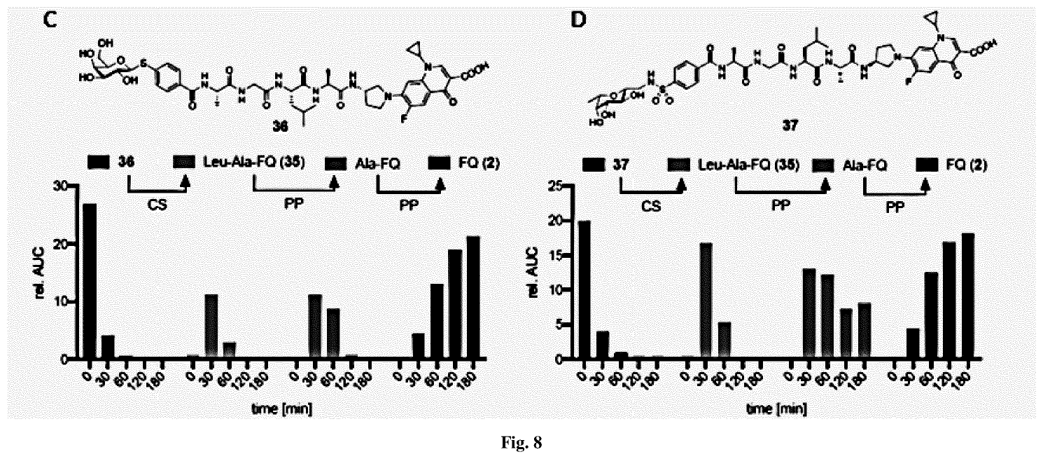

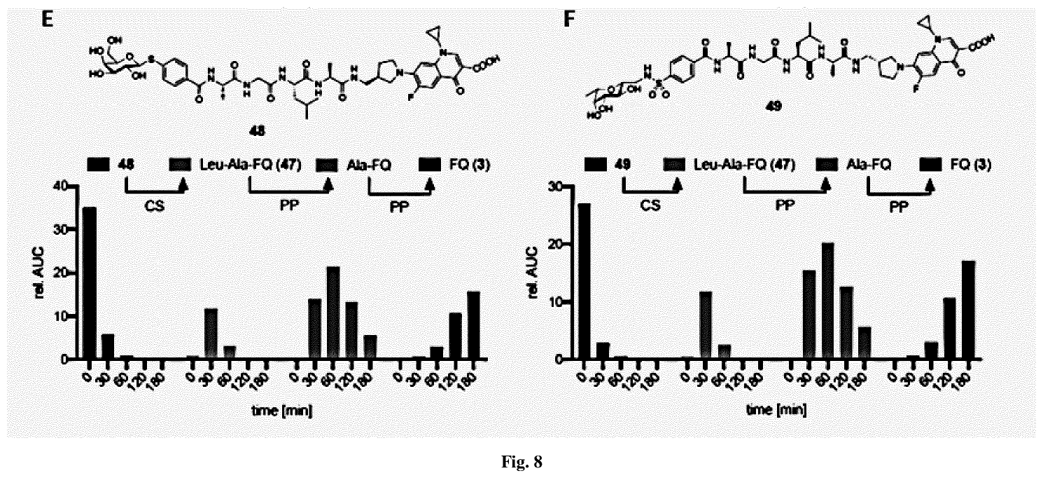

To increase complexity of the biological matrix and to mimic the infection scenario, human blood plasma was added to the cleavage experiments (FIG. 8). Indeed, in presence of PA14-filtrate and human blood plasma, the lectin-targeted prodrugs 36/37 and 48/49 released a significant amount of their antibiotic cargo within 3 h. The ciprofloxacin-based prodrugs 24 and 25 were also processed after initial cleavage by LasB and the resulting dipeptide 19 was further metabolised. However, degradation stopped at the stage of the secondary amide Ala-ciprofloxacin, showing that the ciprofloxacin-based prodrugs are not fully metabolised to release their antibiotic cargo.

When comparing the aminopyrrolidine with the aminomethylpyrrolidine series, only minor differences could be observed. All primary amide-based prodrugs were quickly metabolised and efficiently released the corresponding fluoroquinolones. The aminopyrrolidine-based prodrugs 36 and 37 released their antibiotic cargo 2 faster than aminomethylpyrrolidine 3 was released from prodrugs 48 and 49. This was unexpected, as the aminomethylpyrrolidine-series has an additional CH₂-spacer to increase accessibility for proteolytic enzymes.

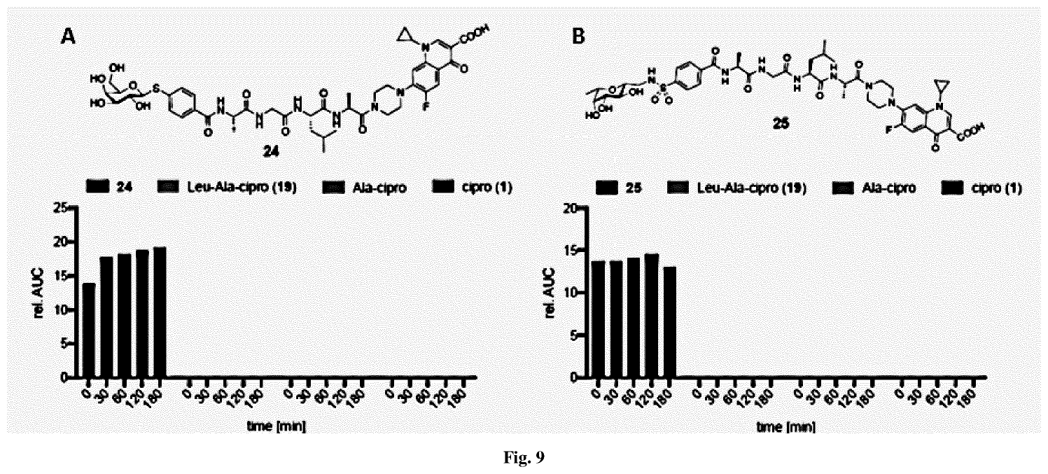

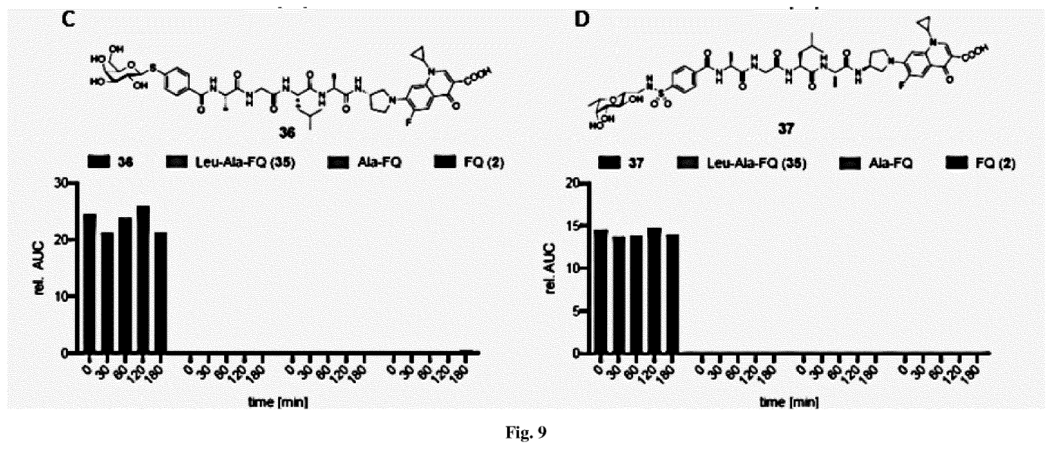

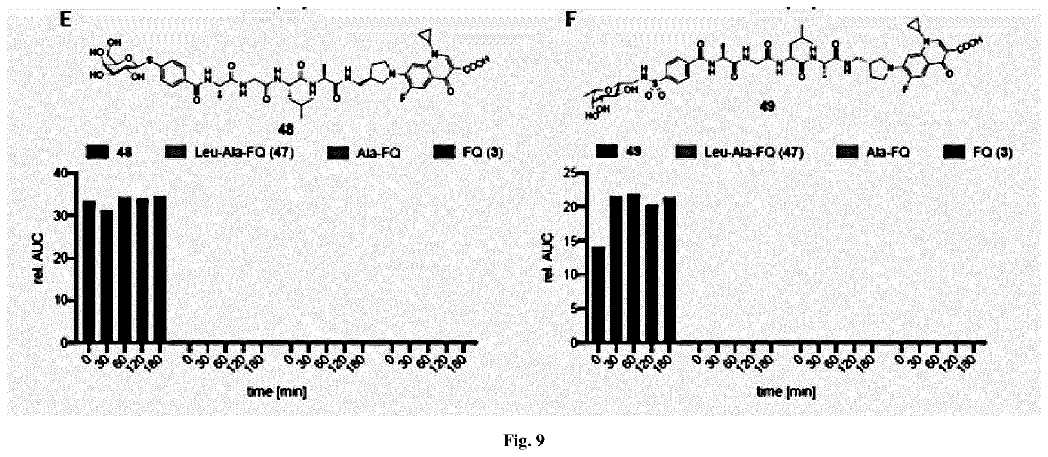

The prodrugs' stability in presence of human blood plasma was assessed in a control experiment in absence of bacterial matrix (FIG. 9). Indeed, the compounds showed no release of fluoroquinolone or peptide-conjugated intermediates within three hours.

In conclusion, activation of the prodrugs is efficiently triggered by the presence of proteases expressed by P. aeruginosa.

III. Antibiotic activity of lectin-targeted drug conjugates
1. Antibiotic susceptibility (MIC assay)

The antibiotic activity of the reference compounds 1, 2, 3, 19, 35 and 47 was determined by broth microdilution assay based on the EUCAST guidelines, according to Wiegand, Hilpert and Han-cock. Serial dilutions in sterile MGller-Hinton broth II (Fluka analytical, cat. no. 90922: 17.5 g/L casein acid hydrolysate, 3 g/L beef extract, 1.5 g/L starch, supplemented with 20-25 mg/L Ca²⁺ and 10-15 mg/L Mg²⁺, pH 7.3) of the conjugates were prepared from 100 mM DMSO stocks (for ciprofloxacin (1) a 10 mM aq. stock of ciprofloxacin·HCl was used), in sterile 96-well plates, yielding a concentration range from 128 g/mL -0.125 g/mL (12.8-0.0125 for ciprofloxacin 1 and fluoroquinolones 2 and 3). Bacterial strains were streaked on LB-agar plates (1% agar) from glycerol stocks and incubated at 37° C. overnight. Colonies were picked from plate and dispersed in fresh MiGler-Hinton broth II (MHB II) to yield an OD₆₀₀ of 0.08-0.13. This dispersion was diluted 1:100 in fresh MHB II, which was then used for the assay to achieve a final inoculum of 5×10⁵ CFU/mL. 50 μL inoculum was mixed with 50 μL of the serial dilution in the corresponding well of the 96-well plate.

For the measurement of the time- and matrix-dependent antibiotic activity of prodrugs 24, 25, 36, 37, 48 and 49, a serial dilution in PBS with 20% DMSO was prepared in sterile 96-well plates, yielding a concentration range of 1 mM -1.9 M (100 M -0.19 M for ciprofloxacin). The different matrices were prepared under sterile conditions: (i) 5 mL human plasma and 1 mL sterile filtrate from an P. aeruginosa PA14 overnight culture in LB were mixed with 4 mL PBS; (ii) 1 mL sterile filtrate from an P. aeruginosa PA14 overnight culture was diluted in 9 mL PBS; (iii) PBS only and (iv) 5 mL human plasma and 1 mL LB were mixed with 4 mL PBS. 6 L of each compound dilution series was diluted in 115 L matrix (˜-1:20 dilution) in a 96-well format at time point T=−3 h or T=−10 min.

The plates prepared at T=−3 h were sealed with gas-permeable foil and incubated at 37° C. in a humid incubator. The other plates were kept at room temperature. At T=0 h, 50 L of each well was mixed with 50 μL incoculum (as described above) in double-concentrated MHBII in a sterile 96-well plate.

The plates were sealed with gas-permeable foil and incubated at 37° C. for 18-20 h in a humid incubator. Growth inhibition was assessed by visual inspection and given MIC values are the lowest concentration of antibiotic at which there was no visible growth.

### 2. Results

The antibiotic activity (MIC) of the synthetic fluoroquinolones 2 and 3 against P. aeruginosa PA14 was analysed by microbroth dilution assay (Table 3). As reported by Sanchez et al., compound 2 (MIC=0.027-0.054 μM) was more active than ciprofloxacin 1 (MIC=0.125-0.25 RM). The potent antibacterial activity of aminomethylpyrrolidine 3 was slightly weaker (MIC=0.29-1.45 μM) than the other two antibiotics. The dipeptidylfluoroquinolone conjugates 19 (ciprofloxacin-based), 35 (amino-pyrrolidine-based) and 47 (aminomethylpyrrolidine-based)-i.e. those that result after initial cleavage by LasB—only showed low antibiotic activity in the micromolar range.

Previous experiments showed, that a majority of free antibiotic drug was released from the prodrugs within 3 h in presence of PA14-filtrate together with human blood plasma (FIG. 8). Therefore, the prodrugs were incubated for 3 h (and <10 min as control) in different matrices before transferring them to the antibiotic susceptibility assay (Table 3): PBS (matrix 1), 50% human blood plasma spiked with 10% PA14-filtrate in PBS (matrix 2), 10% PA14-filtrate in PBS (matrix 3) and 50% human blood plasma spiked with 10% LB in PBS (matrix 4).

In adherence to the definition, the lectin-targeted conjugates 24, 25, 35, 36, 48 and 49 (Table 3, matrix 1) did not show any antibiotic activity below 25 M from PBS (MIC >25 μM). In contrast, a brief pre-incubation of <10 min in a mixture of human blood plasma and PA14-filtrate in PBS (matrix 2) activates the primary amide-based prodrugs 36/37 (MIC=0.195-0.39 M and 0.78-0.156 RM, respectively) and 48/49 (MIC=3.13-12.5 μM and 3.13-6.25 μM, respectively), while the ciproflox-acin-based prodrugs 24 and 25 remained inactive (MIC >25 μM). This trend became even stronger after 3 h of preincubation: while the ciprofloxacin series remained inactive, especially the aminopyrrolidine-based prodrugs 36/37 were highly potent (MIC=0.098-0.195 μM) and almost reached the antibiotic activity of their parent fluoroquinolone 2 (MIC=0.027-0.054 μM), indicating a very efficient drug release during the experiment. Under the same conditions (Matrix 2), the aminomethylpyrrolidine series 48/49 reached low micromolar antibacterial activities around 1.56-3.13 μM, which is close to the activity of parent fluoroquinolone 3 (MIC=0.29-1.45 μM). It has to be noted, that MICs of the parent drugs 1-3 and the dipeptide-conjugates 19, 35, 47 were measured under conventional conditions, i.e. without the addition of a proteolytically active biological matrix like blood plasma or PA14 filtrate. Thus, effects like metabolism or plasma protein binding are drastically reduced, potentially resulting in lower MIC-values. The antibiotic activity difference within the different fluoro-quinolone-series can be explained by the different drug-release kinetics of the prodrugs (FIG. 8) and by the intrinsically lower antibacterial activity of aminomethylpyrrolidine-FQ 3 compared to amino-pyrrolidine-FQ 2 (MIC=0.29-1.45 μM and MIC=0.027-0.054 μM, respectively).

Interestingly, primary amide-based prodrugs 36, 37, 48 and 49 still released a significant amount of active drug in presence of P. aeruginosa culture-filtrate only (Table 3, matrix 3), resulting in antibiotic activities in the low micro molar range (e.g. MIC=0.195-0.78 μM for 36 after 3 h pre-incubation). We reason that the time frame of the experiment itself (18 h incubation at 37° C.) is sufficient to release a significant amount of drug, despite the slower metabolism in culture-filtrate (FIG. 10). This assumption is in coherence to the fact that even with a pre-incubation time of 3 h, the antibiotic activity increased only mildly (e.g. for 36: MIC (<10 min pre-incubation)=0.78-1.56 μM vs MIC (3 h pre-incubation)=0.195-0.78 μM, for 49: MIC (<10 min pre-incubation)=3.13 M vs MIC (3 h pre-incubation)=1.56-3.13 μM).

TABLE 3
Antibacterial activity of the control compounds 1, 2, 3, 19, 35 and 47 and of the lectin-targeted
LasB-cleavable conjugates 24, 25, 36, 37, 48 and 49 against P. aeruginosa PA14. The prodrugs were
tested under different conditions, varying the pre-incubation time (10 min vs. 3h) in different biological
matrices 1-4 before adding the inoculum. ^{a}

| | 1 | 24 | 25 | 36 | 37 | 48 | 49 |
|---|---|---|---|---|---|---|---|
| | control | LecA-targeted | LecB-targeted | LecA-targeted | LecB-targeted | LecA-targeted | LecB-targeted |
| antibiotic cargo | 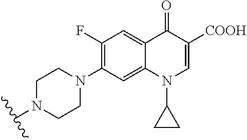 [SMILES: *N1CCN(c2cc3c(cc2F)c(=O)c(C(=O)O)cn3C2CC2)CC1] | | | 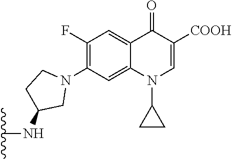 [SMILES: *N[C@H]1CCN(c2cc3c(cc2F)c(=O)c(C(=O)O)cn3C2CC2)C1] | | 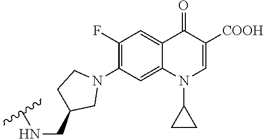 [SMILES: *NC[C@H]1CCN(c2cc3c(cc2F)c(=O)c(C(=O)O)cn3C2CC2)C1] | |
| | Matrix 1: PBS | | | | | | |
| <10 min | 0.125-0.156 | >25 | >25 | >25 | >25 | >25 | >25 |
| 3 h | 0.125-0.25 | >25 | >25 | >25 | >25 | >25 | >25 |

Matrix 2: 50% human blood plasma + 10% P. aeruginosa culture supernatant in PBS

| | | | | | | | |
|---|---|---|---|---|---|---|---|
| <10 min | 0.25-0.313 | ≥25 | ≥25 | 0.195-0.39 | 0.78-1.56 | 3.13-12.5 | 3.13-6.25 |
| 3 h | 0.156 - 0.25 | ≥25 | ≥25 | 0.1 | 0.1-0.195 | 1.56 | 1.56-3.13 |

Matrix 3: 10% P. aeruginosa culture supernatant in PBS

| | | | | | | | |
|---|---|---|---|---|---|---|---|
| <10 min | 0.125-0.313 | ≥25 | ≥25 | 0.78-3.13 | 1.56-12.5 | 1.56-3.13 | 3.13-6.25 |
| 3 h | 0.125-0.313 | ≥25 | ≥25 | 0.195-3.13 | 0.39-3.13 | 1.56-3.13 | 1.56-6.25 |

Matrix 4: 50% human blood plasma + 10% LB in PBS

| | | | | | | | |
|---|---|---|---|---|---|---|---|
| <10 min | 0.156-0.313 | ≥25 | ≥25 | ≥25 | ≥25 | >25 | ≥25 |
| 3 h | 0.156-0.313 | ≥25 | ≥25 | 0.78-6.25 | 0.78-6.25 | 12.5->25 | 12.5->25 |
| MIC (parent | [μM] | 1 | 0.125-0.25 | 2 | 0.027-0.054 | 3 | 0.29-1.45 |
| drug) | | | | | | | |
| MIC (dipep- | | 19 | 7.25-14.5 | 35 | 58 | 47 | 28-56 |
| tide-FQ) | | | | | | | |

^{a} Data is represented as minimal inhibitory concentration (MIC) range from at least three independent experiments (exception: N = 2 for matrix 1, <10 min).

In all cases, the antibiotic activity reached after pre-incubation in human blood plasma alone (Table 3, matrix 4) was significantly lower than from the other biological matrices (matrices 2 & 3). Only the aminopyrrolidine-based prodrugs 36 (LecA-targeted) and 37 (LecB-targeted) reached significant potency, however, it varied extensively between the replicates (MIC=0.78-6.25 RM).

Overall, the antibiotic activity of the lectin-targeted prodrugs correlated well with their metabolic activation in the presence of human blood plasma proteins and PA14-filtrate. The ciprofloxacin-based prodrugs could not be fully activated due to the presence of a stable secondary amide and thus showed only weak antibiotic activity, despite their potent antibiotic cargo. In contrast, the primary am-ide-based prodrugs showed efficient release of their antibiotic cargo, resulting in highly potent antimicrobial activity.

### IV. Metabolic Stability and Cytotoxicity of Lectin-Targeted Drug Conjugates In Vitro

### 1. Cytotoxicity Assay

The epithelial cell line A549 (ATCC(R) CCL-185) was cultivated in Dulbecco's modified Eagle's medium (DMEM) with 10% heat-inactivated fetal calf serum (FCS) and 20 mM L-Glutamine at 37° C. and 5% CO₂. A549 cells were seeded into a 96-well-plate (Nunc, Roskilde, Denmark) and grown to 75% confluency. The following compounds were tested in the cell assay: 36, 37 and 2. Every compound was dissolved in DMSO and diluted in PBS (final DMSO concentration in the cell assay: 1%).

Cells were incubated with the respective compound in concentrations ranging from 0.001-50 μM for 24 h at 37° C. and 5% CO₂. Cells treated with vehicle only (DMSO diluted in PBS, final DMSO concentration in the cell assay: 1%) served as a negative control. Furthermore, pure medium (DMEM+10% FCS) and completely damaged cells served as positive controls. To damage cells, cells were treated with 0.5% Triton X-100 1 h prior to addition of MTT (Sigma). After 24 h cells were washed twice with the respective medium. MTT diluted in PBS (stock solution 5 mg/ml) was added to the wells at a final concentration of 1 mg/ml. The cells were incubated for 4 h at 37° C. and 5% CO₂. Medium was removed and 0.04 M HCl in 2-propanol was added. The cells were incubated at room temperature for 15 min. Then supernatant was transferred to a 96-well-plate. The samples were measured at 560 nm and at 670 nm as a reference wavelength on a Tecan Sunrise ELISA Reader using Magellan software. Data was normalized using the following formula: (A-B)/(C-B) with ‘A’ as the respective data point, ‘B’ as the value of the Triton X-100-treated control and ‘C’ as the vehicle control. The experiment was repeated at least three times. The error bars indicate the standard deviation.

### 2. Plasma Stability Assays

Each compound dissolved in DMSO was added to mouse plasma (pH 7.4, 37° C.) or to human plasma (pH 7.4, 37° C.) to yield a final concentration of 1 μM. In addition, procaine and procainamide (dissolved in DMSO) were added to mouse plasma or to human plasma (pH 7.4, 37° C.) to yield a final concentration of 1 μM. Procaine served as positive control as it is unstable in mouse plasma. Procainamide served as negative control as it is stable in mouse plasma. The samples were incubated for 0 min, 15 min, 30 min, 60 min, 90 min, 120 min and 240 min at 37° C. At each time point, 10 μl of the respective sample was extracted with 90 l acetonitrile and 1 μl of caffeine as internal standard for 5 min at 2000 rpm on a MixMate® vortex mixer (Eppendorf). Acetonitrile and caffeine were dispensed using a Mantis Formulatrix®. The samples were centrifuged for 20 min at 2270 ref at 4° C. and the supernatants were transferred to 96-well Greiner V-bottom plates. Samples were analysed using HPLC-MS/MS analysis as described in the respective section. Peak areas of each compound and of the internal standard were analysed using the MultiQuant 3.0 software (AB Sciex). Peak areas of the respective compound were normalised to the internal standard peak area and to the respective peak areas at time point 0 min: (C/D)/(A/B) with A: peak area of the compound at time point 0 min, B: peak area of the internal standard at time point 0 min, C: peak area of the compound at the respective time point, D: peak area of the internal standard at the respective time point. Every experiment was repeated independently at least three times.

### 3. Microsomal Stability Assay

S9 liver microsomes (mouse and human, Thermo Fisher) were thawed slowly on ice. 20 mg/ml of microsomes, 2 μl of a 100 μM solution of every compound and 183 μl of 100 mM phosphate buffer were incubated 5 min at 37° C. in a water bath. Reactions were initiated using 10 μl of 20 mM NADPH (Roth). Samples were incubated in three replicates at 37° C. under gentle agitation at 150 rpm.

At 0, 5, 15, 30, and 60 min, reactions were terminated by the addition of 180 μl acetonitrile using a Mantis Formulatrix® dispenser. Samples were vortexed for 5 min using an Eppendorf MixMate® vortex mixer and centrifuged at 2270 ref for 20 min at 4° C. The supernatants were transferred to 96-well Greiner V-bottom plates, sealed and analysed according to the section HPLC-MS/MS analysis. Peak areas of the respective time point of the compounds were normalized to the peak area at time point 0 min. Then half-life was calculated using linear regression (Microsoft Excel®). Clint [μl/min/mg protein] was calculated using the following formula:

$$Clint = 0.693 / ( 0.005 \times t 1 / 2 ) .$$

### 4. Plasma Protein Binding Assay

Plasma protein binding was assessed using the rapid equilibrium device (RED) system from ThermoFisher. Compounds 2, 36 or 37 were dissolved in DMSO. Naproxene served as control as it shows high plasma protein binding. Compounds were diluted in murine plasma (from CD-1 mice, pooled) or in human plasma (human donors, both genders, pooled) to a final concentration of 1 RM. Dialysis buffer and plasma samples were added to the respective chambers according the manufacturer's protocol. The RED plate was sealed with a tape and incubated at 37° C. for 2 hours at 800 rpm on an Eppendorf MixMate® vortex-mixer. Then samples were withdrawn from the respective chambers. To 25 μl of each dialysis sample, 25 μl of plasma and to 25 μl of plasma sample, 25 l of dialysis buffer was added. Then 150 μl ice-cold extraction solvent (MeCN/H₂O (90:10) containing 12.5 ng/ml caffeine as internal standard) was added. Samples were incubated for 30 min on ice. Then samples were centrifuged at 4° C. at 2270 ref for 10 min. Supernatants were transferred to Greiner V-bottom 96-well plates and sealed with a tape. Then samples were subjected to HPLC-MS/MS analysis as described in the section ‘HPLC-MS/MS analysis’. The percentage of bound compound was calculated as follows:

$${f}_{u} = ( {c}_{buffer chamber} / {c}_{plasma chamber} ) * 100 {f}_{bound} = 1 - {f}_{u}$$

### 5. Results

Due to their excellent antibiotic activity profile against P. aeruginosa PA14 in-vitro, amino-pyrrolidine-fluoroquinolone 2 and the corresponding lectin-targeted prodrugs 36 and 37 were chosen for further early in-vitro ADMET studies (Table 4).

TABLE 4
In-vitro ADMET data of the two aminopyrrolidine-based lectin-targeted drug conjugates 36
and 37 and their common fluoroquinolone cargo 2. All compounds showed good metabolic stability in
blood plasma an in presence of liver cell microsomal fractions. Acute cytotoxicity against A549-cells
was not observed. ª

| | metabolic stability | | | | | | | | |
|---|---|---|---|---|---|---|---|---|---|
| | | | CL_{mic} | | plasma stability, t_{1/2} | | plasma protein | | cytotoxicity |
| | t_{1/2} [min] | | [μL/min/mg protein] | | [min] | | binding [%] | | A549 |
| | MLM | HLM | S9ₘₒᵤₛₑ | S9ₕᵤₘₐₙ | mouse | human | mouse | human | cells [μM] |
| 36 | 100 | 93 | 14 | 15 | >240 | >240 | 74.0 ± 3.7 | 97.2 ± 4.8 | 21.7 |
| 37 | 216 | 178 | 6.4 | 7.8 | 74 | 135 | 30.1 ± 9.2 | 51.1 ± 13.3 | >50 |
| 2 | >60 | 41 | <23 | 33.49 | >240 | 135 | 77.7 ± 10.9 | 93.5 ± 1.3 | 20.8 |

^{a} Data is presented as mean and standard deviation from at least three independent experiments. S9ₘₒᵤₛₑ, mouse S9 liver fractions; S9ₕᵤₘₐₙ, human S9 liver fractions; CL_{MIC}, microsomal clearance, calculated from t_{1/2}.

Metabolic stability was assessed against human and mouse liver microsomes and blood plasma. High metabolic stability in mouse and human S9 liver fractions was observed for the prodrugs 36 (t_{l/2, MLM}=100 min, t1/2, HLM=93 min) and 37 (t_{1/2, MLM}=216 min, t_{1/2, HLM}=178 min). In contrast, metabolism of the parent fluoroquinolone 2 was twofold faster in human liver microsomes (t_{1/2, HLM}=41 min), which is most likely due the presence of a free primary amine in fluoroquinolone 2, that is masked in the prodrugs. In mouse and human blood plasma, the LecA-targeted prodrug 36 was fully stable (t_{1/2}>240 min) and the LecB-targeted prodrug 37 was somewhat less stable under these conditions (t_{1/2, MBP}=74 min, t_{1/2, HBP}=135 min). High stability showed reference compound 2 in blood plasma (t_{1/2, MBP} >240 min, t_{1/2, HBP}=135 min).

Plasma protein binding was assessed in mouse and human blood plasma since very high plasma protein binding (>99%) can mask prodrugs and prevent e.g. binding to their corresponding lectins and accessibility to LasB. The LecA-targeted prodrug 36 showed comparable protein binding (74% for mouse blood plasma, 97% for human blood plasma) to its parent fluoroquinolone 2 (78% for mouse blood plasma, 94% for human blood plasma). Interestingly, the C-glycoside-based prodrug 37 showed reduced plasma protein binding significantly (30% for mouse blood plasma, 51% for human blood plasma). Cytotoxicity was assessed in A549 cells. Whereas 37 showed no cytotoxicity up to 50 M, 36 and 2 gave IC₅₀ values of 21.7 M and 20.8 M, respectively. However, even for 36 and 2 this is acceptable as these values are more than 20-fold higher compared to the in-vitro activity.

### V. Cellular Permissibility of Lectin-Targeted Drug Conjugates

1. Cell accumulation assay

A549 cells were seed into 96-well-plates as described for the cytotoxicity assay. Cells were cultivated at 37° C. and 5% CO₂ until they reached 95% confluency. Cells were treated with 2, 36 or 37 at a final concentration of 10 g/ml or left untreated. Each condition was assayed in technical dupli-cates with two biological replicates. Cells were treated for 15 min, 30 min and 60 min. After incubation for the respective time point, cells were washed twice with pre-warmed PBS and were then lysed in MeOH and scratched from the surface. Supernatants from medium, wash fluids as well as the cell extracts were subjected to mass spectrometric analysis. For wash fluid and medium samples, calibration and QC samples were prepared using PBS as matrix and spiking the respective compounds into the matrix. For cell extract samples, calibration and QC samples were prepared using MeOH as matrix. For calibration and QC samples compounds were dispensed using a Mantis(R) Formulatrix. Medium, wash fluid, cell extract samples as well as both calibration and QC samples were extracted using MeOH containing 12.5 ng/ml caffeine as internal standard for 10 min at 800 rpm on an Eppendorf (R) Vortex MixMate and then centrifuged at 4000 rpm for 20 min at 4° C. Supernatants were transferred to a Greiner V-bottom plate, sealed and subjected for HPLC-MS/MS analysis.

### 2. HPLC-MS/MS Analysis

Cell samples as well as plasma stability, plasma protein binding and metabolic stability samples were analysed using an Agilent 1290 Infinity II coupled to an AB Sciex 6500plus mass spec-trometer. LC conditions were as follows: column: Agilent Zorbax Eclipse Plus C₁₈, 50×2.1 mm, 1.8 m; temperature: 30° C.; injection volume: 5 l per sample; flow rate: 700 l/min. Solvents: A: water+0.1% formic acid; solvent B: 95% acetonitrile/5% H₂O+0.1% formic acid. Gradient for 2, 36 and 37: 99% A from 0 min until 1 min; 99-0% A from 1.0 until 2.2 min, 0% A until 3.2 min. Gradient for naproxene: 99% A from 0 min until 1 min; 99-0% A from 1.0 until 5.5 min, 0% A until 6.0 min. Gradient for procaine and procaine: 99% A from 0 min until 1.0 min, 99-0% A from 1.0 until 3.5 min, 0% A until 3.7 min. Mass transitions for controls and compounds are depicted in table 5.

TABLE 5
HPLC-MS/MS analysis results

| | Q1 mass | Q3 mass | Time | DP | CE | CXP |
|---|---|---|---|---|---|---|
| ID | [Da] | [Da] | [msec] | [volts] | [volts] | [volts] |
| 2 | 332.917 | 314.9 | 30 | 1 | 29 | 34 |
| | 332.917 | 272.2 | 30 | 1 | 27 | 14 |
| 36 | 940.206 | 778.2 | 30 | −300 | −48 | −37 |
| | 940.206 | 734.3 | 30 | −300 | −62 | −29 |
| 37 | 985.228 | 941.3 | 30 | −300 | −58 | −43 |
| | 985.228 | 527.2 | 30 | −300 | −70 | −23 |
| Naproxene | 231.106 | 185.1 | 50 | 80 | 19 | 10 |
| | 231.106 | 170.2 | 50 | 80 | 33 | 12 |
| Caffeine | 195.024 | 138.0 | 50 | 80 | 25 | 14 |
| | 195.024 | 110.0 | 50 | 80 | 31 | 18 |
| Procaine | 236.773 | 100.0 | 30 | 80 | 21 | 12 |
| | 236.773 | 120.0 | 30 | 80 | 31 | 14 |
| Procainamide | 235.744 | 163.0 | 30 | 80 | 21 | 18 |
| | 235.744 | 120.0 | 30 | 80 | 39 | 12 |

### 3. Results

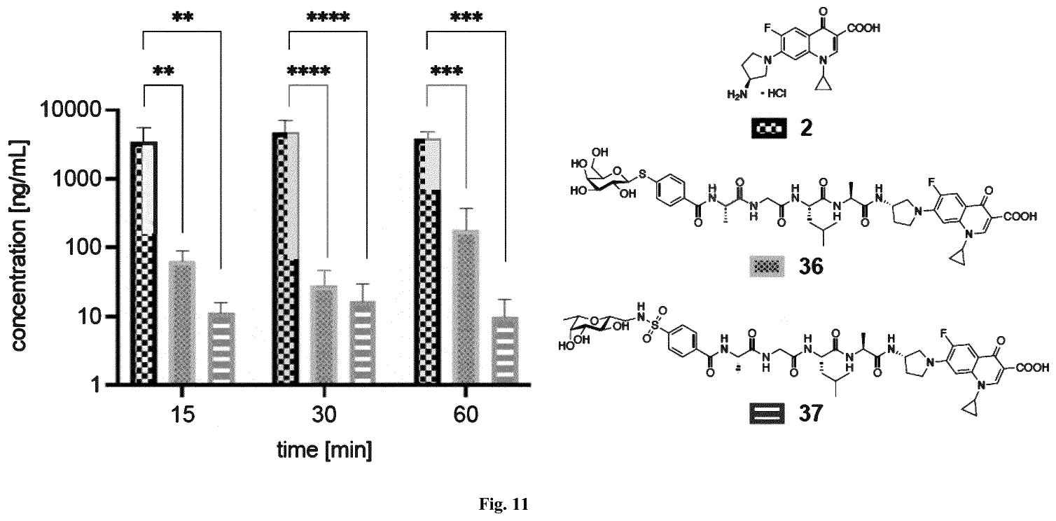

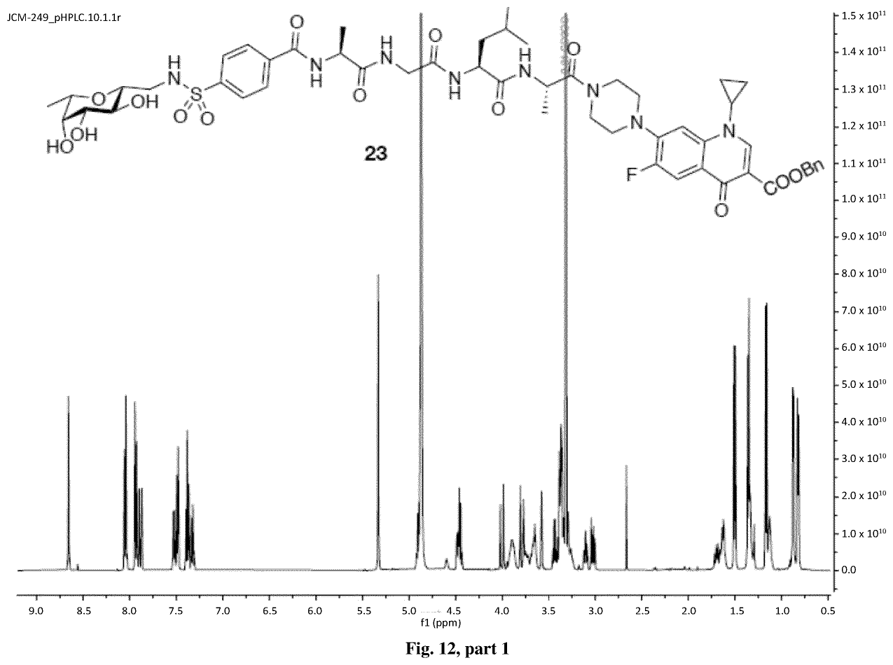

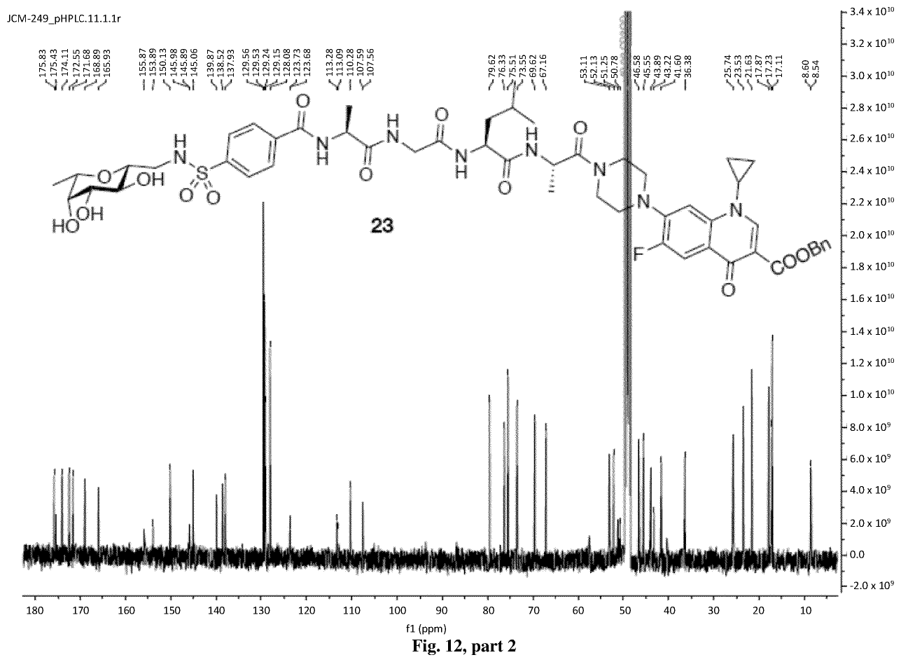

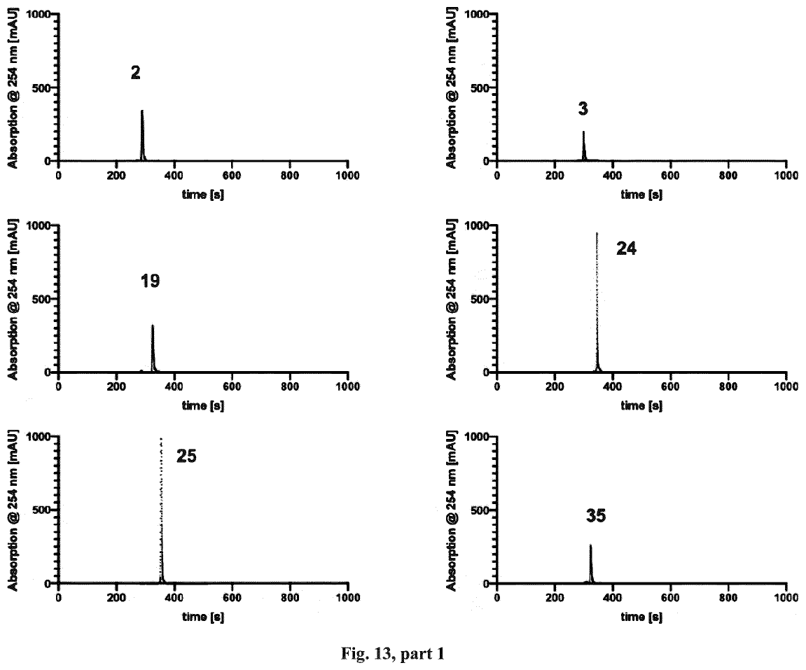

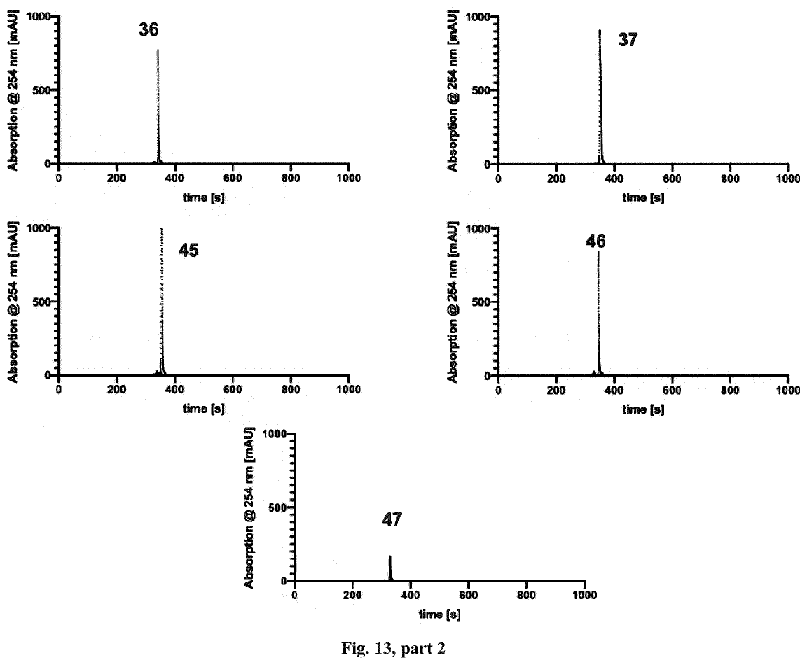

The specific mechanisms of fluoroquinolone-related side effects are not yet fully understood. There is evidence for oxidative stress and the impairment of the mitochondrial DNA replication system induced by ciprofloxacin. In combination with an unspecific drug accumulation in sensitive tissues, the effects described above could lead to severe tissue damage. These intracellular side-effects benefit from the excellent permeation of fluoroquinolone drugs across biological membranes by fluoroquinolone drugs. It is reasonable that lower intracellular availability could reduce these side effects. To test this hypothesis, cell accumulation experiments were performed with prodrugs 36/37 and with their parent fluoroquinolone 2 (FIG. 11). While compound 2 was highly abundant intracellularly (2.9±0.9 g/ml after 60 min incubation and 4.3±0.8 g/ml after 30 min incubation), both prodrugs 36 and 37 showed very low intracellular concentrations (0.013-0.016 μg/ml after 30 and 60 min for 36, 0.016 g/ml after 60 min and 0.027 g/ml after 30 min incubation for 37). It is interesting that all three compounds 2, 36 and 37 showed only very low intracellular drug levels 15 min after incubation suggesting that compounds are not rapidly taken up. Moreover, compound 37 and 2 showed slightly higher intracellular levels for an incubation period of 30 min compared to 60 min. Moreover, it was assessed if 36 and 37 might have been cleaved intracellularly and, thus, looked for compound 2. However, this was not the case. In conclusion, the chemical nature of the prodrugs resulted in a decreased ability to permeate into human cells and reach intracellular off-targets. In combination with the targeted drug delivery approach, this could synergistically lead to drastic reduction of severe side-effects.

### Example 3—Divalent Fluorescent LecA Ligands as Tools to Demonstrate the Biofilm Targeting Efficacy

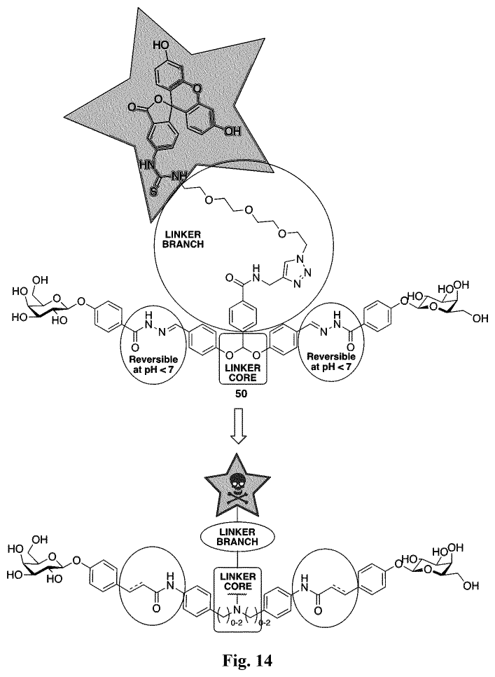

One of the drawbacks of carbohydrate-based ligands is the weak monovalent sugar binding. However, a boost in activity can be achieved by multivalent display of the binding epitopes. High affinity LecA ligands can be modified to efficient delivery scaffolds for a lectin-directed theranostics targeting P. aeruginosa. The divalent fluorescent ligand 50 (FIG. 14) carrying fluorescein as a cargo showed high affinity to LecA in SPR (50 K_{D}=37.2±2.9 nM), but degraded in aqueous buffers probably as a result of acetal linker core and/or the presence of reversible acylhydrazone bonds (EP19306432.6). Here we introduce novel design of LecA targeting divalent scaffold with a nitrogen atom to provide the central branching point. Furthermore, the acylhydrazone motif was replaced with a more stable amide bond.

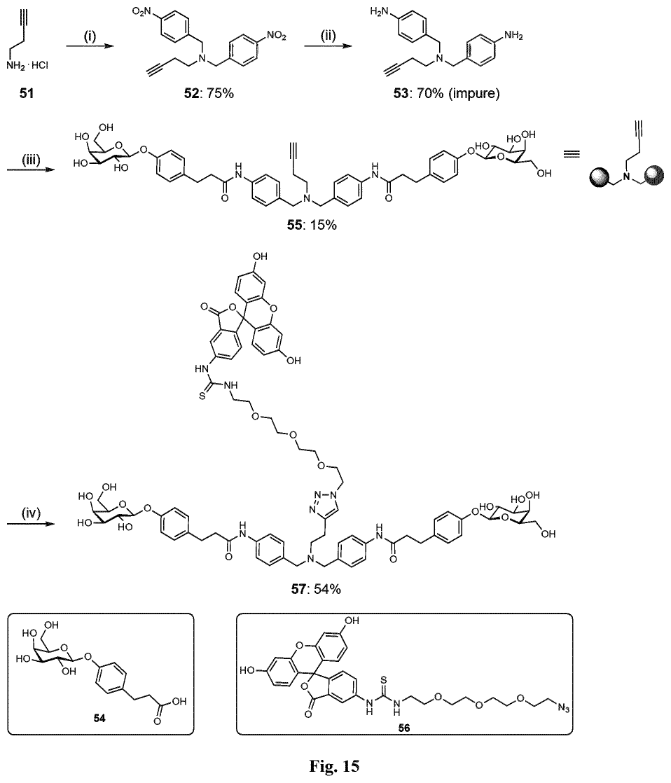

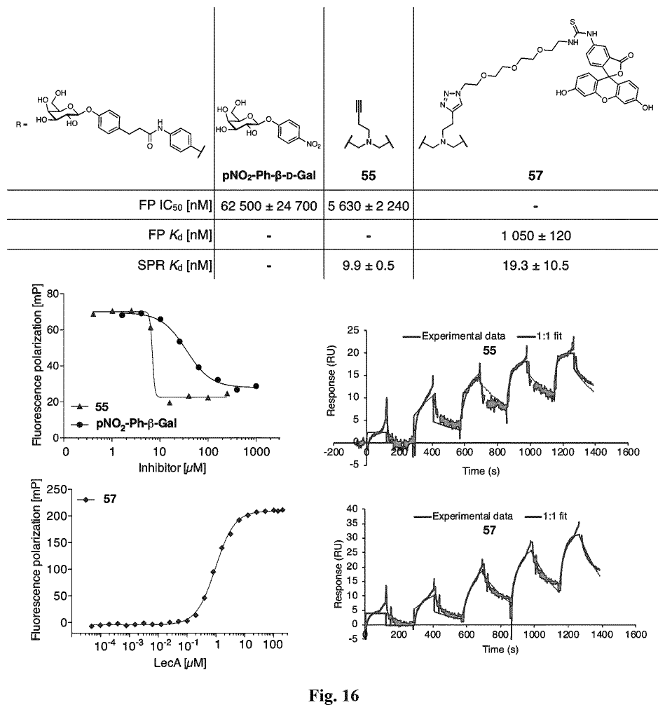

Synthesis of the divalent branched LecA ligand based on a central nitrogen started with a double nucleophilic substitution of the 4-nitrobenzyl bromide with but-3-yn-1-amine (51) (FIG. 15). Selective reduction of the bis-nitro intermediate 52 with iron powder gave the desired bis-aniline linker 53 with impurities. The RP chromatography would probably be able to completely purify 53, but was skipped here and only performed after peptide coupling to the galactoside 54. Galactoside 54 was synthesised as described in EP19306432.6. Divalent ligand 55 was synthesized in poor yield (15%) due to impure starting material as well as side product formation. Despite slow reaction turnover during Huisgen di-polar cycloaddition between divalent ligand 55 and the azide modified fluorescein 56, possibly due to copper coordination to the reactants, the divalent fluorescent ligand 57 was synthesized in 54% yield.

### N,N-Bis(4-nitrobenzyl)but-3-yn-1-amine (52)

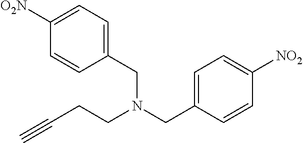 [SMILES: C#CCCN(Cc1ccc([N+](=O)[O-])cc1)Cc1ccc([N+](=O)[O-])cc1]

But-3-yn-1-amine hydrochloride 51 (55.2 mg, 0.55 mmol), 4-nitrobenzyl bromide (327 mg, 1.52 mmol) and potassium carbonate (264 mg, 1.91 mmol) were suspended in dimethylformamide (4 mL) and stirred at room temperature overnight. The reaction was diluted with dichloromethane and water, organic phase was washed with satd. aqueous NaHCO₃ and half satd. brine, dried over anhydrous Na₂SO₄, filtered and concentrated in vacuo. Purification by normal phase MPLC (petrol ether/ethyl acetate, 5-35% ethyl acetate) gave 52 (134 mg, 0.39 mmol, 75%) as a pale-yellow solid.

¹H NMR (500 MHz, Acetone-d₆) δ 8.24-8.16 (m, 4H, ArCH), 7.76 (d, J 8.7 Hz, 4H, ArCH), 3.85 (s, 4H, Ar—CH₂), 2.73 (td, J 7.2, 1.4 Hz, 2H, CH₂CH₂N), 2.52-2.45 (m, 2H, CH₂CH₂N), 2.43 (t, J 2.7 Hz, 1H, C≡CH). ¹³C NMR (126 MHz, Acetone-d₆) δ 148.49 (2C, ArC), 148.07 (2C, ArC), 130.45 (4C, ArCH), 124.19 (4C, ArCH), 83.26 (1C, C≡CH), 71.06 (1C, C≡CH), 58.04 (2C, Ar—CH₂), 53.19 (1C, CH₂CH₂N), 17.40 (1C, CH₂CH₂N).

HPLC-MS: [C₁₈H₁₇N₃O₄+H]⁺ calculated. 340.13, found 340.15.

HRMS: [C₁₈H₁₇N₃O₄+H] calculated. 340.1292, found 340.1289.

### N,N-Bis(4-aminobenzyl)but-3-yn-1-amine (53)

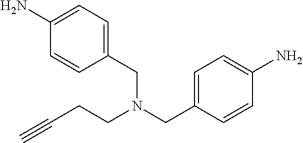 [SMILES: C#CCCN(Cc1ccc(N)cc1)Cc1ccc(N)cc1]

Compound 52 (21.8 mg, 64.2 μmol), iron powder (17.7 mg, 317 gmol) and CaCl₂ (22.4 mg, 202 gmol) were suspended in ethanol/water mixture (1 mL, 8:2) under argon atmosphere. The reaction was stirred at r.t. for 1 day, heated to 40° C. for 2 days and then stirred at r.t. for 1 week. The iron was filtered off and the reaction was dried in vacuo. Purification by normal phase MPLC (petrol ether/ethyl acetate with 2% NH₃0H, 10-50% ethyl acetate) gave product 53 (12.5 mg, 44.7 gmol, 70%, impure) as a yellow powder.

¹H NMR (500 MHz, MeOH-d₄) δ 7.08 (d, J 8.0 Hz, 4H, ArH), 6.70 (d, J 7.9 Hz, 4H, ArH), 3.47 (s, 4H, Ar—CH₂N), 2.60 (t, J 7.6 Hz, 2H, CH═CCH₂CH₂N), 2.33-2.27 (m, 2H, CH═CCH₂CH₂N), 2.19 (t, J 2.7 Hz, 1H, CH≡C).

¹³C NMR (126 MHz, MeOH-d₄) δ 147.61 (2C, ArC), 131.12 (2C, ArCH), 129.44 (2C, ArC), 116.56 (2C, ArCH), 83.46 (1C, CH═C, 70.27 (1C, CH≡C), 58.38 (2C, Ar—CH₂N), 52.61 (1C, CH═CCH₂CH₂N), 17.13 (1C, CH≡CCH₂CH₂N).

HPLC-MS: [C₁₈H₂₁N₃+H]⁺ calculated 280.18, found 280.13.

HRMS: [C₁₈H₂₁N₃+H]⁺ calculated 280.1809, found 280.1804.

### Divalent Ligand (55)

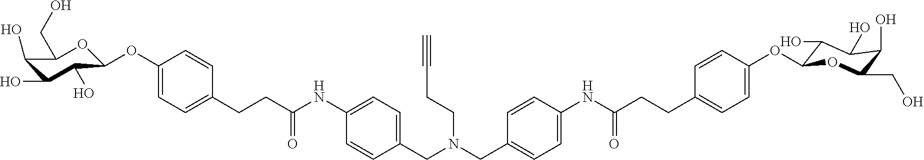

Bis-aniline linker 53 (26.7 mg, 95.6 μmol), galactoside 54 (71.7 mg, 218 gmol, in two portions over 2 days), HBTU (89.9 mg, 237 gmol, in two portions over 2 days) were dissolved in dimethylformamide (2 mL) and DIPEA (70 μL, 402 gmol) was added. Reaction was stirred at r.t. for 2 days, then dried in vacuo. Purification by preparative reverse-phase HPLC (water/acetonitrile supplemented with 0.1% formic acid, gradient of 10-40% acetonitrile) gave 55 (14.6 mg, 16.2 gmol, 17%) as a white solid.

¹H NMR (500 MHz, DMSO-d₆) δ 9.90 (s, 2H, NH), 7.52 (d, J 8.5 Hz, 4H, ArH), 7.26 (d, J 8.4 Hz, 4H, ArH), 7.18-7.12 (m, 4H, ArH), 6.97-6.90 (m, 4H, ArH), 5.15 (s, 2H, OH-2), 4.87 (s, 2H, OH-3), 4.76 (d, J 7.7 Hz, 2H, H-1), 4.66 (s, 2H, OH-6), 4.50 (s, 2H, OH-4), 3.67 (s, 2H, H-4), 3.58-3.41 (m, 14H, H-2, H-3, H-5, H-6, Ar—CH₂N), 2.84 (t, J 7.7 Hz, 4H CH₂CH₂CONH), 2.77 (t, J 2.6 Hz, 1H, CH≡C), 2.57 (t, J 7.7 Hz, 4H, CH₂CH₂CONH), 2.53-2.51 (m, 2H, CH≡CCH₂CH₂N), 2.35-2.29 (m, J 7.5, 2.7 Hz, 2H, CH≡CCH₂CH₂N).

¹³C NMR (126 MHz, DMSO-d₆) δ 170.39 (2C, C═O), 155.88 (2C, ArC), 138.07 (2C, ArC), 134.38 (2C, ArC), 133.75 (2C, ArC), 129.12 (4C, ArCH), 128.92 (4C, ArCH), 118.95 (4C, ArCH), 116.21 (4C, ArCH), 101.12 (2C, C-1), 83.26 (1C, CH≡C), 75.48 (2C, C-5), 73.33 (2C, C-3), 71.99 (1C, CH≡C), 70.32 (2C, C-2), 68.19 (2C, C-4), 60.42 (2C, C-6), 56.51 (2C, Ar—CH₂N), 51.10 (1C, CH≡CCH₂CH₂N), 38.29 (2C, CH₂CH₂CONH), 30.12 (2C, CH₂CH₂CONH), 15.95 (1C, CH≡CCH₂CH₂N).

HPLC-MS: [C₄₈H₅₇N₃O₁₄+H]⁺ calculated 900.39, found 900.34.

HRMS: [C₄₈H₅₇N₃O₁₄+H]⁺ calculated 900.3914, found 900.3911.

### Divalent Fluorescein Ligand (57)

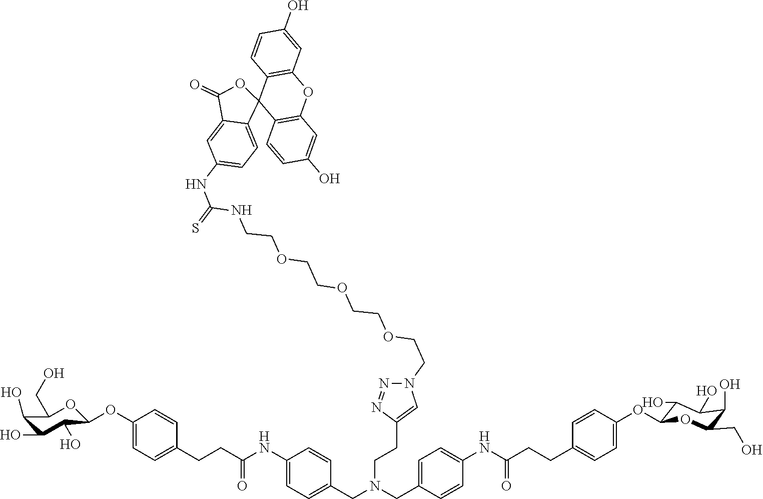

Azide modified fluorescein (56, 4.6 mg, 7.6 gmol) and divalent ligand 55 (4.44 mg, 4.9 gmol) were dissolved in dimethylformamide (500 μL). CuSO₄ solution (30 μL, 100 mM in water, 3 gmol) and sodium ascorbate solution (100 μL, 100 mM in water, 10 gmol) were added. The reaction was stirred at r.t. for 5 days then warmed up to 35° C. for one day. After lyophilization, the product was purified by preparative HPLC (water/acetonitrile with 1% formic acid, 20-40% acetonitrile). The product 57 was obtained as a yellow solid (4 mg, 2.7 gmol, 54%).

¹H NMR (500 MHz, DMSO-d₆) δ 10.21 (s, 2H, NH-thiourea), 9.86 (s, 2H, NHCO), 8.37-8.21 (m, 2H, ArH-fluorescein, NH-thiourea), 7.75 (d, J 8.3 Hz, 1H, ArH-fluorescein), 7.70 (s, 1H, CH-triazole), 7.51 (d, J=8.3 Hz, 4H. ArH), 7.21 (d, J=8.2 Hz, 4H, ArH), 7.17 (s, 1H, ArH-fluorescein), 7.14 (d, J 8.8 Hz, 4H, ArH), 6.96-6.90 (m, 4H ArH), 6.67 (d, J 2.3 Hz, 2H, ArH-fluorescein), 6.62-6.53 (m, 4H, ArH-fluorescein), 5.11 (s, 2H OH-2), 4.83 (s, 2H, OH-3), 4.75 (d, J 7.6 Hz, 2H, H-1), 4.63 (s, 2H, OH-6), 4.52-4.45 (m, 2H OH-4), 4.43 (t, J 5.3 Hz, 2H, CH₂—PEG), 3.76 (t, J 5.3 Hz, 2H, CH₂—PEG), 3.72-3.64 (s, 4H, H-4, CH₂—PEG), 3.61-3.38 (m, 31H, H-2, H-3, H-5, H-6, Ar—CH₂N, CH₂—PEG), 2.88-2.74 (m, 6H, CH₂CH₂CONH, triazole-CH₂CH₂N), 2.62-2.53 (m, 6H, CH₂CH₂CONH, triazole-CH₂CH₂N).

¹³C NMR (126 MHz, DMSO-d₆) δ 180.56(1C, C═S), 170.30 (2C, NHC═O), 168.55 (1C, C═O), 159.57 (2C, ArC-fluorescein), 155.84 (2C, ArC), 151.91 (2C, ArC-fluorescein), 144.97 (1C, C═CH-triazole), 141.43 (1C, ArC-fluorescein), 137.96 (2C, ArC), 134.35 (2C, ArC), 133.81 (2C, ArC), 129.04 (6C, ArCH, ArCH-fluorescein), 128.85 (4C, ArCH), 124.06 (1C, ArCH-fluorescein), 122.38 (1C, C═CH-triazole), 118.87 (4C, ArCH), 116.19 (5C, ArCH, ArC-fluorescein), 112.64 (1C, ArCH-fluorescein), 109.74 (1C, ArC-fluorescein), 102.24 (2C, ArCH-fluorescein), 101.13 (2C, C-1), 75.44 (2C, C-5), 73.31 (2C, C-3), 70.30 (2C, C-2), 69.72 (1C, CH₂—PEG), 69.64 (2C, CH₂—PEG), 69.55 (1C, CH₂—PEG), 68.81 (1C, CH₂—PEG), 68.41 (1C, CH₂—PEG), 68.15 (2C, C-4), 60.39 (2C, C-6), 56.74 (2C, Ar—CH₂N), 52.40 (1C, triazole-CH₂CH₂N), 49.16 (1C, CH₂—PEG), 43.67 (1C, CH₂—PEG), 38.24 (2C, CH₂CH₂CONH), 30.08 (2C, CH₂CH₂CONH), 22.87 (1C, triazole-CH₂CH₂N).

HPLC-MS: [C₇₇H₈₆N₈O₂₂S+2H]²⁺ calculated 754.29, found 754.72.

HRMS: [C₇₇H₈₆N₈O₂₂S+2H]²⁺ calculated 754.2862, found 754.2836.

### Divalent sulfoCy7 ligand (59)

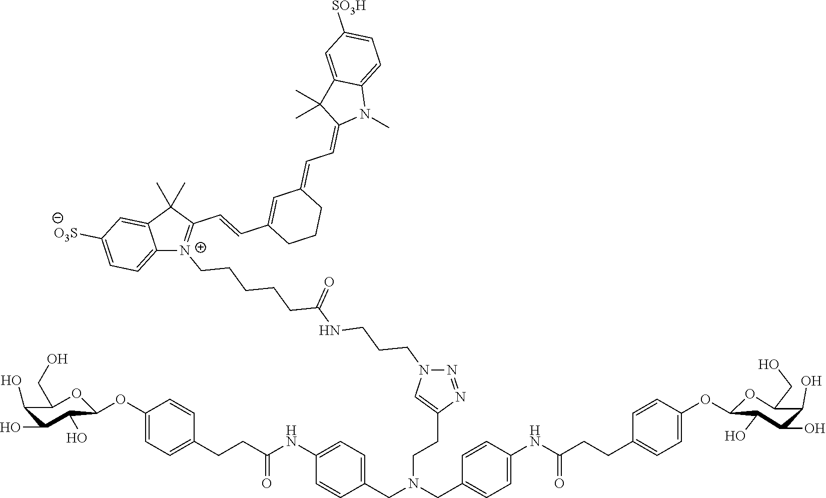

Divalent alkyne precursor 55 (6.75 mg, 7.5 gmol) and sulfoCy7-azide (4.33 mg, 5.2 gmol) were dissolved in DMF (300 μL). CuSO₄ solution (241 μL, 100 mM in H₂O, 24.1 gmol), sodium ascorbate solution (181 μL, 100 mM in H₂O, 18.1 gmol) and DIPEA (5 μL, 28.7 gmol) were added. The reaction was stirred at r.t. for 3 h. After lyophilization, the product was purified by preparative HPLC (water/acetonitrile with 1% formic acid, 20-45% acetonitrile). The product 59 was obtained as dark green solid (6.8 mg, 4.0 gmol, 77%).

¹H NMR (700 MHz, DMSO-d₆) δ 10.09 (s, 2H, CONH), 9.65 (s, 1H, SO₃H), 7.93 (s, 1H, CH-triazole), 7.84 (t, J=5.7 Hz, 1H, CONH), 7.75 (dd, J=8.9, 1.6 Hz, 2H, ArH), 7.73-7.58 (m, 9H, ArH, 2×CH═CH), 7.44 (s-broad, 4H, ArH), 7.31 (d, J=8.3 Hz, 1H, ArH), 7.24 (d, J=8.3 Hz, 1H, ArH), 7.18-7.10 (m, 4H, ArH), 6.98-6.90 (m, 4H, ArH), 6.15 (dd, J=19.2, 14.0 Hz, 2H, 2×CH═CH), 5.10 (s, 2H, OH), 4.83 (s, 2H, OH), 4.75 (d, J=7.7 Hz, 2H, H-1), 4.62 (s, 2H, OH), 4.47 (s, 2H, OH), 4.34 (s-broad, 4H, Ar—CH₂N), 4.26 (t, J=7.0 Hz, 2H, CH₂), 4.11 (t, J=7.1 Hz, 2H, CH₂), 3.68 (d, J=3.3 Hz, 2H, H-4), 3.61 (s, 3H, NCH₃), 3.56-3.44 (m, 8H, H-2, H-5, H-6), 3.41-3.37 (m, 2H, H-3), 3.20 (s-broad, 2H, CH₂), 3.12 (s-broad, 2H, CH₂), 2.96 (q, J=6.6 Hz, 2H, CH₂), 2.84 (t, J=7.7 Hz, 4H, CH₂CH₂CONH), 2.60 (t, J=7.5 Hz, 4H, CH₂CH₂CONH), 2.49-2.46 (m, 2H, CH₂), 2.04 (t, J=7.1 Hz, 2H, CH₂), 1.84-1.77 (m, 4H, 2×CH₂), 1.71 (q, J=7.8, 7.2 Hz, 2H, CH₂), 1.64 (s, 6H, CH₃), 1.62 (s, 6H, CH₃), 1.54 (p, J=7.3 Hz, 2H, CH₂), 1.30 (p, J=7.9 Hz, 2H, CH₂). ¹³C NMR (176 MHz, DMSO-d₆) δ 172.03, 170.80, 170.39, 155.86, 148.27, 147.17, 145.21, 144.57, 142.86, 142.48, 141.86, 140.42, 140.26, 140.12, 134.21, 132.29, 132.03, 129.06, 126.08, 123.82, 122.86, 119.78, 119.07, 116.19, 110.01, 109.81, 109.54, 101.12, 100.66, 99.78, 75.45, 73.32, 70.30, 68.15, 60.41, 55.79, 49.87, 48.64, 48.43, 47.17, 43.24, 38.22, 35.63, 35.00, 31.29, 29.96, 27.14, 26.92, 26.50, 25.65, 24.84, 23.33, 21.06.

HPLC-MS: [C₈₈H₁₀₇N₉O₂₁S₂+2H]⁺ calculated. 845.86, found 846.25.

### Divalent BODIPY ligand (60)

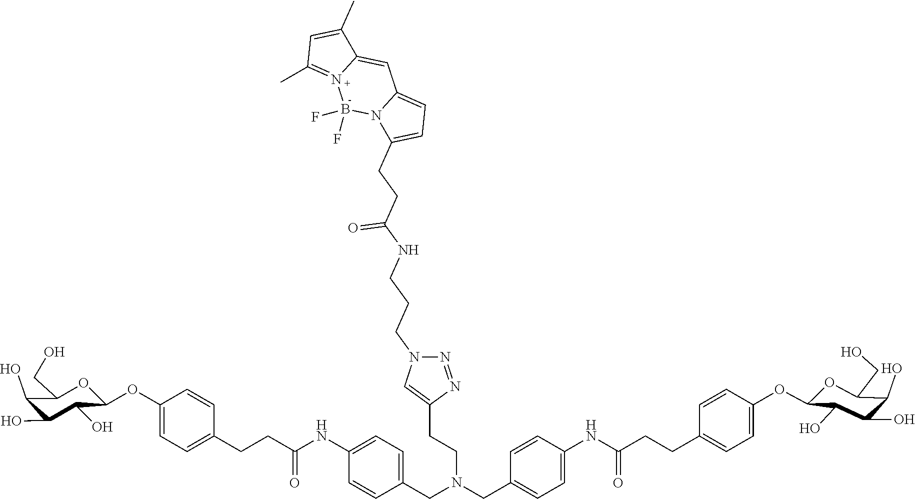

Divalent alkyne precursor 55 (6.5 mg, 7.2 gmol) was dissolved in DMF (1.5 mL). BDP-FL-azide solution (80 μL, 100 mM in DMF, 8.0 μmol), CuSO₄ solution (120 μL, 100 mM in H₂O, 12 gmol), sodium ascorbate solution (120 μL, 100 mM in H₂O, 12 gmol) and DIPEA (5 μL, 28.7 gmol) were added. The reaction was stirred at r.t. for overnight. After lyophilization, the product was purified by preparative HPLC (water/acetonitrile with 1% formic acid, 20-45% acetonitrile). The product 60 was obtained as dark green solid (5.6 mg, 4.4 gmol, 61%).

¹H NMR (500 MHz, DMSO-d₆) δ 9.86 (s, 2H, NHCO), 8.01 (t, J=5.6 Hz, 1H, NHCO), 7.72 (s, 1H, CH-triazole), 7.67 (s, 1H, ArH-BODIPY), 7.51 (d, J=8.2 Hz, 4H, ArH), 7.21 (d, J=8.3 Hz, 4H, ArH), 7.14 (d, J=8.4 Hz, 4H, ArH), 7.07 (d, J=4.0 Hz, 1H, ArH-BODIPY), 6.97-6.89 (m, 4H, ArH), 6.35 (d, J=4.0 Hz, 1H, ArH-BODIPY), 6.29 (s, 1H, ArH-BODIPY), 5.11 (s, 2H, OH-2), 4.83 (s, 2H, OH-3), 4.75 (d, J=7.7 Hz, 2H, H-1), 4.63 (s, 2H, OH-6), 4.48 (s, 2H, OH-4), 4.27 (t, J=7.0 Hz, 2H, CH₂), 3.68 (d, J=3.3 Hz, 2H, H-4), 3.57-3.42 (m, 14H, H-2, H-3, H-5, H-6, Ar—CH₂N), 3.11-3.02 (m, 4H, 2×CH₂), 2.87-2.74 (m, 6H, CH₂CH₂CONH, CH₂), 2.65-2.53 (m, 6H, CH₂CH₂CONH, CH₂), 2.53-2.51 (m, 2H CH₂), 2.46 (s, 3H, CH₃), 2.25 (s, 3H, CH₃), 1.91 (t, J=6.9 Hz, 2H, CH₂). ¹³C NMR (126 MHz, DMSO-d₆) δ 171.00 (1C, NHC═O), 170.33 (2C, NHC═O), 159.19 (1C, ArC-BODIPY), 157.77 (1C, ArC-BODIPY), 155.84 (2C, ArC), 145.04 (1C, C═CH-triazole), 144.13 (1C, ArC-BODIPY), 137.96 (2C, ArC), 134.47 (1C, ArC-BODIPY), 134.36 (2C, ArC), 133.83 (2C, ArC), 132.98 (1C, ArC-BODIPY), 129.04 (4C, ArCH), 128.93 (1C, ArCH-BODIPY), 128.87 (4C, ArCH), 125.37 (1C, ArCH-BODIPY), 122.08 (1C, C═CH-triazole), 120.30 (1C, ArCH-BODIPY), 118.90 (4C, ArCH), 116.59 (4C, ArCH), 116.20 (1C, ArCH-BODIPY), 101.14 (2C, C-1), 75.44 (2C, C-5), 73.32 (2C, C-3), 70.30 (2C, C-2), 68.16 (2C, C-4), 60.40 (2C, C-6), 56.75 (2C, CH₂), 52.39 (1C, CH₂), 46.99 (1C, CH₂), 38.25 (2C, CH₂CH₂CONH), 35.86 (1C, CH₂), 33.77 (1C, CH₂), 30.08 (2C, CH₂CH₂CONH), 29.96 (1C, CH₂), 23.99 (1C, CH₂), 22.90 (1C, CH₂), 14.53 (1C, CH₃), 11.01 (1C, CH₃).

HPLC-MS: [C₆₅H₇₈NF₂N₉O₁₅+H]⁺ calculated 1274.58, found 1274.73.

### Biological Evaluation

Expression and purification of LecA as well as competitive binding by fluorescence polarization was performed as described by Joachim et al. 2016. The assay was performed in TBS/Ca²⁺ buffer (20 mM Tris, 137 mM NaCl, 2.6 mM KCl at pH 7.4 supplemented with 1 mM CaCl₂) in presence of 25% DMSO. Averages and standard deviations were calculated from at least three independent experiments.

Isothermal titration calorimetry was performed on an iTC200 (Malvern Panalytical) and the data were analyzed using Microcal Origin software (Malvern Panalytical). LecA in the cell was titrated with ligand in TBS/Ca²⁺ buffer supplemented with 5% DMSO at 25° C. Steep titration slopes were the result of high amount of protein present and the high binding affinity of the ligand (high ‘value of c’). However, LecA protein concentration lower than 50 μM resulted in insufficient signal as a consequence of the low heat released upon binding.

Surface plasmon resonance experiments were performed on a BIACORE X100 instrument (GE Healthcare) at 25° C. as described by Zahorska et. al. 2020. Averages and standard deviations were calculated from three independent experiments.

Testing divalent LecA ligand in the flow cell

Colonies of PAO1 wt labelled with the red fluorescent protein mCherry (pMP7605) were picked from a LB agar plate and transferred to 10 mL LB medium with Gentamicin 60 μg/ml to start an overnight culture. Incubation was done at 37° C. and 180 rpm overnight. A fresh culture was inoculated to an OD₆₀₀ₙₘ of 0.02 (10 mL LB with Gentamicin 60 μg/mL) and incubated until the exponential growth phase was reached (OD₆₀₀ₙₘ of 0.4 to 0.8). The bacterial suspension was diluted again to an OD₆₀₀ₘ of 0.1 and 300 μL were injected into a six-channel μ-slide (ibidi, Germany). The settling time was 30 min. The flow of 1:5 diluted LB medium with Gentamicin 60 μg/ml was 3 mL/h. The biofilms were grown in a heat system (ibidi, Germany) at 30° C. and for 48 h.

After 48 h of biofilm growth, aggregates were stained by supplementing the divalent LecA ligand 57 and Fluorescein disodium salt 58 as a control to the medium. The final concentration of the supplements in the medium was 500 nM and constantly accumulated within the biofilm for 4 h by a flow rate of 3 mL/h. Washing was performed for 30 min with LB 1:5 diluted with Gentamicin 60 μg/mL.

Analysis was done by using a fluorescence microscope Leica DMi8. Several snapshots were taken before adding any fluorophore and after 4 h of a constant flow within the medium. mCherry signals were detected by an excitation wavelength of 540 to 580 nm and an emission wavelength of 592 to 668 nm. The green fluorescein signal was detected by an excitation wavelength of 460 to 500 nm and an emission wavelength of 512 to 542 nm. Snapshots were normalized by using the Fiji software.

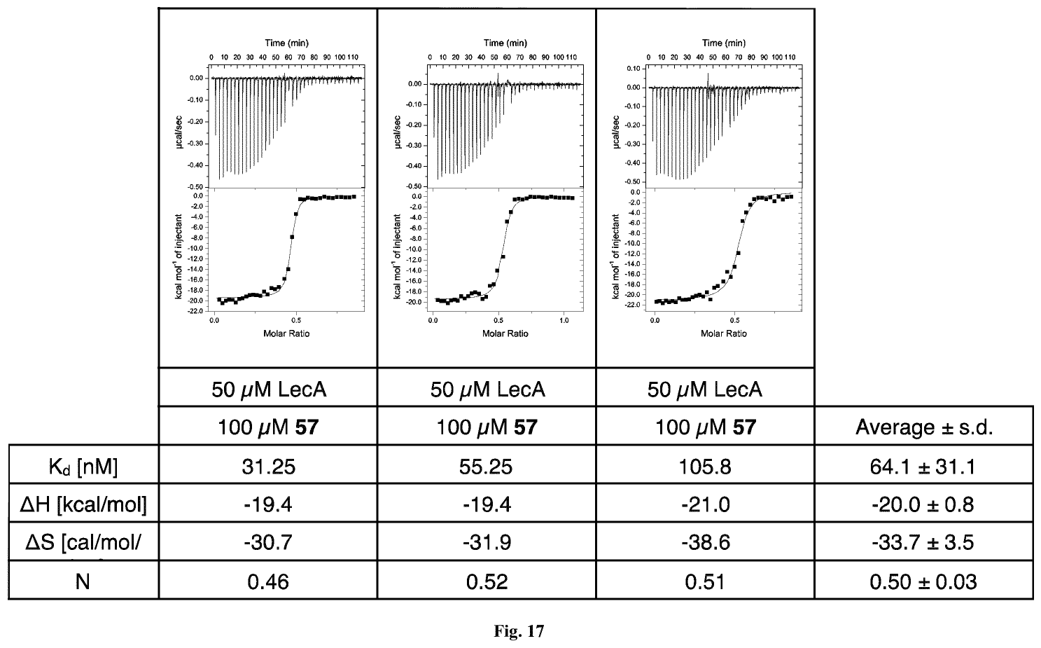

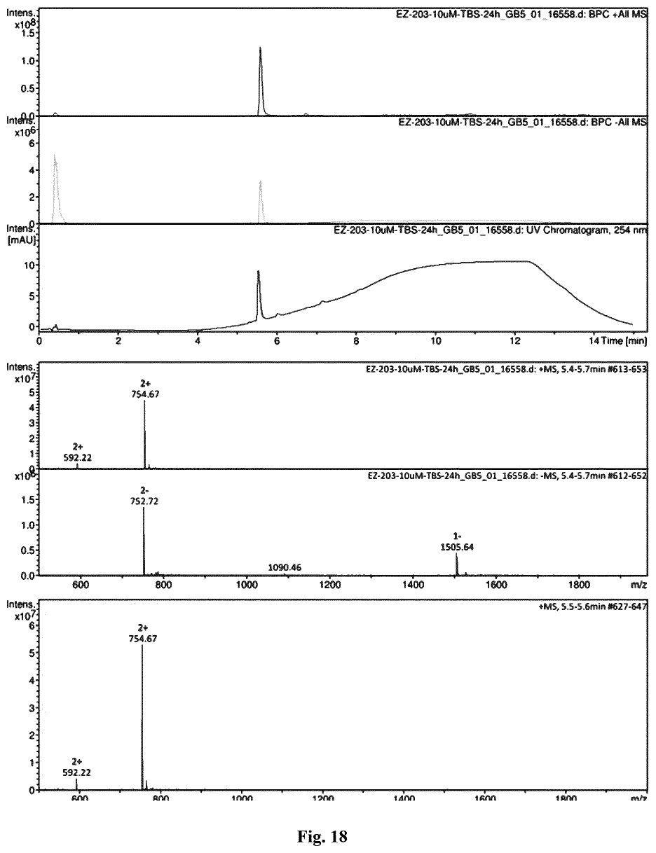

Low nanomolar binding affinities for divalent ligands 55 (K_{D}=9.9±0.5 nM) and 57 (K_{D}=19.3 ±10.5 nM) were measured by SPR (FIG. 16). In analogy to other divalent LecA ligands (Zahorska et. al. 2020), compound 55 (IC₅₀=5.6±2.2 μM) reached the lower assay limit in the competitive binding assay based on FP. The direct titration of the fluorescent ligand 57 with LecA in fluorescence polarization assay (FP) gave K_{d} of 1.05 μM—that is fifty-fold higher compared to the SPR result. It is possible, that crosslinking of LecA tetramers and/or their aggregation caused by the divalent ligand influenced fluorescence polarization and thus a lower binding affinity was observed, whereas crosslinking/aggregation of LecA immobilized on a SPR chip is unlikely. High affinity of the divalent fluorescent probe 57 was validated by isothermal titration microcalorimetry ITC (FIG. 17), therefore proving that the divalent LecA targeting scaffold with central nitrogen can carry a large substituent as a cargo (i.e. fluorophore) and retain its high on target affinity. Compound 57 did not show any degradation in Tris buffer after 24 h and was considered as stable (FIG. 18).

### Example 4—Divalent LecA targeted prodrug

I. Chemical synthesis of divalent prodrugs

### 7-(3-(tert-butyloxycarbonyl)-amino)pyrrolidinyl-1-cyclopropyl-6-fluoro-4-oxo-1,4-dihydroquinoline-3-carboxylic acid methylester (61)

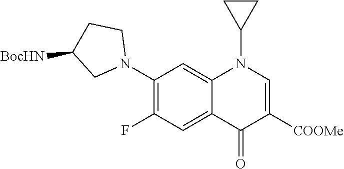 [SMILES: COC(=O)c1cn(C2CC2)c2cc(N3CC[C@H](NC(=O)OC(C)(C)C)C3)c(F)cc2c1=O]

Boc-protected fluoroquinolone 28 (300 mg, 0.7 mmol, 1 eq.), TBTU (558 mg, 1.7 mmol, 2.5 eq.) and DMAP (8 mg, 0.1 mmol, 10 mol %) were dispersed in dry CH₂Cl₂ (7 mL). Methanol (422 μl, 10.4 mmol, 15 eq.) and DIPEA (363 μl, 2.1 mmol, 3 eq.) were subsequently added under vigorous stirring and reacted over night at room temperature. The reaction was diluted with CH₂Cl₂ and washed with satd. aq. NaHCO₃, KHSO4 (1 M) and satd. brine. After drying over NaSO₄ the solvent was evaporated in vacuo and the product was purified by NP-MPLC (CH₂Cl₂/MeOH, 1-11%). The product was obtained as a beige amorphous solid (194 mg, 63%). H NMR (500 MHz, DMSO-d6) δ 8.37 (s, 1H, FQ-H-2), 7.67 (d, J=14.5 Hz, 1H, FQ-H-5), 7.28 (d, J=6.7 Hz, 1H, Boc-NH), 6.94 (d, J=7.8 Hz, 1H, FQ-H-8), 4.15 (q, J=5.5 Hz, 1H, pyrr-CH), 3.74 (d, J=6.7 Hz, 1H, pyrr-CH₂), 3.71 (s, 3H, COOCH₃), 3.65 (q, J=7.4 Hz, 1H, pyrr-—CH₂), 3.58 (m, 1H, cPr-CH), 3.53 (m, 1H, pyrr-CH₂), 3.48 (m, 1H, pyrr-CH₂), 2.13 (m, 1H, pyrr-CH₂), 1.91 (m, 1H, pyrr-CH₂), 1.39 (s, 9H, Boc-CH₃), 1.23 (m, 2H, cPr-CH₂), 1.05 (m, 2H, cPr-CH₂). ¹³C-NMR (126 MHz, DMSO-d6) δ 171.42 (C═O), 165.15 (C═O), 155.26 (Boc-C═O), 149.42 (d, J=243.6 Hz, FQ-C-6), 147.87 (FQ-C-2), 140.34 (d, J=11.9 Hz, FQ-C-7), 138.60 (FQ-C-8a), 117.76 (d, J=5.5 Hz, FQ-C-4a), 111.44 (d, J=23.0 Hz, FQ-C-5), 108.64 (FQ-C), 100.48 (d, J=5.5 Hz, FQ-C-8), 77.94 (Boc-C), 55.06 (d, J=5.1 Hz, pyrr-C), 51.17 (COOCH₃), 49.82 (pyrr-C), 47.93 (d, J=5.2 Hz, pyrr-C), 34.60 (cPr-CH), 30.42 (pyrr-C), 28.23 (Boc-CH₃), 7.52 (cPr-CH₂), 7.47 (cPr-CH₂). LR-MS: m/z=446.2, [M+H].

### 7-aminopyrrolidinyl-1-cyclopropyl-6-fluoro-4-oxo-1,4-dihydroquinoline-3-carboxylic acid (62)

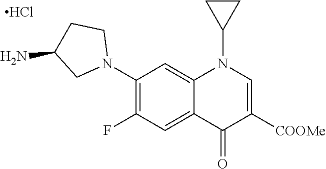 [SMILES: Cl ; COC(=O)c1cn(C2CC2)c2cc(N3CC[C@H](N)C3)c(F)cc2c1=O]

61 (116 mg, 0.26 mmol, 1 eq.) was dispersed in dioxane (5 mL) and HCl (5.15 mL, 4 M in dioxane, 20.5 mmol, 79 eq.) was added dropwise while cooling on ice. The reaction was allowed to warm to r.t. and stirred for 22 h. The consumption of the starting material was monitored by TLC (CH₂Cl₂: MeOH, 95: 5). After evaporation of the solvent in vacuo, the product was obtained as a yellow amorphous solid (269 mg, quant.). ¹HNMR (500 MHz, MeOH-d4) δ 9.10 (s, 1H, FQ-H-2), 8.07 (d, J=14.0 Hz, 1H, FQ-H-5), 7.32 (d, J=7.4 Hz, 1H, FQ-H-8), 4.19 (m, 2H, pyrr-CH), 4.06 (s, 3H, COOCH₃), 4.0 (m, 3H, Pyrr-H-5+cPr-CH), 3.90 (m, 1H, pyrr-H-5), 3.66 (s, 1H, pyrr-H-2), 2.56 (m, 1H, pyrr-H-4), 2.31 (m, 1H, pyrr-H-4), 1.52 (m, 2H, cPr-CH₂), 1.32 (m, 2H, cPr-CH₂). ¹³C NMR (126 MHz, MeOH-d4) δ 170.40 (d, J=4.4 Hz, C═O), 168.24 (C═O), 153.20 (d, J=252.1 Hz, FQ-C-6), 149.95 (FQ-C-2), 145.14 (d, J=12.8 Hz, FQ-C-7), 142.35 (FQ-C-8a), 113.88 (d, J=8.7 Hz, FQ-C-4a), 111.84 (d, J=24.8 Hz, FQ-C-5), 105.30 (FQ-C), 102.14 (d, J=6.4 Hz, FQ-C-8), 54.91 (pyrr-C), 54.83 (pyrr-C), 53.84 (COOCH₃), 51.58 (d, J=2.7 Hz, pyrr-C), 38.73 (cPr-CH), 29.99 (pyrr-C), 8.75 (cPr-CH₂), 8.71 (cPr-CH₂). LR-MS: m/z=346.2, [M+H]⁺.

Benzyl-protected alkyl-peptide linker 64:

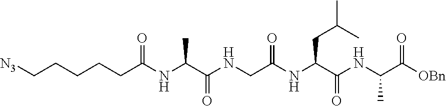 [SMILES: CC(C)C[C@H](NC(=O)CNC(=O)[C@H](C)NC(=O)CCCCCN=[N+]=[N-])C(=O)N[C@@H](C)C(=O)OCc1ccccc1]

8 (500 mg, 1.09 mmol, 1 eq.), TBTU (700 mg, 2.18 mmol, 2 eq.) and ω-Azido-hexanoic acid (160 μL, 1.09 mmol, 1 eq.) were dissolved in dry CH₂Cl2 (10 mL). DIPEA (571 μL, 3.27 mmol, 3 eq.) was added dropwise at roomtemperature. Upon the addition of base, a gel formed, which was redissolved by addition of dry DMF (3 mL). The reaction was stirred over night at r.t. and then diluted with CH₂Cl2 (90 mL). The organic phase was washed with KHSO4 (1 M), aq. satd. NaHCO₃ and brine. After drying over Na₂SO₄, the solvent was evaporated in vacuo and the product was purified by NP-MPLC (CH₂Cl2: MeOH/EtOH (1:1), 1-10%) to yield a white amorphous solid (407 mg, 68%). ¹H NMR (500 MHz, MeOH-d4) δ 7.39-7.28 (m, 5H, Ar—H), 5.17 (d, J=12.3 Hz, 1H, Bn-CH₂), 5.13 (d, J=12.3 Hz, 1H, Bn-CH₂), 4.51-4.40 (m, 2H, Leu-Cα-H, Ala-Cα-H), 4.23 (q, J=7.1 Hz, 1H, Ala-Cα-H), 3.90 (d, J=16.8 Hz, 1H, Gly-CH₂), 3.79 (d, J=16.8 Hz, 1H, Gly-CH₂), 3.28 (d, J=6.8 Hz, 2H, linker-CH₂), 2.26 (t, J=7.6 Hz, 2H, linker-CH₂), 1.85-1.50 (m, 7H, Leu-CH, Leu-CH₂, linker-CH₂, linker-CH₂), 1.41 (m, 5H, Ala-CH₃, linker-CH₂), 1.35 (d, J=7.1 Hz, 3H, Ala-CH₃), 0.91 (d, J=6.4 Hz, 3H, Leu-CH₃), 0.88 (d, J=6.4 Hz, 3H, Leu-CH₃). ¹³C NMR (126 MHz, MeOH-d4) δ 176.08 (C═O), 176.01 (C═O), 174.59 (C═O), 173.75 (C═O), 171.47 (C═O), 137.27 (Ar—C), 129.57 (Ar—C), 129.29 (Ar—C), 129.23 (Ar—C), 67.91 (Bn-CH₂), 52.96 (Ala-Cu), 52.27 (linker-CH₂), 51.11 (Ala-Cu), 49.67 (Leu-Cu), 43.61 (Gly-Cu), 41.82 (Leu-CH₂), 36.41 (linker-CH₂), 29.64 (linker-CH₂), 27.40 (linker-CH₂), 26.21 (linker-CH₂), 25.71 (Leu-CH), 23.52 (Leu-CH₃), 21.85 (Leu-CH₃), 17.44 (Ala-CH₃), 17.22 (Ala-CH₃).

LR-MS: 560.4 [M+H]⁺.

Alkyl-peptide linker (65)

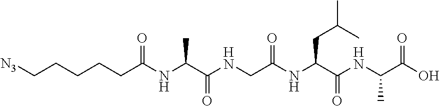 [SMILES: CC(C)C[C@H](NC(=O)CNC(=O)[C@H](C)NC(=O)CCCCCN=[N+]=[N-])C(=O)N[C@@H](C)C(=O)O]

64 (400 mg, 0.85 mmol, 1 eq.) was dissolved in a mixture of THF/MeOH/H₂O (8 mL, 3:2:2) at room temperature. LiOH (61 mg, 2.55 mmol, 3 eq.) was dissolved in H₂O (1 mL) and added dropwise to the starting material. The reaction was stirred at room temperature until disappearance of the starting material (10 min), monitored by TLC (CH₂Cl2, MeOH 95:5). The reaction was cooled on ice and neutralised with Amberlite IR120/H⁺. After filtration of the ion exchange resin, solvent was removed in vacuo. The product was purified by NP-MPLC (CH₂Cl2: MeOH, 1-10%) and obtained as a white amorphous solid (234 mg, 59%). ¹H NMR (500 MHz, MeOH-d4) 4.46 (dd, J=9.1, 5.5 Hz, 1H, Leu-Cu—H), 4.38 (q, J=7.3 Hz, 1H, Ala-Cα-H), 4.27 (q, J=7.1 Hz, 1H, Ala-Cα-H), 3.93 (d, J=16.7 Hz, 1H, Gly-Cu—H), 3.81 (d, J=16.7 Hz, 1H, Gly-Cu—H), 3.28 (t, J=6.8 Hz, 1H, linker-CH₂), 2.26 (t, J=7.6 Hz, 1H, linker-CH₂), 1.82-1.54 (m, 7H, Leu-CH, Leu-CH₂, linker-CH₂, linker-CH₂), 1.48-1.41 (m, 1H, linker-CH₂), 1.40 (d, J=7.3 Hz, 3H, Ala-CH₃), 1.36 (d, J=7.2 Hz, 3H, Ala-CH₃), 0.95 (d, J=6.1 Hz, 3H, Leu-CH₃), 0.92 (d, J=6.1 Hz, 3H, Leu-CH₃). ¹³C NMR (126 MHz, MeOH-d4) δ 176.53 (C═O), 176.26 (C═O), 176.06 (C═O), 174.31 (C═O), 171.74 (C═O), 53.26 (Ala-Cu), 52.28 (linker-CH₂), 51.19 (linker-CH₂), 49.87 (Leu-Cu), 43.66 (Gly-Cu), 41.73 (Leu-CH₂), 36.41 (linker-CH₂), 29.64 (linker-CH₂), 27.41 (linker-CH₂), 26.25 (linker-CH₂), 25.77 (Leu-CH), 23.56 (Leu-CH₃), 21.81 (Leu-CH₃), 17.78 (Ala-CH₃), 17.38 (Ala-CH₃). LRMS: 470.3 [M+H].

Methyl-protected alkyl-peptidyl fluoroquinolone building block (11)

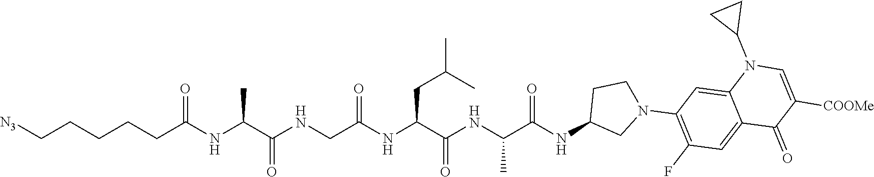 [SMILES: COC(=O)c1cn(C2CC2)c2cc(N3CC[C@H](NC(=O)[C@H](C)NC(=O)[C@H](CC(C)C)NC(=O)CNC(=O)[C@H](C)NC(=O)CCCCCN=[N+]=[N-])C3)c(F)cc2c1=O]

65 (70 mg, 0.15 mmol, 1 eq.), 62 (69 mg, 0.18 mmol, 1.2 eq.) and TBTU (96 mg, 0.3 mmol, 2 eq.) were dissolved in dry DMF (2 mL). DIPEA (79 μL, 0.45 mmol, 3 eq.) was added dropwise and the reaction was stirred for 4 h, monitored by TLC. The reaction was diluted with CH₂Cl2 (25 mL) and the organic phase was washed with KHSO4 (1 M), aq. satd. NaHCO₃ and brine. After evaporation of the solvent, the product was purified by NP-MPLC (CH₂Cl2: MeOH, 1-10%). The combined elution fractions were further purified by precipitation from MeOH with Et₂O and the product was isolated as a beige amorphous solid (65 mg, 54%, 90% purity according to ¹H-NMR). ¹H NMR (500 MHz, MeOH-d4) δ 8.58 (s, 1H, FQ-H-2), 7.78 (d, J=14.6 Hz, 1H, FQ-H-5), 7.03 (d, J=7.6 Hz, 1H, FQ-H-8), 4.53-4.43 (m, 1H, pyrr-CH), 4.37-4.22 (m, 2H, Ala-Cu, Leu-Cu), 4.16 (q, J=7.2 Hz, 1H, Ala-Cu), 3.94-3.84 (m, 1H, pyrr-CH₂), 3.84 (s, 3H, COOMe), 3.84-3.77 (m, 1H, pyrr-CH₂), 3.72 (d, J=16.6 Hz, 1H, Gly-CH₂), 3.69-3.66 (m, 1H, pyrr-CH₂), 3.63 (d, J=16.6 Hz, 1H, Gly-CH₂), 3.61-3.54 (m, 2H, cPr-CH, pyrr-CH₂), 3.28 (t, J=6.9 Hz, 2H, linker-CH₂), 2.36-2.19 (m, 3H, linker-CH₂, pyrr-CH₂), 2.14 (dt, J=12.7, 2.9 Hz, 1H, pyrr-CH₂), 1.90-1.49 (m, 7H, Leu-CH, Leu-CH₂, linker-CH₂, linker-CH₂), 1.45-1.38 (m, 5H, Ala-CH₃, linker-CH₂), 1.36 (d, J=7.2 Hz, 3H, Ala-CH₃), 1.34-1.27 (m, 2H, cPr-CH₂), 1.13 (dd, J=9.0, 3.8 Hz, 2H, cPr-CH₂), 0.94 (d, J=6.3 Hz, 3H, Leu-CH₃), 0.87 (d, J=6.3 Hz, 3H, Leu-CH₃). ¹³C NMR (126 MHz, MeOH-d4) δ 176.64 (C═O), 176.48 (C═O), 175.29 (C═O), 175.11 (C═O), 174.83 (C═O), 172.44 (C═O), 166.99 (C═O), 151.79 (d, J=244.8 Hz, FQ-C-6), 149.68 (FQ-C), 142.73 (d, J=11.5 Hz, FQ-C-7), 140.51 (FQ-C), 119.20 (d, J=6.1 Hz, FQ-C-8a), 112.90 (d, J=23.0 Hz, FQ-C-4a), 109.80 (FQ-C), 101.59 (d, J=5.4 Hz, FQ-C-8), 55.95 (d, J=6.0 Hz, pyrr-C), 54.09 (Leu-Cu), 52.28 (linker-CH₂), 52.07 (COOMe), 51.85 (Ala-Cu), 50.86 (pyrr-C), 50.81 (Ala-Cu), 44.13 (Gly-CH₂), 41.16 (Leu-CH₂), 36.33 (linker-CH₂), 36.16 (cPr-CH), 31.89 (pyrr-C), 29.67 (linker-CH₂), 27.45 (linker-CH₂), 26.12 (linker-CH₂), 25.88 (Leu-CH), 23.55 (Leu-CH₃), 21.58 (Leu-CH₃), 17.80 (Ala-CH₃), 17.36 (Ala-CH₃), 8.53 (cPr-CH₂), 8.50 (cPr-CH₂). LR-MS: 797.6 [M+H]⁺.

Alkyl-peptidyl fluoroquinolone building block (67)

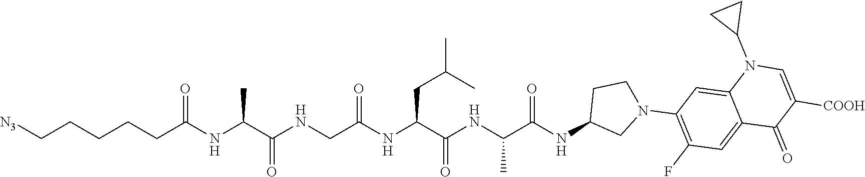 [SMILES: CC(C)C[C@H](NC(=O)CNC(=O)[C@H](C)NC(=O)CCCCCN=[N+]=[N-])C(=O)N[C@@H](C)C(=O)N[C@H]1CCN(c2cc3c(cc2F)c(=O)c(C(=O)O)cn3C2CC2)C1]

66 (31 mg, 0.039 mmol, 1 eq.) was dissolved in a mixture of THF/MeOH/H₂O (1 mL, 3:1:1) at room temperature. LiOH (4 mg, 0.16 mmol, 4 eq.) was added to the starting material at room temperature and the reaction was stirred at room temperature until disappearance of the starting material (24 h), monitored by TLC (CH₂Cl2: MeOH 90: 10, 1% NH₄OH). The reaction was cooled on ice and neutralised with Amberlite IR120/H⁺. After filtration of the ion exchange resin, the solvent was evaporated in vacuo. The product was obtained as a beige amorphous solid (30 mg, 98%). ¹H NMR (500 MHz, DMSO-d6) δ 15.52 (s, 1H, COOH), 8.58 (s, 1H, FQ-H-2), 8.30-8.22 (m, 1H, Ar—H), 8.16 (d, J=6.5 Hz, 1H, NH), 8.01 (d, J=6.7 Hz, 1H, NH), 7.96 (d, J=7.4 Hz, 1H, NH), 7.87 (d, J=7.8 Hz, 1H, NH), 7.81 (d, J=14.1 Hz, 1H, FQ-H), 7.06 (d, J=7.5 Hz, 1H, FQ-H), 4.42-4.34 (m, 1H, pyrr-CH), 4.29-4.09 (m, 3H, Ala-Cα-H, Ala-Cα-H, Leu-Cα-H), 3.85 (br s, 1H, pyrr-CH₂), 3.78-3.72 (m, 2H, cPr-CH, pyrr-CH₂), 3.71-3.56 (m, 3H, Gly-CH₂ pyrr-CH₂), 3.48-3.43 (m, 1H, pyrr-CH₂), 3.30 (t, J=6.9 Hz, 2H, linker-CH₂), 2.38-2.10 (m, 3H, linker-CH₂, pyrr-CH₂), 2.03-1.89 (m, 1H, pyrr-CH₂), 1.68-1.42 (m, 7H, Leu-CH₂, Leu-CH, Linker-CH₂, Linker-CH₂), 1.36-1.25 (m, 4H, cPr-CH₂, linker-CH₂), 1.23 (d, J=7.2 Hz, 3H, Ala-CH₃), 1.20 (d, J=7.1 Hz, 3H, Ala-CH₃), 1.18-1.10 (m, 2H, cPr-CH₂), 0.86 (d, J=6.5 Hz, 3H, Leu-CH₃), 0.81 (d, J=6.5 Hz, 3H, Leu-CH₃). ¹³C NMR (126 MHz, DMSO-d6) δ 175.86 (C═O), 173.29 (C═O), 172.47 (C═O), 172.19 (C═O), 171.59 (C═O), 169.22 (C═O), 166.30 (C═O), 149.96 (d, J=246.5 Hz, FQ-C-6), 147.44 (Ar—C), 141.62 (d, J=11.2 Hz, FQ-C-7), 139.81 (Ar—C), 110.74 (d, J=22.9 Hz, FQ-C-4a), 100.50 (d, J=5.3 Hz, FQ-C-8), 69.80 (Gly-Cu), 54.97 (pyrr-C), 51.31 (Leu-Cu), 50.53 (linker-CH₂), 48.81 (Ala-Cu), 48.67 (pyrr-C), 48.38 (Ala-Cu), 48.01 (pyrr-C), 42.30 (Gly-Cu), 40.43 (Leu-CH₂), 35.69 (cPr-CH), 34.85 (linker-C), 30.37 (pyrr-C), 28.03 (linker-C), 25.82 (linker-C), 24.56 (linker-C), 24.08 (Leu-CH), 23.03 (Leu-CH₃), 21.44 (Leu-CH₃), 17.87 (Ala-CH₃), 17.70 (Ala-CH₃), 7.57 (cPr-CH₂), 7.51 (cPr-CH₂). LR-MS: 783.5 [M+H]

Bivalent prodrug (68)

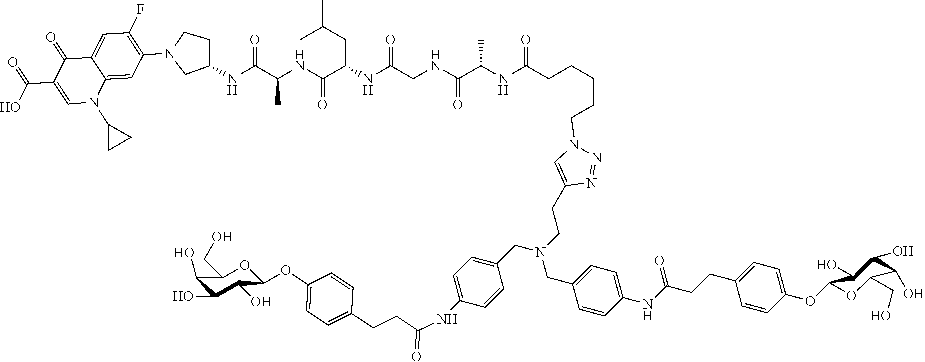

55 (10 mg, 11 μmol, 1 eq.) 67 (8.7 mg, 11 μmol, 1 eq.) and DIPEA (2 μL, 11 μmol, 1 eq.) were dissolved in dry DMF (400 μL). CuSO₄ (56 μL of a 100 mM solution in H₂O, 5.5 μmol, 50 mol %) and sodium ascorbate (66 μL of a 100 mM solution in H₂O, 6.6 μmol, 60 mol %) were added and the reaction was stirred at r.t. for 24 h. The solvent was evaporated in vacuo and purified by preparative HPLC (MeCN: H₂O, 21-35%). The product was obtained as a white amorphous solid (8 mg, 43%). ¹H NMR (500 MHz, DMSO-d6) δ 15.52 (s, 1H, COOH), 9.86 (s, 2H, glyco-probe-NH), 8.58 (s, 1H, FQ-H-2), 8.21 (t, J=5.5 Hz, 1H, NH), 8.09 (d, J=6.4 Hz, 1H, NH), 7.97 (d, J=6.7 Hz, 1H, NH), 7.91 (d, J=7.5 Hz, 1H, NH), 7.84 (d, J=7.6 Hz, 1H, NH), 7.81 (d, J=14.1 Hz, 1H, FQ-H-5), 7.69 (s, 1H, triazole-H), 7.51 (d, J=8.1 Hz, 4H, Ar—H), 7.20 (d, J=8.3 Hz, 4H, Ar—H), 7.14 (d, J=8.5 Hz, 4H, Ar—H), 7.05 (d, J=7.6 Hz, 1H, FQ-H-8), 6.93 (d, J=8.6 Hz, 4H, Ar—H), 5.11 (br s, 2H, OH), 4.83 (br s, 2H, OH), 4.75 (d, J=7.7 Hz, 2H, Gal-H-1), 4.62 (br s, 2H, OH), 4.47 (br s, 2H, OH), 4.40-4.31 (m, 1H, pyrr-CH), 4.27-4.12 (m, 5H, linker-CH₂, Ala-Cα-H, Ala-Cα-H, Leu-Cα-H), 3.90-3.79 (m, 1H, pyrr-CH₂), 3.80-3.71 (m, 2H, cPr-CH, pyrr-CH₂), 3.68 (d, J=3.3 Hz, 2H, Gal-H-4), 3.67-3.57 (m, 3H, Gly-CH₂, pyrr-CH₂), 3.58-3.35 (m, 13H, Gal-H-2, Gal-H-3, Gal-H-5, Gal-H-6, pyrr-CH₂, pyrr-CH₂), 2.83 (t, J=7.7 Hz, 4H, CH₂—CH₂—N), 2.78 (t, J=7.6 Hz, 2H, triazole-CH₂—CH₂—N), 2.64-2.59 (m, 2H, triazole-CH₂—CH₂—N), 2.56 (t, J=7.8 Hz, 4H, CH₂—CH₂—CONH), 2.23-2.13 (m, 1H, pyrr-CH₂), 2.13-2.06 (m, 2H, linker-CH₂), 2.01-1.92 (m, 1H, pyrr-CH₂), 1.79-1.70 (m, 2H, linker-CH₂), 1.64-1.54 (m, 1H, Leu-CH), 1.50 (d, J=7.2 Hz, 2H, linker-CH₂), 1.48-1.40 (m, 2H, Leu-CH₂), 1.28 (d, J=7.3 Hz, 2H, cPr-CH₂), 1.21 (d, J=7.1 Hz, 3H, Ala-CH₃), 1.18 (d, J=7.2 Hz, 3H, Ala-CH₃), 1.17-1.10 (m, 4H, cPr-CH₂+linker-CH₂), 0.84 (d, J=6.6 Hz, 6H, Leu-CH₃), 0.79 (d, J=6.4 Hz, 3H, Leu-CH₃). ¹³C NMR (126 MHz, DMSO-d6) δ 175.89 (d, J=2.7 Hz, FQ-C-4), 173.26 (C═O), 172.41 (C═O), 172.14 (C═O), 171.56 (C═O), 170.31 (C═O), 169.17 (C═O), 166.26 (COOH), 155.84 (glyco-probe-Ar), 149.96 (d, J=246.1 Hz, FQ-C-6), 147.46 (FQ-C), 145.00 (triazole-C), 141.61 (d, J=11.5 Hz, FQ-C-7), 139.82 (FQ-C), 137.96 (glyco-probe-Ar), 134.34 (glyco-probe-Ar), 133.80 (glyco-probe-Ar), 129.03 (glyco-probe-Ar), 128.84 (glyco-probe-Ar), 121.84 (triazole-C), 118.87 (glyco-probe-Ar), 116.19 (glyco-probe-Ar), 114.52 (d, J=6.2 Hz, FQ-C-4a), 110.75 (d, J=23.0 Hz, FQ-C-5), 106.20 (FQ-C), 101.13 (Gal-C-1), 100.50 (d, J=5.5 Hz, FQ-C-8), 75.44 (Gal-C-5), 73.31 (Gal-C-3), 70.30 (Gal-C-3), 68.14 (Gal-C-4), 60.39 (Gal-C-6), 56.73 (Ar—CH₂—N), 54.93 (pyrr-CH₂), 52.39 (triazole-CH₂—CH₂—N), 51.23 (Leu-Cu), 48.98 (linker-CH₂), 48.73 (Ala-Cu), 48.64 (pyrr-C), 48.31 (Ala-Cu), 48.02 (d, J=5.2 Hz, pyrr-C), 42.26 (Gly-CH₂), 40.45 (Leu-CH₂), 38.25 (CH₂—CH₂—CONH), 35.69 (cPr-CH), 34.78 (linker-CH₂), 30.35 (pyrr-C), 30.09 (CH₂—CH₂—CONH), 29.56 (linker-CH₂), 25.55 (linker-CH₂), 24.41 (linker-CH₂), 24.06 (Leu-CH), 23.02 (Leu-CH₃), 22.91 (triazole-CH₂—CH₂—N), 21.43 (Leu-CH₃), 17.86 (Ala-CH₃), 17.73 (Ala-CH₃), 7.56 (cPr-CH₂), 7.49 (cPr-CH₂). HRMS calculated [C₈₅H₁₁₀FN₁₃O₂₂]²⁺: 841.8931 found: 841.8925.

II. Biophysical evaluation of divalent prodrugs

### II.1 Methods

Isothermal titration calorimetry (ITC)

Isothermal titration calorimetry was performed on an iTC200 (Malvern Panalytical) and the data were analyzed using Microcal Origin software (Malvern Panalytical). LecA (50 μM) in the cell was titrated with ligand 68 in MES/Ca²⁺ buffer (50 mM MES, 1 mM CaCl₂, pH 6) supplemented with 15% DMSO at 298.15 K. For divalent alkyne ligand 55, the titration was carried in TBS/Ca²⁺ (pH 7.4) supplemented with 5% DMSO at 298.15 K. Steep titration slopes resulted from the product of protein concentration and the ligands' high binding affinity of the (c=[LecA]/K_{d}=50 μM/36 nM >1000). However, LecA protein concentration lower than 50 μM resulted in insufficient signal as a consequence of the low heat released upon binding.

### Peptide Cleavage Assays

P. aeruginosa PA14 was streaked on LB-agar plates (1% agar) from glycerol stocks and incubated at 37° C. overnight. 2-5 colonies were picked and dispersed in 10 mL LB (10 g/L trypton, 10 g/L NaCl, 5 g/L yeast extracts). This culture was grown overnight at 37° C., 180 rpm under high humidity. The culture was centrifuged (4000 rcf, 22° C., 10 min), and the supernatant was filtered (0.22 μm pore size).

MMP-2 (50 μL, 0.2 μg/mL in 20 mM tris pH 7.5, 8 mM CaCl₂, 119 mM NaCl, 20% Glycerol, 0.05% Brij 35, Cat #SAE0174-50UG, Sigma-Aldrich) was activated with 1 mM APMA (from 100 mM DMSO stock, Cat #AS—71158, AnaSpec) for 1 h at 37° C. Then, dilution series of the activated MMP-2 and of PA14-filtrate were prepared in PBS. SensoLyte 520 generic MMP substrate (Cat #AS—71158, AnaSpec) was diluted 1:100 in PBS. On the other hand, LasB substrate Abz-Ala-Gly-Leu-Ala-Nba (in DMSO, Cat #SAG-3905-PI, Biosynth) was diluted in PBS to reach 300 μM. 10 μL dilution series was mixed with 10 μL MMP substrate or LasB substrate and fluorescence (λₑₓ=490 mn, λₑₘ=520 nm and λₑₓ=320 mn, λₑₘ=420 nm, for MMP substrate and LasB substrate, respectively) was recorded for 10 min (10 s interval).

P. aeruginosa biofilm staining

The method for P. aeruginosa biofilm staining has been described above in Example 3, sub-chapter “Testing divalent LecA ligand in the flow cell”.

### II.2 Results

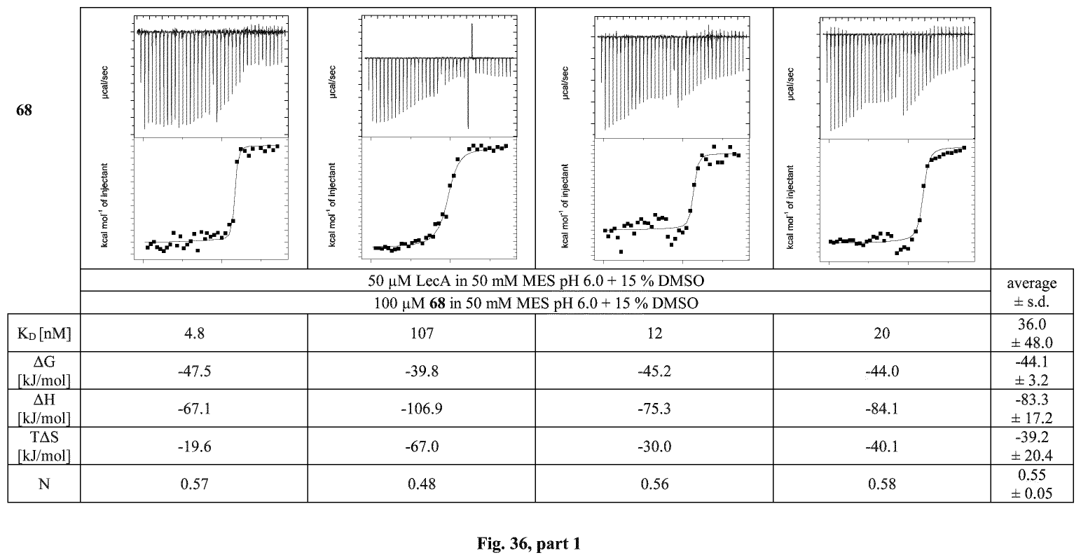

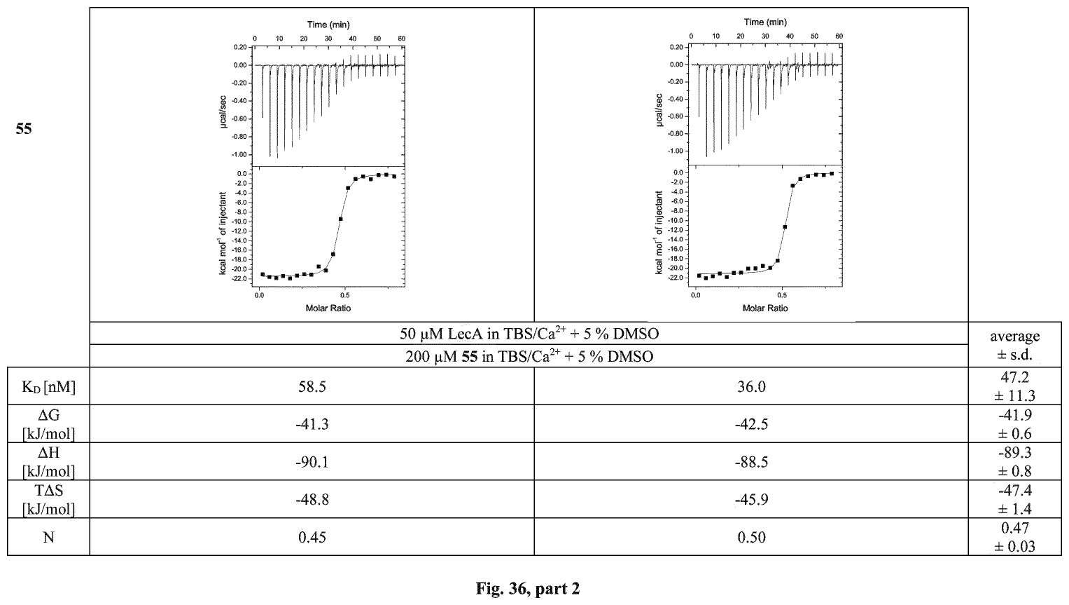

Affinity measurement of bivalent LecA inhibitor 55 and bivalent prodrug 68 by ITC On-target affinity of bivalent LecA inhibitor 55 and the corresponding prodrug 68 was determined by isothermal titration calorimetry (ITC). Both compounds show high affinity on LecA with a nanomolar K_{d} (36.0±48.0 nM for 68, FIG. 36, part 1; 47.2±11.3 nM for 55, FIG. 36, part 2), making clear that the cargo does not significantly influence binding.

### Stability Studies of Peptidic Prodrug Linker

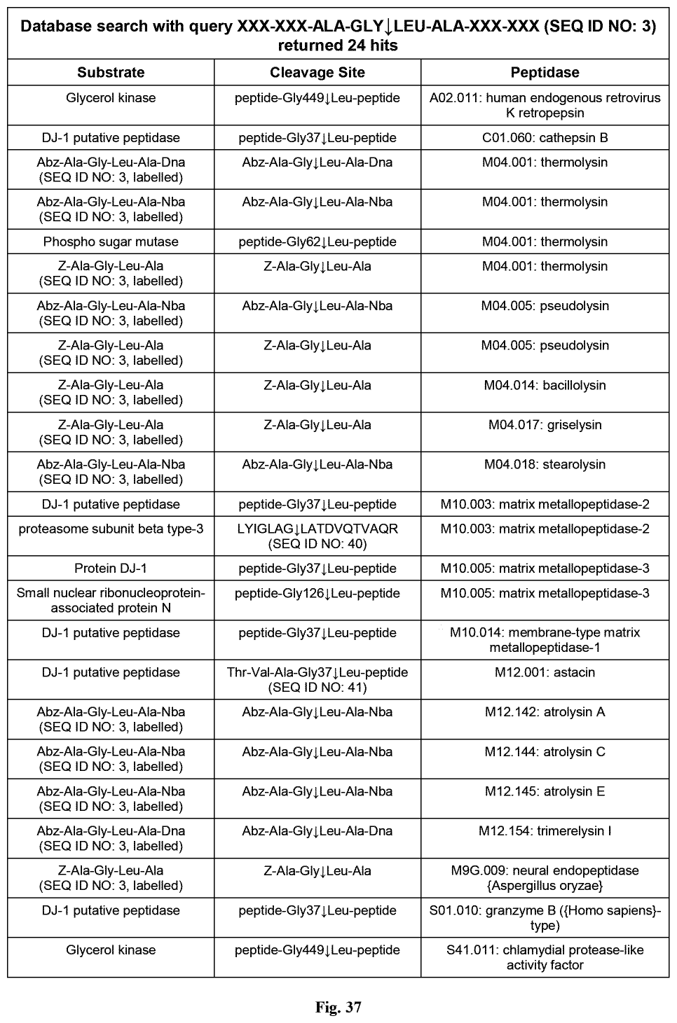

A MEROPS database search with the peptidic prodrug linker Ala-Gly-Leu-Ala (SEQ ID NO 3) revealed 18 enzymes that could potentially cleave the prodrug. Although most of these hits are from non-mammalian origin and are thus unproblematic (i.e. thermolysin, pseudolysin, bacillolysin, griselysin, stearolysin, astacin, atrolysin A/C/E trimerelysin I, neutral endopeptidase from Aspergillus ory-zae, chlamydial protease-like activity factor), matrix-metalloproteases 2 and 3 (MMP-2 and MMP-3, respectively) raised our attention (FIG. 37).

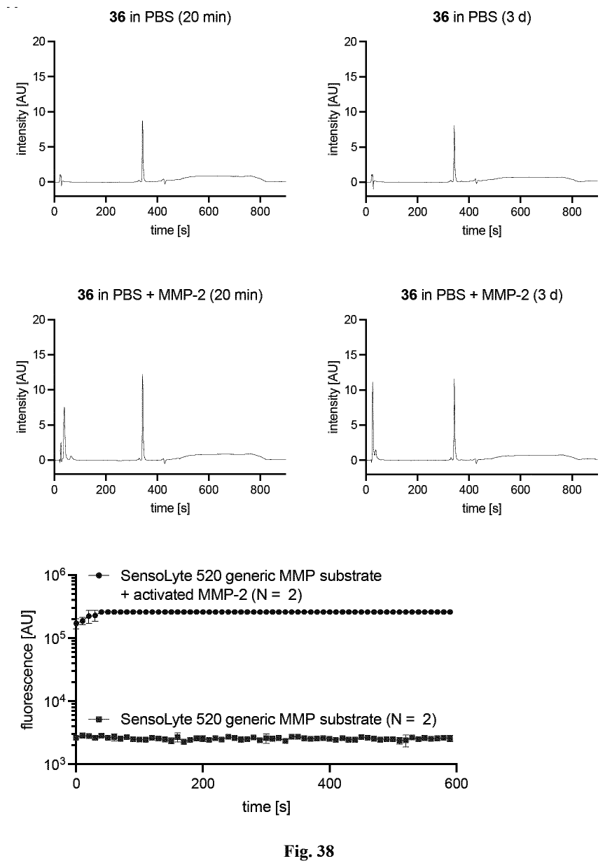

As a starting point, stability of the peptide-based prodrugs against MMP-2 was studied. MMP-2 has to be activated with 1 mM 4-aminophenyl mercuric acetate (APMA). We used the SensoLyte 520 generic MMP substrate (structure not disclosed, Anaspec, USA) that is conjugated to a fluorescent dye (5-FAM) and a fluorescent quencher (QXL-520) to validate proper activation by a FRET-based cleavage assay. Stability of prodrug 36 in presence of activated MMP-2 was monitored by HPLC-MS (FIG. 38). No detectable metabolism was observed after 20 min or 3 d at 37° C., indicating that prodrug 36 is not a good substrate for MMP-2.

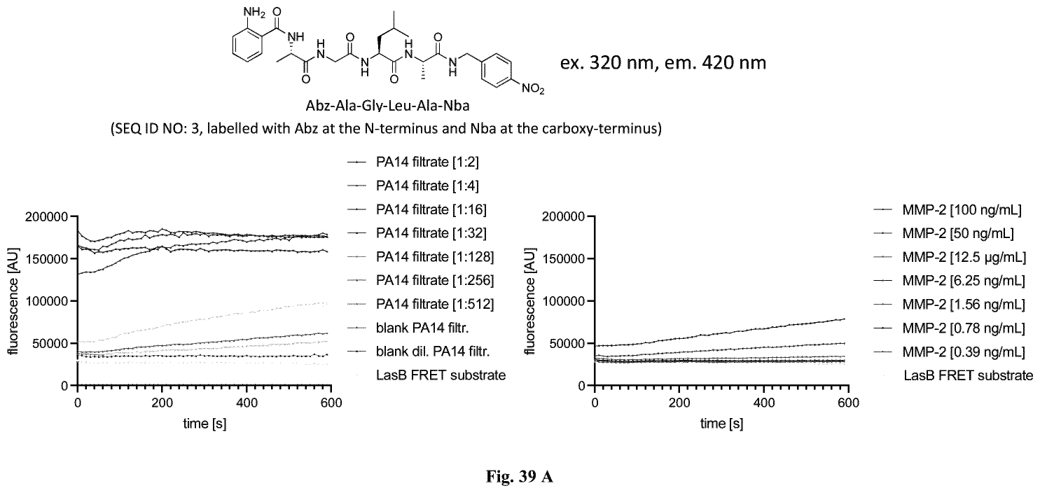

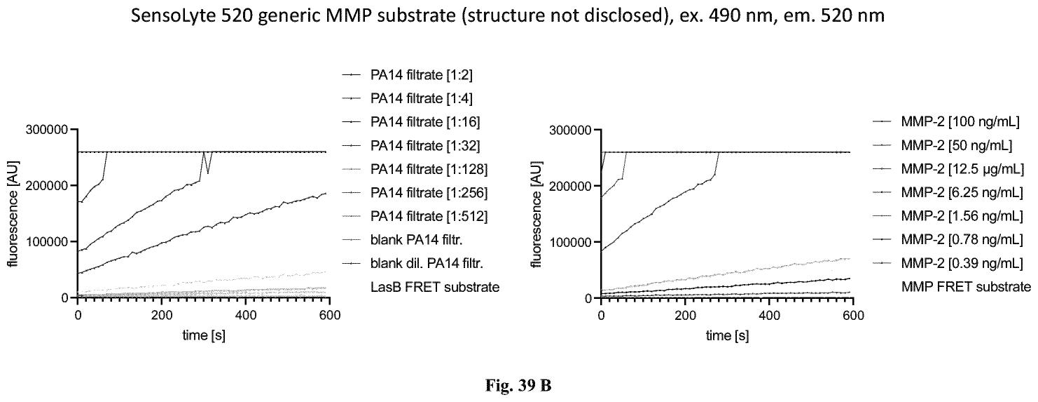

As an alternative control experiment, a LasB cleavable substrate (Abz-Ala-Gly-Leu-Ala-Nba; SEQ ID NO 3, labelled) conjugated to fluorescent aminobenzoic acid (Abz) and respective fluorescent quencher nitrobenzyl amine (Nba) was treated with a serial dilution of activated MMP-2 or PA14 filtrate as positive control (FIG. 39A). Only a very weak turnover of the LasB-substrate was observed in a FRET-based cleavage assay at very high concentrations (>50 ng/mL final) of MMP-2. On the other hand, PA14 filtrate rapidly cleaved the substrate even at the lowest dilution. This finding further proves the rather poor proteolytic activity of MMP-2 on the Ala-Gly-Leu-Ala (SEQ ID NO: 3) motif. Interestingly, the generic MMP substrate reported above was readily cleaved by both matrices (FIG. 39B).

### Divalent LecA Targeting Biofilm Imaging

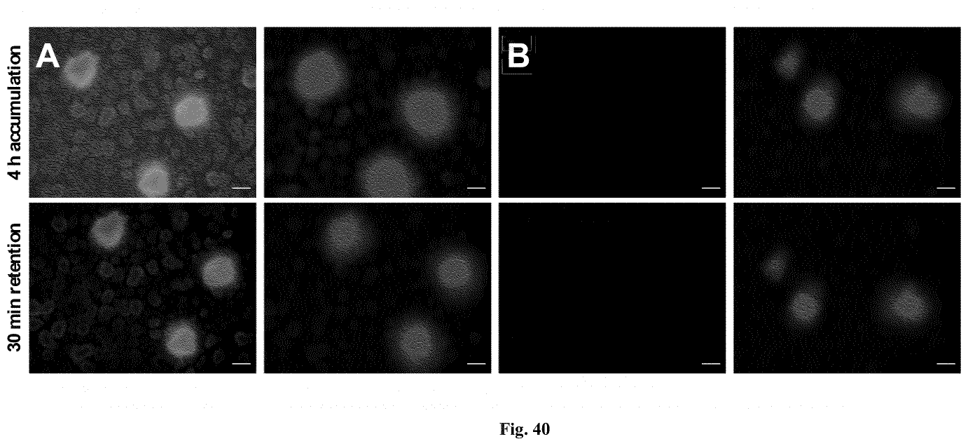

Biofilm staining with divalent LecA imaging probe bearing BODIPY fluorophore (60) was tested in the flow cell experiment in order to validate that staining is independent of imaging moiety. For this, divalent LecA ligand 60 and the corresponding control BDP FL azide were supplemented to the medium and accumulated to P. aeruginosa biofilms. Strong staining was observed for divalent LecA targeting compound 60 as a result of its 4 h accumulation at P. aeruginosa biofilm (FIG. 40A). Im-portantly, high contrast was achieved and biofilm structures were visualized at better contrast. After 30 min wash period, a strong signal was still observed, indicating good retention of 60 at P. aeruginosa biofilm. The corresponding control BDP FL azide did not show any enrichment, demonstrating that the lectin targeting moiety is essential for biofilm staining (FIG. 40B).

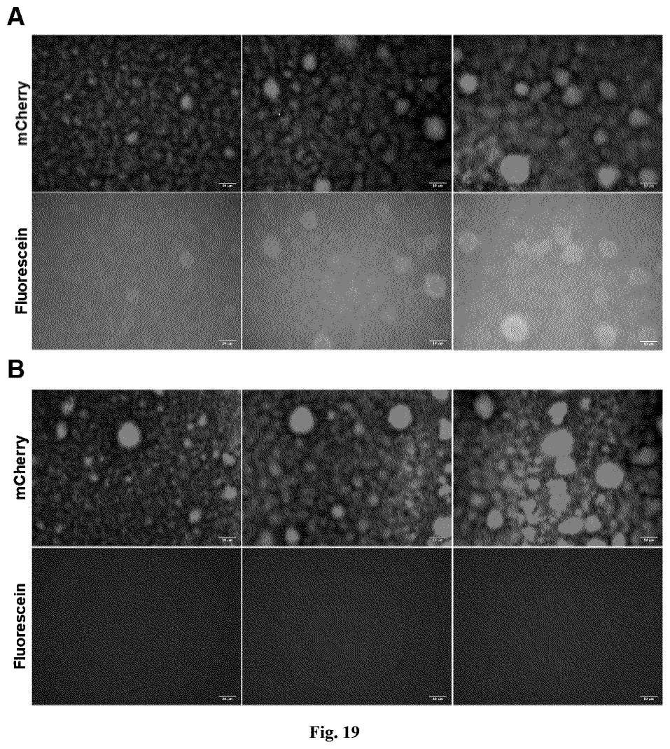

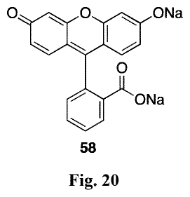

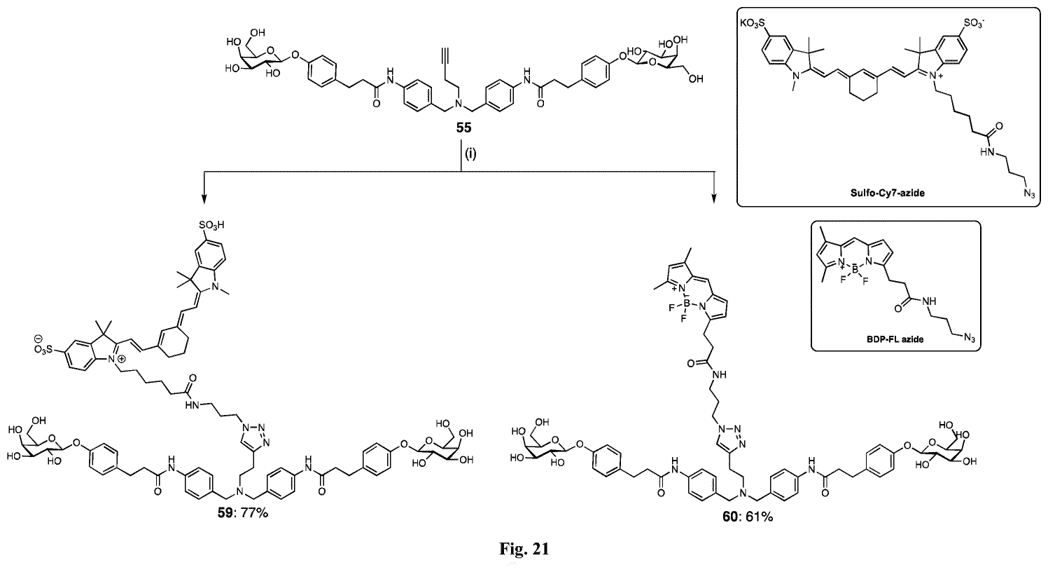

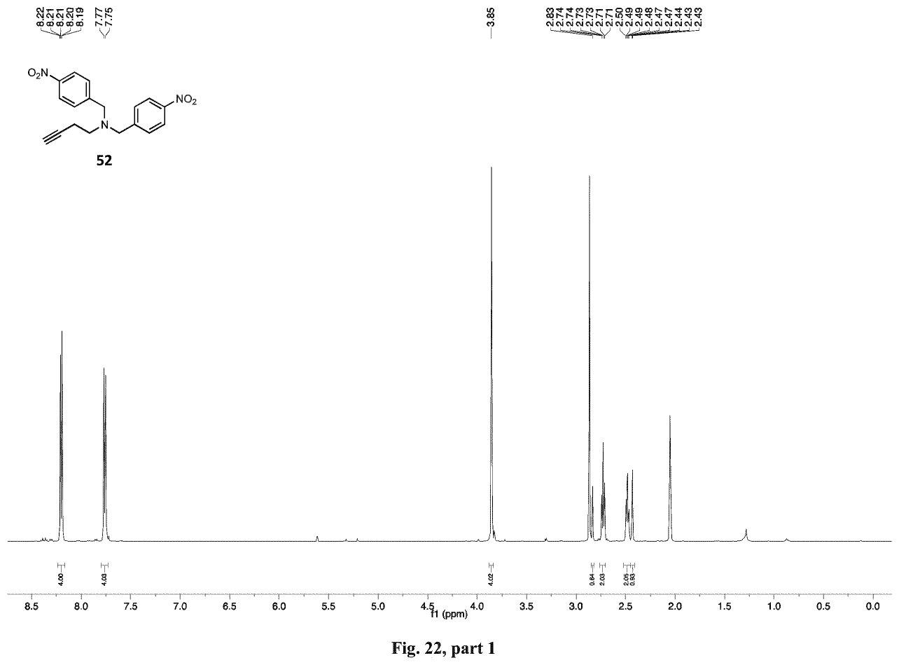

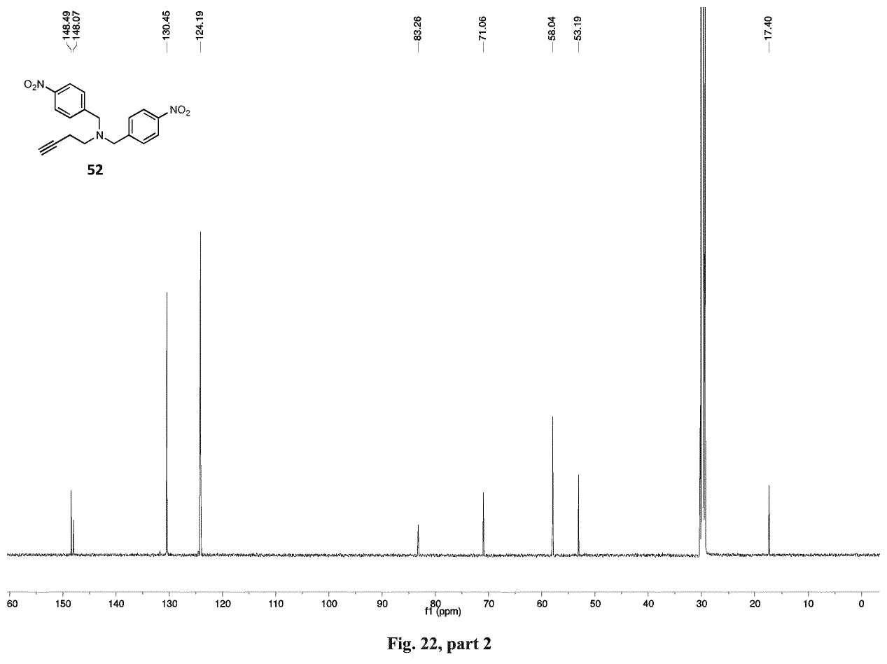

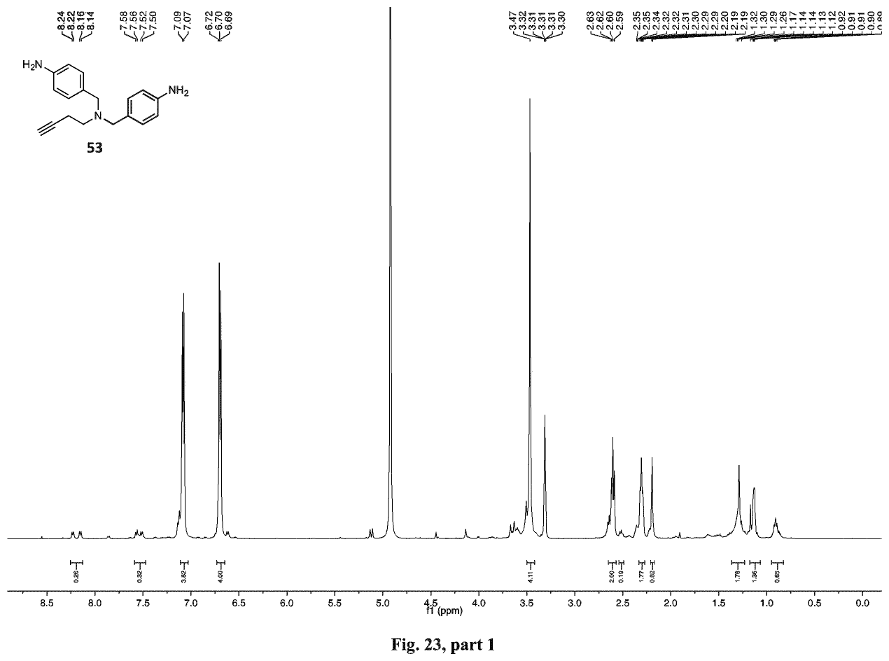

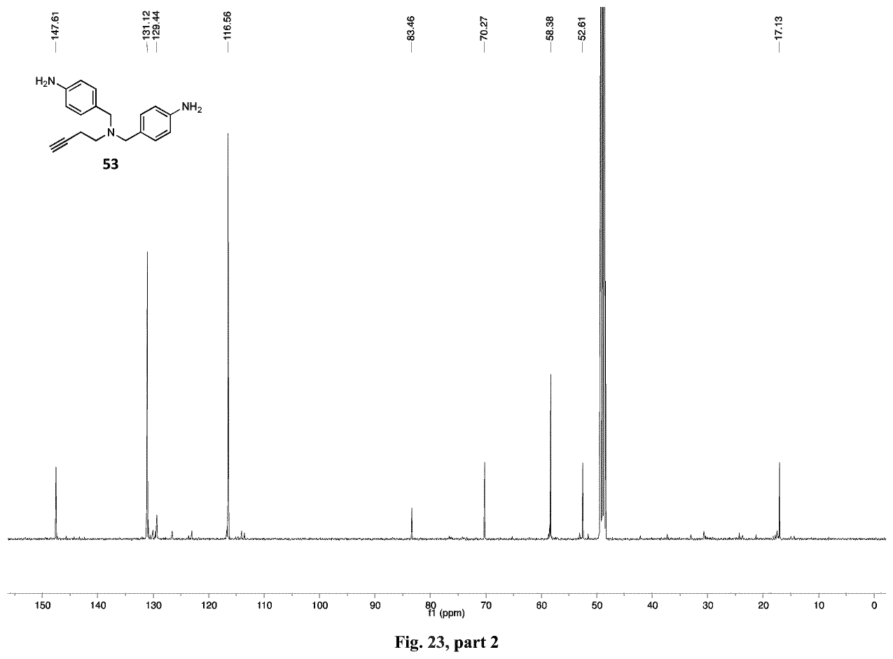

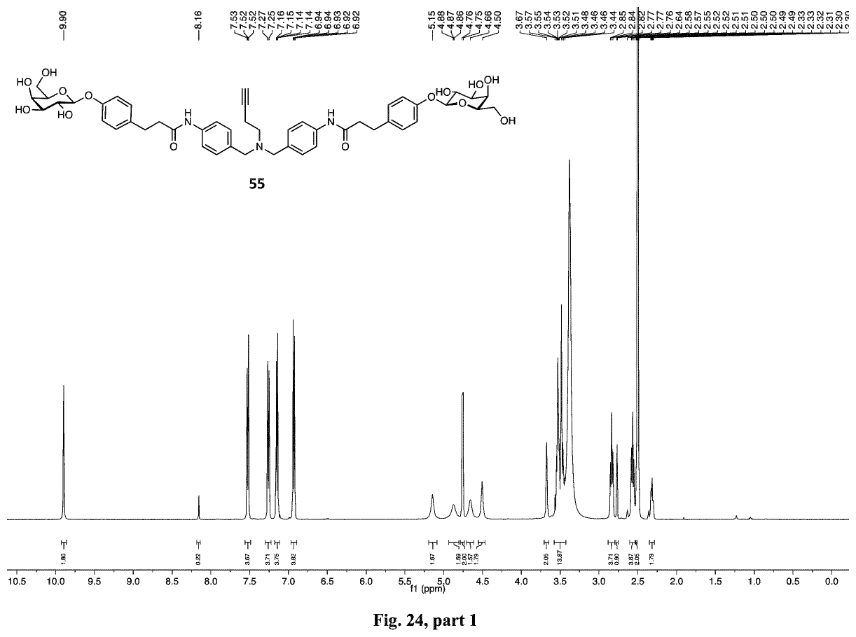

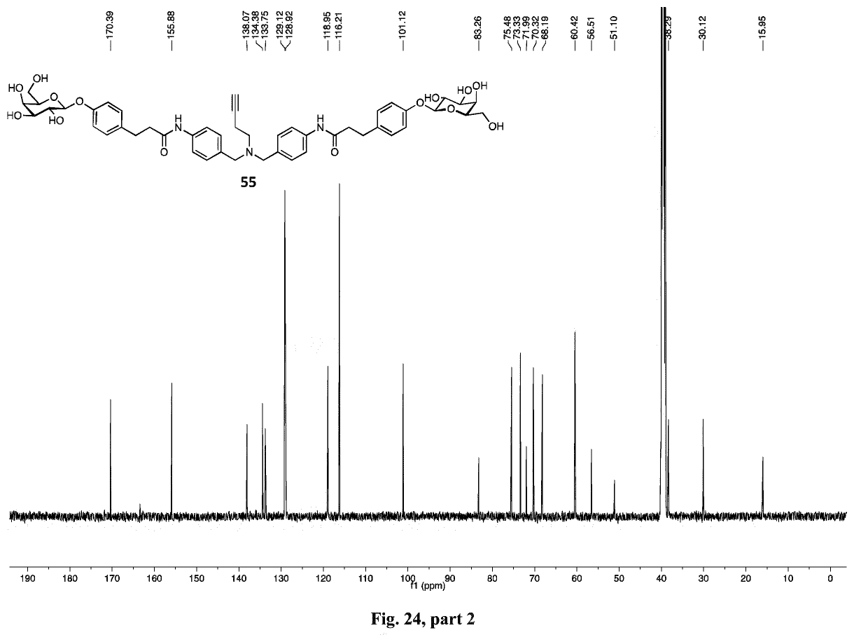

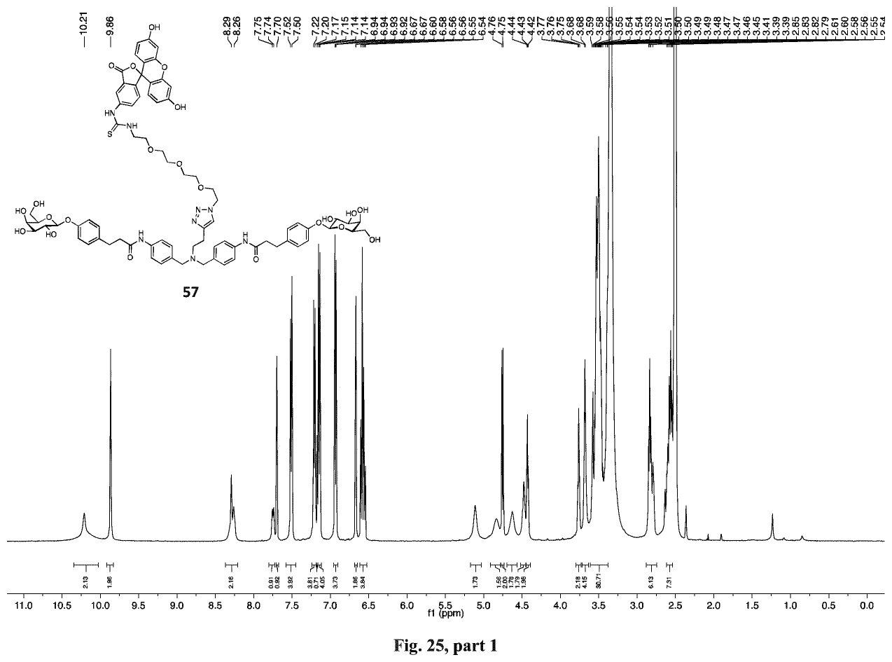

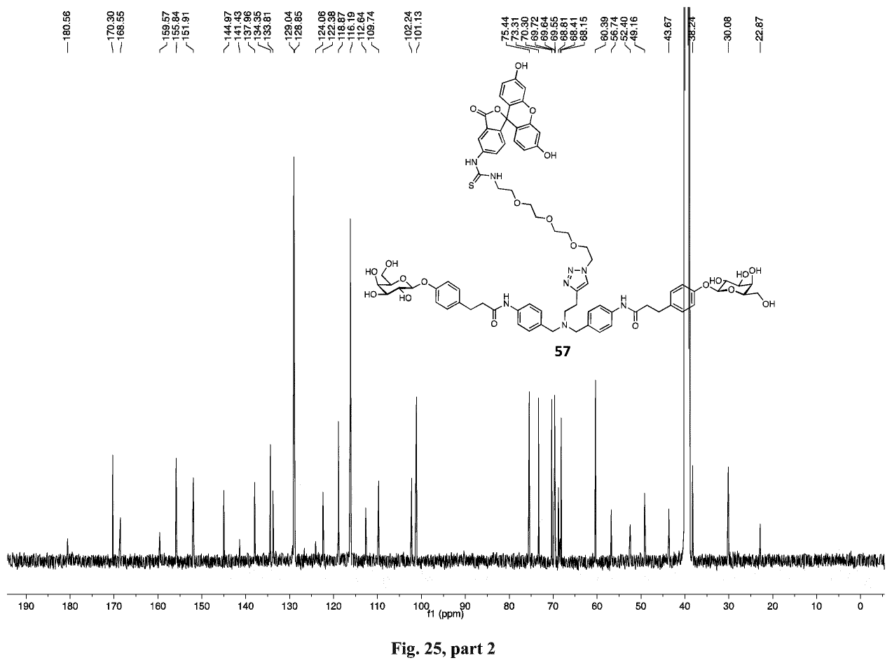

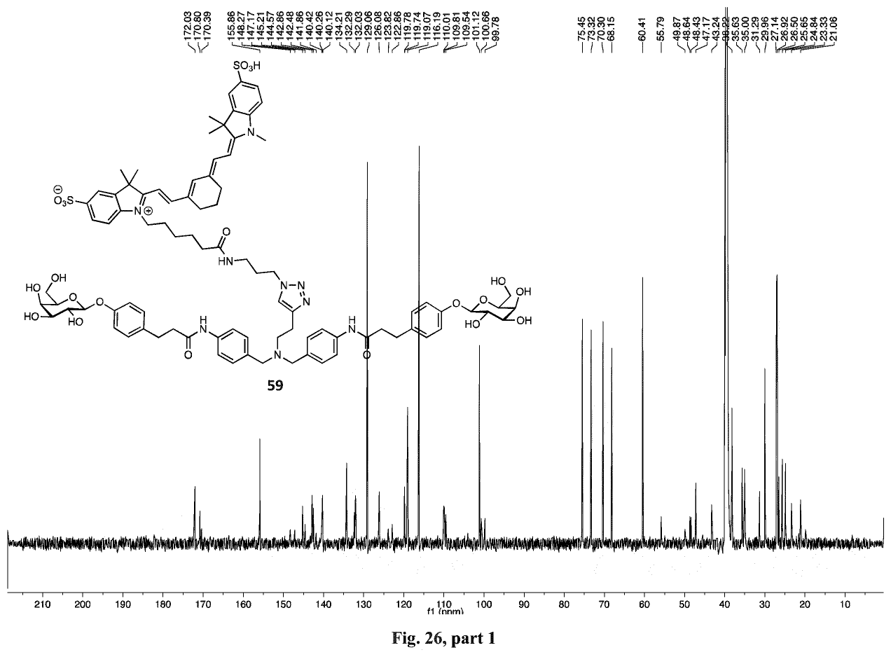

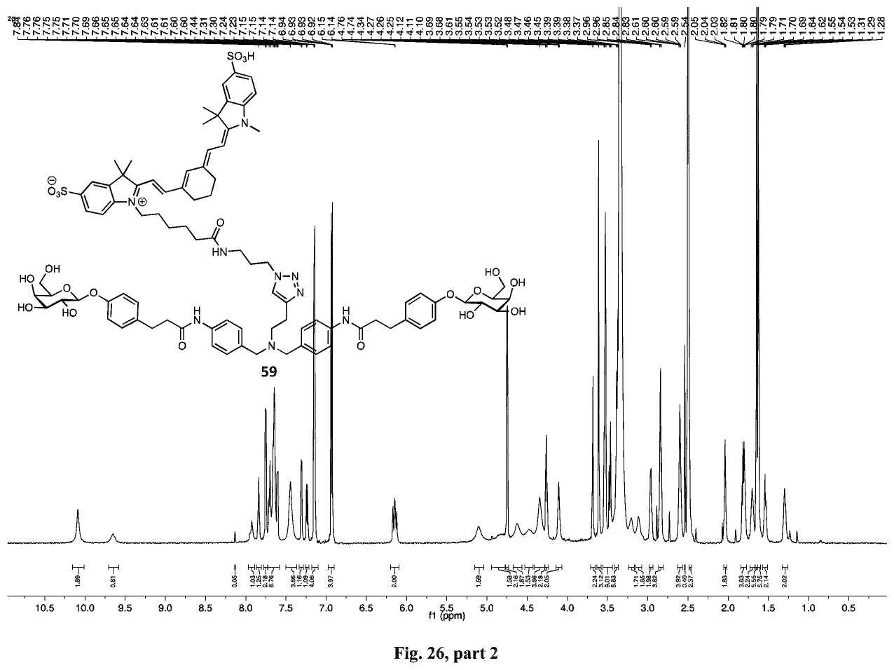

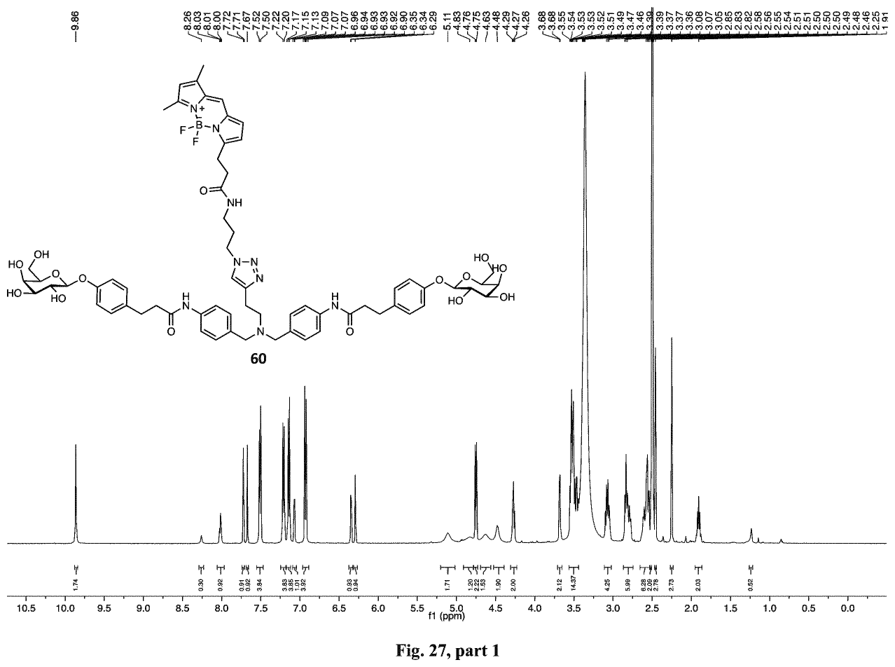

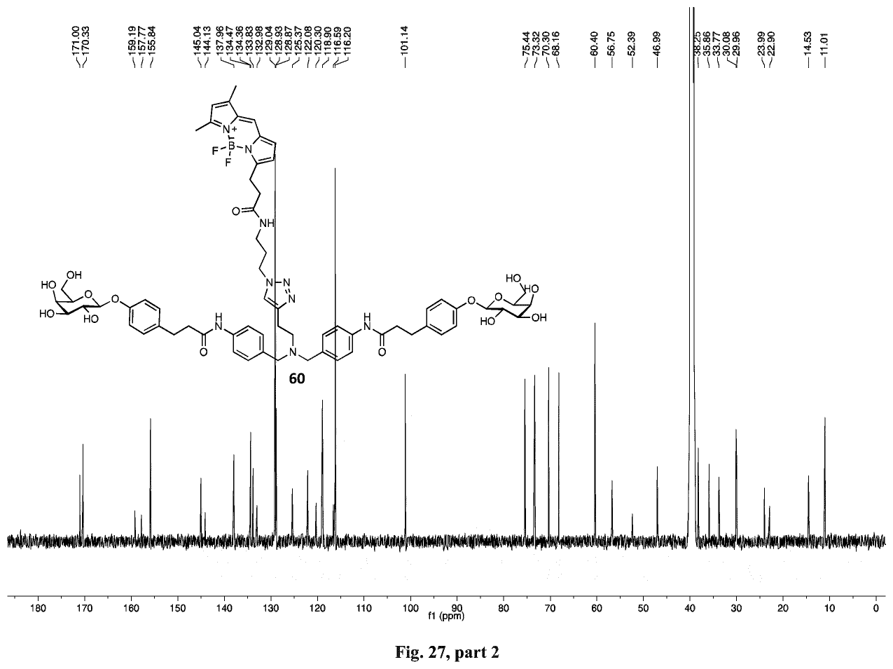

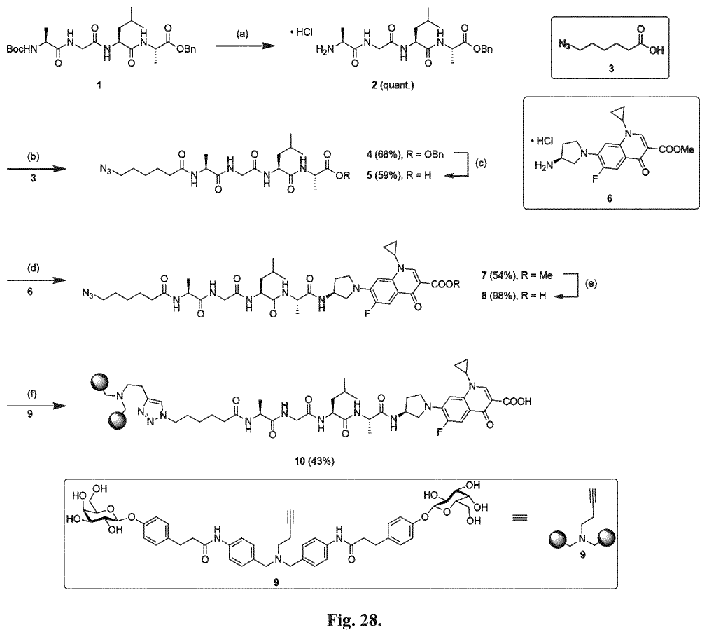

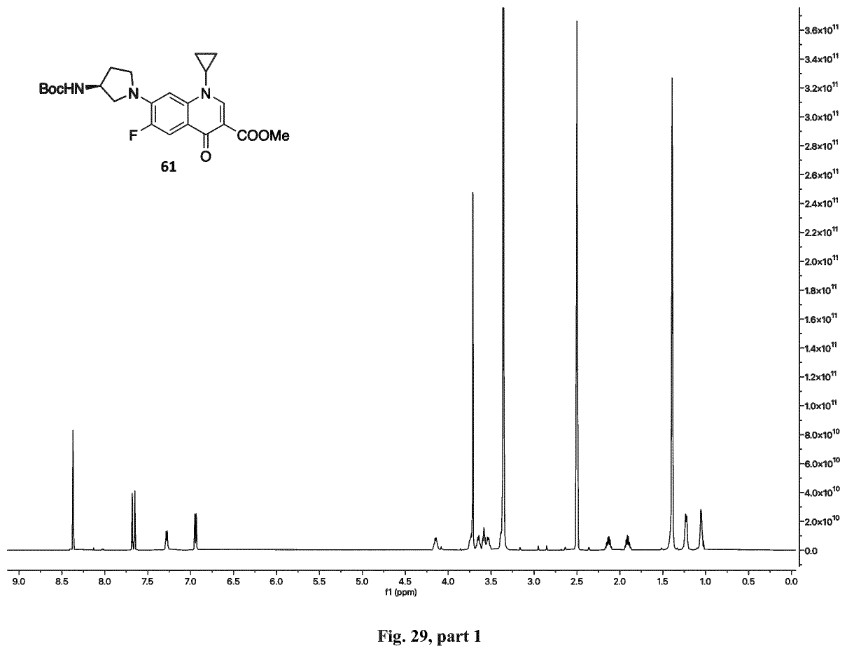

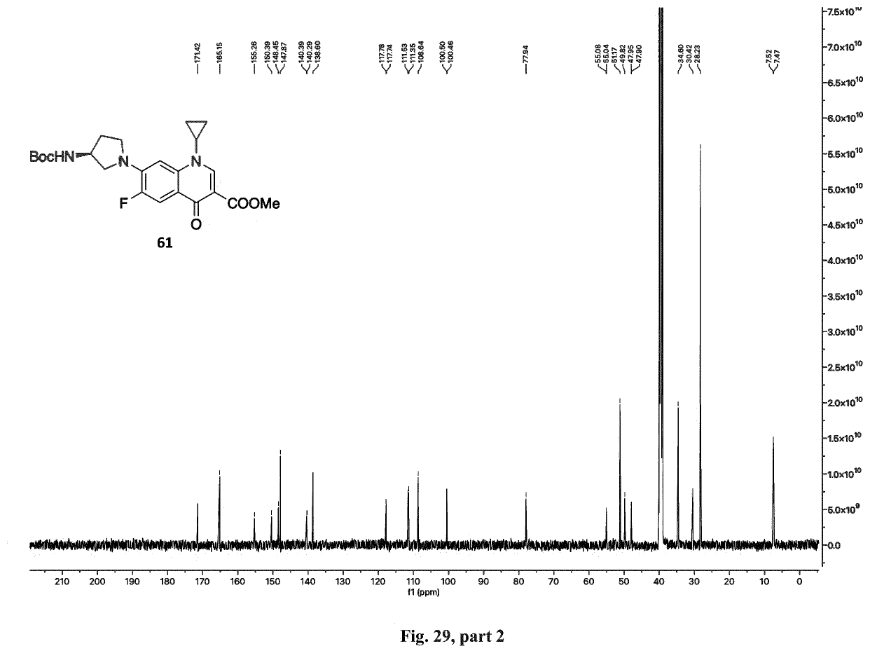

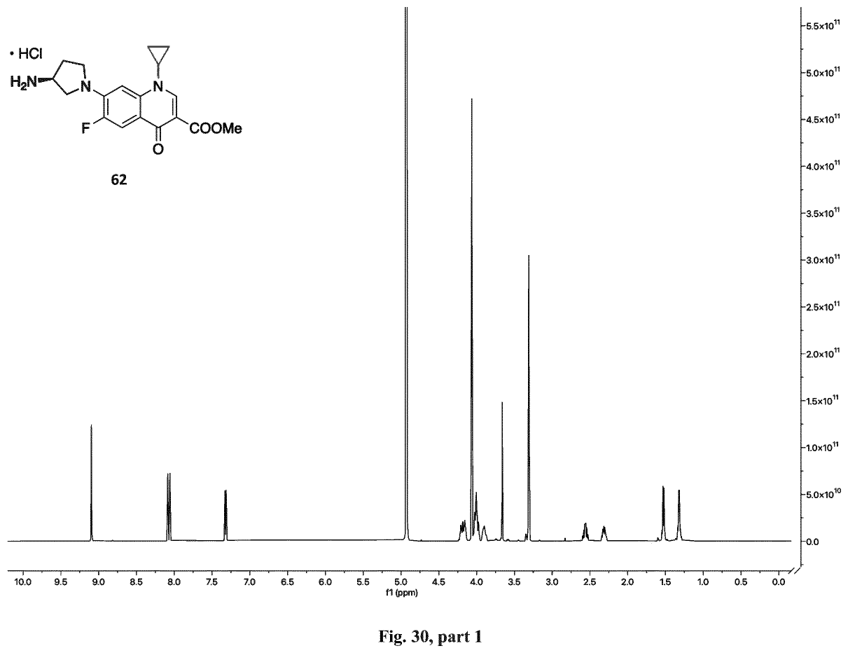

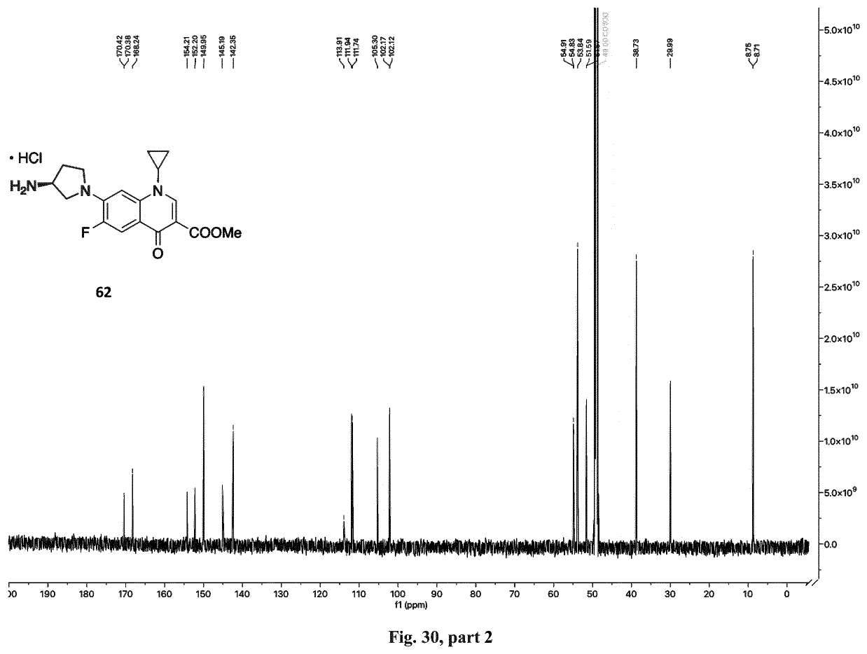

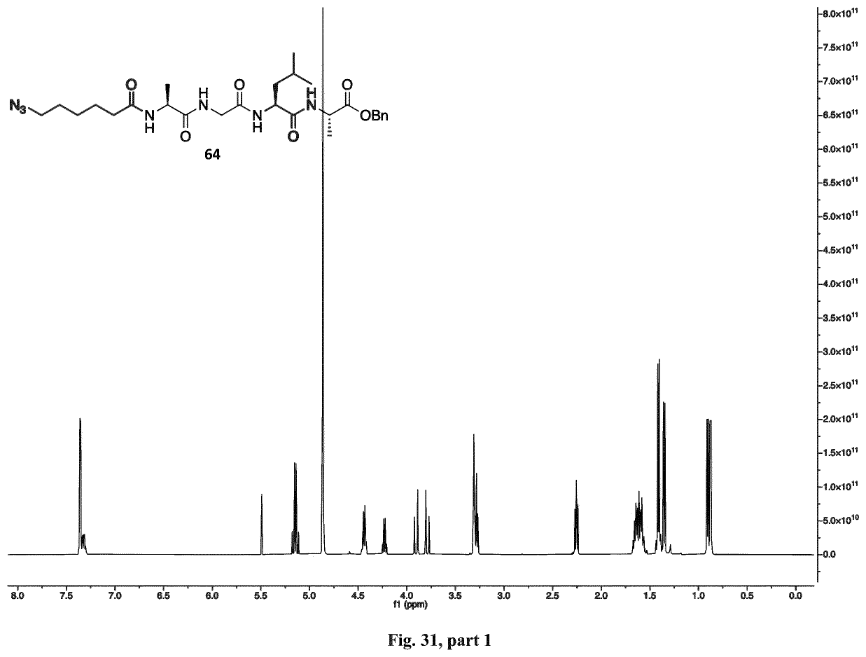

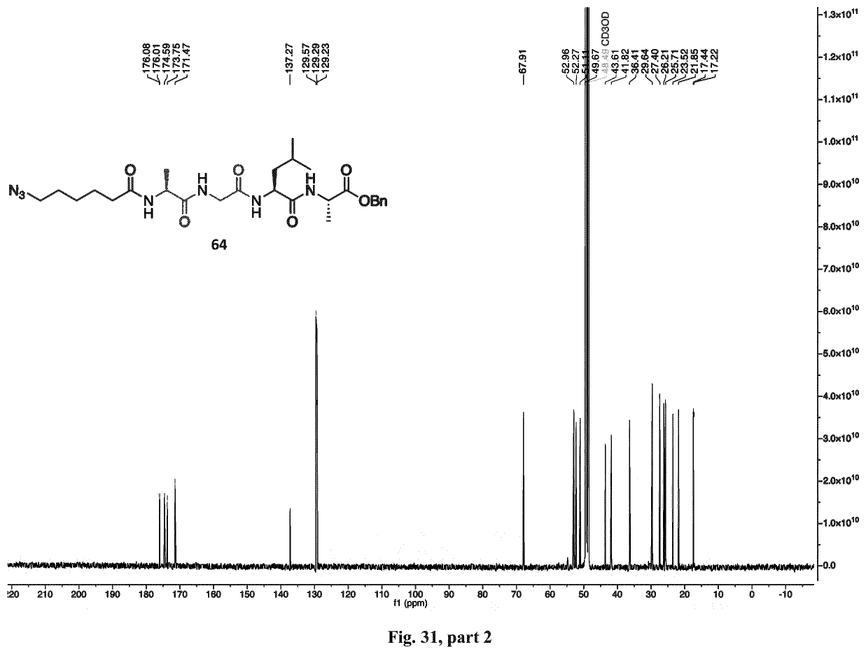

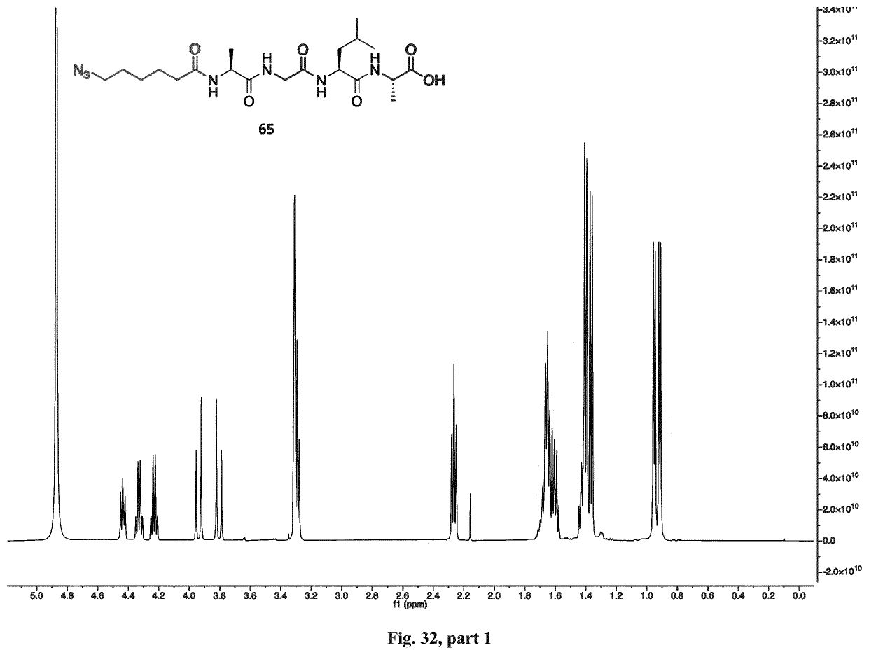

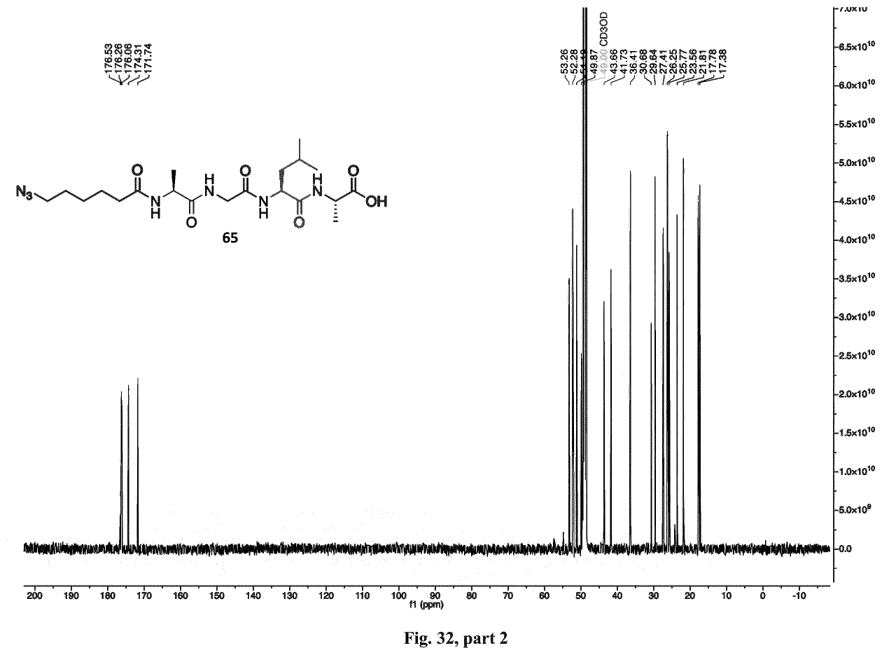

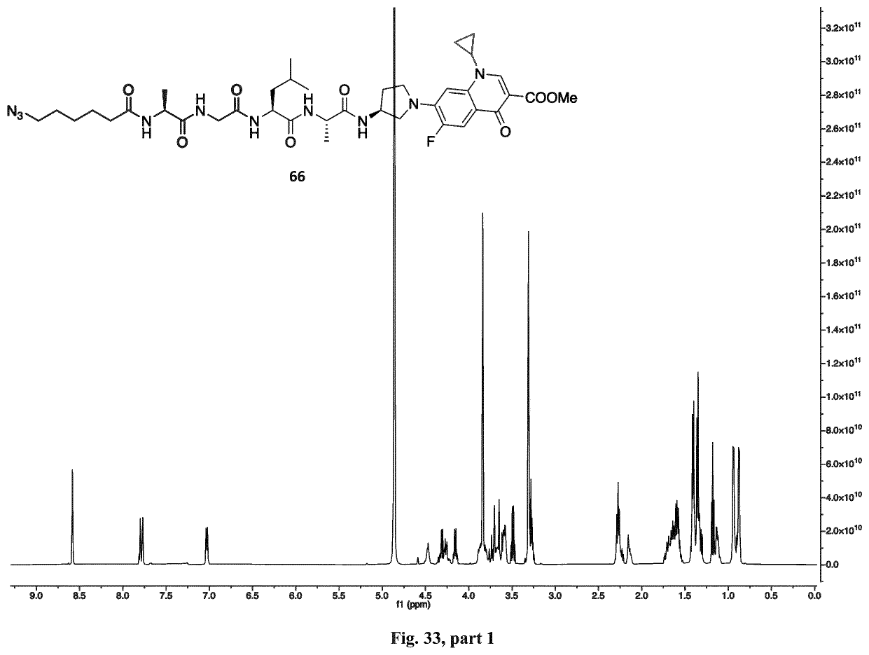

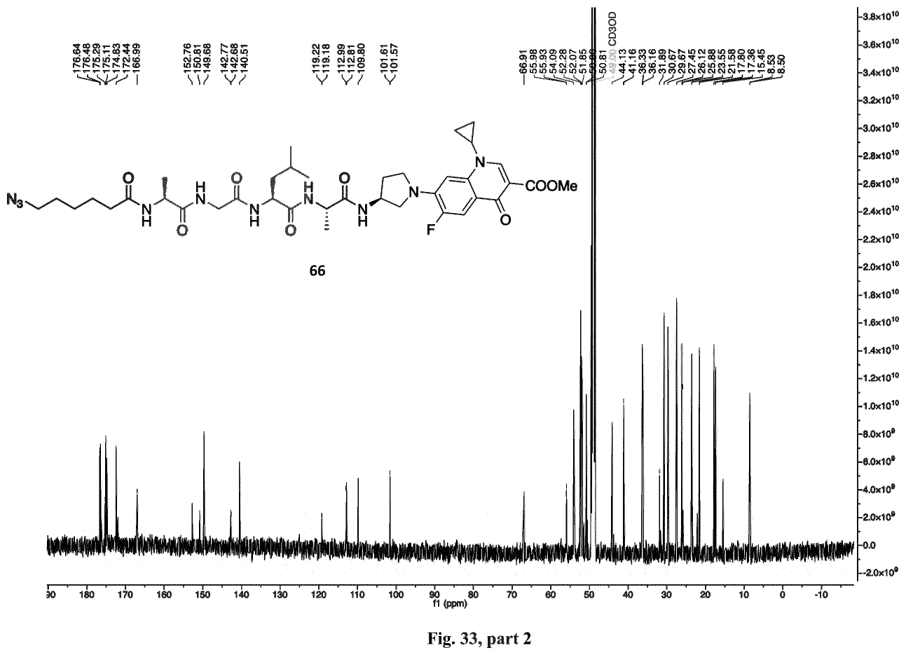

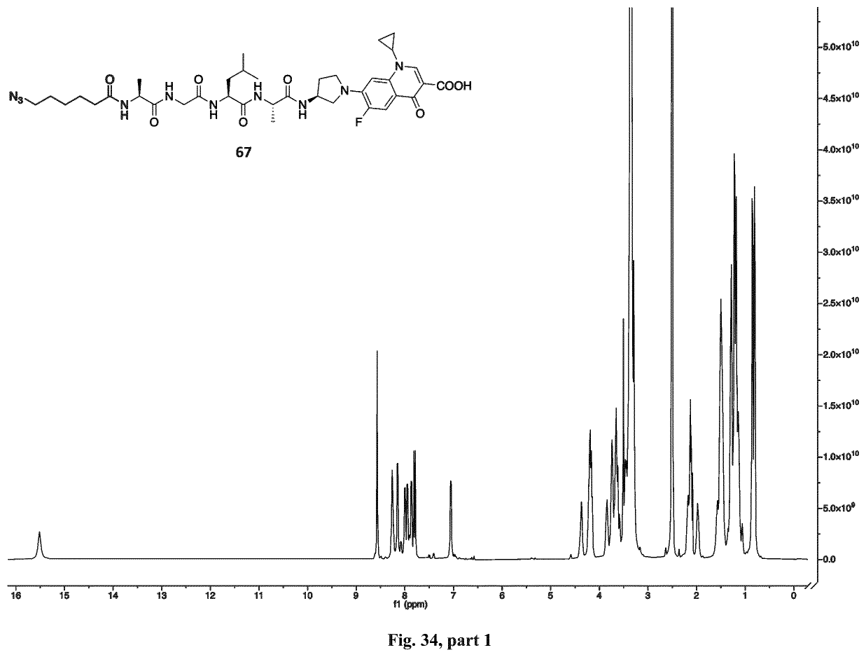

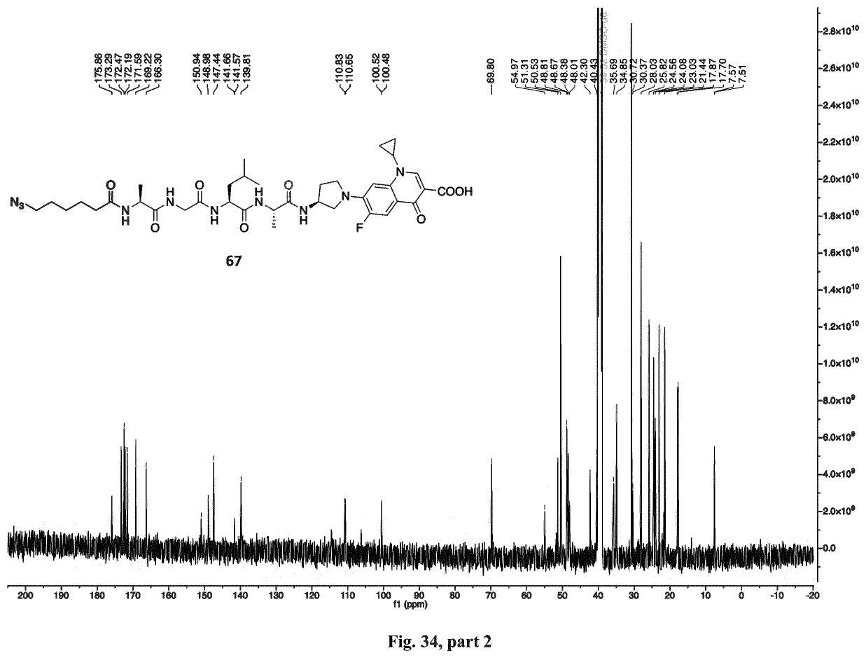

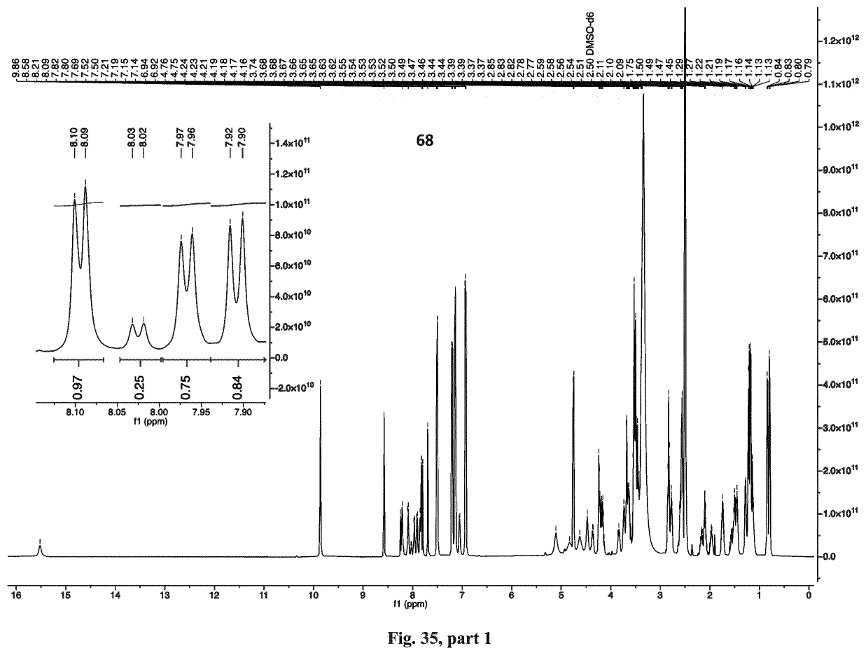

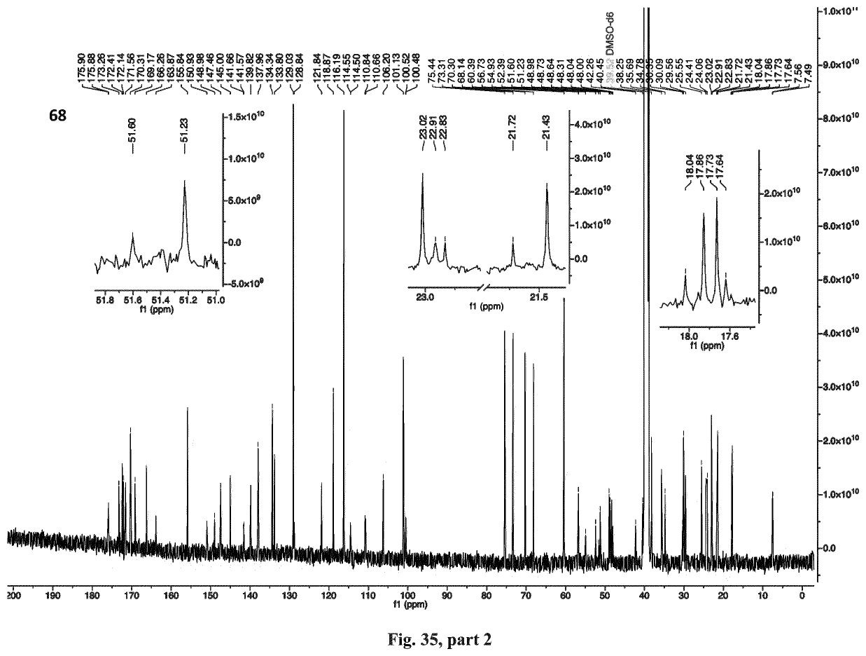

Compared to imaging probe 57 bearing fluorescein (FIG. 19A), more intense staining was observed for 60 after 4 h accumulation as well as after 30 min wash, indicating its better accumulation as well as retention at P. aeruginosa biofilms. Thus, the BODIPY divalent imaging probe 60 outper-formed imaging probe 57 bearing fluorescein.

### CONCLUSIONS AND OUTLOOK

Chronic infections with P. aeruginosa can lead to life-threatening conditions, especially in vulnerable patients. Bacterial biofilms are a major contributor to pathogenicity and antibiotic resistance. The large discovery void of antibiotics with new mode of actions for in last 30 years culminated in the current antibiotic resistance crisis.

Here the first P. aeruginosa biofilm-targeted prodrugs are presented. Carbohydrate probes, to target the two soluble lectins LecA and LecB of P. aeruginosa, were conjugated via a peptide linker to an antibiotic cargo. The linker was designed as substrate of LasB, the major secreted endopeptidase by P. aeruginosa. Three different fluoroquinolones were conjugated to the biofilm-targeted lectin probes and analysed in various assays. Meiers et al. 2010 reported an assay to determine biofilm-accumulation in-vitro. For the prodrugs reported above, the high analytic complexity of this assay is further increased by the described instability of the prodrugs towards the biofilm-component LasB. However, we assume that biofilm-accumulation is also plausible for these prodrugs due to the affinity towards their corresponding lectins.

All prodrugs showed effective target-binding to LecA and both homologs of LecB from P. aeruginosa PA14 and PAO1, thus covering a broad range of clinical isolates. Further, stability and activation of the prodrugs in different biological matrices were characterised. While unspecific activation by human blood plasma was not observed, the initial cleavage in a sterile filtrate of P. aeruginosa PA14 culture supernatant containing LasB was very fast for all prodrugs. When bacterial enzymes and human blood plasma were present, the primary amide-based prodrugs 36, 37, 48 and 49 efficiently released their antibiotic cargo within 3 h. In contrast, proteolysis of ciprofloxacin-based prodrugs was halted at the stage of the secondary amide, resulting in poor release of ciprofloxacin.

In antimicrobial activity assays, we could show that the inactivated prodrugs were inactive, while proteolytic activation leads to very potent antibiotic drugs. Especially in the case of aminopyrrolidine-based prodrugs, compounds 36 and 37 reached high antibiotic activities (0.098-0.195 μM), comparable to their parent fluoroquinolone 2 (0.027-0.054 μM). The ciprofloxacin-based prodrugs showed no significant antibiotic activity, independent of the activating biological matrix, which was consistent with our cleavage data (FIG. 8).

In-vitro ADMET analysis of the most active aminopyrrolidine-based series 2, 36, and 37 proved their metabolic stability in microsomal liver fractions and blood plasma in both species, human and mouse. For both prodrugs, the stability in presence of human liver microsomes was enhanced compared to parent fluoroquinolone 2. Acute cytotoxicity was not observed against A549 lung carcinoma cells using an MTT cytotoxicity assay for assessment. In cell accumulation experiments, the prodrugs showed strongly reduced cell permeability. This is a major improvement compared to the parent drugs, due to the absence intracellular off-target inhibition and formation of ROS. In conclusion, this work defines the starting point for the first P. aeruginosa biofilm-targeted antibiotic prodrugs.

1. A conjugate having a structure according to formula (I)
R¹₍ₙ₎—Y—R² (I)
or a structure according to formula (II) 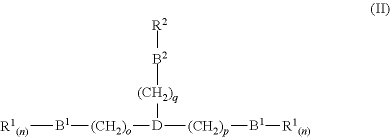
wherein
R¹ comprises one or more ligands specifically binding to a bacterial lectin;
Y is a linker comprising a peptide (Pep) cleavable by a bacterial protease;
R² is an anti-bacterial therapeutic agent in formula (I) or an anti-bacterial therapeutic agent or imaging agent in formula (II);
wherein n is between 1 to 10 and if n is 2 to 10 each R¹ can be the same or different B¹ in each case is independently selected from a first bridging moiety;
B² is a second bridging moiety;
D is selected from the group consisting of amine, ammonium, phosphate, phosphine, phosphonate, tricarboxybenzoic acid, triaminomethyl benzene, citric acid, glycerol, trishydroxymethyl amine, lysine, cyclo oligolysine, 1,4,7,10-tetrazacyclododecan, 1,4,7-triazacyclononan, and 1,5,9-triazacyclododecan, and
o, p and q are independent of each other selected from 0 to 4.

2. The conjugate according to claim 1, wherein the bacterial lectin is:
(i) a galactose-binding lectin, preferably a galactose-binding lectin of a bacterium selected from the genus Chromobacterium, Collimonas sp., Enterobacter sp., Klebsiella, Jejubacter, Mesorhizobium sp., Mycobacterium, Ochrobactrum sp., Photorhabdus sp., Pseudomonas sp., and Xenorhabdus sp. or a homologue of LecA from Pseudomonas aeruginosa that has a sequence identity according to SEQ ID NO: 1 of at least 60%; or
(ii) a mannose- or fucose-binding lectin, preferably a mannose or fucose binding lectin of a bacterium selected from the genus Burkholderia sp., Klebsiella, Chromobacterium, Citrobacter, Diaphorobacter sp. Paraburkholderida, Ralstonia, Pseudomonas sp., Vibrio, Halomonas, and Listeria or a homologue thereof that has a sequence homology to LecB Pseudomonas aeruginosa according to SEQ ID NO: 2 of at least 60%.

3. The conjugate according to claim 1, wherein the ligand comprises one or more saccharides or consists of a saccharide, preferably a mono-, di- or trisaccharide, wherein the —O-forming the glycosidic linkage between R¹ and Y in formula (I) or R¹ and B¹ in formula (II) may be replaced independently from each other by Z, wherein Z is [saccharide]—S—, [saccharide]—CH₂—NH—SO₂—, [saccharide]—CH₂—NH—CO—, [saccharide]-NH—SO₂—, [saccharide]—NH—CO—, [saccharide]—NH— group, or [saccharide]—CH₂—.

4. The conjugate according to claim 3, wherein the monosaccharide is selected from the group consisting of galactose, fucose, mannose, xylose or a derivative thereof, wherein a preferred galactose derivative is selected from the group consisting of galactose, N-acetylgalactosamine (GalNAc), 2-deoxy-galactose, and epoxides of galactoheptose and esters, preferably acetates; a preferred mannose derivative is selected from the group consisting of 6-deoxymannose, 6-deoxy-6-sulfonamido mannose, or N-acetylmannosamine (ManNAc), and a preferred fucose derivative is selected from the group consisting of N-acetyl-L-fucosamine (FucNAc), 2-deoxy-L-fucose, and 6-hydroxy-L-fucose,
wherein in each case the —O— group forming the glycosidic linkage between R¹ and Y in formula (I) or R¹ and B¹ in formula (II) may be replaced by Z.

5. The conjugate according to claim 1, wherein R¹ is or is independently from each other selected from the group consisting of the formulae (III) to (XIX) 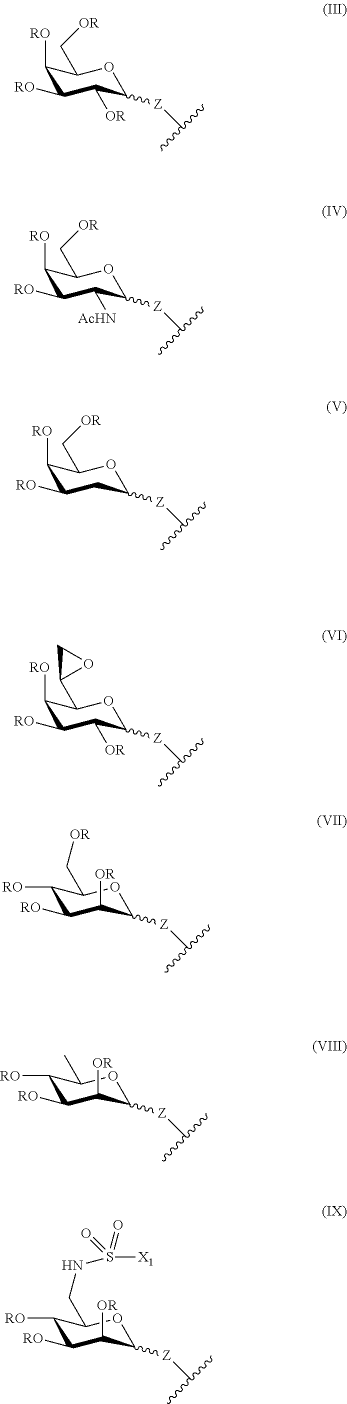 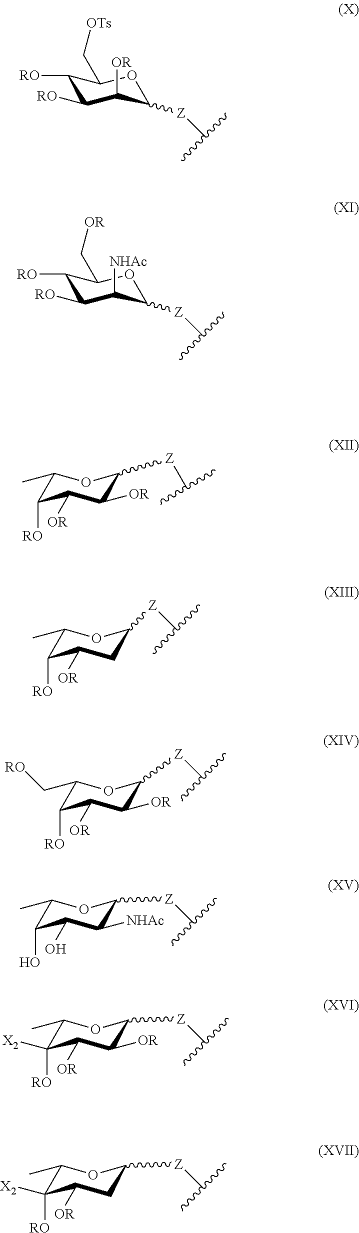 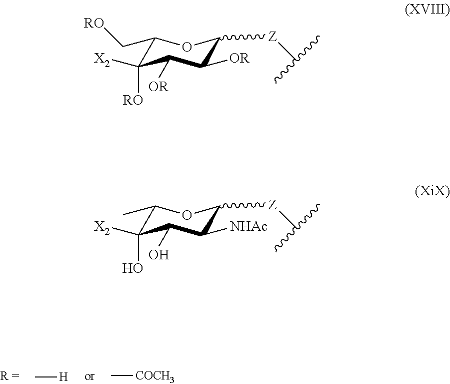
wherein
Z is selected from the group consisting of O, S, NH, —CH₂—NH—CO—, —NH—CO—, —NH—SO₂—, and CH₂, and
X¹ and X² are independently selected from alkyl, aryl, alkylaryl, heteroaryl, or alkylheteroaryl, preferably aryl; optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR⁴R⁵ wherein R⁴ and R⁵ is independent of each other a C₁-C₄ alkyl group, —NO₂, —CN, —COOH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H.

6. The conjugate according to claim 1, wherein Y comprises or consists of:
(i) -Pep-;
(ii) —Bi-Pep-;
(iii) —B¹-Pep-B²—
(iv) or comprises, one, two or three X, Pep and optionally at least one B¹ and/or at least one B², wherein X is directly or indirectly via B¹ bound to at least two R¹, and Pep is directly or indirectly via B² bound to R²;
preferably the targeted delivery conjugate according to formula (I) has the following structure: 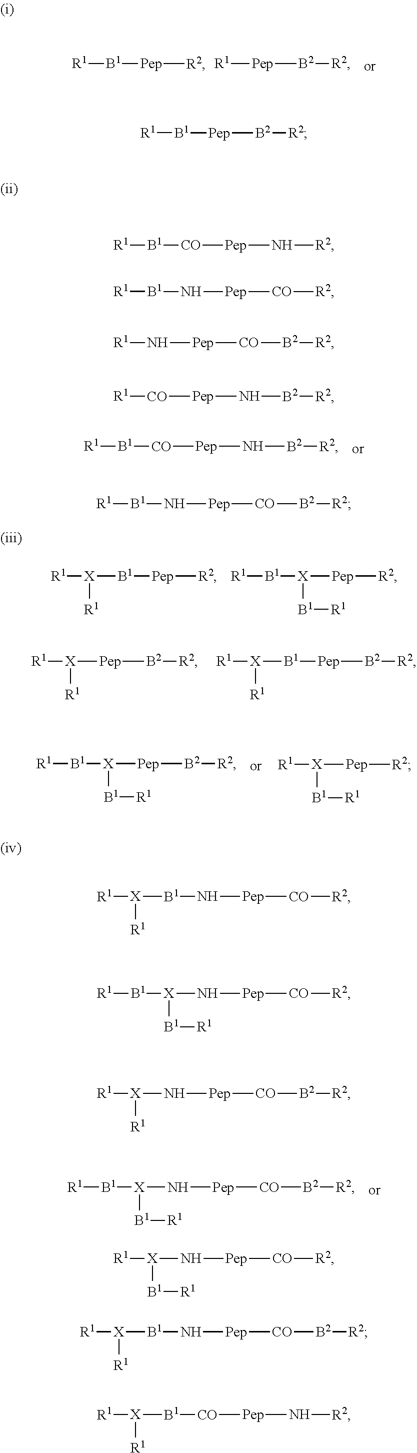 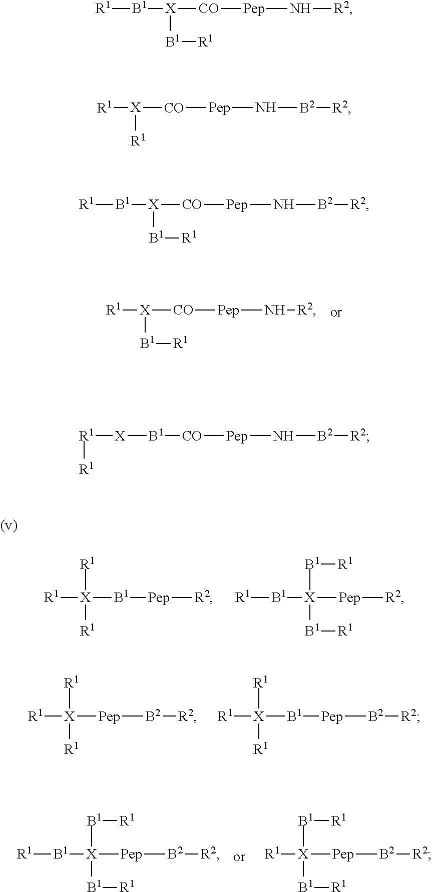
wherein
B¹ in each case is independently selected from a first bridging moiety;
B² in each case is independently selected from a second bridging moiety;
X is a branching moiety capable of forming at least three covalent bonds; and
NH is the amino group of the N-terminal amino acid of Pep and CO is the carboxy group of the C-terminal amino acid of Pep.

7. The conjugate according to claim 1, wherein
B¹ comprises or consists of A, R³, R³-A, A-R⁶, R³-A-R⁶, -A-R⁷-A-, R³-A-A-R⁶, -A-R⁷-A-R⁶, or R³-A-R⁷-A-R⁶, wherein each A may be the same or different, preferably is A-R⁶;
B² comprises or consists of A, R¹, R¹-A, A-R⁹, or R¹-A-R⁹, -A-A-, -A-A-R⁹, R¹-A-A-, -A-A-R⁹, R¹-A-A-R⁹ or R¹-A-R⁷-A-R⁹, wherein each A may be the same or different, preferably is A-R⁹;
wherein
A is selected from the group consisting of:
(a) an carbocyclic group that is optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR⁴R¹ wherein R⁴ and R¹ is independent of each other a C₁-C₄ alkyl group, —NO₂, —CN, —COOH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H, and
(b) a heterocarbocyclic group that is optionally substituted by one, two or three substituents that are independent of each other selected from the group consisting of a C₁-C₄ alkyl group, halogen, a C₁-C₄ haloalkyl group, —OH, a C₁-C₄ alkoxy group, —NH₂, —NHR⁴ wherein R⁴ is a C₁-C₄ alkyl group, —NR⁴R⁵ wherein R⁴ and R⁵ is independent of each other a C₁-C₄ alkyl group, —NO₂, —CN, —COOH, —COOR⁴ wherein R⁴ is a C₁-C₄ alkyl group, —CONH₂, —CONHR⁴ wherein R⁴ is a C₁-C₄ alkyl group, —CONR⁴R⁵ wherein R⁴ and R⁵ is independent of each other a C₁-C₄ alkyl group, and —SO₃H;
(c) [O—CH₂—CH₂]m-, wherein m is between 1 to 10; or
R³ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—; —CH═CH—NH—CO—; —NH—CO—CH═CH—; and —CH₂—CH₂—NH—CO—, wherein R³, if present is bound to R¹;
R⁶ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—CH═CH—NH—CO—; —NH—CO—CH═CH—; and —CH₂—CH₂—NH—CO—, preferably —CO— and —NH—, wherein R⁶, if present is bound to X or Pep;
R⁷ is selected from the group consisting of —CO—, NH—, —NH—CO—, —CO—NH—, CO—NH—(C₁-C₄ alkyl), —NH—CO—(C₁-C₄ alkyl), —(C₁-C₄ alkyl)-CO—NH—; or —CO—NH—(C₁-C₄ alkyl);
R⁸ is selected from the group consisting of —CO—; —NH—, —CO—NH—N═CH—; —CH₂—CH₂—CO—NH—; —CO—NH—CH═CH—; —CH═CH—CO—NH—; —CO—NH—CH₂—CH₂—; —NH—CO—CH₂—CH₂—; —CH═CH—NH—CO—; —NH—CO—CH═CH—; and —CH₂—CH₂—NH—CO—, wherein R⁸, if present is bound to Pep or (CH₂)q;
R⁹ is selected from the group consisting of —CO—NH—(C₁-C₄ alkyl), —NH—CO—(C₁-C₄ alkyl), —(C₁-C₄ alkyl)-CO—NH—; or —CO—NH—(C₁-C₄ alkyl)-CO—; —NH—, NH—S—NH, preferably —CO— and —NH—, wherein R⁹, if present is bound to R²;
and/or
X is selected from the group amine, ammonium, phosphate, phosphine, phosphonate, tertiary or quaternary carbon, tricarboxybenzoic acid, triaminomethyl benzene, citric acid, glycerol, trishydroxymethyl amine, lysine, cyclo oligolysine, 1,4,7,10-tetrazacyclododecan, 1,4,7-triazacyclononan, and 1,5,9-triazacyclododecan.

8. The conjugate according to claim 7, wherein A is selected from the group consisting of
(i) a naphthalenediyl group;
(ii) a five-membered aromatic or nonaromatic monocyclic ring, wherein 1, 2, 3, or 4 of the ring atoms are the same or different heteroatoms, said heteroatoms being selected from O, N, or S, preferably triazolyl,
(iii) a six-membered aromatic or nonaromatic monocyclic ring, wherein 1, 2, 3, 4, or 5 of the ring atoms are the same or different heteroatoms, said heteroatoms being selected from O, N, or S, preferably a phenylene group; or
(iv) an aromatic or nonaromatic bicyclic ring system with 8 to 12 members, wherein 1, 2, 3, 4, 5, or 6 of the ring atoms are the same or different heteroatoms, said heteroatoms being selected from O, N, or S;
wherein each one of the above mentioned groups (i) to (v) is optionally substituted by one, two or three substituents that are independently from each other selected from the group consisting of a C₁-C₄ alkyl group, halogen, a C₁-C₄ haloalkyl group, —OH, a C₁-C₄ alkoxy group, —NH₂, —NHR¹³ with R¹³ being a C₁-C₄ alkyl group, —NR¹³R¹⁴ with R¹³ and R¹⁴ each being independently from each other a C₁-C₄ alkyl group, —NO₂, —CN, —COOH, —COOR¹³ with R¹³ being a C₁-C₄ alkyl group, —CONH₂, —CONHR¹³ with R¹³ being a C₁-C₄ alkyl group, —CONR¹³R¹⁴ with R¹³ and R¹⁴ each being independently from each other a C₁-C₄ alkyl group, and —SO₃H.

9. The conjugate according to claim 1, wherein Pep comprised in the linker:
(i) has a length between 2 to 20 amino acids; and/or
(ii) has a direct covalent bond to the anti-bacterial therapeutic agent; and/or
(iii) comprises or consists of an amino acid sequence selected from the group consisting of FFA, ALA, GGG, GGF, GGA, AGLA (SEQ ID NO: 3), FGLA (SEQ ID NO: 4), FGAK (SEQ ID NO: 5), LVLGA (SEQ ID NO: 6), LVLGGS (SEQ ID NO: 7), LLLGGS (SEQ ID NO: 8), VVLGGS (SEQ ID NO: 9), VLGS (SEQ ID NO: 10), LVFGGS (SEQ ID NO: 11), LVMTSG (SEQ ID NO: 12), RVRGHF (SEQ ID NO: 13), IAAG (SEQ ID NO: 14), LAFGA (SEQ ID NO:15), IVFGGS (SEQ ID NO: 16), VVGGSG according to SEQ ID NO: 17, VVAGGS according to SEQ ID NO: 18, IFGA (SEQ ID NO: 19), ITYGAS (SEQ ID NO: 20), GSFGAR (SEQ ID NO: 21), LVFGA (SEQ ID NO: 22), IAKD (SEQ ID NO:23), KGPA (SEQ ID NO: 24), PLGPDR (SEQ ID NO: 25), AGPPGP (SEQ ID NO: 26), PRPPAPVFY (SEQ ID NO: 27), RRKKVYP (SEQ ID NO: 28), TPIQL (SEQ ID NO: 29), LPAL (SEQ ID NO: 30), LRIS (SEQ ID NO: 31), LKIS (SEQ ID NO: 32), LKLN (SEQ ID NO: 33), ARFT (SEQ ID NO: 34), LQLP (SEQ ID NO: 35), LGLP (SEQ ID NO: 36), LFGA (SEQ ID NO: 37), LYGA (SEQ ID NO: 38) and IYGA (SEQ ID NO: 37); and/or
(iv) wherein one or more of the peptide bonds between the amino acids of Pep that are not cleaved by the bacterial protease are bioisosters of the peptide bonds, preferably N-alkyl/cycloalykl peptides, retropeptides, 5-membered heteroaromatic rings, preferably triazoles/oxadiazoles/thiazoles/, ureas, thioureas, beta-peptides, sulfonamides, thioamides, and carbamates.

10. The conjugate according to claim 1, wherein the anti-bacterial therapeutic agent is an antibiotic, preferably selected from the group consisting of fluoroquinolones, carbapenems, penicillins, monobactams, cephalosporins, aminoglycosides, polymyxins, argyrins, cystobactamids and a prodrug thereof.

11. The conjugate according to claim 1, wherein the imaging agent is a chemical group detectable by fluorescence spectroscopy, by positron-emission tomography (PET), or by magnetic resonance imaging (MRI), preferably fluorescein, sulfoCy7, TAMRA, BODIPY, 18F, Gd(III)-DOTA.

12. The conjugate according to claim 1, with a structure selected from the group consisting of: 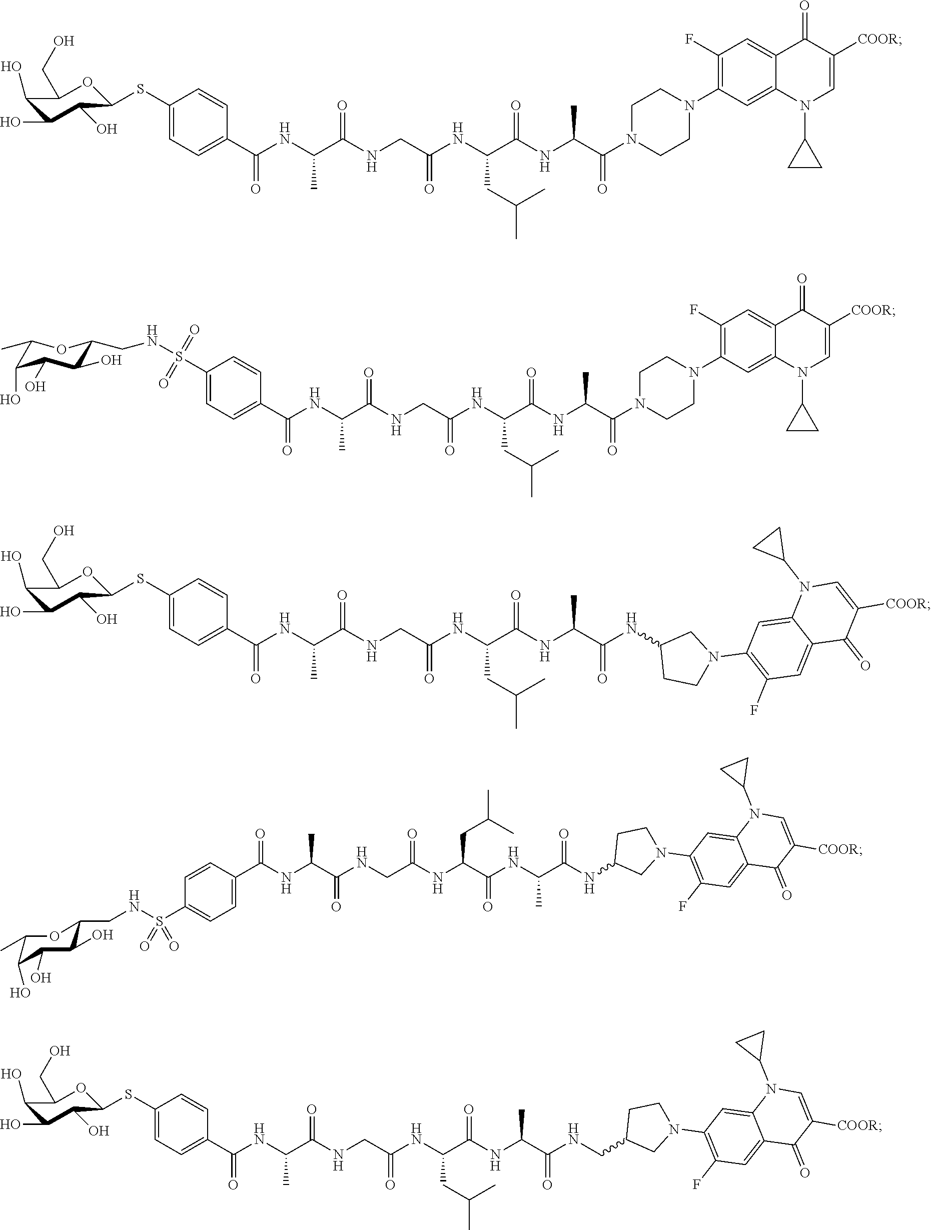 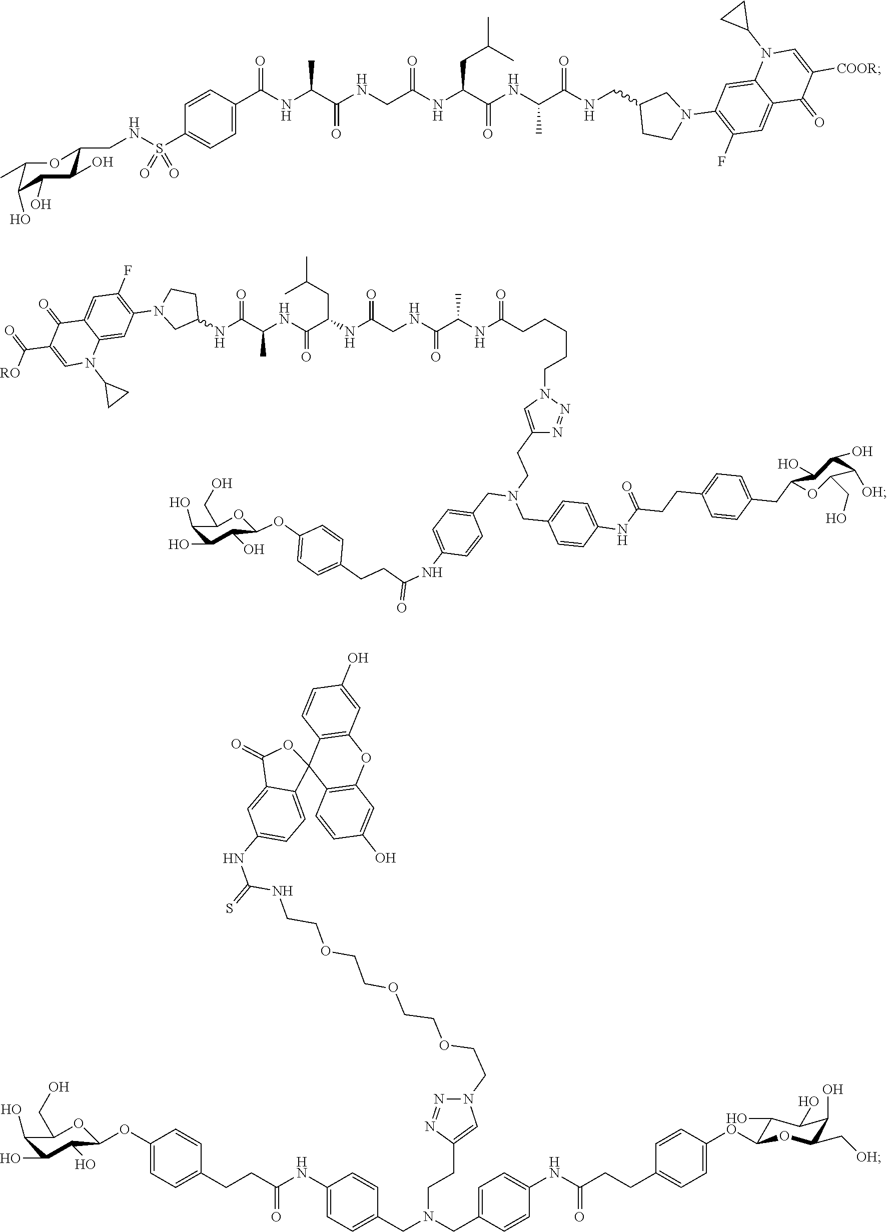 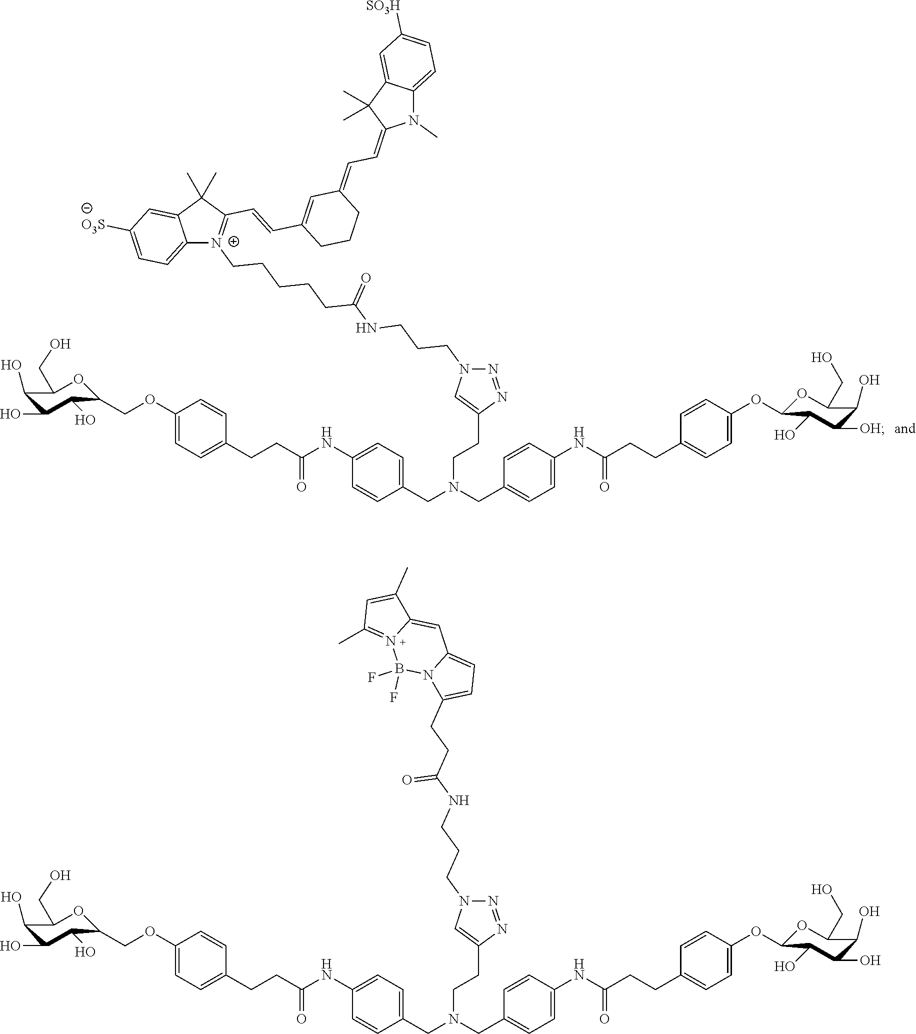
wherein R is H or an alkyl group, preferably C₄ to C₁-alkyl.

13. A pharmaceutical or diagnostic composition comprising the conjugate according to claim 1, and optionally comprising one or more constituents selected from the group consisting of a pharmaceutically acceptable carrier, a diluent and an excipient.

14. (canceled)

15. A method for treating or preventing a disease or infection associated with a bacterium of the phylum Firmicutes, preferably of the class of Bacilli or Clostridia; the phylum Actinobacteria, preferably of the order Corynebacteriales; or the phylum Proteobacteria, preferably of the class of Alphaproteobacteria, Betaproteobacteria or Gammaproteobacteria, said method comprising administering an effective amount of the conjugate according to claim 1 to a subject in need thereof.

16. A method for treating or preventing a disease or infection associated with a bacterium of the phylum Firmicutes, preferably of the class of Bacilli or Clostridia; the phylum Actinobacteria, preferably of the order Corynebacteriales; or the phylum Proteobacteria, preferably of the class of Alphaproteobacteria, Betaproteobacteria or Gammaproteobacteria; said method comprising administering an effective amount of the pharmaceutical composition of claim 13 to a subject in need thereof.

17. A method for diagnosing a disease or infection associated with a bacterium of the phylum Firmicutes, preferably of the class of Bacilli or Clostridia; the phylum Actinobacteria, preferably of the order Corynebacteriales; or the phylum Proteobacteria, preferably of the class of Alphaproteobacteria, Betaproteobacteria or Gammaproteobacteria; said method comprising administering an effective amount of the diagnostic composition of claim 13 to a subject in need thereof.
